(19) United States
(12) Patent Application Publication
(10) Pub. No.: US 2010/0093670 A1
(43) Pub. Date: Apr. 15, 2010

(54) Compounds for the treatment of angiogenesis

(76) Inventors: Yaming Wu, Lexington, MA (US); Kevin Foley, Waltham, MA (US); Christopher Borella, Bedford, MA (US)

Correspondence Address: Foley & Lardner LLP, 111 HUNTINGTON AVENUE, 26TH FLOOR, BOSTON, MA, 02199-7610, US

(21) Appl. No.: 12/310,950

(22) PCT Filed: Sep. 13, 2007
(86) PCT No.: PCT/US2007/019905

§ 371 Date: Sep. 11, 2009

Related U.S. Application Data
(60) Provisional application No. 60/844,550, filed on Sep. 14, 2006.

(51) Int. Cl. A61K 31/675; A61K 31/42; A61K 31/4192; A61K 31/425

(52) U.S. Cl. 514/93; 514/378; 514/359; 514/372; 514/92

(57) ABSTRACT

The invention relates to isoxazole, isothiazole, and triazole compounds that are useful for treating or inhibiting angiogenesis.

### RELATED APPLICATIONS

This application claims the benefit of U.S. Provisional Application No. 60/844,550, filed Sep. 14, 2006, the entire teachings of which are incorporated herein by reference.

### FIELD OF THE INVENTION

This invention relates to biologically active chemical compounds, namely isoxazole, isothiazole, and triazole derivatives that may be used for treating or inhibiting angiogenesis.

### BACKGROUND OF THE INVENTION

Angiogenesis is a fundamental process of generating new blood vessels (neovasculature) in tissues or organs. Although angiogenesis is necessary for organ growth and repair, uncontrolled angiogenesis is involved with or associated with many diseases or disorders. (e.g. cancers, macular degeneration, autoimmune diseases, etc.) As such, angiogenesis has become a target for the treatment of these diseases. Ferrara, N., et al., Nature 438:15 967-974 (2005).

Angiogenesis is controlled by a number of growth factors and cell-adhesion molecules in endothelial and mural cells. Ferrara, N., et al., Nature 438:15 967-974 (2005). Among these, VEGF-A (vascular endothelial growth factor-A) and its receptors have been widely studied and characterized. Ferrara, N., et al., Nature 438:15 967-974 (2005). A number of VEGF inhibitors are approved or currently in clinical trials. Carmeliet, P., Nature 438:15 932-936 (2005). Clinical trials have shown that the current angiogenesis therapies have a number of limitations, including being ineffective as a monotherapy and anti-angiogenic resistance. Carmeliet, Nature 438:15 932-936 (2005). Therefore, a need exists for new therapeutics that reduce or overcome the limitations of currently used anti-angiogenic agents.

### SUMMARY OF THE INVENTION

This invention meets the above-mentioned needs by providing certain isoxazole, isothiazole, and triazole derivatives that may be used to treat or inhibit angiogenesis.

In one embodiment, the invention relates to compounds of formula (I):

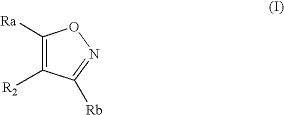

or a pharmaceutically acceptable salt, solvate, clathrate, or prodrug thereof, wherein:

one of Rₐ or R_{b} is —H and the other is an optionally substituted aryl, or an optionally substituted heteroaryl; and

R₂ is an optionally substituted phenyl, an optionally substituted 2,3-dihydro-benzo[1,4]dioxinyl, an optionally substituted benzo[1,3]dioxolyl, an optionally substituted biphenyl, an optionally substituted 4-pyridinyl-phenyl, an optionally substituted quinolinyl, an optionally substituted isoquinolinyl, an optionally substituted 1H-indolyl, an optionally substituted pyridinyl, an optionally substituted oxazolyl, an optionally substituted isoxazolyl, an optionally substituted thiazolyl, an optionally substituted isothiazolyl, an optionally substituted imidazolyl, an optionally substituted pyrrolyl, an optionally substituted pyrazolyl, an optionally substituted furanyl, an optionally substituted thiophenyl, an optionally substituted thiadiazolyl, an optionally substituted oxadiazolyl, an optionally substituted chromanyl, an optionally substituted isochromanyl, an optionally substituted pyridazinyl, an optionally substituted pyrimidinyl, an optionally substituted pyrazinyl, an optionally substituted benzothiophenyl, an optionally substituted 2,3-dihydro-benzothiophenyl, an optionally substituted benzofuranyl, an optionally substituted 2,3-dihydro-benzofuranyl, an optionally substituted 1H-benzoimidazolyl, an optionally substituted benzothiazolyl, an optionally substituted benzooxazolyl, an optionally substituted 1H-benzotriazolyl, an optionally substituted 1H-indazolyl, an optionally substituted 9H-purinyl, an optionally substituted pyrrolopyrimidinyl, an optionally substituted pyrrolopyrazinyl, an optionally substituted pyrrolopyridazinyl, an optionally substituted imidazopyrazinyl, or an optionally substituted imidazolpyridazinyl.

In another embodiment, the invention relates to compounds of formula (II):

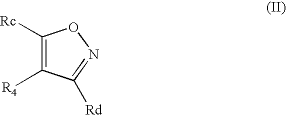

or a pharmaceutically acceptable salt, solvate, clathrate, and prodrug thereof, wherein: one of Rₑ or R_{d} is —H and the other is an optionally substituted heteroaryl, an unsubstituted phenyl, or a substituted phenyl represented by one of the following formulas:

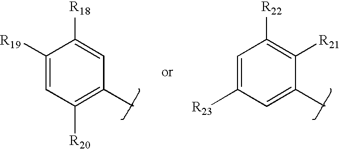

R₄ is an optionally substituted aryl or an optionally substituted heteroaryl;

R₁₈, R₁₉, R₂₂, and R₂₃, are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₂₀ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₂₁ is halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₁₇, for each occurrence, is independently, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; and

p is 1 or 2.

In one embodiment, the invention relates to compounds of formula (XI):

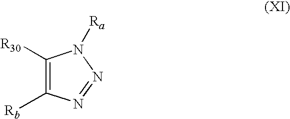

or a pharmaceutically acceptable salt, solvate, clathrate, or prodrug thereof, wherein:

one of Rₐ or R_{b} is —H and the other is an optionally substituted aryl or an optionally substituted heteroaryl. In one aspect of this embodiment, Rₐ is not acridinyl; and

R₃₀ is an optionally substituted aryl or an optionally substituted heteroaryl.

In one embodiment, the invention relates to compounds of formula (XIA):

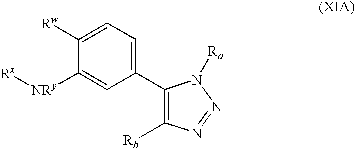

or a pharmaceutically acceptable salt, solvate, clathrate, or prodrug thereof, wherein one of Rₐ or R_{b} is —H and the other is an optionally substituted aryl or an optionally substituted heteroaryl;

R^{x} is (R^{aa})ₘ, —R^{aa}—C(O)(CH₂)ₙC(O)OH, —C(O)(CH₂)ₙC(O)OH, —C(O)YR^{z}, —C(O)NH—R^{aa}, or —(R^{aa})_{q}C(O)(Y₁);

R^{y} is —H or lower alkyl;

R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl;

R₇ for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R^{aa} is an amino acid residue or an amino acid residue analog;

Y is CH₂, O, or NH;

R^{z} is Alk-NH₂, Alk-C(O)OH, Het, or Y₁;

Alk is an optionally substituted alkylene;

Het is an optionally substituted heteroalkyl;

Y₁ is a water soluble polymer with a molecular weight less than 60,000 daltons;

n is 1, 2, 3, or 4;

m is an integer from 1 to 10; and

q is 0 or 1.

In another embodiment, in the compounds represented by formula (XIA), neither Rₐ or R_{b} is acridinyl.

In one embodiment, the invention relates to compounds of formula (XIB):

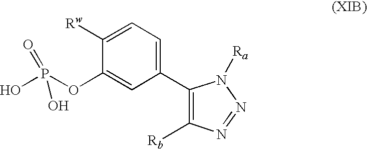

or a pharmaceutically acceptable salt, solvate, clathrate, or prodrug thereof, wherein one of Rₐ or R_{b} is —H and the other is an optionally substituted aryl or an optionally substituted heteroaryl;

R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl;

R₇ for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl. In another embodiment, in the compounds represented by formula (XIB), neither Rₐ or R_{b} is acridinyl.

In one embodiment, the invention relates to compounds of formula (XXXI):

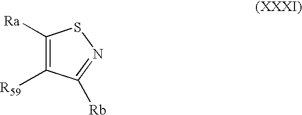

or a pharmaceutically acceptable salt, solvate, clathrate, or prodrug thereof, wherein:

one of Rₐ or R_{b} is —H and the other is an optionally substituted aryl or an optionally substituted heteroaryl; and

R₅₉ is an optionally substituted aryl or an optionally substituted heteroaryl, provided that R₅₉ is not an unsubstituted phenyl.

In another embodiment, the invention relates to compounds of formula (XXXIA):

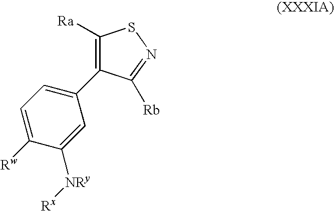

or a pharmaceutically acceptable salt, solvate, clathrate, or prodrug thereof, wherein:

one of Rₐ or R_{b} is —H and the other is an optionally substituted aryl, or an optionally substituted heteroaryl; and

R^{x} is (R^{aa})ₘ, —R^{aa}—C(O)(CH₂)ₙC(O)OH, —C(O)(CH₂)ₙC(O)OH, —C(O)YR^{z}, —C(O)NH—R^{aa}, or —(R^{aa})_{q}C(O)(Y₁);

R^{y} is —H or lower alkyl;

R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl;

R₇ for each occurrence, is independently —H, an optionally substituted alkyl, an optionally substituted

alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R^{aa} is an amino acid residue or an amino acid residue analog;

Y is CH₂, O, or NH;

R^{z} is Alk-NH₂, Alk-C(O)OH, Het, or Y₁;

Alk is an optionally substituted alkylene;

Het is an optionally substituted heteroalkyl;

Y₁ is a water soluble polymer with a molecular weight less than 60,000 daltons;

n is 1, 2, 3, or 4;

m is an integer from 1 to 10; and

q is 0 or 1.

In another embodiment, the invention relates to compounds of formula (XXXIB):

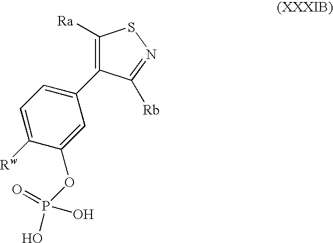

or a pharmaceutically acceptable salt, solvate, clathrate, or prodrug thereof, wherein:

R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl;

R₇ for each occurrence, is independently —H, an optionally substituted alkyl, an optionally substituted

alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

one of Rₐ or R_{b} is —H and the other is an optionally substituted aryl or an optionally substituted heteroaryl.

Compounds of the invention or pharmaceutically acceptable salts, solvates, clathrates, or prodrugs thereof are potent antimitotic agents which inhibiting tubulin polymerization, and thus can inhibit microtubule growth. In order for cells to undergo mitosis, microtubules must be able to assemble and disassemble, in a process known as dynamic instability. Thus, in one embodiment, the compounds of the invention can be used to inhibit tubulin polymerization in a cell by contacting the cell with an effective amount of a compound of the invention or a pharmaceutically acceptable salt, solvate, clathrate, or prodrug thereof.

All of the methods of this invention may be practiced with a compound of the invention alone, or in combination with other agents, such as other anti-angiogenesis agents.

### DESCRIPTION OF THE FIGURES

FIG. 1 shows HUVEC cells (20× objective) at 0 min of treatement with DMSO, 1 nM Compound 249, 1 nM CA4, and 10 nM CA4.

FIG. 2 shows HUVEC cells (20× objective) at 50 min of treatement with DMSO, 1 nM Compound 249, 1 nM CA4, and 10 nM CA4.

FIG. 3 shows HUVEC cells (20× objective) at 100 min of treatement with DMSO, 1 nM Compound 249, 1 nM CA4, and 10 nM CA4.

FIG. 4 shows the time sequence (0 h, 24 h, 48 h, and 72 h) of HUVEC cel migration under treatment with DMSO, 1 nM Compound 249, 5 nM Compound 249, 1 nM CA4, and 5 nM CA4. Gray lines show the front line of the cells after scraping and red lines show the front lines of cells after migration for 24 h, 48 h, and 72 h.

FIG. 5 shows the quantitative analysis of the data from FIG. 4.

FIG. 6 shows the quantification of the effect of 1 nM Compound 249 and 1 nM CA4 on HUVEC cell migration during early treatment (up to 12 h).

FIG. 7 shows the effect of DMSO, 0.1 nM Compound 249, 1 nM Compound 249, and 10 nM Compound 249 on VE-cadherin junction between HUVEC cells.

### DETAILED DESCRIPTION OF THE INVENTION

### Definitions

Unless otherwise specified, the below terms used herein are defined as follows:

As used herein, the term an “aromatic ring” or “aryl” means a monocyclic or polycyclic-aromatic ring or ring radical comprising carbon and hydrogen atoms. Typically, aryl groups have about 6 to about 14 carbon atom ring members. Examples of suitable aryl groups include, but are not limited to, phenyl, tolyl, anthacenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more substituents (including without limitation alkyl (preferably, lower alkyl or alkyl substituted with one or more halo), hydroxy, alkoxy (preferably, lower alkoxy), alkylsulfanyl, cyano, halo, amino, and nitro. In certain embodiments, the aryl group is a monocyclic ring, wherein the ring comprises 6 carbon atoms.

As used herein, the term “alkyl” means a saturated straight chain or branched non-cyclic hydrocarbon typically having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethythexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. Alkyl groups included in compounds of this invention may be optionally substituted with one or more substituents. Examples of substituents include, but are not limited to, amino, alkylamino, alkoxy, alkylsulfanyl, oxo, halo, acyl, nitro, hydroxyl, cyano, aryl, alkylaryl, aryloxy, arylsulfanyl, arylamino, carbocyclyl, carbocyclyloxy, carbocyclylthio, carbocyclylamino, heterocyclyl, heterocyclyloxy, heterocyclylamino, heterocyclylthio, and the like. In addition, any carbon in the alkyl segment may be substituted with oxygen (═O), sulfur (═S), or nitrogen (═NR³², wherein R³² is —H, an alkyl, acetyl, or aralkyl). Lower alkyls are typically preferred for the compounds of this invention.

The term alkylene refers to an alkyl group or a cycloalkyl group that has two points of attachment to two moieties (e.g., {—CH₂—}, —{CH₂CH₂—},

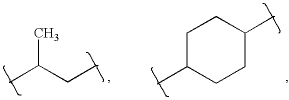

etc., wherein the brackets indicate the points of attachment). Alkylene groups may be optionally substituted with one or more substituents.

An aralkyl group refers to an aryl group that is attached to another moiety via an alkylene linker. Aralkyl groups can be optionally substituted with one or more substituents.

The term “alkoxy,” as used herein, refers to an alkyl group which is linked to another moiety though an oxygen atom. Alkoxy groups can be optionally substituted with one or more substituents.

The term “alkylsulfanyl,” as used herein, refers to an alkyl group which is linked to another moiety though a divalent sulfur atom. Alkylsulfanyl groups can be optionally substituted with one or more substituents.

The term “arylsulfanyl,” as used herein, refers to an aryl group which is linked to another moiety though a divalent sulfur atom. Arylsulfanyl groups can be optionally substituted with one or more substituents.

The term “alkyl ester” as used herein, refers to a group represented by the formula —C(O)OR₃₂, wherein R₃₂ is an alkyl group. A lower alkyl ester is a group represented by the formula —C(O)OR₃₂, wherein R₃₂ is a lower alkyl group.

The term “heteroalkyl,” as used herein, refers to an alkyl group which has one or more carbons in the alkyl chain replaced with an —O—, —S— or —NR₃₃—, wherein R₃₃ is H or a lower alkyl. Heteroalkyl groups can be optionally substituted with one or more substituents.

The term “alkylamino,” as used herein, refers to an amino group in which one hydrogen atom attached to the nitrogen has been replaced by an alkyl group. The term “dialkylamino,” as used herein, refers to an amino group in which two hydrogen atoms attached to the nitrogen have been replaced by alkyl groups, in which the alkyl groups can be the same or different. Alkylamino groups and dialkylamino groups can be optionally substituted with one or more substituents.

As used herein, the term “alkenyl” means a straight chain or branched, hydrocarbon radical typically having from 2 to 10 carbon atoms and having at least one carbon-carbon double bond. Representative straight chain and branched alkenyls include vinyl, allyl, 1-butenyl, 2-butenyl, isobutylenyl, 1-pentenyl, 2-pentenyl, 3-methyl-1-butenyl, 1-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl and the like. Alkenyl groups can be optionally substituted with one or more substituents.

As used herein, the term “alkynyl” means a straight chain or branched, hydrocarbon radical typically having from 2 to 10 carbon atoms and having at lease one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl-1,-1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl and the like. Alkynyl groups can be optionally substituted with one or more substituents.

As used herein, the term “cycloalkyl” means a saturated, mono- or polycyclic alkyl radical typically having from 3 to 14 carbon atoms. Representative cycloalkyls include cyclopropyl, cyclobutyl, cyclopentyl, cyclohexyl, cycloheptyl, cyclooctyl, cyclononyl, cyclodecyl, adamantly, decahydronaphthyl, octahydropentalene, bicycle[1.1.1]pentanyl, and the like. Cycloalkyl groups can be optionally substituted with one or more substituents.

As used herein, the term “cycloalkenyl” means a cyclic non-aromatic alkenyl radical having at least one carbon-carbon double bond in the cyclic system and typically having from 5 to 14 carbon atoms. Representative cycloalkenyls include cyclopentenyl, cyclopentadienyl, cyclohexenyl, cyclohexadienyl, cycloheptenyl, cycloheptadienyl, cycloheptatrienyl, cyclooctenyl, cyclooctadienyl, cyclooctatrienyl, cyclooctatetraenyl, cyclononenyl, cyclononadienyl, cyclodecenyl, cyclodecadienyl and the like. Cycloalkenyl groups can be optionally substituted with one or more substituents.

As used herein, the term “heterocycle” or “heterocyclyl” means a monocyclic or polycyclic heterocyclic ring (typically having 3- to 14-members) which is either a saturated ring or an unsaturated non-aromatic ring. A 3-membered heterocycle can contain from 1 to 3 heteroatoms, and a 4- to 14-membered heterocycle can contain from 1 to about 8 heteroatoms. Each heteroatom is independently selected from nitrogen, which can be quaternized; oxygen; and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, 4H-pyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, the hydrogen on a nitrogen may be substituted with a tert-butoxycarbonyl group. Furthermore, the heterocyclyl may be optionally substituted with one or more substituents (including without limitation a halo, an alkyl, a haloalkyl, or aryl). Only stable isomers of such substituted heterocyclic groups are contemplated in this definition.

As used herein, the term “heteroaromatic” or “heteroaryl” means a monocyclic or polycyclic heteroaromatic ring (or radical thereof) comprising carbon atom ring members and one or more heteroatom ring members (such as, for example, oxygen, sulfur or nitrogen). Typically, the heteroaromatic ring has from 5 to about 14 ring members in which at least 1 ring member is a heteroatom selected from oxygen, sulfur and nitrogen. In another embodiment, the heteroaromatic ring is a 5 or 6 membered ring and may contain from 1 to about 4 heteroatoms. In another embodiment, the heteroaromatic ring system has a 7 to 14 ring members and may contain from 1 to about 7 heteroatoms. Representative heteroaryls include pyridyl, furyl, thienyl, pyrrolyl, oxazolyl, imidazolyl, indolizinyl, thiazolyl, isoxazolyl, pyrazolyl, isothiazolyl, pyridazinyl, pyrimidinyl, pyrazinyl, triazinyl, triazolyl, pyridinyl, thiadiazolyl, pyrazinyl, quinolyl, isoquniolyl, indazolyl, benzoxazolyl, benzofuryl, benzothiazolyl, indolizinyl, imidazopyridinyl, isothiazolyl, tetrazolyl, benzo[1,3]dioxolyl, 2,3-dihydro-benzo[1,4]dioxinyl, benzimidazolyl, benzoxazolyl, benzothiazolyl, benzothiadiazolyl, benzoxadiazolyl, indolyl, tetrahydroindolyl, azaindolyl, imidazopyridyl, qunizaolinyl, purinyl, pyrrolo[2,3]pyrimidyl, pyrazolo[3,4]pyrimidyl or benzo(b)thienyl and the like. Heteroaryl groups may be optionally substituted with one or more substituents

A heteroaralkyl group refers to a heteroaryl group that is attached to another moiety via an alkylene linker. Heteroaralkyl groups can be substituted or unsubstituted with one or more substituents.

As used herein, the term “halogen” or “halo” means —F, —Cl, —Br or —I.

As used herein, the term “haloalkyl” means an alkyl group in which one or more —H is replaced with a halo group. Examples of haloalkyl groups include —CF₃, —CHF₂, —CCl₃, —CH₂CH₂Br, —CH₂CH(CH₂CH₂Br)CH₃, —CHICH₃, and the like.

As used herein, the term “haloalkoxy” means an alkoxy group in which one or more —H is replaced with a halo group. Examples of haloalkoxy groups include —OCF₃ and —OCHF₂.

The terms “bioisostere” and “bioisosteric replacement” have the same meanings as those generally recognized in the art. Bioisosteres are atoms, ions, or molecules in which the peripheral layers of electrons can be considered substantially identical. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape and electron density. Preferred bioisosteres of esters, amides or carboxylic acids are compounds containing two sites for hydrogen bond acceptance. In one embodiment, the ester, amide or carboxylic acid bioisostere is a 5-membered monocyclic heteroaryl ring, such as an optionally substituted 1H-imidazolyl, an optionally substituted oxazolyl, 1H-tetrazolyl, [1,2,4]triazolyl, or an optionally substituted [1,2,4]oxadiazolyl.

As used herein, the terms “subject”, “patient” and “animal”, are used interchangeably and include, but are not limited to, a cow, monkey, horse, sheep, pig, mini pig, chicken, turkey, quail, cat, dog, mouse, rat, rabbit, guinea pig and human. The preferred subject, patient or animal is a human.

As used herein, the term “lower” refers to a group having up to four carbon atoms. For example, a “lower alkyl” refers to an alkyl radical having from 1 to 4 carbon atoms, and a “lower alkenyl” or “lower alkynyl” refers to an alkenyl or alkynyl radical having from 2 to 4 carbon atoms, respectively. A lower alkoxy or a lower alkylsulfanyl refers to an alkoxy or an alkylsulfanyl having from 1 to 4 carbon atoms. Lower substituents are typically preferred.

Where a particular substituent, such as an alkyl substituent, occurs multiple times in a given structure or moeity, the identity of the substitutent is independent in each case and may be the same as or different from other occurrences of that substituent in the structure or moiety. Furthermore, individual substituents in the specific embodiments and exemplary compounds of this invention are preferred in combination with other such substituents in the compounds of this invention, even if such individual substituents are not expressly noted as being preferred or not expressly shown in combination with other substituents.

The compounds of the invention are defined herein by their chemical structures and/or chemical names. Where a compound is referred to by both a chemical structure and a chemical name, and the chemical structure and chemical name conflict, the chemical structure is determinative of the compound's identity.

Suitable substituents for an alkyl, alkoxy, alkylsulfanyl, alkylamino, dialkylamino, alkylene, alkenyl, alkynyl, cycloalkyl, cycloalkenyl, heterocyclyl, aryl, aralkyl, heteroaryl, and heteroaralkyl groups include any substituent which will form a stable compound of the invention. Examples of substituents for an alkyl, alkoxy, alkylsulfanyl, alkylamino, dialkylamino, alkylene, alkenyl, alkynyl, cycloalkyl, cycloalkenyl, heterocyclyl, aryl, aralkyl, heteroaryl, and heteroaralkyl include an alkyl, an alkoxy, an alkylsulfanyl, an alkylamino, a dialkylamino, an alkenyl, an alkynyl, a cycloalkyl, a cycloalkenyl, a heterocyclyl, an aryl, a heteroaryl, an aralkyl, a heteraralkyl, a haloalkyl, —C(O)NR₃₄R₃₅, —NR₃₆C(O)R₃₇, halo, —OR₃₆, cyano, nitro, haloalkoxy, —C(O)R₃₆, —NR₃₄R₃₅, —SR₃₆, —C(O)OR₃₆, —OC(O)R₃₆, —NR₃₆C(O)NR₃₄R₃₅, —OC(O)NR₃₄R₃₅, —NR₃₆C(O)OR₃₇, —S(O)ₚR₃₆, or —S(O)ₚNR₃₄R₃₅, wherein R₃₄ and R₃₅, for each occurrence are, independently, H, an alkyl, an alkenyl, an alkynyl, a cycloalkyl, a cycloalkenyl, a heterocyclyl, an aryl, a heteroaryl, an aralkyl, or a heteraralkyl; or R₃₄ and R₃₅ taken together with the nitrogen to which they are attached is a heterocyclyl or a heteroaryl; and R₃₆ and R₃₇ for each occurrence are, independently, H, an alkyl, an alkenyl, an alkynyl, a cycloalkyl, a cycloalkenyl, a heterocyclyl, an aryl, a heteroaryl, an aralkyl, or a heteraralkyl;

In addition, alkyl, cycloalkyl, alkylene, a heterocyclyl, and any saturated portion of a alkenyl, cycloalkenyl, alkynyl, aralkyl, and heteroaralkyl groups, may also be substituted with ═O, ═S, ═N—R₃₂.

When a heterocyclyl, heteroaryl, or heteroaralkyl group contains a nitrogen atom, it may be substituted or unsubstituted. When a nitrogen atom in the aromatic ring of a heteroaryl group has a substituent the nitrogen may be a quaternary nitrogen.

Choices and combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject). Typically, such compounds are stable at a temperature of 40° C. or less, in the absence of excessive moisture, for at least one week. Such choices and combinations will be apparent to those of ordinary skill in the art and may be determined without undue experimentation.

Unless indicated otherwise, the compounds of the invention containing reactive functional groups (such as, without limitation, carboxy, hydroxy, and amino moieties) also include protected derivatives thereof. “Protected derivatives” are those compounds in which a reactive site or sites are blocked with one ore more protecting groups. Suitable protecting groups for carboxy moieties include benzyl, tert-butyl, and the like. Suitable protecting groups for amino and amido groups include acetyl, tert-butoxycarbonyl, benzyloxycarbonyl, and the like. Suitable protecting groups for hydroxy include benzyl, trimethyl silyl (TMS) and the like. Other suitable protecting groups are well known to those of ordinary skill in the art and include those found in T. W. Greene, Protecting Groups in Organic Synthesis, John Wiley & Sons, Inc. 1981, the entire teachings of which are incorporated herein by reference.

As used herein, the term “compound(s) of this invention” and similar terms refers to a compound of any one of formulas (I)-(XXIX), (XXXI), (XXXV)-(XL), (IA)-(XXIA), (XXVIIA)-(XXIXA), (XXXIA), (XXXVA)-(XLA), (IB)-(XXIB), (XXVIIB)-(XXIXB), (XXXIB), (XXXVB)-(XLB), or of Table 1, or a pharmaceutically acceptable salt, solvate, clathrate, or prodrug thereof and also include protected derivatives thereof.

As used herein, the term “amino acid residue” refers to what is left of an amino acid (losing a H⁺ from the nitrogenous side, an OH⁻ from the carboxylic side, or a H⁺ from the nitrogenous side and an OH⁻ from the carboxylic side) in the formation of a peptide bond(s). An “amino acid analog” includes D or L amino acids having the following formula: NH₂—CHR—C(O)OH, wherein R is an optionally substituted alkyl group, an optionally substituted heteroalkyl group, an optionally substituted aromatic group, or an optionally substituted heteroaromatic group, and wherein R does not correspond to the side chain of a naturally-occurring amino acid. An “amino acid residue analog” refers to what is left of an amino acid analog (losing a H⁺ from the nitrogenous side, an OH⁻ from the carboxylic side, or a H⁺ from the nitrogenous side and an OH⁻ from the carboxylic side) in the formation of a peptide bond(s).

As used herein and unless otherwise indicated, the term “prodrug” means a derivative of a compound that can hydrolyze, oxidize, or otherwise react under biological conditions (in vitro or in vivo) to provide a compound of this invention. Prodrugs may only become active upon such reaction under biological conditions, but they may have activity in their unreacted forms. Examples of prodrugs contemplated in this invention include, but are not limited to, analogs or derivatives of compounds of any one of formulas (I)-(XXIX), (XXXI), (XXXV)-(XL), (IA)-(XXIA), (XXVIIA)-(XXIXA), (XXXIA), (XXXVA)-(XLA), (IB)-(XXIB), (XXVIIB)-(XXIXB), (XXXIB), (XXXVB)-(XLB), or of Table 1 that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of any one of formulas (I)-(XXIX), (XXXI), (XXXV)-(XL), (IA)-(XXIA), (XXVIIA)-(XXIXA), (XXXIA), (XXXVA)-(XLA), (IB)-(XXIB), (XXVIIB)-(XXIXB), (XXXIB), (XXXVB)-(XLB), or of Table 1 that comprise —NO, —NO₂, —ONO, or —ONO₂ moieties. Prodrugs can typically be prepared using well-known methods, such as those described by 1 BURGER'S MEDICINAL CHEMISTRY AND DRUG DISCOVERY (1995) 172-178, 949-982 (Manfred E. Wolff ed., 5^{th} ed), the entire teachings of which are incorporated herein by reference.

As used herein and unless otherwise indicated, the terms “biohydrolyzable amide”, “biohydrolyzable ester”, “biohydrolyzable carbamate”, “biohydrolyzable carbonate”, “biohydrolyzable ureide” and “biohydrolyzable phosphate analogue” mean an amide, ester, carbamate, carbonate, ureide, or phosphate analogue, respectively, that either: 1) does not destroy the biological activity of the compound and confers upon that compound advantageous properties in vivo, such as uptake, duration of action, or onset of action; or 2) is itself biologically inactive but is converted in vivo to a biologically active compound. Examples of biohydrolyzable amides include, but are not limited to, lower alkyl amides, α-amino acid amides, alkoxyacyl amides, and alkylaminoalkylcarbonyl amides. Examples of biohydrolyzable esters include, but are not limited to, lower alkyl esters, alkoxyacyloxy esters, alkyl acylamino alkyl esters, and choline esters. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein, the term “pharmaceutically acceptable salt,” is a salt formed from an acid and a basic group of one of the compounds of any one of formulas (I)-(XXIX), (XXXI), (XXXV)-(XL), (IA)-(XXIA), (XXVIIA)-(XXIXA), (XXXIA), (XXXVA)-(XLA), (IB)-(XXIB), (XXVIIB)-(XXIXB), (XXXIB), (XXXVB)-(XLB), or of Table 1. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The term “pharmaceutically acceptable salt” also refers to a salt prepared from a compound of any one of formulas (I)-(XXIX), (XXXI), (XXXV)-(XL), (IA)-(XXIA), (XXVIIA)-(XXIXA), (XXXIA), (XXXVA)-(XLA), (IB)-(XXIB), (XXVIIB)-(XXIXB), (XXXIB), (XXXVB)-(XLB), or of Table 1 having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)-amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)-amine, or tri-(2-hydroxyethyl)amine; N-methyl-D-glucamine; and amino acids such as arginine, lysine, and the like. The term “pharmaceutically acceptable salt” also refers to a salt prepared from a compound of any one of formulas (I)-(XXIX), (XXXI), (XXXV)-(XL), (IA)-(XXIA), (XXVIIA)-(XXIXA), (XXXIA), (XXXVA)-(XLA), (IB)-(XXIB), (XXVIIB)-(XXIXB), (XXXIB), (XXXVB)-(XLB), or of Table 1 having a basic functional group, such as an amino functional group, and a pharmaceutically acceptable inorganic or organic acid. Suitable acids include, but are not limited to, hydrogen sulfate, citric acid, acetic acid, oxalic acid, hydrochloric acid, hydrogen bromide, hydrogen iodide, nitric acid, phosphoric acid, isonicotinic acid, lactic acid, salicylic acid, tartaric acid, ascorbic acid, succinic acid, maleic acid, besylic acid, fumaric acid, gluconic acid, glucaronic acid, saccharic acid, formic acid, benzoic acid, glutamic acid, methanesulfonic acid, ethanesulfonic acid, benzenesulfonic acid,and p-toluenesulfonic acid.

As used herein, the term “pharmaceutically acceptable solvate,” is a solvate formed from the association of one or more solvent molecules to one or more molecules of a compound of any one of formulas (I)-(XXIX), (XXXI), (XXXV)-(XL), (IA)-(XXIA), (XXVIIA)-(XXIXA), (XXXIA), (XXXVA)-(XLA), (IB)-(XXIB), (XXVIIB)-(XXIXB), (XXXIB), (XXXVB)-(XLB), or of Table 1. The term solvate includes hydrates (e.g., hemi-hydrate, mono-hydrate, dihydrate, trihydrate, tetrahydrate, and the like).

As used herein, the term “clathrate” means a compound of the present invention or a salt thereof in the form of a crystal lattice that contains spaces (e.g., channels) that have a guest molecule (e.g., a solvent or water) trapped within.

Inhibition of tubulin polymerization can be determined by any method known to those skilled in the art, such as the method described herein in Example 7. In addition the amount of a tubulin polymerization inhibitor that inhibits 50% of tubulin polymerization that occurs in the absence of the inhibitor (i.e., the IC₅₀) can be determined by pre-incubating purified tubulin with various amounts of an inhibitor for 15 minutes at 37° C. The mixture is then cooled to room temperature and GTP is added to induce tubulin polymerization. The polymerization can be monitored in a spectrophotometer at 350 nm. A typical reaction mixtures (0.25 mL) contains 1.5 mg/mL tubulin, 0.6 mg/mL microtubule-associated proteins (MAPs), 0.5 mM GTP, 0.5 mlM MgCl.sub.2, 4% DMSO and 0.1M 4-morpholineethanesulfonate buffer (MES, pH 6.4).

As used herein, a “proliferative disorder” or a “hyperproliferative disorder,” and other equivalent terms, means a disease or medical condition involving pathological growth of cells. Proliferative disorders include cancer, smooth muscle cell proliferation, systemic sclerosis, cirrhosis of the liver, adult respiratory distress syndrome, idiopathic cardiomyopathy, lupus erythematosus, retinopathy (e.g., diabetic retinopathy or other retinopathies), choroidal neovascularisation (e.g., macular degeneration), cardiac hyperplasia, reproductive system associated disorders such as benign prostatic hyperplasia and ovarian cysts, pulmonary fibrosis, endometriosis, fibromatosis, harmatomas, lymphangiomatosis, sarcoidosis, and desmoid tumors.

Smooth muscle cell proliferation includes hyperproliferation of cells in the vasculature, for example, intimal smooth muscle cell hyperplasia, restenosis and vascular occlusion, particularly stenosis following biologically- or mechanically-mediated vascular injury, e.g., vascular injury associated with angioplasty. Moreover, intimal smooth muscle cell hyperplasia can include hyperplasia in smooth muscle other than the vasculature, e.g., bile duct blockage, bronchial airways of the lung in patients with asthma, in the kidneys of patients with renal interstitial fibrosis, and the like.

Non-cancerous proliferative disorders also include hyperproliferation of cells in the skin such as psoriasis and its varied clinical forms, Reiter's syndrome, pityriasis rubra pilaris, and hyperproliferative variants of disorders of keratinization (e.g., actinic keratosis, senile keratosis), scleroderma, and the like.

In a preferred embodiment, the proliferative disorder is cancer. Cancers that can be treated or prevented by the methods of the present invention include, but are not limited to human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease.

Other examples of leukemias include acute and/or chronic leukemias, e.g., lymphocytic leukemia (e.g., as exemplified by the p388 (murine) cell line), large granular lymphocytic leukemia, and lymphoblastic leukemia; T-cell leukemias, e.g., T-cell leukemia (e.g., as exemplified by the CEM, Jurkat, and HSB-2 (acute), YAC-1(murine) cell lines), T-lymphocytic leukemia, and T-lymphoblastic leukemia; B cell leukemia (e.g., as exemplified by the SB (acute) cell line), and B-lymphocytic leukemia; mixed cell leukemias, e.g., B and T cell leukemia and B and T lymphocytic leukemia; myeloid leukemias, e.g., granulocytic leukemia, myelocytic leukemia (e.g., as exemplified by the HL-60 (promyelocyte) cell line), and myelogenous leukemia (e.g., as exemplified by the K562(chronic)cell line); neutrophilic leukemia; eosinophilic leukemia; monocytic leukemia (e.g., as exemplified by the THP-1(acute) cell line); myelomonocytic leukemia; Naegeli-type myeloid leukemia; and nonlymphocytic leukemia. Other examples of leukemias are described in Chapter 60 of The Chemotherapy Sourcebook, Michael C. Perry Ed., Williams & Williams (1992) and Section 36 of Holland Frie Cancer Medicine 5th Ed., Bast et al. Eds., B. C. Decker Inc. (2000). The entire teachings of the preceding references are incorporated herein by reference.

An “effective amount” is the quantity of compound in which a beneficial outcome is achieved when the compound is administered to a subject or alternatively, the quantity of compound that possess a desired activity in vivo or in vitro. In the case of proliferative disorders, a beneficial clinical outcome includes reduction in the extent or severity of the symptoms associated with the disease or disorder and/or an increase in the longevity and/or quality of life of the subject compared with the absence of the treatment. For example, for a subject with cancer, a “beneficial clinical outcome” includes a reduction in tumor mass, a reduction in the rate of tumor growth, a reduction in metastasis, a reduction in the severity of the symptoms associated with the cancer and/or an increase in the longevity of the subject compared with the absence of the treatment. The precise amount of compound administered to a subject will depend on the type and severity of the disease or condition and on the characteristics of the subject, such as general health, age, sex, body weight and tolerance to drugs. It will also depend on the degree, severity and type of proliferative disorder. The skilled artisan will be able to determine appropriate dosages depending on these and other factors. Effective amounts of the disclosed compounds typically range between about 1 mg/mm² per day and about 10 grams/mm² per day, and preferably between 10 mg/mm² per day and about 1 gram/mm².

As used herein, the term “angiogenesis” refers to a fundamental process of generating new blood vessels in tissues or organs. Angiogenesis is involved with or associated with many diseases or conditions, including, but not limited to: cancer; ocular neovascular disease; age-related macular degeneration; diabetic retinopathy, retinopathy of prematurity; corneal graft rejection; neovascular glaucoma; retrolental fibroplasias; epidemic keratoconjunctivitis; Vitamin A deficiency; contact lens overwear; atopic keratitis; superior limbic keratitis; pterygium keratitis sicca; sjogrens; acne rosacea; warts; eczema; phylectenulosis; syphilis; Mycobacteria infections; lipid degeneration; chemical burns; bacterial ulcers; fungal ulcers; Herpes simplex infections; Herpes zoster infections; protozoan infections; Kaposi's sarcoma; Mooren's ulcer; Terrien's marginal degeneration; mariginal keratolysis; rheumatoid arthritis; systemic lupus; polyarteritis; trauma; Wegener's sarcoidosis; scleritis; Stevens-Johnson disease; pemphigoid; radial keratotomy; corneal graph rejection; diabetic retinopathy; macular degeneration; sickle cell anemia; sarcoid; syphilis; pseudoxanthoma elasticum; Paget's disease; vein occlusion; artery occlusion; carotid obstructive disease; chronic uveitis/vitritis; mycobacterial infections; Lyme's disease; systemic lupus erythematosis; retinopathy of prematurity; Eales' disease; Behcet's disease; infections causing a retinitis or choroiditis; presumed ocular histoplasmosis; Best's disease; myopia; optic pits; Stargardt's disease; pars planitis; chronic retinal detachment; hyperviscosity syndromes; toxoplasmosis; trauma and post-laser complications; diseases associated with rubeosis (neovasculariation of the angle); diseases caused by the abnormal proliferation of fibrovascular or fibrous tissue including all forms of proliferative vitreoretinopathy; rheumatoid arthritis; osteoarthritis; ulcerative colitis; Crohn's disease; Bartonellosis; atherosclerosis; Osler-Weber-Rendu disease; hereditary hemorrhagic telangiectasia; pulmonary hemangiomatosis; pre-eclampsia; endometriosis; fibrosis of the liver and of the kidney; developmental abnormalities (organogenesis); skin disclolorations (e.g., hemangioma, nevus flammeus, or nevus simplex); wound healing; hypertrophic scars, i.e., keloids; wound granulation; vascular adhesions; cat scratch disease (Rochele ninalia quintosa); ulcers (Helicobacter pylori); keratoconjunctivitis; gingivitis; periodontal disease; epulis; hepatitis; tonsillitis; obesity; rhinitis; laryngitis; tracheitis; bronchitis; bronchiolitis; pneumonia; interstitial pulmonary fibrosis; pulmonary edema; neurodermitis; thyroiditis; thyroid enlargement; endometriosis; glomerulonephritis; gastritis; inflammatory bone and cartilage destruction; thromboembolic disease; and Buerger's disease.

Anti-angiogenesis can be demonstrated by any method known to those skilled in the art, such as the method described herein in Examples 2 and 3.

Anti-angiogenesis agents that can be co-administered with the compounds of the invention include Dalteparin, Suramin, ABT-510, Combretastatin A4 Phosphate, Lenalidomide, LY317615 (Enzastaurin), Soy Isoflavone (Genistein; Soy Protein Isolate), Thalidomide, AMG-706, Anti-VEGF Antibody (Bevacizumab; Avastin™), AZD2171, Bay 43-9006 (Sorafenib tosylate), PI-88, PTK787/ZK 222584 (Vatalanib), SU11248 (Sunitinib malate), VEGF-Trap, XL184, ZD6474, ATN-161, EMD 121974 (Cilenigtide), Celecoxib, Angiostatin, Endostatin, Regranex, Apligraf, Paclitaxel, tetracyclines, clarithromycin, lasix, captopril, aspirin, Vitamin D3 analogs, retinoids, Imiquomod, Interferon alfa2a, Minocycline, copper peptide containing dressings, Lucentis™, ATG002, Pegaptanib Sodium, Tryptophanyl-tRNA synthetase, squalamine lactate, anecortave acetate, AdPEDF, AG-013958, JSM6427, TG100801, Veglin, ascorbic acid ethers (and their analogs), and Pamidronate.

The compounds of the invention may contain one or more chiral centers and/or double bonds and, therefore, may exist as stereoisomers, such as double-bond isomers (i.e., geometric isomers), enantiomers, or diastereomers. According to this invention, the chemical structures depicted herein, including the compounds of this invention, encompass all of the corresponding compounds' enantiomers and stereoisomers, that is, both the stereomerically pure form (e.g., geometrically pure, enantiomerically pure, or diastereomerically pure) and enantiomeric, diastereomeric, and geometric isomeric mixtures. In some cases, one enantiomer, diastereomer, or geometric isomer will possess superior activity or an improved toxicity or kinetic profile compared to others. In those cases, such enantiomers, diastereomers, and geometric isomers of a compound of this invention are preferred.

As used herein, a composition that “substantially” comprises a compound means that the composition contains more than about 80% by weight, more preferably more than about 90% by weight, even more preferably more than about 95% by weight, and most preferably more than about 97% by weight of the compound.

As used herein, a composition that is “substantially free” of a compound means that the composition contains less than about 20% by weight, more preferably less than about 10% by weight, even more preferably less than about 5% by weight, and most preferably less than about 3% by weight of the compound.

As used herein, a reaction that is “substantially complete” means that the reaction contains more than about 80% by weight of the desired product, more preferably more than about 90% by weight of the desired product, even more preferably more than about 95% by weight of the desired product, and most preferably more than about 97% by weight of the desired product.

As used herein, a racemic mixture means about 50% of one enantiomer and about 50% of is corresponding enantiomer relative to all chiral centers in the molecule. The invention encompasses all enantiomerically-pure, enantiomerically-enriched, diastereomerically pure, diastereomerically enriched, and racemic mixtures of the compounds of any one of formulas (I)-(XXIX), (XXXI), (XXXV)-(XL), (IA)-(XXIA), (XXVIIA)-(XXIXA), (XXXIA), (XXXVA)-(XLA), (IB)-(XXIB), (XXVIIB)-(XXIXB), (XXXIB), (XXXVB)-(XLB), or of Table 1.

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

When administered to a patient, e.g., to a non-human animal for veterinary use or for improvement of livestock, or to a human for clinical use, the compounds of the invention are typically administered in isolated form or as the isolated form in a pharmaceutical composition. As used herein, “isolated” means that the compounds of the invention are separated from other components of either (a) a natural source, such as a plant or cell, preferably bacterial culture, or (b) a synthetic organic chemical reaction mixture. Preferably, via conventional techniques, the compounds of the invention are purified. As used herein, “purified” means that when isolated, the isolate contains at least 95%, preferably at least 98%, of a single compound of the invention by weight of the isolate.

Only those choices and combinations of substituents that result in a stable structure are contemplated. Such choices and combinations will be apparent to those of ordinary skill in the art and may be determined without undue experimentation.

The invention can be understood more fully by reference to the following detailed description and illustrative examples, which are intended to exemplify non-limiting embodiments of the invention.

### Specific Embodiments

The invention relates to compounds and pharmaceutical compositions that are useful for treating or inhibiting angiogenesis.

In one embodiment, the invention relates to compounds of formula (I):

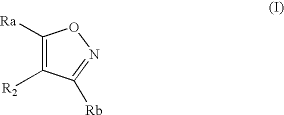

or a pharmaceutically acceptable salt, solvate, clathrate, or prodrug thereof, wherein:

one of Rₐ or R_{b} is —H and the other is an optionally substituted aryl, or an optionally substituted heteroaryl; and

R₂ is an optionally substituted phenyl, an optionally substituted 2,3-dihydro-benzo[1,4]dioxinyl, an optionally substituted benzo[1,3]dioxolyl, an optionally substituted biphenyl, an optionally substituted 4-pyridinyl-phenyl, an optionally substituted quinolinyl, an optionally substituted isoquinolinyl, an optionally substituted 1H-indolyl, an optionally substituted pyridinyl, an optionally substituted oxazolyl, an optionally substituted isoxazolyl, an optionally substituted thiazolyl, an optionally substituted isothiazolyl, an optionally substituted imidazolyl, an optionally substituted pyrrolyl, an optionally substituted pyrazolyl, an optionally substituted furanyl, an optionally substituted thiophenyl, an optionally substituted thiadiazolyl, an optionally substituted oxadiazolyl, an optionally substituted chromanyl, an optionally substituted isochromanyl, an optionally substituted pyridazinyl, an optionally substituted pyrimidinyl, an optionally substituted pyrazinyl, an optionally substituted benzothiophenyl, an optionally substituted 2,3-dihydro-benzothiophenyl, an optionally substituted benzofuranyl, an optionally substituted 2,3-dihydro-benzofuranyl, an optionally substituted 1H-benzoimidazolyl, an optionally substituted benzothiazolyl, an optionally substituted benzooxazolyl, an optionally substituted 1H-benzotriazolyl, an optionally substituted 1H-indazolyl, an optionally substituted 9H-purinyl, an optionally substituted pyrrolopyrimidinyl, an optionally substituted pyrrolopyrazinyl, an optionally substituted pyrrolopyridazinyl, an optionally substituted imidazopyrazinyl, or an optionally substituted imidazolpyridazinyl.

In another embodiment, the invention relates to compounds of formula (II):

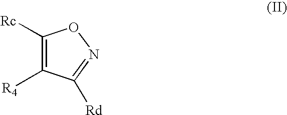

or a pharmaceutically acceptable salt, solvate, clathrate, and prodrug thereof, wherein:

one of R_{c} or R_{d} is —H and the other is an optionally substituted heteroaryl, an unsubstituted phenyl, or a substituted phenyl represented by one of the following formulas:

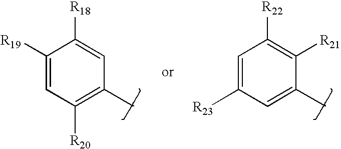

R₄ is an optionally substituted aryl or an optionally substituted heteroaryl;

R₁₈, R₁₉, R₂₂, and R₂₃, are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₂₀ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₂₁ is halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₁₇, for each occurrence, is independently, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; and

p is 1 or 2.

In another embodiment, the invention relates to compounds of formula (III):

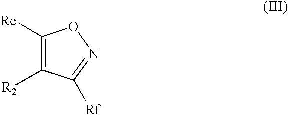

or a pharmaceutically acceptable salt, solvate, clathrate, and prodrug thereof, wherein:

one of Rₑ or R_{f} is —H and the other is an optionally substituted aryl or an optionally substituted heteroaryl selected from the group consisting of an optionally substituted 2,3-dihydro-benzo[1,4]dioxinyl, an optionally substituted benzo[1,3]dioxolyl, an optionally substituted quinolinyl, an optionally substituted isoquinolinyl, an optionally substituted 1H-indolyl, an optionally substituted pyridinyl, an optionally substituted oxazolyl, an optionally substituted isoxazolyl, an optionally substituted thiazolyl, an optionally substituted isothiazolyl, an optionally substituted imidazolyl, an optionally substituted pyrazolyl, an optionally substituted furanyl, an optionally substituted thiophenyl, an optionally substituted thiadiazolyl, an optionally substituted oxadiazolyl, an optionally substituted chromanyl, an optionally substituted isochromanyl, an optionally substituted pyridazinyl, an optionally substituted pyrimidinyl, an optionally substituted pyrazinyl, an optionally substituted benzothiophenyl, an optionally substituted 2,3-dihydro-benzothiophenyl, an optionally substituted benzofuranyl, an optionally substituted 2,3-dihydro-benzofuranyl, an optionally substituted 1H-benzoimidazolyl, an optionally substituted benzothiazolyl, an optionally substituted benzooxazolyl, an optionally substituted 1H-benzotriazolyl, an optionally substituted 1H-indazolyl, an optionally substituted 9H-purinyl, an optionally substituted pyrrolopyrimidinyl, an optionally substituted pyrrolopyrazinyl, an optionally substituted pyrrolopyridazinyl, an optionally substituted imidazopyrazinyl, or an optionally substituted imidazolpyridazinyl; and

R₂ is defined as for formula (I).

In another embodiment, the invention relates to compounds of formula (IV):

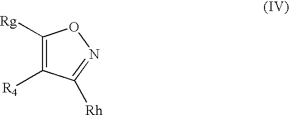

or a pharmaceutically acceptable salt, solvate, clathrate, and prodrug thereof, wherein:

one of R_{g} or Rₕ is —H and the other is:
i) an unsubstituted phenyl or a substituted phenyl represented by one of the following formulas:

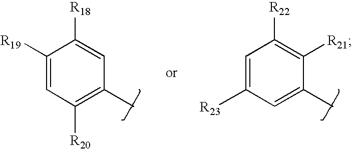

or
ii) an optionally substituted heteroaryl selected from the group consisting of an optionally substituted 2,3-dihydro-benzo[1,4]dioxinyl, an optionally substituted benzo[1,3]dioxolyl, an optionally substituted quinolinyl, an optionally substituted isoquinolinyl, an optionally substituted 1H-indolyl, an optionally substituted pyridinyl, an optionally substituted oxazolyl, an optionally substituted isoxazolyl, an optionally substituted thiazolyl, an optionally substituted isothiazolyl, an optionally substituted imidazolyl, an optionally substituted pyrazolyl, an optionally substituted furanyl, an optionally substituted thiophenyl, an optionally substituted thiadiazolyl, an optionally substituted oxadiazolyl, an optionally substituted chromanyl, an optionally substituted isochromanyl, an optionally substituted pyridazinyl, an optionally substituted pyrimidinyl, an optionally substituted pyrazinyl, an optionally substituted benzothiophenyl, an optionally substituted 2,3-dihydro-benzothiophenyl, an optionally substituted benzofuranyl, an optionally substituted 2,3-dihydro-benzofuranyl, an optionally substituted 1H-benzoimidazolyl, an optionally substituted benzothiazolyl, an optionally substituted benzooxazolyl, an optionally substituted 1H-benzotriazolyl, an optionally substituted 1H-indazolyl, an optionally substituted 9H-purinyl, an optionally substituted pyrrolopyrimidinyl, an optionally substituted pyrrolopyrazinyl, an optionally substituted pyrrolopyridazinyl, an optionally substituted imidazopyrazinyl, or an optionally substituted imidazolpyridazinyl; and

R₄, R₁₈, R₁₉, R₂₀, R₂₁, R₂₂, and R₂₃ are defined as for formula (II).

In another embodiment, the invention relates to compounds of formula (V):

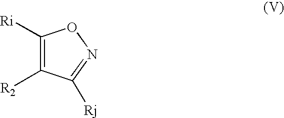

and pharmaceutically acceptable salts, solvates, clathrates, or prodrugs thereof, wherein:

one of Rᵢ, or Rⱼ is —H and the other is represented by the following formula:

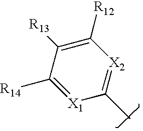

X₁ and X₂ are each, independently, CH or N;

R₁₂, R₁₃ and R₁₄ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₂ is defined as for formula (I); and

R₇, R₈, R₁₀, R₁₁, and p are defined as for formula (II).

In another embodiment, the invention relates to compounds of formula (VI):

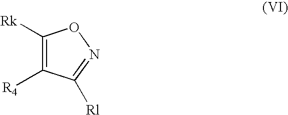

or a pharmaceutically acceptable salt, solvate, clathrate, or prodrug thereof, wherein:

one of Rₖ or R₁ is —H and the other is represented by the following formula:

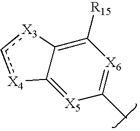

the dashed line indicates that the bond is a single bond or a double bond;

X₃ and X₄ are each, independently, CH, N, CH₂, NR₁₆, O, or S;

X₅ and X₆ are each, independently, CR₂₉ or N;

R₁₅ is H, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₁₆ is H, an alkyl, a cycloalkyl, an aralkyl, —C(O)R, wherein R is an alkyl, a cycloalkyl, or an aralkyl;

R₂₉, for each occurrence, is independently, H or a substituent; and

R₇, R₈, R₁₀, R₁₁, R₁₇, and p are defined as for formula (II).

In another embodiment, the invention relates to compounds of formula (VII):

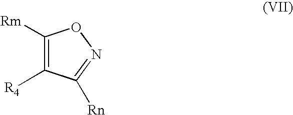

and pharmaceutically acceptable salts, solvates, clathrates, or prodrugs thereof, wherein:

one of Rₘ or Rₙ is —H and the other is represented by the following formula:

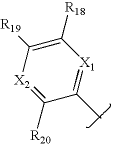

R₄, R₁₈, R₁₉, and R₂₀ are defined as for formula (II); and

X₁ and X₂ are defined as for formula (V).

In another embodiment, the invention relates to compounds of formula (VIII):

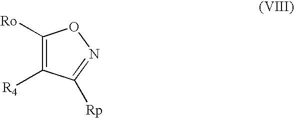

and pharmaceutically acceptable salts, solvates, clathrates, or prodrugs thereof, wherein:

one of Ro or Rp is —H and the other is represented by the following formula:

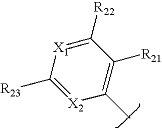

R₄, R₂₁, R₂₂, and R₂₃ are defined as for formula (II); and

X₁ and X₂ are defined as for formula (V).

In another embodiment, the invention relates to compounds of formula (IX):

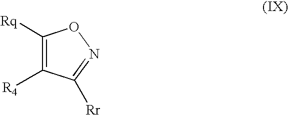

or a pharmaceutically acceptable salt, solvate, clathrate, or prodrug thereof, wherein:

one of R_{q} or Rᵣ is —H and the other is represented by the following formula:

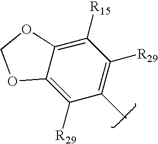

R₄ is defined as for formula (II); and

R₁₅ and R₂₉ are defined as for formula (VI).

In another embodiment, the invention relates to compounds of formula (X):

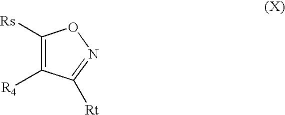

or a pharmaceutically acceptable salt, solvate, clathrate, or prodrug thereof, wherein:

one of Rₛ or Rₜ is —H and the other is represented by the following formula:

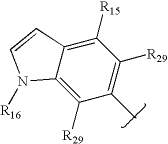

R₄ is defined as for formula (II); and

R₁₅, R₁₆, and R₂₉ are defined as for formula (VI).

In another embodiment, the invention relates to compounds of formula (IA):

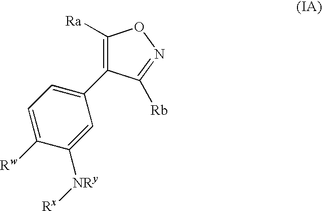

or a pharmaceutically acceptable salt, solvate, clathrate, or prodrug thereof, wherein:

one of Rₐ or R_{b} is —H and the other is an optionally substituted aryl, or an optionally substituted heteroaryl; and

R^{x} is (R^{aa})ₘ, —R^{aa}—C(O)(CH₂)ₙC(O)OH, —C(O)(CH₂)ₙC(O)OH, —C(O)YR^{z}, —C(O)NH—R^{aa}, or —(R^{aa})_{q}C(O)(Y₁);

R^{y} is —H or lower alkyl;

R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl;

R₇ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R^{aa} is an amino acid residue or an amino acid residue analog;

Y is CH₂, O, or NH;

R^{z} is Alk-NH₂, Alk-C(O)OH, Het, or Y₁;

Alk is an optionally substituted alkylene;

Het is an optionally substituted heteroalkyl;

Y₁ is a water soluble polymer with a molecular weight less than 60,000 daltons;

n is 1, 2, 3, or 4;

m is an integer from 1 to 10; and

q is 0 or 1.

In another embodiment, the invention relates to compounds of formula (IIA):

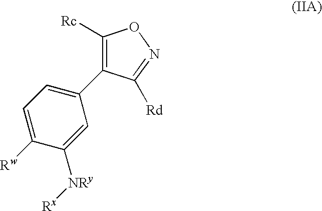

or a pharmaceutically acceptable salt, solvate, clathrate, or prodrug thereof, wherein:

one of R_{c} or R_{d} is —H and the other is an optionally substituted heteroaryl, an unsubstituted phenyl, a substituted phenyl represented by one of the following formulas:

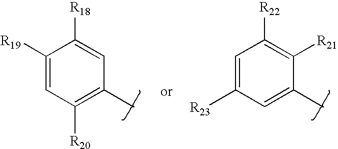

R₁₈, R₁₉, R₂₂, and R₂₃, are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₂₀ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₂₁ is halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R¹¹, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₁₇, for each occurrence, is independently, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

p is 1 or 2; and

R^{x}, R^{y}, and R^{r} are defined as for formula (IA).

In another embodiment, the invention relates to compounds of formula (IIIA):

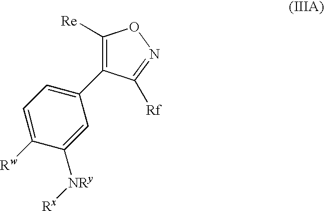

or a pharmaceutically acceptable salt, solvate, clathrate, or prodrug thereof, wherein:

one of Rₑ or R_{f} is —H and the other is an optionally substituted aryl or an optionally substituted heteroaryl selected from the group consisting of an optionally substituted 2,3-dihydro-benzo[1,4]dioxinyl, an optionally substituted benzo[1,3]dioxolyl, an optionally substituted quinolinyl, an optionally substituted isoquinolinyl, an optionally substituted 1H-indolyl, an optionally substituted pyridinyl, an optionally substituted oxazolyl, an optionally substituted isoxazolyl, an optionally substituted thiazolyl, an optionally substituted isothiazolyl, an optionally substituted imidazolyl, an optionally substituted pyrazolyl, an optionally substituted furanyl, an optionally substituted thiophenyl, an optionally substituted thiadiazolyl, an optionally substituted oxadiazolyl, an optionally substituted chromanyl, an optionally substituted isochromanyl, an optionally substituted pyridazinyl, an optionally substituted pyrimidinyl, an optionally substituted pyrazinyl, an optionally substituted benzothiophenyl, an optionally substituted 2,3-dihydro-benzothiophenyl, an optionally substituted benzofuranyl, an optionally substituted 2,3-dihydro-benzofuranyl, an optionally substituted 1H-benzoimidazolyl, an optionally substituted benzothiazolyl, an optionally substituted benzooxazolyl, an optionally substituted 1H-benzotriazolyl, an optionally substituted 1H-indazolyl, an optionally substituted 9H-purinyl, an optionally substituted pyrrolopyrimidinyl, an optionally substituted pyrrolopyrazinyl, an optionally substituted pyrrolopyridazinyl, an optionally substituted imidazopyrazinyl, or an optionally substituted imidazolpyridazinyl; and R^{x}, R^{y}, and R^{w} are defined as for formula (IA).

In another embodiment, the invention relates to compounds of formula (IVA):

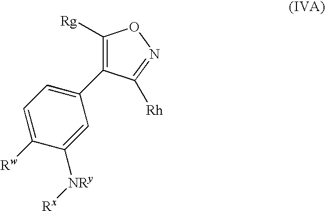

or a pharmaceutically acceptable salt, solvate, clathrate, or prodrug thereof, wherein:

one of R_{g} or Rₕ is —H and the other is:
i) an unsubstituted phenyl or a substituted phenyl represented by one of the following formulas:

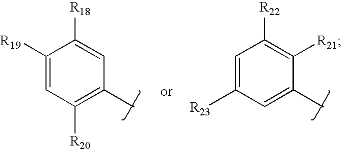

or
ii) an optionally substituted heteroaryl selected from the group consisting of an optionally substituted 2,3-dihydro-benzo[1,4]dioxinyl, an optionally substituted benzo[1,3]dioxolyl, an optionally substituted quinolinyl, an optionally substituted isoquinolinyl, an optionally substituted 1H-indolyl, an optionally substituted pyridinyl, an optionally substituted oxazolyl, an optionally substituted isoxazolyl, an optionally substituted thiazolyl, an optionally substituted isothiazolyl, an optionally substituted imidazolyl, an optionally substituted pyrazolyl, an optionally substituted furanyl, an optionally substituted thiophenyl, an optionally substituted thiadiazolyl, an optionally substituted oxadiazolyl, an optionally substituted chromanyl, an optionally substituted isochromanyl, an optionally substituted pyridazinyl, an optionally substituted pyrimidinyl, an optionally substituted pyrazinyl, an optionally substituted benzothiophenyl, an optionally substituted 2,3-dihydro-benzothiophenyl, an optionally substituted benzofuranyl, an optionally substituted 2,3-dihydro-benzofuranyl, an optionally substituted 1H-benzoimidazolyl, an optionally substituted benzothiazolyl, an optionally substituted benzooxazolyl, an optionally substituted 1H-benzotriazolyl, an optionally substituted 1H-indazolyl, an optionally substituted 9H-purinyl, an optionally substituted pyrrolopyrimidinyl, an optionally substituted pyrrolopyrazinyl, an optionally substituted pyrrolopyridazinyl, an optionally substituted imidazopyrazinyl, or an optionally substituted imidazolpyridazinyl; and

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₁₈, R₁₉, R₂₂, and R₂₃, are defined as for formula (IIA);

p is 1 or 2; and

R^{x}, R^{y}, and R^{w} are defined as for formula (IA).

In another embodiment, the invention relates to compounds of formula (VA):

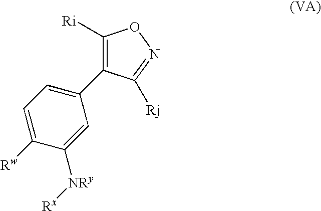

or a pharmaceutically acceptable salt, solvate, clathrate, or prodrug thereof, wherein:

one of Rᵢ or Rⱼ is —H and the other is represented by the following formula:

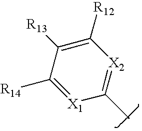

X₁ and X₂ are each, independently, CH or N;

R₁₂, R₁₃ and R₁₄ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₇, R₈, R₁₀, R₁₁, and p are defined as for formula (IIA); and

R^{x}, R^{y}, and R^{w} are defined as for formula (IA).

In another embodiment, this invention relates to compounds of formula (VIA):

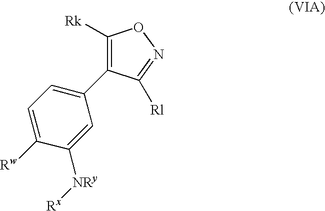

or a pharmaceutically acceptable salt, solvate, clathrate, or prodrug thereof, wherein:

one of Rₖ or R₁ is —H and the other is represented by the following formula:

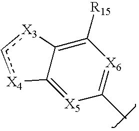

the dashed line indicates that the bond is a single bond or a double bond;

X₃ and X₄ are each, independently, CH, N, CH₂, NR₁₆, O, or S;

X₅ and X₆ are each, independently, CR₂₉ or N;

R₁₅ is H, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₁₆ is H, an alkyl, a cycloalkyl, an aralkyl, —C(O)R, wherein R is an alkyl, a cycloalkyl, or an aralkyl;

R₂₉, for each occurrence, is independently, H or a substituent

R₇, R₈, R₁₀, R₁₁, R₁₇, and p are defined as for formula (IIA); and

R^{x}, R^{y}, and R^{w} are defined as for formula (IA).

In another embodiment, the invention relates to compounds of formula (VIIA):

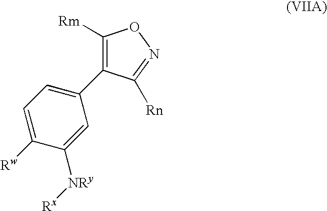

or a pharmaceutically acceptable salt, solvate, clathrate, or prodrug thereof,

wherein:

one of Rₘ or Rₙ is —H and the other is represented by the following formula:

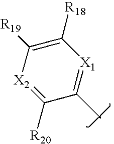

X₁ and X₂ are each, independently, CH or N;

R₁₈, R₁₉, and R₂₀ are defined as for formula (IIA); and

R^{x}, R^{y}, and R^{w} are defined as for formula (IA).

In another embodiment, the invention relates to compounds of formula (VIIIA):

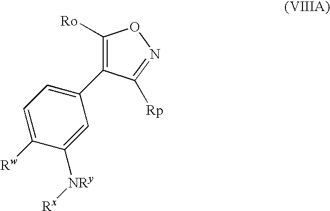

or a pharmaceutically acceptable salt, solvate, clathrate, or prodrug thereof, wherein:

one of Rₒ or Rₚ is —H and the other is represented by the following formula:

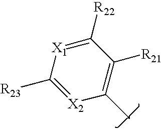

X₁ and X₂ are each, independently, CH or N;

R₂₁, R₂₂, and R₂₃ are defined as for formula (IIA); and

R^{x}, R^{y}, and R^{w} are defined as for formula (IA).

In another embodiment, the invention relates to compounds of formula (IXA):

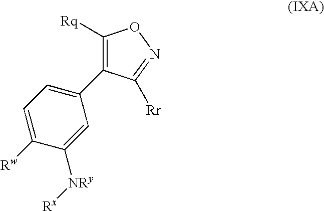

or a pharmaceutically acceptable salt, solvate, clathrate, or prodrug thereof, wherein:

one of R_{q} or Rᵣ is —H and the other is represented by the following formula:

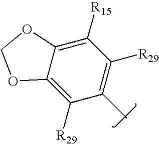

R₁₅ and R₁₉ are defined as for formula (VIA); and

R^{x}, R^{y}, and R^{w} are defined as for formula (IA).

In another embodiment, the invention relates to compounds of formula (XA):

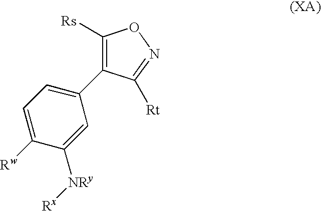

or a pharmaceutically acceptable salt, solvate, clathrate, or prodrug thereof, wherein:

one of Rₛ or Rₜ is —H and the other is represented by the following formula:

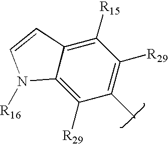

R₁₅, R₁₆, and R₂₉ are defined as for formula (VIA); and

R^{x}, R^{y}, and R^{w} are defined as for formula (IA).

In another embodiment, the invention relates to compounds of formula (IB):

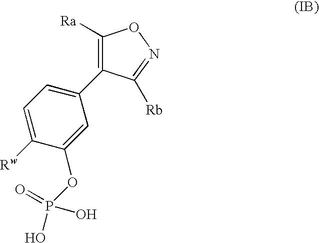

or a pharmaceutically acceptable salt, solvate, or clathrate, thereof, wherein:

R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl;

R₇ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

one of Rₐ or R_{b} is —H and the other is an optionally substituted aryl or an optionally substituted heteroaryl.

In another embodiment, the invention relates to compounds of formula (IIB):

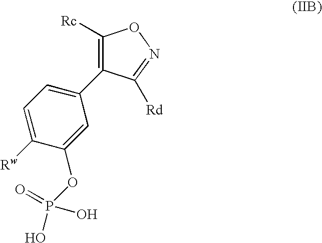

or a pharmaceutically acceptable salt, solvate, clathrate, or prodrug thereof, wherein:

one of R_{c} or R_{d} is —H and the other is an optionally substituted heteroaryl, an unsubstituted phenyl, or a substituted phenyl represented by one of the following formulas:

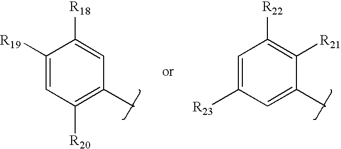

R₁₈, R₁₉, R₂₂, and R₂₃, are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₂₀ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₂₁ is halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₁₇, for each occurrence, is independently, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

p is 1 or 2; and

R^{w} is defined as for formula (IB).

In another embodiment, the invention relates to compounds of formula (IIB):

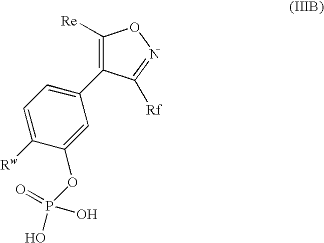

or a pharmaceutically acceptable salt, solvate, clathrate, or prodrug thereof, wherein:

one of Rₑ or R_{f} is —H and the other is an optionally substituted aryl or an optionally substituted heteroaryl selected from the group consisting of an optionally substituted 2,3-dihydro-benzo[1,4]dioxinyl, an optionally substituted benzo[1,3]dioxolyl, an optionally substituted quinolinyl, an optionally substituted isoquinolinyl, an optionally substituted 1H-indolyl, an optionally substituted pyridinyl, an optionally substituted oxazolyl, an optionally substituted isoxazolyl, an optionally substituted thiazolyl, an optionally substituted isothiazolyl, an optionally substituted imidazolyl, an optionally substituted pyrazolyl, an optionally substituted furanyl, an optionally substituted thiophenyl, an optionally substituted thiadiazolyl, an optionally substituted oxadiazolyl, an optionally substituted chromanyl, an optionally substituted isochromanyl, an optionally substituted pyridazinyl, an optionally substituted pyrimidinyl, an optionally substituted pyrazinyl, an optionally substituted benzothiophenyl, an optionally substituted 2,3-dihydro-benzothiophenyl, an optionally substituted benzofuranyl, an optionally substituted 2,3-dihydro-benzofuranyl, an optionally substituted 1H-benzoimidazolyl, an optionally substituted benzothiazolyl, an optionally substituted benzooxazolyl, an optionally substituted 1H-benzotriazolyl, an optionally substituted 1H-indazolyl, an optionally substituted 9H-purinyl, an optionally substituted pyrrolopyrimidinyl, an optionally substituted pyrrolopyrazinyl, an optionally substituted pyrrolopyridazinyl, an optionally substituted imidazopyrazinyl, or an optionally substituted imidazolpyridazinyl; and R^{w} is defined as for formula (IB).

In another embodiment, the invention relates to compounds of formula (IVB):

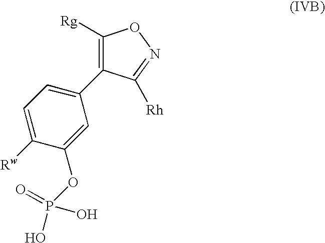

or a pharmaceutically acceptable salt, solvate, clathrate, or prodrug thereof, wherein:

one of R_{g} or Rₕ is —H and the other is:
i) an unsubstituted phenyl or a substituted phenyl represented by one of the following formulas:

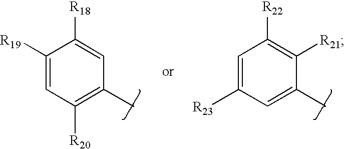

or
ii) an optionally substituted heteroaryl selected from the group consisting of an optionally substituted 2,3-dihydro-benzo[1,4]dioxinyl, an optionally substituted benzo[1,3]dioxolyl, an optionally substituted quinolinyl, an optionally substituted isoquinolinyl, an optionally substituted 1H-indolyl, an optionally substituted pyridinyl, an optionally substituted oxazolyl, an optionally substituted isoxazolyl, an optionally substituted thiazolyl, an optionally substituted isothiazolyl, an optionally substituted imidazolyl, an optionally substituted pyrazolyl, an optionally substituted furanyl, an optionally substituted thiophenyl, an optionally substituted thiadiazolyl, an optionally substituted oxadiazolyl, an optionally substituted chromanyl, an optionally substituted isochromanyl, an optionally substituted pyridazinyl, an optionally substituted pyrimidinyl, an optionally substituted pyrazinyl, an optionally substituted benzothiophenyl, an optionally substituted 2,3-dihydro-benzothiophenyl, an optionally substituted benzofuranyl, an optionally substituted 2,3-dihydro-benzofuranyl, an optionally substituted 1H-benzoimidazolyl, an optionally substituted benzothiazolyl, an optionally substituted benzooxazolyl, an optionally substituted 1H-benzotriazolyl, an optionally substituted 1H-indazolyl, an optionally substituted 9H-purinyl, an optionally substituted pyrrolopyrimidinyl, an optionally substituted pyrrolopyrazinyl, an optionally substituted pyrrolopyridazinyl, an optionally substituted imidazopyrazinyl, or an optionally substituted imidazolpyridazinyl; and

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₁₈, R₁₉, R₂₀, R₂₁, R₂₂, and R₂₃, are defined as for formula (IIB);

R^{w} is defined as for formula (IB); and

p is 1 or 2.

In another embodiment, the invention relates to compounds of formula (VB):

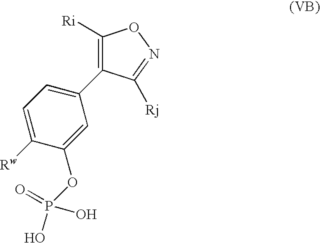

or a pharmaceutically acceptable salt, solvate, clathrate, or prodrug thereof, wherein:

one of Rᵢ or Rⱼ is —H and the other is represented by the following formula:

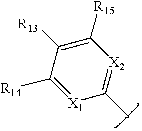

X₁ and X₂ are each, independently, CH or N;

R₁₂, R₁₃ and R₁₄ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R^{w} is defined as for formula (IB); and

R_{7,} R₈, R₁₀, R₁₁, and p are defined as for formula (IIB).

In another embodiment, the invention relates to compounds of formula (VIB):

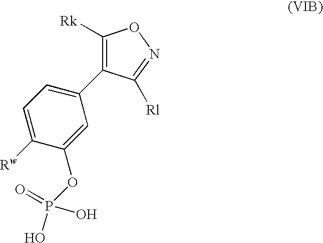

or a pharmaceutically acceptable salt, solvate, clathrate, or prodrug thereof, wherein:

one of Rₖ or Rₗ is —H and the other is represented by the following formula:

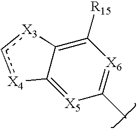

the dashed line indicates that the bond is a single bond or a double bond;

X₃ and X₄ are each, independently, CH, N, CH₂, NR₁₆, O, or S;

X₅ and X₆ are each, independently, CR₂₉ or N;

R₁₅ is H, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₇, R₈, R₁₀, R₁₁, R₁₇, and p are defined as for formula (IIB);

R₁₆ is H, an alkyl, a cycloalkyl, an aralkyl, —C(O)R, wherein R is an alkyl, a cycloalkyl, or an aralkyl;

R^{w} is defined as for formula (IB); and

R₂₉, for each occurrence, is independently, H or a substituent.

In another embodiment, the invention relates to compounds of formula (VIIB):

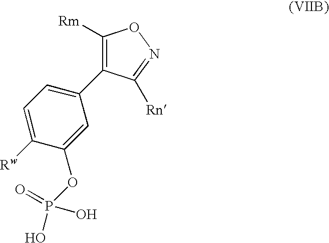

or a pharmaceutically acceptable salt, solvate, clathrate, or prodrug thereof, wherein:

one of Rₘ or Rₙ, is —H and the other is represented by the following formula:

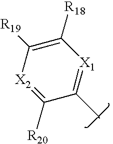

X₁ and X₂ are each, independently, CH or N;

R^{w} is defined as for formula (IB); and

R₁₈, R₁₉, and R₂₀ are defined as for formula (IIB).

In another embodiment, the invention relates to compounds of formula (VIIIB):

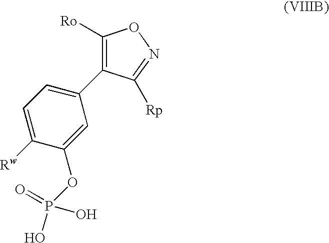

or a pharmaceutically acceptable salt, solvate, clathrate, or prodrug thereof, wherein:

one of Rₒ or Rₚ is —H and the other is represented by the following formula:

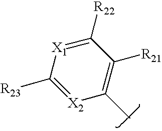

X₁ and X₂ are each, independently, CH or N;

R^{w} is defined as for formula (IB); and

R₂₁, R₂₂, and R₂₃ are defined as for formula (IIB).

In another embodiment, the invention relates to compounds of formula (IXB):

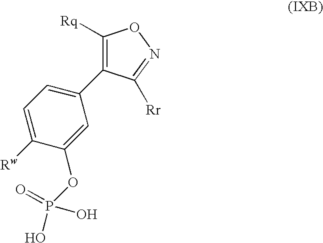

or a pharmaceutically acceptable salt, solvate, clathrate, or prodrug thereof, wherein:

one of R_{q} or Rᵣ is —H and the other is represented by the following formula:

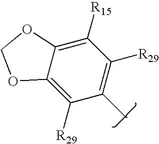

R^{w} is defined as for formula (IB); and

R₁₅ and R₁₉ are defined as for formula (VIB).

In another embodiment, the invention relates to compounds of formula (XB):

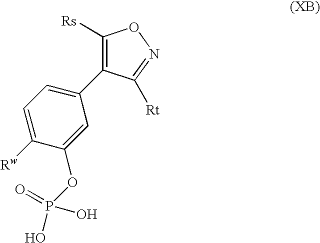

or a pharmaceutically acceptable salt, solvate, clathrate, or prodrug thereof, wherein:

one of Rₛ or Rₜ is —H and the other is represented by the following formula:

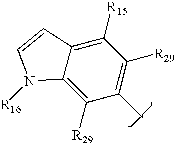

R^{w} is defined as for formula (IB); and

R₁₅, R₁₆, and R₂₉ are defined as for formula (VIB).

In some embodiments, in the compounds represented by formula (I), (IA), or (IB), one of Rₐ or R_{b} is —H and the other is an optionally substituted phenyl. In one aspect of this embodiment, the phenyl group represented by Rₐ or R_{b} is unsubstituted. In another aspect of this embodiment, the phenyl group represented by Rₐ or R_{b} is substituted with from one to five substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the phenyl group represented by Rₐ or R_{b} is substituted with from one to five substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the phenyl group represented by Rₐ or R_{b} is substituted with from one to three substituents. More preferably, the phenyl group represented by Rₐ or R_{b} is substituted with three substituents.

In some embodiments, in the compounds represented by formula (I), (IA), (IB), one of Rₐ or R_{b} is —H and the other is an optionally substituted pyridinyl. In one aspect of this embodiment, the pyridinyl group represented by Rₐ or R_{b} is unsubstituted. In another aspect of this embodiment, the pyridinyl group represented by Rₐ or R_{b} is substituted with one or more substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the pyridinyl group represented by Rₐ or R_{b} is substituted with one or more substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the pyridinyl group represented by Rₐ or R_{b} is substituted with from one to three substituents. More preferably, the pyridinyl group represented by Rₐ or R_{b} is substituted with three substituents.

In some embodiments, in the compounds represented by formula (I), (IA), or (IB), one of Rₐ or R_{b} is —H and the other is an optionally substituted benzo[1,3]dioxolyl. In one aspect of this embodiment, the benzo[1,3]dioxolyl group represented by Rₐ or R_{b} is unsubstituted. In another aspect of this embodiment, the benzo[1,3]dioxolyl group represented by Rₐ or R_{b} is substituted with one or more substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the benzo[1,3]dioxolyl group represented by Rₐ or R_{b} is substituted with one or more substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the benzo[1,3]dioxolyl group represented by Rₐ or R_{b} is substituted with from one to three substituents. More preferably, the benzo[1,3]dioxolyl group represented by Rₐ or R_{b} is substituted with one substituent.

In some embodiments, in the compounds represented by formula (I), (IA), or (IB), Rₐ or R_{b} is —H and the other is an optionally substituted 1H-indolyl. In one aspect of this embodiment, the 1H-indolyl group represented by Rₐ or R_{b} is unsubstituted. In another aspect of this embodiment, the 1H-indolyl group represented by Rₐ or R_{b} is substituted with one or more substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the 1H-indolyl group represented by Rₐ or R_{b} is substituted with one or more substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the 1H-indolyl group represented by Rₐ or R_{b} is substituted with from one to three substituents. More preferably, the 1H-indolyl group represented by Rₐ or R_{b} is substituted with one substituent.

In some embodiments, in the compounds represented by formulas (II), (IIA), or (IIB), R_{c} or R_{d} is —H and the other is an optionally substituted pyridinyl. In one aspect of this embodiment, the pyridinyl group represented by R_{c} or R_{d} is unsubstituted. In another aspect of this embodiment, the pyridinyl group represented by R_{c} or R_{d} is substituted with one or more substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the pyridinyl group represented by R_{c} or R_{d} is substituted with one or more substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the pyridinyl group represented by R_{c} or R_{d} is substituted with from one to three substituents. More preferably, the pyridinyl group represented by R_{c} or R_{d} is substituted with three substituents.

In some embodiments, in the compounds represented by formulas (II), (IIA), or (IIB), R_{c} or R_{d} is —H and the other is an optionally substituted benzo[1,3]dioxolyl. In one aspect of this embodiment, the benzo[1,3]dioxolyl group represented by R_{c} or R_{d} is unsubstituted. In another aspect of this embodiment, the benzo[1,3]dioxolyl group represented by R_{c} or R_{d} is substituted with one or more substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the benzo[1,3]dioxolyl group represented by R_{c} or R_{d} is substituted with one or more substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the benzo[1,3]dioxolyl group represented by R_{c} or R_{d} is substituted with from one to three substituents. More preferably, the benzo[1,3]dioxolyl group represented by R_{c} or R_{d} is substituted with one substituent.

In some embodiments, in the compounds represented by formulas (II), (IIA), or (IIB), R_{c} or R_{d} is —H and the other is an optionally substituted 1H-indolyl. In one aspect of this embodiment, the 1H-indolyl group represented by R_{c} or R_{d} is unsubstituted. In another aspect of this embodiment, the 1H-indolyl group represented by R_{c} or R_{d} is substituted with one or more substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the 1H-indolyl group represented by R_{c} or R_{d} is substituted with one or more substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the 1H-indolyl group represented by R_{c} or R_{d} is substituted with from one to three substituents. More preferably, the 1H-indolyl group represented by R_{c} or R_{d} is substituted with one substituent.

In some embodiments, in the compounds represented by formulas (III), (IIIA), or (IIIB), Rₑ or R_{f} is —H and the other is an optionally substituted phenyl. In one aspect of this embodiment, the phenyl group represented by Rₑ or R_{f} is unsubstituted. In another aspect of this embodiment, the phenyl group represented by Rₑ or R_{f} is substituted with from one to five substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the phenyl group represented by Rₑ or R_{f} is substituted with from one to five substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the phenyl group represented by Rₑ or R_{f} is substituted with from one to three substituents. More preferably, the phenyl group represented by Rₑ or R_{f} is substituted with three substituents.

In some embodiments, in the compounds represented by formulas (III), (IIIA), or (IIIB), Rₑ or R_{f} is —H and the other is an optionally substituted pyridinyl. In one aspect of this embodiment, the pyridinyl group represented by Rₑ or R_{f} is unsubstituted. In another aspect of this embodiment, the pyridinyl group represented by Rₑ or R_{f} is substituted with one or more substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the pyridinyl group represented by Rₑ or R_{f} is substituted with one or more substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the pyridinyl group represented by Rₑ or R_{f} is substituted with from one to three substituents. More preferably, the pyridinyl group represented by Rₑ or R_{f} is substituted with three substituents.

In some embodiments, in the compounds represented by formulas (III), (IIIA), or (IIIB), Rₑ or R_{f} is —H and the other is an optionally substituted benzo[1,3]dioxolyl. In one aspect of this embodiment, the benzo[1,3]dioxolyl group represented by Rₑ or R_{f} is unsubstituted. In another aspect of this embodiment, the benzo[1,3]dioxolyl group represented by Rₑ or R_{f} is substituted with one or more substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the benzo[1,3]dioxolyl group represented by Rₑ or R_{f} is substituted with one or more substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the benzo[1,3]dioxolyl group represented by Rₑ or R_{f} is substituted with from one to three substituents. More preferably, the benzo[1,3]dioxolyl group represented by Rₑ or R_{f} is substituted with one substituent.

In some embodiments, in the compounds represented by formulas (III), (IIIA), or (IIIB), Rₑ or R_{f} is —H and the other is an optionally substituted 1H-indolyl. In one aspect of this embodiment, the 1H-indolyl group represented by Rₑ or R_{f} is unsubstituted. In another aspect of this embodiment, the 1H-indolyl group represented by Rₑ or R_{f} is substituted with one or more substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the 1H-indolyl group represented by Rₑ or R_{f} is substituted with one or more substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the 1H-indolyl group represented by Rₑ or R_{f} is substituted with from one to three substituents. More preferably, the 1H-indolyl group represented by Rₑ or R_{f} is substituted with one substituent.

In some embodiments, in the compounds represented by formulas (IV), (IVA), or (IVB), R_{g} or Rₕ is —H and the other is an optionally substituted pyridinyl. In one aspect of this embodiment, the pyridinyl group represented by R_{g} or Rₕ is unsubstituted. In another aspect of this embodiment, the pyridinyl group represented by R_{g} or Rₕ is substituted with one or more substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the pyridinyl group represented by R_{g} or Rₕ is substituted with one or more substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the pyridinyl group represented by R_{g} or Rₕ is substituted with from one to three substituents. More preferably, the pyridinyl group represented by R_{g} or Rₕ is substituted with three substituents.

In some embodiments, in the compounds represented by formulas (IV), (IVA), or (IVB), R_{g} or Rₕ is —H and the other is an optionally substituted benzo[1,3]dioxolyl. In one aspect of this embodiment, the benzo[1,3]dioxolyl group represented by R_{g} or Rₕ is unsubstituted. In another aspect of this embodiment, the benzo[1,3]dioxolyl group represented by R_{g} or Rₕ is substituted with one or more substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the benzo[1,3]dioxolyl group represented by R_{g} or Rₕ is substituted with one or more substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the benzo[1,3]dioxolyl group represented by R_{g} or Rₕ is substituted with from one to three substituents. More preferably, the benzo[1,3]dioxolyl group represented by R_{g} or Rₕ is substituted with one substituent.

In some embodiments, in the compounds represented by formulas (IV), (IVA), or (IVB), R_{g} or Rₕ is —H and the other is an optionally substituted 1H-indolyl. In one aspect of this embodiment, the 1H-indolyl group represented by R_{g} or Rₕ is unsubstituted. In another aspect of this embodiment, the 1H-indolyl group represented by R_{g} or Rₕ is substituted with one or more substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the 1H-indolyl group represented by R_{g} or Rₕ is substituted with one or more substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the 1H-indolyl group represented by R_{g} or Rₕ is substituted with from one to three substituents. More preferably, the 1H-indolyl group represented by R_{g} or Rₕ is substituted with one substituent.

In some embodiments, in the compounds represented by formulas (I), (III), or (V), R₂ is an optionally substituted phenyl. In one aspect of this embodiment, the phenyl group represented by R₂ is unsubstituted. In another aspect of this embodiment, the phenyl group represented by R₂ is substituted with from one to five groups independently selected from alkoxy, halo, alkyl, haloalkyl, haloalkoxy, nitro, cyano, oxazolyl, 1H-tetrazolyl, 1-methyl-1H-tetrazolyl, —OR₂₄, —SR₂₄, —C(O)R₂₄, —C(O)OR₂₄, —OC(O)R₂₄, —C(O)NR₂₅R₂₆, —NR₂₄C(O)R₂₇, —NR₂₄C(O)OR₂₇, —OC(O)NR₂₅R₂₆, guanidino, amino, alkyl amino, dialkylamino, —NR₂₄S(O)ₚR₂₈, —S(O)ₚR₂₈, —S(O)ₚOR₂₇, —OS(O)ₚR₂₈, —OS(O)ₚOR₂₇, —OP(O)(OR₂₇)₂, or —SP(O)(OR₂₇)₂, wherein:

p is defined as above;

R₂₄ and R₂₇, for each occurrence are, independently, H, an alkyl, or a cycloalkyl;

R₂₅ and R₂₆, for each occurrence are, independently, H, an alkyl, or a cycloalkyl; or R₂₅ and R₂₆, together with the nitrogen to which they are attached are a heterocyclyl or a heteroaryl; and

R₂₈, for each occurrence, is an alkyl or a cycloalkyl.

In one aspect of this embodiment, the phenyl group represented by R₂ is substituted with from one to three substituents. In one aspect of this embodiment, the phenyl group represented by R₂ is substituted with two substituents. In one aspect, the phenyl is substituted with one amino group and one alkoxy group. In one aspect of this embodiment, the phenyl represented by R₂ is substituted with one substituent.

In some embodiments, in the compounds represented by formulas (I), (III), or (V), R₂ is an optionally substituted pyridinyl. In one aspect of this embodiment, the pyridinyl group represented by R₂ is unsubstituted. In another aspect of this embodiment, the pyridinyl group represented by R₂ is substituted with one or more substituents independently selected from alkoxy, halo, alkyl, haloalkyl, haloalkoxy, nitro, cyano, oxazolyl, 1H-tetrazolyl, 1-methyl -1H-tetrazolyl, —OR₂₄, —SR₂₄ , —C(O)R₂₄, —C(O)OR₂₄, —OC(O)R₂₄, —C(O)NR₂₅R₂₆, —NR₂₄C(O)R₂₇, —NR₂₄C(O)OR₂₇, —OC(O)NR₂₅R₂₆, guanidino, amino, alkyl amino, dialkylamino, —NR₂₄S(O)ₚR₂₈, —S(O)ₚR₂₈, —S(O)ₚOR₂₇, —OS(O)ₚR₂₈, —OS(O)ₚOR₂₇, —OP(O)(OR₂₇)₂, or —SP(O)(OR₂₇)₂, wherein R₂₄, R₂₅, R₂₆, R₂₇, R₂₈ and p are defined as above. In one aspect of this embodiment, the pyridinyl group represented by R₂ is substituted with from one to three substituents. Preferably, the pyridinyl represented by R₂ is substituted with one substituent.

In some embodiments, in the compounds represented by formulas (I), (III), or (V), R₂ is an optionally substituted 2,3-dihydro-benzo[1,4]dioxinyl, an optionally substituted biphenyl, an optionally substituted pyridinyl-phenyl, an optionally substituted pyridinyl, an optionally substituted quinolinyl, an optionally substituted isoquinolinyl, an optionally substituted 1H-indolyl, an optionally substituted oxazolyl, an optionally substituted benzo[1,3]dioxinyl, an optionally substituted pyridazinyl, an optionally substituted pyrimidinyl, or an optionally substituted benzofuranyl. In one aspect of this embodiment, R₂ is unsubstituted. In another aspect of this embodiment, R₂ is substituted with one or more substituents independently selected from alkoxy, halo, alkyl, haloalkyl, haloalkoxy, nitro, cyano, oxazolyl, 1H-tetrazolyl, 1-methyl -1H-tetrazolyl, —O₂₄, —SR₂₄, —C(O)R₂₄, —C(O)OR₂₄, —OC(O)R₂₄, —C(O)NR₂₅R₂₆, —NR₂₄C(O)R₂₇, —NR₂₄C(O)OR₂₇, —OC(O)NR₂₅R₂₆, guanidino, amino, alkyl amino, dialkylamino, —NR₂₄S(O)ₚR₂₈, —S(O)ₚR₂₈, —S(O)ₚOR₂₇, —OS(O)ₚR₂₈, —OS(O)ₚOR₂₇, —OP(O)(OR₂₇)₂, or —SP(O)(OR₂₇)_{2,} wherein R₂₄, R₂₅, R₂₆, R₂₇, R₂₈ and p are defined as above. In one aspect of this embodiment, R₂ is substituted with from one to three substituents. Preferably, R₂ is substituted with one substituent.

In some embodiments, in the compounds represented by formula (II), (IV), (VI), (VII), (VIII), (IX), or (X), R₄ is an optionally substituted phenyl. In one aspect of this embodiment, the phenyl group represented by R₄ is unsubstituted. In another aspect of this embodiment, the phenyl group represented by R₄ is substituted with from one to five groups independently selected from alkoxy, halo, alkyl, haloalkyl, haloalkoxy, nitro, cyano, oxazolyl, 1H-tetrazolyl, 1-methyl-1H-tetrazolyl, —OR₂₄, —SR₂₄, —C(O)R₂₄, —C(O)OR₂₄, —OC(O)R₂₄, —C(O)NR₂₅R₂₆, —NR₂₄C(O)R₂₇, —NR₂₄C(O)OR₂₇, —OC(O)NR₂₅R₂₆, guanidino, amino, alkyl amino, dialkylamino, —NR₂₄S(O)ₚR₂₈, —S(O)ₚR₂₈, —S(O)ₚOR₂₇, —OS(O)ₚR₂₈, —OS(O)ₚOR₂₇, —OP(O)(OR₂₇)₂, or —SP(O)(OR₂₇)₂, wherein R₂₄, R₂₅, R₂₆, R₂₇, R₂₈ and p are defined as above. In one aspect of this embodiment, the phenyl group represented by R₄ is substituted with from one to three substituents. In one aspect of this embodiment, the phenyl group represented by R₄ is substituted with two substituents. In one aspect, the phenyl is substituted with one amino group and one alkoxy group. In one aspect, the phenyl represented by R₄ is substituted with one substituent.

In some embodiments, in the compounds represented by formula (II), (IV), (VI), (VII), (VIII), (IX), or (X), R₄ is an optionally substituted pyridinyl. In one aspect of this embodiment, the pyridinyl group represented by R₄ is unsubstituted. In another aspect of this embodiment, the pyridinyl group represented by R₄ is substituted with one or more substituents independently selected from alkoxy, halo, alkyl, haloalkyl, haloalkoxy, nitro, cyano, oxazolyl, 1H-tetrazolyl, 1-methyl-1H-tetrazolyl, —OR₂₄, —SR₂₄, —C(O)R₂₄, —C(O)OR₂₄, —OC(O)R₂₄, —C(O)NR₂₅R₂₆, —NR₂₄C(O)R₂₇, —NR₂₄C(O)OR₂₇, —OC(O)NR₂₅R₂₆, guanidino, amino, alkyl amino, dialkylamino, —NR₂₄S(O)ₚR₂₈, —S(O)ₚR₂₈, —S(O)ₚOR₂₇, —OS(O)ₚR₂₈, —OS(O)ₚOR₂₇, —OP(O)(OR₂₇)₂, or —SP(O)(OR₂₇)₂, wherein R₂₄, R₂₅, R₂₆, R₂₇, R₂₈ and p are defined as above. In one aspect of this embodiment, the pyridinyl group represented by R₄ is substituted with from one to three substituents. Preferably, the pyridinyl represented by R₄ is substituted with one substituent.

In some embodiments, in the compounds represented by formula (I), (IV), (VI), (VII), (VIII), (IX), or (X), R₄ is an optionally substituted 2,3-dihydro-benzo[1,4]dioxinyl, an optionally substituted biphenyl, an optionally substituted pyridinyl-phenyl, an optionally substituted pyridinyl, an optionally substituted quinolinyl, an optionally substituted isoquinolinyl, an optionally substituted 1H-indolyl, an optionally substituted oxazolyl, an optionally substituted benzo[1,3]dioxinyl, an optionally substituted pyridazinyl, an optionally substituted pyrimidinyl, or an optionally substituted benzofuranyl. In one aspect of this embodiment, R₄ is unsubstituted. In another aspect of this embodiment, R₄ is substituted with one or more substituents independently selected from alkoxy, halo, alkyl, haloalkyl, haloalkoxy, nitro, cyano, oxazolyl, 1H-tetrazolyl, 1-methyl-1H-tetrazolyl, —OR₂₄, —SR₂₄, —C(O)R₂₄, —C(O)OR₂₄, —OC(O)R₂₄, —C(O)NR₂₅R₂₆, —NR₂₄C(O)R₂₇, —NR₂₄C(O)OR₂₇, —OC(O)NR₂₅R₂₆, guanidino, amino, alkyl amino, dialkylamino, —NR₂₄S(O)ₚR₂₈, —S(O)ₚR₂₈, —S(O)ₚOR₂₇, —OS(O)ₚR₂₈, —OS(O)ₚOR₂₇, —OP(O)(OR₂₇)₂, or —SP(O)(OR₂₇)₂, wherein R₂₄, R₂₅, R₂₆, R₂₇, R₂₈ and p are defined as above. In one aspect of this embodiment, R₄ is substituted with from one to three substituents. Preferably, R₄ is substituted with one substituent.

In some embodiments, in the compounds represented by formulas (V), (VA), or (VB), R₁₂, R₁₃, and R₁₄ are each, independently, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. In one aspect of this embodiment, R₁₂, R₁₃, and R₁₄ are each, independently, an alkoxy. In another aspect of this embodiment, R₁₂, R₁₃, and R₁₄ are each methoxy.

In some embodiments, in the compounds represented by formulas (V), (VA), (VB), (VII), (VIIA), (VIIB), (VIII), (VIIIA), or (VIIIB), X₁ and X₂ are CH.

In some embodiments, in the compounds represented by formulas (V), (VA), (VB), (VII), (VIIA), (VIIB), (VIII), (VIIIA), or (VIIIB), X₁ and X₂ are N.

In some embodiments, in the compounds represented by formulas (V), (VA), (VB), (VII), (VIIA), (VIIB), (VIIIA), or (VIIIB), X₁ is N and X₂ is CH.

In some embodiments, in the compounds represented by formulas (V), (VA), (VB), (VII), (VIIA), (VIIB), (VIII), (VIIIA), or (VIIIB), X₁ is CH and X₂ is N.

In some embodiments, in the compounds represented by formulas (VI), (VIA), or (VIB), X₃ and X₄ are O and X₅ and X₆ are CH. In one aspect of this embodiment, X₃ and X₄ are O; X₅ and X₆ are CH; and R₁₅ is an alkoxy, such as methoxy.

In some embodiments, in the compounds represented by formulas (VI), (VIA), or (VIB), X₃ is CH; X₄ are NR₁₆; and X₅ and X₆ are CH. In one aspect of this embodiment, X₃ is CH; X₄ are NR₁₆; X₅ and X₆ are CH; and R₁₆ is H. In one aspect of this embodiment, X₃ is CH; X₄ are NR₁₆; X₅ and X₆ are CH; and R₁₆ is a lower alkyl.

In some embodiments, in the compounds represented by formulas (VI), (VIA), (VIB), (IX), (IXA), (IXB), (X), (XA), or (XB), R₁₅ is H, alkoxy, halo, alkyl, haloalkyl, haloalkoxy, nitro, cyano, —SR₂₄, —C(O)R₂₄, —C(O)OR₂₄, —OC(O)R₂₄, —C(O)NR₂₅R₂₆, —NR₂₄C(O)R₂₇, —NR₂₄C(O)OR₂₇, —OC(O)NR₂₅R₂₆, guanidino, amino, alkylamino, dialkylamino, —NR₂₄S(O)ₚR₂₈, —S(O)ₚR₂₈, —S(O)ₚOR₂₇, —OS(O)ₚR₂₈, —OS(O)ₚOR₂₇, —OP(O)(OR₂₇)₂, or —SP(O)(OR₂₇)₂; wherein R₂₄, R₂₅, R₂₆, R₂₇, R₂₈, and p are defined as above.

In some embodiments, in the compounds represented by formulas (IX), (IXA), (IXB), (X), (XA) or (XB), R₁₅ is H, alkoxy, halo, alkyl, haloalkyl, haloalkoxy, nitro, cyano, —SR₂₄, —C(O)R₂₄, —C(O)OR₂₄, —OC(O)R₂₄, —C(O)_{NR}₂₅R₂₆, —NR₂₄C(O)R₂₇, —NR₂₄C(O)OR₂₇, —OC(O)NR₂₅R₂₆, guanidino, amino, alkylamino, dialkylamino, —NR₂₄S(O)ₚR₂₈, —S(O)ₚR₂₈, —S(O)ₚR₂₇, —OS(O)ₚR₂₈, —OS(O)ₚOR₂₇, OP(O)(OR₂₇)₂, or —SP(O)(OR₂₇)₂; and R₂₉, for each occurrence, is independently, H, alkoxy, halo, alkyl, haloalkyl, haloalkoxy, nitro, cyano, —OR₂₄, —SR₂₄, —C(O)R₂₄, —C(O)OR₂₄, —OC(O)R₂₄, —C(O)NR₂₅R₂₆, —NR₂₄C(O)R₂₇, —NR₂₄C(O)OR₂₇, —OC(O)NR₂₅R₂₆, guanidino, amino, alkyl amino, dialkylamino, —NR₂₄S(O)ₚR₂₈, —S(O)ₚR₂₈, —S(O)ₚOR₂₇, —OS(O)ₚR₂₈, —OS(O)ₚOR₂₇, —OP(O)(OR₂₇)₂, or —SP(O)(OR₂₇)_{2;} wherein R₂₄, R₂₅, R₂₆, R₂₇, R₂₈, and p are defined as above.

In some embodiments, in the compounds represented by formulas (VII), (VIIA), or (VIIB), R₁₈ and R₁₉ are each, independently, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl; and R₂₀ is an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, or an alkyl ester; wherein R₇ is defined as above.

In some embodiments, in the compounds represented by formulas (II), (IIA) or (IIB), R_{c} or R_{d} is —H and the other is a substituted phenyl represented by the following structural formula:

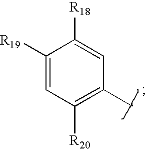

and R₁₈ and R₁₉ are each, independently, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl; and R₂₀ is an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, or an alkyl ester; wherein R₇ is defined as above and “}” represents the point of attachment of the phenyl ring to the isoxazole ring.

In some embodiments, in the compounds represented by formulas (IV), (IVA), or (IVB), R_{g} or Rₕ is —H and the other is a substituted phenyl represented by the following structural formula:

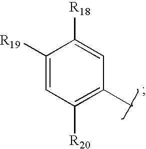

and R₁₈ and R₁₉ are each, independently, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl; and R₂₀ is an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, or an alkyl ester; wherein R₇ is defined as above and “}” represents the point of attachment of the phenyl ring to the isoxazole ring.

In some embodiments, in the compounds represented by formulas (VIII), (VIIIA), or (VIIIB), R₂₂ and R₂₃ are each, independently, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl; and R₂₁ is an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, or an alkyl ester, wherein R₇ is defined as above.

In some embodiments, in the compounds represented by formulas (II), (IIA), or (IIB), R_{c} or R_{d} is —H and the other is a substituted phenyl represented by the following structural formula:

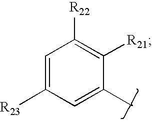

and R₂₂ and R₂₃ are each, independently, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl; and R₂₁ is an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, or an alkyl ester, wherein R₇ is defined as above and “}” represents the point of attachment of the phenyl ring to the isoxazole ring.

In some embodiments, in the compounds represented by formulas (IV), (IVA), or (IVB), R_{g} or Rₕ is —H and the other is a substituted phenyl represented by the following structural formula:

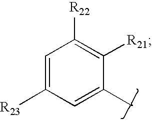

and R₂₂ and R₂₃ are each, independently, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl; and R₂₁ is an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, or an alkyl ester, wherein R₇ is defined as above and “}” represents the point of attachment of the phenyl ring to the isoxazole ring.

In some embodiments, in the compounds represented by formula (IA), (IIA), (IIIA), (IVA), (VA), (VIA), (VIIA), or (VIIIA), R^{x} is R^{aa}, —C(O)YR^{z}, or —C(O)NH—R^{aa}. In one aspect, R^{x} is R^{aa}. In another aspect, R^{x} is —C(O)YR^{z}. R^{aa}, R^{z}, and Y are defined as for formula (IA).

In some embodiments, in the compounds represented by formula (IA), (IIA), (IIIA), (IVA), (VA), (VIA), (VIIA), or (VIIIA), R^{x} is (R^{aa})ₘ. In one aspect, m is 3.

In some embodiments, in the compounds represented by formula (IA), (IIA), (ILIA), (IVA), (VA), (VIA), (VIIA), or (VIIIA), R^{x} is R^{aa} and R^{aa} is defined as for formula (IA). In one aspect, R^{aa} is glycine, serine, alanine, phenylalanine, leucine, or methionine.

In some embodiments, in the compounds represented by formula (IA), (IIA), (IIIA), (IVA), (VA), (VIA), (VIIA), or (VIIIA), R^{x} is R^{aa} and R^{aa} is a D-amino acid residue or a D-amino acid residue analog. In one aspect, R^{aa} is D-alanine, D-valine, D-leucine, D-isoleucine, D-serine, D-threonine, D-cysteine, D-methionine, D-phenylalanine, D-tyrosine, D-tryptophan, D-aspartic acid, D-asparagine, D-glutamic acid, D-glutamine, D-arginine, D-histidine, D-lysine, or D-proline.

In some embodiments, in the compounds represented by formula (IA), (IIA), (IIIA), (IVA), (VA), (VIA), (VIIA), or (VIIIA), R^{x} is R^{aa} and R^{aa} is an L-amino acid residue or an L-amino acid residue analog. In one aspect, R^{aa} is L-alanine, L-valine, L-leucine, L-isoleucine, L-serine, L-threonine, L-cysteine, L-methionine, L-phenylalanine, L-tyrosine, L-tryptophan, L-aspartic acid, L-asparagine, L-glutamic acid, L-glutamine, L-arginine, L-histidine, L-lysine, or L-proline.

In some embodiments, in the compounds represented by formula (IA), (IIA), (IIIA), (IVA), (VA), (VIA), (VIIA), or (VIIIA), R^{x} is R^{aa} and R^{y} is —H, wherein R^{aa} is defined as for formula (IA). In one aspect, R^{aa} is glycine, alanine, valine, leucine, isoleucine, serine, threonine, cysteine, methionine, phenylalanine, tyrosine, tryptophan, aspartic acid, asparagine, glutamic acid, glutamine, arginine, histidine, lysine, or proline. In another aspect, R^{aa} is glycine, serine, alanine, phenylalanine, leucine, or methionine.

In some embodiments, in the compounds represented by formula (IA), (IIA), (IIIA), (IVA), (VA), (VIA), (VIIA), or (VIIIA), R^{x} is —C(O)YR^{z} and Y and R^{z} are defined as for formula (IA). In one aspect, Y is CH₂. In another aspect, Y is O. In another aspect, Y is NH. In one aspect, R^{z} is Y₁ and Y₁ is defined as for formula (IA). In another aspect, R^{z} is Alk-NH₂. In another aspect, R^{z} is Alk-C(O)OH. In another aspect, R² is Het. Alk and Het and defined as for formula (IA).

In some embodiments, in the compounds represented by formula (IA), (IIA), (IIIA), (IVA), (VA), (VIA), (VIIA), or (VIIIA), m is 1, 2 or 3.

In some embodiments, in the compounds represented by formula (IA), (IIA), (IIIA), (IVA), (VA), (VIA), (VIIA), or (VIIIA), Y₁ is PEG, HPMA
copolymer-methacryloyl-Gly-Phe-Leu-Gly-ethylenediamine, or HPMA
copolymer-methacryloyl-Gly-Phe-Leu-Gly-OH. In one aspect, Y₁ is PEG.

In some embodiments, in the compounds represented by formula (IA), (IIA), (IIIA), (IVA), (VA), (VIA), (VIIA), or (VIIIA), R^{y} is —H.

In some embodiments, in the compounds represented by formula (IA), (IIA), (IIIA), (IVA), (VA), (VIA), (VIIA), or (VIIIA), R^{y} is a lower alkyl.

In some embodiments, in the compounds represented by formula (IA), (IIA), (IIIA), (IVA), (VA), (VIA), (VIIA), or (VIIIA), Y₁ has a molecular weight greater than 20,000 daltons. In one aspect, Y₁ has a molecular weight of less than 40,000 daltons, but greater than 25,000 daltons.

In some embodiments, in the compounds represented by formula (IA), (IIA), (IIIA), (IVA), (VA), (VIA), (VIIA), or (VIIIA), Alk is an optionally substituted lower alkylene.

In some embodiments, in the compounds represented by formula (IA), (IIA), (IIIA), (IVA), (VA), (VIA), (VIIA), or (VIIIA), Het is an optionally substituted lower heteroalkyl.

In some embodiments, in the compounds represented by formula (VA), X₁ and X₂ are CH and R₁₂, R₁₃, and R₁₄ are each methoxy. In one aspect, R^{x} is R^{aa}. In another aspect, R^{x} is (R^{aa})ₘ. In another aspect, R^{x} is —R^{aa}—C(O)(CH₂)ₙC(O)OH. In another aspect, R^{x} is —C(O)(CH₂)ₙC(O)OH. In another aspect, R^{x} is —C(O)YR^{z}. In another aspect, R^{x} is —C(O)NH—R^{aa}. In another aspect, R^{x} is —(R^{aa})_{q}C(O)(Y₁). R^{aa}, Y, R^{z}, Y₁, m, n, and q are defined as for formula (IA).

In some embodiments, in the compounds represented by formula (VA), X₁ and X₂ are CH and R₁₂, R₁₃, and R₁₄ are each methoxy. In one aspect, R^{x} is R^{aa} and R^{w} is alkoxy. In another aspect, R^{x} is R^{aa} and R^{y} is —H. In another aspect, R^{x} is R^{aa}, R^{w} is alkoxy, and R^{y} is —H. In another aspect, R^{x} is R^{aa}, R^{w} is alkoxy, and R^{y} is —H. In another aspect, R^{x} is R^{aa}, R^{w} is methoxy, and R^{y} is —H. R^{aa} is defined as for formula (IA).

In some embodiments, in the compounds represented by formula (VA), X₁ and X₂ are CH; R₁₂, R₁₃ and R₁₄ are methoxy; Rⱼ is —H; R^{w} is methoxy; R^{y} is —H; and R^{x} is R^{aa}. R^{aa} is defined as for formula (IA).

In some embodiments, in the compounds represented by formula (VB), X₁ and X₂ are CH; R₁₂, R₁₃, and R₁₄ are each methoxy; and R^{w} is alkoxy. In one aspect, R^{w} is methoxy.

In some embodiments, in the compounds represented by formula (IA or B), (IIA or B), (IIIA or B), (IVA or B), (VA or B), (VIA or B), (VIIA or B), (VIIIA or B), (IXA or B), or (XA or B), R^{w} is alkoxy. In one aspect, R^{w} is methoxy.

In some embodiments represented by formula (I), (IA), or (IB), Rₐ is —H. In some embodiments represented by formula (I), (IA), or (IB), R_{b}, is —H. In some embodiments represented by formula (V), (VA), or (VB), Rᵢ is —H. In some embodiments represented by formula (V), (VA), or (VB), Rⱼ is —H.

In another embodiment, the invention relates to compounds selected from the group consisting of:
4-(4-Bromo-phenyl)-5-(3,4,5-trimethoxy-phenyl)-isoxazole;
4-(Naphthalen-2-yl)-5-(2-hydroxy-4-methoxy-5-ethyl-phenyl)-isoxazole;
4-(4-Methoxy-phenyl)-5-(3,4,5-trimethoxy-phenyl)-isoxazole;
4-(4-Iodo-phenyl)-5-(2-hydroxy-4-methoxy-5-ethyl-phenyl)-isoxazole;
4-Phenyl-5-(2-hydroxy-4-methoxy-5-propyl-phenyl)-isoxazole;
4-(4-Bromo-phenyl)-5-(2-hydroxy-4-methoxy-5-ethyl-phenyl)-isoxazole;
4-(2,3-Dihydro-benzo[1,4]di-oxin-6-yl)-5-(2-hydroxy-4-methoxy-5-propyl-phenyl)-isoxazole;
4-(4-hydroxy-phenyl)-5-(3,4,5-trihydroxy-phenyl)-isoxazole;
4-(4-Iodo-phenyl)-5-(3,4,5-trimethoxy-phenyl)-isoxazole;
4-(3-Fluoro-4-methoxy-phenyl)-5-(3,4,5-trimethoxy-phenyl)-isoxazole;
4-(4-Nitro-phenyl)-5-(3,4,5-trimethoxy-phenyl)-isoxazole;
4-(4-Amino-phenyl)-5-(3,4,5-trimethoxy-phenyl)-isoxazole;
4-(4′-Methoxy-biphenyl-4-yl)-5-(3,4,5-trimethoxy-phenyl)-isoxazole;
4-[4-(pyridine-3-yl)-phenyl]-5-(3,4,5-trimethoxy-phenyl)-isoxazole;
4-[4-(pyridine-4-yl)-phenyl]-5-(3,4,5-trimethoxy-phenyl)-isoxazole;
4-[4-(pyridine-2-yl)-phenyl]-5-(3,4,5-trimethoxy-phenyl)-isoxazole;
4-(Quinolin-7-yl)-5-(3,4,5-trimethoxy-phenyl)-isoxazole;
4-(Pyridin-4-yl)-5-(3,4,5-trimethoxy-phenyl)-isoxazole;
4-(Isoquinolin-7-yl)-5-(3,4,5-trimethoxy-phenyl)-isoxazole;
4-(1-Methyl -1H-indol-5-yl)-5-(3,4,5-trimethoxy-phenyl)-isoxazole;
4-(4-Methoxy-phenyl)-5-(benzo[1,3]dioxol-5-yl)-isoxazole;
4-(4-Methoxy-phenyl)-5-(1-ethyl-1H-indol-6-yl)-isoxazole;
4-(4-Carboxy-phenyl)-5-(3,4,5-trimethoxy-phenyl)-isoxazole;
4-(4-Methoxycarbonyl-phenyl)-5-(3,4,5-trimethoxy-phenyl)-isoxazole;
4-[4-(Oxazol-2-yl)-phenyl]-5-(3,4,5-trimethoxy-phenyl)-isoxazole;
4-(4-Methoxy-phenyl)-5-(3,4,5-triethyl-phenyl)-isoxazole;
4-(4-Iodo-phenyl)-5-(3,4,5-triethyl-phenyl)-isoxazole;
4-(3-Fluoro-4-methoxy-phenyl)-5-(3,4,5-triethyl-phenyl)-isoxazole;
4-(4-Nitro-phenyl)-5-(3,4,5-triethyl-phenyl)-isoxazole;
4-(4-N,N-dimethylamino-phenyl)-5-(3,4,5-triethyl-phenyl)-isoxazole;
4-(4-Methoxy-phenyl)-5-(3,4,5-trimethyl-phenyl)-isoxazole;
4-[4-(Pyridin-3-yl)-phenyl]-5-(3,4,5-triethyl-phenyl)-isoxazole;
4-[4-(Pyridin-4-yl)-phenyl]-5-(3,4,5-triethyl-phenyl)-isoxazole;
4-[4-(Pyridin-2-yl)-phenyl]-5-(3,4,5-triethyl-phenyl)-isoxazole;
4-(Quinolin-7-yl)-5-(3,4,5-triethyl-phenyl)-isoxazole;
4-(Pyridin-4-yl)-5-(3,4,5-triethyl-phenyl)-isoxazole;
4-(Isoquinolin-7-yl)-5-(3,4,5-triethyl-phenyl)-isoxazole;
4-(1H-Indol-5-yl)-5-(3,4,5-triethyl-phenyl)-isoxazole;
4-(4-Methoxy-phenyl)-5-(benzo[1,3]dioxol-5-yl)-isoxazole;
4-(4-Methoxy-phenyl)-5-[1-isopropyl -1H-indol-6-yl)-isoxazole;
4-(4-Methoxy-phenyl)-5-(2,3,4-trimethoxy-phenyl)-isoxazole;
4-(3-Hydroxy-4-methoxy-phenyl)-5-(3,4,5-trimethoxy-phenyl)-isoxazole;
4-[3-(Ethyl-hydroxy-phosphoryloxy)-4-methoxy-phenyl]-5-(3,4,5-trimethoxy-phenyl)-isoxazole;
4-(4-Methoxy-phenyl)-5-(2-hydroxy-4-methoxy-5-ethyl-phenyl)-isoxazole;
4-(4-Isopropyl-phenyl)-5-(3,4,5-trimethoxy-phenyl)-isoxazole;
4-(2,3-Dihydro-benzo[1,4]dioxin-6-yl)-5-(3,4,5-trimethoxy-phenyl)-isoxazole;
4-(4-Ethyl-phenyl)-5-(3,4,5-trimethoxy-phenyl)-isoxazole;
4-(5-Methoxy-pyridin-2-yl)-5-(3,4,5-trimethoxy-phenyl)-isoxazole;
4-(4-Methoxy-phenyl)-5-(2,3,4-trimethoxy-pyridin-6-yl)-isoxazole;
4-(4-Methoxy-phenyl)-5-(3,5-dimethoxy-4-methoxycarbonyl-phenyl)-isoxazole;
4-(4-Methoxy-phenyl)-5-(3,5-diacetoxy-phenyl)-isoxazole;
4-(2-Methoxy-pyridin-5-yl)-5-(3,4,5-trimethoxy-phenyl)-isoxazole;
4-(4-Methoxy-phenyl)-5-(1-methyl-5-methoxy-1H-indol -7-yl)-isoxazole;
4-(4-Methoxy-phenyl)-5-(1-ethyl-1H-indol-7-yl)-isoxazole;
4-(4-Methoxy-phenyl)-5-(benzo[1,3]dioxol-4-yl)-isoxazole;
4-(2-Hydroxy-4-methoxy-phenyl)-5-(3,4,5-trimethoxy)-isoxazole;
4-[2-(Ethyl-hydroxy-phosphoryloxy)-4-methoxy-phenyl]-5-(3,4,5-trimethoxy-phenyl)-isoxazole;
4-(Pyridazin-4-yl)-5-(3,4,5-trimethoxy-phenyl)-isoxazole;
4-(Pyrimidin-5-yl)-5-(3,4,5-trimethoxy-phenyl)-isoxazole;
4-(Pyridin-3-yl)-5-(3,4,5-trimethoxy-phenyl)-isoxazole, hydrochloric acid salt;
4-(3-Mercapto-4-methoxy-phenyl)-5-(3,4,5-trimethoxy-phenyl)-isoxazole;
4-(3-Phosphonosulfanyl-4-methoxy-phenyl)-5-(3,4,5-trimethoxy-phenyl)-isoxazole, disodium salt;
4-(3-Acetylamino-4-methoxy-phenyl)-5-(3,4,5-trimethoxy-phenyl)-isoxazole;
4-(3-Amino-4-methoxy-phenyl)-5-(3,4,5-trimethoxy-phenyl)-isoxazole, hydrochloric acid salt;
4-(2-Hydroxy-4-methoxy-phenyl)-5-(3,4,5-trimethoxy-phenyl)-isoxazole;
4-(2-Methoxy-pyridine-5-yl)-5-(3,4,5-trimethoxy-phenyl)-isoxazole;
4-(5-Methoxy-pyridine-2-yl)-5-(3,4,5-trimethoxy-phenyl)-isoxazole;
4-(3-Carboxy-4-methoxy-phenyl)-5-(3,4,5-trimethoxy-phenyl)-isoxazole, sodium salt;
4-(3-Methoxycarbonyl-4-methoxy-phenyl)-5-(3,4,5-trimethoxy-phenyl)-isoxazole;
4-(3-Sulfooxy-4-methoxy-phenyl)-5-(3,4,5-trimethoxy-phenyl)-isoxazole, sodium salt;
4-(2-Amino-4-methoxy-phenyl)-5-(3,4,5-trimethoxy-phenyl)-isoxazole;
4-(3,4-Dimethoxy-5-phosphonooxy-phenyl)-5-(3,4,5-trimethoxy-phenyl)-isoxazole, disodium salt;
4-(2-Phosphonooxy-4-methoxy-phenyl)-5-(3,4,5-trimethoxy-phenyl)-isoxazole, disodium salt;
4-(4-Methylsulfanyl-phenyl)-5-(3,4,5-trimethoxy-phenyl)-isoxazole;
4-(3-Phosphonooxy-4-methylsulfanyl-phenyl)-5-(3,4,5-trimethoxy-phenyl)-isoxazole, disodium salt;
4-(3-Amino-4-methylsulfanyl-phenyl)-5-(3,4,5-trimethoxy-phenyl)-isoxazole;
4-(2,3-Dihydro-benzofuran-6-yl)-5-(3,4,5-trimethoxy-phenyl)-isoxazole;
4-(4-Hydroxy-phenyl)-5-(3,4,5-trimethoxy-phenyl)-isoxazole, sodium salt;
4-(4-Phosphonooxy-phenyl)-5-(3,4,5-trimethoxy-phenyl)-isoxazole, disodium salt;
4-(4-1H-Tetrazol-5-yl-phenyl)-5-(3,4,5-trimethoxy-phenyl)-isoxazole;
4-[4-(1-Methyl-1H-tetrazol-5-yl)-phenyl]-5-(3,4,5-trimethoxy-phenyl)-isoxazole;
4-(1-Methyl-1H-indol-5-yl)-5-(3,4,5-trimethoxy-phenyl)-isoxazole;
4-(Pyridazin-4-yl)-5-(4-methoxy-benzo[1,3]dioxol-6-yl)-isoxazole;
4-(Pyrimidin-5-yl)-5-(4-methoxy-benzo[1,3]dioxol-6-yl)-isoxazole;
4-(Pyridin-3-yl)-5-(4-methoxy-benzo[1,3]dioxol-6-yl)-isoxazole, hydrochloric acid salt;
4-(3-Mercapto-4-methoxy-phenyl)-5-(4-methoxy-benzo[1,3]dioxol-6-yl)-isoxazole;
4-(3-Phosphonosulfanyl-4-methoxy-phenyl)-5-(4-methoxy-benzo[1,3]dioxol-6-yl)-isoxazole;
4-(3-Acetylamino-4-methoxy-phenyl)-5-(4-methoxy-benzo[1,3]dioxol-6-yl)-isoxazole;
4-(3-Amino-4-methoxy-phenyl)-5-(4-methoxy-benzo[1,3]dioxol-6-yl)-isoxazole, hydrochloric acid salt;
4-(2-Hydroxy-4-methoxy-phenyl)-5-(4-methoxy-benzo[1,3]dioxol-6-yl)-isoxazole;
4-(2-Methoxy-pyridin-5-yl)-5-(4-methoxy-benzo[1,3]dioxol-6-yl)-isoxazole;
4-(5-Methoxy-pyridin-2-yl)-5-(4-methoxy-benzo[1,3]dioxol-6-yl)-isoxazole;
4-(3-Carboxy-4-methoxy-phenyl)-5-(4-methoxy-benzo[1,3]dioxol-6-yl)-isoxazole, sodium salt;
4-(3-Methoxycarbonyl-4-methoxy-phenyl)-5-(4-methoxy-benzo[1,3]dioxol-6-yl)-isoxazole;
4-(3-Sulfooxy-4-methoxy-phenyl)-5-(4-methoxy-benzo[1,3]dioxol-6-yl)-isoxazole, sodium salt;
4-(3-Amino-4-methoxy-phenyl)-5-(4-methoxy-benzo[1,3]dioxol-6-yl)-isoxazole;
4-(3,4-Dimethoxy-5-phosphonooxy-phenyl)-5-(4-methoxy-benzo[1,3]dioxol-6-yl)-isoxazole, disodium salt;
4-(2-Phosphonooxy-4-methoxy-phenyl)-5-(4-methoxy-benzo[1,3]dioxol -6-yl)-isoxazole, disodium salt;
4-(4-Methylsulfanyl-phenyl)-5-(4-methoxy-benzo[1,3]dioxol-6-yl)-isoxazole;
4-(3-Phosphonooxy-4-methylsulfanyl-phenyl)-5-(4-methoxy-benzo[1,3]dioxol-6-yl)-isoxazole, disodium salt;
4-(3-Amino-4-methylsulfanyl-phenyl)-5-(4-methoxy-benzo[1,3]dioxol-6-yl)-isoxazole;
4-(2,3-Dihydro-benzofuran-6-yl)-5-(4-methoxy-benzo[1,3]dioxol-6-yl)-isoxazole;
4-(4-Hydroxy-phenyl)-5-(4-methoxy-benzo[1,3]dioxol-6-yl)-isoxazole, sodium salt;
4-(4-Phosphonooxy-phenyl)-5-(4-methoxy-benzo[1,3]dioxol-6-yl)-isoxazole;
4-(4-1H-Tetrazol-5-yl-phenyl)-5-(4-methoxy-benzo[1,3]dioxol-6-yl)-isoxazole;
4-[4-(1-Methyl-1H-tetrazol-5-yl)-phenyl]-5-(4-methoxy-benzo[1,3]dioxol-6-yl)-isoxazole;
4-(1-Methyl-1H-indol -5-yl)-5-(4-methoxy-benzo[1,3]dioxol-6-yl)-isoxazole;
4-(3,4,5-Trimethoxy-phenyl)-5-(1-methyl-1H-indol-5-yl)-isoxazole;
4-(3,4,5-Trimethoxy-phenyl)-5-(3-phosphonooxy-4-methoxy-phenyl)-isoxazole, disodium salt;
4-(3,4,5-Trimethoxy-phenyl)-5-(N,N-dimethylamino-phenyl)-isoxazole;
4-(3,4,5-Trimethoxy-phenyl)-5-(3-amino-4-methoxy-phenyl)-isoxazole, hydrochloric acid salt;
4-(3,4,5-Trimethoxy-phenyl)-5-[3-(3-hydroxy-2S-amino-propionamido)-4-methoxy-phenyl]-isoxazole, hydrochloric acid salt;
4-(4-Methoxy-phenyl)-5-(2,4,5-trimethoxy-phenyl)-isoxazole;
4-(4-Methyl-phenyl)-5-(2,4,5-trimethoxy-phenyl)-isoxazole;
4-(4-Ethoxy-phenyl)-5-(2,4,5-trimethoxy-phenyl)-isoxazole;
4-(4-Ethyl-phenyl)-5-(2,4,5-trimethoxy-phenyl)-isoxazole;
4-(4-Propoxy-phenyl)-5-(2,4,5-trimethoxy-phenyl)-isoxazole;
4-(4-Propyl-phenyl)-5-(2,4,5-trimethoxy-phenyl)-isoxazole;
4-(4-Butoxy-phenyl)-5-(2,4,5-trimethoxy-phenyl)-isoxazole;
4-(4-Butyl-phenyl)-5-(2,4,5-trimethoxy-phenyl)-isoxazole;
4-(4-Bromo-phenyl)-5-(2,4,5-trimethoxy-phenyl)-isoxazole;
4-(4-Chloro-phenyl)-5-(2,4,5-trimethoxy-phenyl)-isoxazole;
4-(4-Fluoro-phenyl)-5-(2,4,5-trimethoxy-phenyl)-isoxazole;
4-(4-Nitro-phenyl)-5-(2,4,5-trimethoxy-phenyl)-isoxazole;
4-[4-(N,N-Dimethylamino)-phenyl]-5-(2,4,5-trimethoxy-phenyl)-isoxazole;
4-(3,4-Dimethoxy-phenyl)-5-(2,4,5-trimethoxy-phenyl)-isoxazole;
4-(3-Hydroxy-4-methoxy-phenyl)-5-(2,4,5-trimethoxy-phenyl)-isoxazole;
4-(3,4,5-Trimethoxy-phenyl)-5-(2,4,5-trimethoxy-phenyl)-isoxazole;
4-(4-Methoxy-phenyl)-5-(2,3,5-trimethoxy-phenyl)-isoxazole;
4-(4-Methyl-phenyl)-5-(2,3,5-trimethoxy-phenyl)-isoxazole;
4-(4-Ethoxy-phenyl)-5-(2,3,5-trimethoxy-phenyl)-isoxazole;
4-(4-Ethyl-phenyl)-5-(2,3,5-trimethoxy-phenyl)-isoxazole;
4-(4-Propoxy-phenyl)-5-(2,3,5-trimethoxy-phenyl)-isoxazole;
4-(4-Propyl-phenyl)-5-(2,3,5-trimethoxy-phenyl)-isoxazole;
4-(4-Butoxy-phenyl)-5-(2,3,5-trimethoxy-phenyl)-isoxazole;
4-(4-Butyl-phenyl)-5-(2,3,5-trimethoxy-phenyl)-isoxazole;
4-(4-Bromo-phenyl)-5-(2,3,5-trimethoxy-phenyl)-isoxazole;
4-(4-Chloro-phenyl)-5-(2,3,5-trimethoxy-phenyl)-isoxazole;
4-(4-Fluoro-phenyl)-5-(2,3,5-trimethoxy-phenyl)-isoxazole;
4-(4-Nitro-phenyl)-5-(2,3,5-trimethoxy-phenyl)-isoxazole;
4-[4-(N,N-Dimethylamino)-phenyl]-5-(2,3,5-trimethoxy-phenyl)-isoxazole;
4-(3,4-Dimethoxy-phenyl)-5-(2,3,5-trimethoxy-phenyl)-isoxazole;
4-(3-Hydroxy-4-methoxy-phenyl)-5-(2,3,5-trimethoxy-phenyl)-isoxazole;
4-(3,4,5-Trimethoxy-phenyl)-5-(2,3,5-trimethoxy-phenyl)-isoxazole;
4-(2,3,4,5-Tetramethoxy-phenyl)-5-(4-methoxy-phenyl)-isoxazole;
4-(2,3,4,5-Tetramethoxy-phenyl)-5-(4-methyl-phenyl)-isoxazole;
4-(2,3,4,5-Tetramethoxy-phenyl)-5-(4-ethoxy-phenyl)-isoxazole;
4-(2,3,4,5-Tetramethoxy-phenyl)-5-(4-ethyl-phenyl)-isoxazole;
4-(2,3,4,5-Tetramethoxy-phenyl)-5-(4-propoxy-phenyl)-isoxazole;
4-(2,3,4,5-Tetramethoxy-phenyl)-5-(4-propyl-phenyl)-isoxazole;
4-(2,3,4,5-Tetramethoxy-phenyl)-5-(4-butoxy-phenyl)-isoxazole;
4-(2,3,4,5-Tetramethoxy-phenyl)-5-(4-butyl-phenyl)-isoxazole;
4-(2,3,4,5-Tetramethoxy-phenyl)-5-(4-bromo-phenyl)-isoxazole;
4-(2,3,4,5-Tetramethoxy-phenyl)-5-(4-chloro-phenyl)-isoxazole;
4-(2,3,4,5-Tetramethoxy-phenyl)-5-(4-fluoro-phenyl)-isoxazole;
4-(2,3,4,5-Tetramethoxy-phenyl)-5-(4-nitro-phenyl)-isoxazole;
4-(2,3,4,5-Tetramethoxy-phenyl)-5-[4-(N,N,-dimethylamino)-phenyl]-isoxazole;
4-(2,3,4,5-Tetramethoxy-phenyl)-5-(3,4-dimethoxy-phenyl)-isoxazole;
4-(2,3,4,5-Tetramethoxy-phenyl)-5-(3-hydroxy-4-methoxy-phenyl)-isoxazole;
4-(2,3,4,5-Tetramethoxy-phenyl)-5-(3,4,5-trimethoxy-phenyl)-isoxazole;
4-(2,3-Dihydro-benzo[1,4]dioxin-6-yl)-5-(3,4-dimethoxy-phenyl)-isoxazole;
4-(3,4-Dimethy-phenyl)-5-(2-Hydroxy-4-methoxy-5-ethyl-phenyl)-isoxazole;
4-(4-Chloro-phenyl)-5-(2-hydroxy-4-methoxy-5-ethyl-phenyl)-isoxazole;
4-(4-Methyl-phenyl)-5-(2-hydroxy-4-methoxy-5-ethyl-phenyl)-isoxazole;
4-(4-Amino-phenyl)-5-(2-hydroxy-4-methoxy-5-ethyl-phenyl)-isoxazole;
4-(4-Trifluoromethyl-phenyl)-5-(2-hydroxy-4-methoxy-5-ethyl-phenyl)-isoxazole;
4-(4-Methoxy-phenyl)-5-(2-hydroxy-4-methoxy-5-ethyl-phenyl)-isoxazole;
4-(4-Bromo-phenyl)-5-(3,4,5-trimethoxy-phenyl)-isoxazole;
4-(Naphthalen-2-yl)-5-(2-hydroxy-4-methoxy-5-ethyl-phenyl)-isoxazole;
4-(4-Methoxy-phenyl)-5-(3,4,5-trimethoxy-phenyl)-isoxazole;
4-(4-Iodo-phenyl)-5-(2-hydroxy-4-methoxy-5-ethyl-phenyl)-isoxazole;
4-(4-hydroxy-phenyl)-5-(3,4,5-trihydroxy-phenyl)-isoxazole;
4-(4-Iodo-phenyl)-5-(3,4,5-trimethoxy-phenyl)-isoxazole;
4-(3-Fluoro-4-methoxy-phenyl)-5-(3,4,5-trimethoxy-phenyl)-isoxazole;
4-(4-Nitro-phenyl)-5-(3,4,5-trimethoxy-phenyl)-isoxazole;
4-(4-Amino-phenyl)-5-(3,4,5-trimethoxy-phenyl)-isoxazole;
4-(4′-Methoxy-biphenyl-4-yl)-5-(3,4,5-trimethoxy-phenyl)-isoxazole;
4-[4-(pyridine-3-yl)-phenyl]-5-(3,4,5-trimethoxy-phenyl)-isoxazole;
4-[4-(pyridine-4-yl)-phenyl]-5-(3,4,5-trimethoxy-phenyl)-isoxazole;
4-[4-(pyridine-2-yl)-phenyl]-5-(3,4,5-trimethoxy-phenyl)-isoxazole;
4-(Quinolin-7-yl)-5-(3,4,5-trimethoxy-phenyl)-isoxazole;
4-(Pyridin-4-yl)-5-(3,4,5-trimethoxy-phenyl)-isoxazole;
4-(Isoquinolin-7-yl)-5-(3,4,5-trimethoxy-phenyl)-isoxazole;
4-(1-Methyl-1H-indol -5-yl)-5-(3,4,5-trimethoxy-phenyl)-isoxazole;
4-(4-Methoxy-phenyl)-5-(benzo[1,3]dioxol -5-yl)-isoxazole;
4-(4-Methoxy-phenyl)-5-(1-ethyl-1H-indol -6-yl)-isoxazole;
4-(4-Carboxy-phenyl)-5-(3,4,5-trimethoxy-phenyl)-isoxazole;
4-(4-Methoxycarbonyl-phenyl)-5-(3,4,5-trimethoxy-phenyl)-isoxazole;
4-[4-(Oxazol-2-yl)-phenyl]-5-(3,4,5-trimethoxy-phenyl)-isoxazole;
4-(4-Methoxy-phenyl)-5-(3,4,5-triethyl-phenyl)-isoxazole;
4-(4-Iodo-phenyl)-5-(3,4,5-triethyl-phenyl)-isoxazole;
4-(3-Fluoro-4-methoxy-phenyl)-5-(3,4,5-triethyl-phenyl)-isoxazole;
4-(4-Nitro-phenyl)-5-(3,4,5-triethyl-phenyl)-isoxazole;
4-(4-N,N-dimethylamino-phenyl)-5-(3,4,5-triethyl-phenyl)-isoxazole;
4-(4-Methoxy-phenyl)-5-(3,4,5-trimethyl-phenyl)-isoxazole;
4-[4-(Pyridin-3-yl)-phenyl]-5-(3,4,5-triethyl-phenyl)-isoxazole;
4-[4-(Pyridin-4-yl)-phenyl]-5-(3,4,5-triethyl-phenyl)-isoxazole;
4-[4-(Pyridin-2-yl)-phenyl]-5-(3,4,5-triethyl-phenyl)-isoxazole;
4-(Quinolin-7-yl)-5-(3,4,5-triethyl-phenyl)-isoxazole;
4-(Pyridin-4-yl)-5-(3,4,5-triethyl-phenyl)-isoxazole;
4-(Isoquinolin-7-yl)-5-(3,4,5-triethyl-phenyl)-isoxazole;
4-(1H-Indol-5-yl)-5-(3,4,5-triethyl-phenyl)-isoxazole;
4-(4-Methoxy-phenyl)-5-(benzo[1,3]dioxol -5-yl)-isoxazole;
4-(4-Methoxy-phenyl)-5-[1-isopropyl-1H-indol-6-yl)-isoxazole;
4-(4-Methoxy-phenyl)-5-(2,3,4-trimethoxy-phenyl)-isoxazole;
4-(3-Hydroxy-4-methoxy-phenyl)-5-(3,4,5-trimethoxy-phenyl)-isoxazole;
4-[3-(Ethyl-hydroxy-phosphoryloxy)-4-methoxy-phenyl]-5-(3,4,5-trimethoxy-phenyl)-isoxazole;
4-(4-Isopropyl-phenyl)-5-(3,4,5-trimethoxy-phenyl)-isoxazole;
4-(2,3-Dihydro-benzo[1,4]dioxin-6-yl)-5-(3,4,5-trimethoxy-phenyl)-isoxazole;
4-(4-Ethyl-phenyl)-5-(3,4,5-trimethoxy-phenyl)-isoxazole;
4-(5-Methoxy-pyridin-2-yl)-5-(3,4,5-trimethoxy-phenyl)-isoxazole;
4-(4-Methoxy-phenyl)-5-(2,3,4-trimethoxy-pyridin-6-yl)-isoxazole;
4-(4-Methoxy-phenyl)-5-(3,5-dimethoxy-4-methoxycarbonyl-phenyl)-isoxazole;
4-(4-Methoxy-phenyl)-5-(3,5-diacetoxy-phenyl)-isoxazole;
4-(2-Methoxy-pyridin-5-yl)-5-(3,4,5-trimethoxy-phenyl)-isoxazole;
4-(4-Methoxy-phenyl)-5-(1-methyl-5-methoxy-1H-indol-7-yl)-isoxazole;
4-(4-Methoxy-phenyl)-5-(1-ethyl-1H-indol -7-yl)-isoxazole;
4-(4-Methoxy-phenyl)-5-(benzo[1,3]dioxol -4-yl)-isoxazole;
4-(2-Hydroxy-4-methoxy-phenyl)-5-(3,4,5-trimethoxy)-isoxazole;
4-[2-(Ethyl-hydroxy-phosphoryloxy)-4-methoxy-phenyl]-5-(3,4,5-trimethoxy-phenyl)-isoxazole;
4-(Pyridazin-4-yl)-5-(3,4,5-trimethoxy-phenyl)-isoxazole;
4-(Pyrimidin-5-yl)-5-(3,4,5-trimethoxy-phenyl)-isoxazole;
4-(Pyridin-3-yl)-5-(3,4,5-trimethoxy-phenyl)-isoxazole, hydrochloric acid salt;
4-(3-Mercapto-4-methoxy-phenyl)-5-(3,4,5-trimethoxy-phenyl)-isoxazole;
4-(3-Phosphonosulfanyl-4-methoxy-phenyl)-5-(3,4,5-trimethoxy-phenyl)-isoxazole, disodium salt;
4-(3-Acetylamino-4-methoxy-phenyl)-5-(3,4,5-trimethoxy-phenyl)-isoxazole;
4-(3-Amino-4-methoxy-phenyl)-5-(3,4,5-trimethoxy-phenyl)-isoxazole, hydrochloric acid salt;
4-(2-Hydroxy-4-methoxy-phenyl)-5-(3,4,5-trimethoxy-phenyl)-isoxazole;
4-(2-Methoxy-pyridine-5-yl)-5-(3,4,5-trimethoxy-phenyl)-isoxazole;
4-(5-Methoxy-pyridine-2-yl)-5-(3,4,5-trimethoxy-phenyl)-isoxazole;
4-(3-Carboxy-4-methoxy-phenyl)-5-(3,4,5-trimethoxy-phenyl)-isoxazole, sodium salt;
4-(3-Methoxycarbonyl-4-methoxy-phenyl)-5-(3,4,5-trimethoxy-phenyl)-isoxazole;
4-(3-Sulfooxy-4-methoxy-phenyl)-5-(3,4,5-trimethoxy-phenyl)-isoxazole, sodium salt;
4-(2-Amino-4-methoxy-phenyl)-5-(3,4,5-trimethoxy-phenyl)-isoxazole;
4-(3,4-Dimethoxy-5-phosphonooxy-phenyl)-5-(3,4,5-trimethoxy-phenyl)-isoxazole, disodium salt;
4-(2-Phosphonooxy-4-methoxy-phenyl)-5-(3,4,5-trimethoxy-phenyl)-isoxazole, disodium salt;
4-(4-Methylsulfanyl-phenyl)-5-(3,4,5-trimethoxy-phenyl)-isoxazole;
4-(3-Phosphonooxy-4-methylsulfanyl-phenyl)-5-(3,4,5-trimethoxy-phenyl)-isoxazole, disodium salt;
4-(3-Amino-4-methylsulfanyl-phenyl)-5-(3,4,5-trimethoxy-phenyl)-isoxazole;
4-(2,3-Dihydro-benzofuran-6-yl)-5-(3,4,5-trimethoxy-phenyl)-isoxazole;
4-(4-Hydroxy-phenyl)-5-(3,4,5-trimethoxy-phenyl)-isoxazole, sodium salt;
4-(4-Phosphonooxy-phenyl)-5-(3,4,5-trimethoxy-phenyl)-isoxazole, disodium salt;
4-(4-1H-Tetrazol-5-yl-phenyl)-5-(3,4,5-trimethoxy-phenyl)-isoxazole;
4-[4-(1-Methyl -1H-tetrazol-5-yl)-phenyl]-5-(3,4,5-trimethoxy-phenyl)-isoxazole;
4-(1-Methyl -1H-indol-5-yl)-5-(3,4,5-trimethoxy-phenyl)-isoxazole;
4-(Pyridazin-4-yl)-5-(4-methoxy-benzo[1,3]dioxol -6-yl)-isoxazole;
4-(Pyrimidin-5-yl)-5-(4-methoxy-benzo[1,3]dioxol -6-yl)-isoxazole;
4-(Pyridin-3-yl)-5-(4-methoxy-benzo[1,3]dioxol -6-yl)-isoxazole, hydrochloric acid salt;
4-(3-Mercapto-4-methoxy-phenyl)-5-(4-methoxy-benzo[1,3]dioxol-6-yl)-isoxazole;
4-(3-Phosphonosulfanyl-4-methoxy-phenyl)-5-(4-methoxy-benzo[1,3]dioxol-6-yl)-isoxazole;
4-(3-Acetylamino-4-methoxy-phenyl)-5-(4-methoxy-benzo[1,3]dioxol-6-yl)-isoxazole;
4-(3-Amino-4-methoxy-phenyl)-5-(4-methoxy-benzo[1,3]dioxol-6-yl)-isoxazole, hydrochloric acid salt;
4-(2-Hydroxy-4-methoxy-phenyl)-5-(4-methoxy-benzo[1,3]dioxol-6-yl)-isoxazole;
4-(2-Methoxy-pyridin-5-yl)-5-(4-methoxy-benzo[1,3]dioxol-6-yl)-isoxazole;
4-(5-Methoxy-pyridin-2-yl)-5-(4-methoxy-benzo[1,3]dioxol-6-yl)-isoxazole;
4-(3-Carboxy-4-methoxy-phenyl)-5-(4-methoxy-benzo[1,3]dioxol-6-yl)-isoxazole, sodium salt;
4-(3-Methoxycarbonyl-4-methoxy-phenyl)-5-(4-methoxy-benzo[1,3]dioxol-6-yl)-isoxazole;
4-(3-Sulfooxy-4-methoxy-phenyl)-5-(4-methoxy-benzo[1,3]dioxol-6-yl)-isoxazole, sodium salt;
4-(3-Amino-4-methoxy-phenyl)-5-(4-methoxy-benzo[1,3]dioxol-6-yl)-isoxazole;
4-(3,4-Dimethoxy-5-phosphonooxy-phenyl)-5-(4-methoxy-benzo[1,3]dioxol-6-yl)-isoxazole, disodium salt;
4-(2-Phosphonooxy-4-methoxy-phenyl)-5-(4-methoxy-benzo[1,3]dioxol-6-yl)-isoxazole, disodium salt;
4-(4-Methylsulfanyl-phenyl)-5-(4-methoxy-benzo[1,3]dioxol-6-yl)-isoxazole;
4-(3-Phosphonooxy-4-methylsulfanyl-phenyl)-5-(4-methoxy-benzo[1,3]dioxol-6-yl)-isoxazole, disodium salt;
4-(3-Amino-4-methylsulfanyl-phenyl)-5-(4-methoxy-benzo[1,3]dioxol-6-yl)-isoxazole;
4-(2,3-Dihydro-benzofuran-6-yl)-5-(4-methoxy-benzo[1,3]dioxol-6-yl)-isoxazole;
4-(4-Hydroxy-phenyl)-5-(4-methoxy-benzo[1,3]dioxol-6-yl)-isoxazole, sodium salt;
4-(4-Phosphonooxy-phenyl)-5-(4-methoxy-benzo[1,3]dioxol-6-yl)-isoxazole;
4-(4-1H-Tetrazol-5-yl-phenyl)-5-(4-methoxy-benzo[1,3]dioxol-6-yl)-isoxazole;
4-[4-(1-Methyl-1H-tetrazol-5-yl)-phenyl]-5-(4-methoxy-benzo[1,3]dioxol-6-yl)-isoxazole;
4-(1-Methyl-1H-indol -5-yl)-5-(4-methoxy-benzo[1,3]dioxol-6-yl)-isoxazole;
4-(3,4,5-Trimethoxy-phenyl)-5-(1-methyl-1H-indol -5-yl)-isoxazole;
4-(3,4,5-Trimethoxy-phenyl)-5-(3-phosphonooxy-4-methoxy-phenyl)-isoxazole, disodium salt;
4-(3,4,5-Trimethoxy-phenyl)-5-(N,N-dimethylamino-phenyl)-isoxazole;
4-(3,4,5-Trimethoxy-phenyl)-5-(3-amino-4-methoxy-phenyl)-isoxazole, hydrochloric acid salt;
4-(3,4,5-Trimethoxy-phenyl)-5-[3-(3-hydroxy-2S-amino-propionamido)-4-methoxy-phenyl]-isoxazole, hydrochloric acid salt;
4-(4-Methoxy-phenyl)-5-(2,4,5-trimethoxy-phenyl)-isoxazole;
4-(4-Methyl-phenyl)-5-(2,4,5-trimethoxy-phenyl)-isoxazole;
4-(4-Ethoxy-phenyl)-5-(2,4,5-trimethoxy-phenyl)-isoxazole;
4-(4-Ethyl-phenyl)-5-(2,4,5-trimethoxy-phenyl)-isoxazole;
4-(4-Propoxy-phenyl)-5-(2,4,5-trimethoxy-phenyl)-isoxazole;
4-(4-Propyl-phenyl)-5-(2,4,5-trimethoxy-phenyl)-isoxazole;
4-(4-Butoxy-phenyl)-5-(2,4,5-trimethoxy-phenyl)-isoxazole;
4-(4-Butyl-phenyl)-5-(2,4,5-trimethoxy-phenyl)-isoxazole;
4-(4-Bromo-phenyl)-5-(2,4,5-trimethoxy-phenyl)-isoxazole;
4-(4-Chloro-phenyl)-5-(2,4,5-trimethoxy-phenyl)-isoxazole;
4-(4-Fluoro-phenyl)-5-(2,4,5-trimethoxy-phenyl)-isoxazole;
4-(4-Nitro-phenyl)-5-(2,4,5-trimethoxy-phenyl)-isoxazole;
4-[4-(N,N-Dimethylamino)-phenyl]-5-(2,4,5-trimethoxy-phenyl)-isoxazole;
4-(3,4-Dimethoxy-phenyl)-5-(2,4,5-trimethoxy-phenyl)-isoxazole;
4-(3-Hydroxy-4-methoxy-phenyl)-5-(2,4,5-trimethoxy-phenyl)-isoxazole;
4-(3,4,5-Trimethoxy-phenyl)-5-(2,4,5-trimethoxy-phenyl)-isoxazole;
4-(4-Methoxy-phenyl)-5-(2,3,5-trimethoxy-phenyl)-isoxazole;
4-(4-Methyl-phenyl)-5-(2,3,5-trimethoxy-phenyl)-isoxazole;
4-(4-Ethoxy-phenyl)-5-(2,3,5-trimethoxy-phenyl)-isoxazole;
4-(4-Ethyl-phenyl)-5-(2,3,5-trimethoxy-phenyl)-isoxazole;
4-(4-Propoxy-phenyl)-5-(2,3,5-trimethoxy-phenyl)-isoxazole;
4-(4-Propyl-phenyl)-5-(2,3,5-trimethoxy-phenyl)-isoxazole;
4-(4-Butoxy-phenyl)-5-(2,3,5-trimethoxy-phenyl)-isoxazole;
4-(4-Butyl-phenyl)-5-(2,3,5-trimethoxy-phenyl)-isoxazole;
4-(4-Bromo-phenyl)-5-(2,3,5-trimethoxy-phenyl)-isoxazole;
4-(4-Chloro-phenyl)-5-(2,3,5-trimethoxy-phenyl)-isoxazole;
4-(4-Fluoro-phenyl)-5-(2,3,5-trimethoxy-phenyl)-isoxazole;
4-(4-Nitro-phenyl)-5-(2,3,5-trimethoxy-phenyl)-isoxazole;
4-[4-(N,N-Dimethylamino)-phenyl]-5-(2,3,5-trimethoxy-phenyl)-isoxazole;
4-(3,4-Dimethoxy-phenyl)-5-(2,3,5-trimethoxy-phenyl)-isoxazole;
4-(3-Hydroxy-4-methoxy-phenyl)-5-(2,3,5-trimethoxy-phenyl)-isoxazole;
4-(3,4,5-Trimethoxy-phenyl)-5-(2,3,5-trimethoxy-phenyl)-isoxazole;
4-(2,3,4,5-Tetramethoxy-phenyl)-5-(4-methoxy-phenyl)-isoxazole;
4-(2,3,4,5-Tetramethoxy-phenyl)-5-(4-methyl-phenyl)-isoxazole;
4-(2,3,4,5-Tetramethoxy-phenyl)-5-(4-ethoxy-phenyl)-isoxazole;
4-(2,3,4,5-Tetramethoxy-phenyl)-5-(4-ethyl-phenyl)-isoxazole;
4-(2,3,4,5-Tetramethoxy-phenyl)-5-(4-propoxy-phenyl)-isoxazole;
4-(2,3,4,5-Tetramethoxy-phenyl)-5-(4-propyl-phenyl)-isoxazole;
4-(2,3,4,5-Tetramethoxy-phenyl)-5-(4-butoxy-phenyl)-isoxazole;
4-(2,3,4,5-Tetramethoxy-phenyl)-5-(4-butyl-phenyl)-isoxazole;
4-(2,3,4,5-Tetramethoxy-phenyl)-5-(4-bromo-phenyl)-isoxazole;
4-(2,3,4,5-Tetramethoxy-phenyl)-5-(4-chloro-phenyl)-isoxazole;
4-(2,3,4,5-Tetramethoxy-phenyl)-5-(4-fluoro-phenyl)-isoxazole;
4-(2,3,4,5-Tetramethoxy-phenyl)-5-(4-nitro-phenyl)-isoxazole;
4-(2,3,4,5-Tetramethoxy-phenyl)-5-[4-(N,N,-dimethylamino)-phenyl]-isoxazole;
4-(2,3,4,5-Tetramethoxy-phenyl)-5-(3,4-dimethoxy-phenyl)-isoxazole;
4-(2,3,4,5-Tetramethoxy-phenyl)-5-(3-hydroxy-4-methoxy-phenyl)-isoxazole;
4-(2,3,4,5-Tetramethoxy-phenyl)-5-(3,4,5-trimethoxy-phenyl)-isoxazole;
4-(2,3-Dihydro-benzo[1,4]dioxin-6-yl)-5-(3,4-dimethoxy-phenyl)-isoxazole;
4-(3,4-Dimethy-phenyl)-5-(2-Hydroxy-4-methoxy-5-ethyl-phenyl)-isoxazole;
4-(4-Chloro-phenyl)-5-(2-hydroxy-4-methoxy-5-ethyl-phenyl)-isoxazole;
4-(4-Methyl-phenyl)-5-(2-hydroxy-4-methoxy-5-ethyl-phenyl)-isoxazole;
4-(4-Amino-phenyl)-5-(2-hydroxy-4-methoxy-5-ethyl-phenyl)-isoxazole; and
4-(4-Trifluoromethyl-phenyl)-5-(2-hydroxy-4-methoxy-5-ethyl-phenyl)-isoxazole; or
pharmaceutically acceptable salts, solvates, clathrates, or prodrugs thereof.

In another embodiment, the invention relates to compounds selected from the group consisting of:
4-(4-Bromo-phenyl)-3-(3,4,5-trimethoxy-phenyl)-isoxazole;
4-(Naphthalen-2-yl)-3-(2-hydroxy-4-methoxy-5-ethyl-phenyl)-isoxazole;
4-(4-Methoxy-phenyl)-3-(3,4,5-trimethoxy-phenyl)-isoxazole;
4-(4-Iodo-phenyl)-3-(2-hydroxy-4-methoxy-5-ethyl-phenyl)-isoxazole;
4-Phenyl-3-(2-hydroxy-4-methoxy-5-propyl-phenyl)-isoxazole;
4-(4-Bromo-phenyl)-3-(2-hydroxy-4-methoxy-5-ethyl-phenyl)-isoxazole;
4-(2,3-Dihydro-benzo[1,4]di-oxin-6-yl)-3-(2-hydroxy-4-methoxy-5-propyl-phenyl)-isoxazole;
4-(4-hydroxy-phenyl)-3-(3,4,5-trihydroxy-phenyl)-isoxazole;
4-(4-Iodo-phenyl)-3-(3,4,5-trimethoxy-phenyl)-isoxazole;
4-(3-Fluoro-4-methoxy-phenyl)-3-(3,4,5-trimethoxy-phenyl)-isoxazole;
4-(4-Nitro-phenyl)-3-(3,4,5-trimethoxy-phenyl)-isoxazole;
4-(4-Amino-phenyl)-3-(3,4,5-trimethoxy-phenyl)-isoxazole;
4-(4′-Methoxy-biphenyl-4-yl)-3-(3,4,5-trimethoxy-phenyl)-isoxazole;
4-[4-(pyridine-3-yl)-phenyl]-3-(3,4,5-trimethoxy-phenyl)-isoxazole;
4-[4-(pyridine-4-yl)-phenyl]-3-(3,4,5-trimethoxy-phenyl)-isoxazole;
4-[4-(pyridine-2-yl)-phenyl]-3-(3,4,5-trimethoxy-phenyl)-isoxazole;
4-(Quinolin-7-yl)-3-(3,4,5-trimethoxy-phenyl)-isoxazole;
4-(Pyridin-4-yl)-3-(3,4,5-trimethoxy-phenyl)-isoxazole;
4-(Isoquinolin-7-yl)-3-(3,4,5-trimethoxy-phenyl)-isoxazole;
4-(1-Methyl-1H-indol -5-yl)-3-(3,4,5-trimethoxy-phenyl)-isoxazole;
4-(4-Methoxy-phenyl)-3-(benzo[1,3]dioxol-5-yl)-isoxazole;
4-(4-Methoxy-phenyl)-3-(1-ethyl-1H-indol-6-yl)-isoxazole;
4-(4-Carboxy-phenyl)-3-(3,4,5-trimethoxy-phenyl)-isoxazole;
4-(4-Methoxycarbonyl-phenyl)-3-(3,4,5-trimethoxy-phenyl)-isoxazole;
4-[4-(Oxazol-2-yl)-phenyl]-3-(3,4,5-trimethoxy-phenyl)-isoxazole;
4-(4-Methoxy-phenyl)-3-(3,4,5-triethyl-phenyl)-isoxazole;
4-(4-Iodo-phenyl)-3-(3,4,5-triethyl-phenyl)-isoxazole;
4-(3-Fluoro-4-methoxy-phenyl)-3-(3,4,5-triethyl-phenyl)-isoxazole;
4-(4-Nitro-phenyl)-3-(3,4,5-triethyl-phenyl)-isoxazole;
4-(4-N,N-dimethylamino-phenyl)-3-(3,4,5-triethyl-phenyl)-isoxazole;
4-(4-Methoxy-phenyl)-3-(3,4,5-trimethyl-phenyl)-isoxazole;
4-[4-(Pyridin-3-yl)-phenyl]-3-(3,4,5-triethyl-phenyl)-isoxazole;
4-[4-(Pyridin-4-yl)-phenyl]-3-(3,4,5-triethyl-phenyl)-isoxazole;
4-[4-(Pyridin-2-yl)-phenyl]-3-(3,4,5-triethyl-phenyl)-isoxazole;
4-(Quinolin-7-yl)-3-(3,4,5-triethyl-phenyl)-isoxazole;
4-(Pyridin-4-yl)-3-(3,4,5-triethyl-phenyl)-isoxazole;
4-(Isoquinolin-7-yl)-3-(3,4,5-triethyl-phenyl)-isoxazole;
4-(1H-Indol-5-yl)-3-(3,4,5-triethyl-phenyl)-isoxazole;
4-(4-Methoxy-phenyl)-3-(benzo[1,3]dioxol-5-yl)-isoxazole;
4-(4-Methoxy-phenyl)-3-[1-isopropyl-1H-indol-6-yl)-isoxazole;
4-(4-Methoxy-phenyl)-3-(2,3,4-trimethoxy-phenyl)-isoxazole;
4-(3-Hydroxy-4-methoxy-phenyl)-3-(3,4,5-trimethoxy-phenyl)-isoxazole;
4-[3-(Ethyl-hydroxy-phosphoryloxy)-4-methoxy-phenyl]-3-(3,4,5-trimethoxy-phenyl)-isoxazole;
4-(4-Methoxy-phenyl)-3-(2-hydroxy-4-methoxy-5-ethyl-phenyl)-isoxazole;
4-(4-Isopropyl-phenyl)-3-(3,4,5-trimethoxy-phenyl)-isoxazole;
4-(2,3-Dihydro-benzo[1,4]dioxin-6-yl)-3-(3,4,5-trimethoxy-phenyl)-isoxazole;
4-(4-Ethyl-phenyl)-3-(3,4,5-trimethoxy-phenyl)-isoxazole;
4-(5-Methoxy-pyridin-2-yl)-3-(3,4,5-trimethoxy-phenyl)-isoxazole;
4-(4-Methoxy-phenyl)-3-(2,3,4-trimethoxy-pyridin-6-yl)-isoxazole;
4-(4-Methoxy-phenyl)-3-(3,5-dimethoxy-4-methoxycarbonyl-phenyl)-isoxazole;
4-(4-Methoxy-phenyl)-3-(3,5-diacetoxy-phenyl)-isoxazole;
4-(2-Methoxy-pyridin-5-yl)-3-(3,4,5-trimethoxy-phenyl)-isoxazole;
4-(4-Methoxy-phenyl)-3-(1-methyl-5-methoxy-1H-indol-7-yl)-isoxazole;
4-(4-Methoxy-phenyl)-3-(1-ethyl-1H-indol -7-yl)-isoxazole;
4-(4-Methoxy-phenyl)-3-(benzo[1,3]dioxol-4-yl)-isoxazole;
4-(2-Hydroxy-4-methoxy-phenyl)-3-(3,4,5-trimethoxy)-isoxazole;
4-[2-(Ethyl-hydroxy-phosphoryloxy)-4-methoxy-phenyl]-3-(3,4,5-trimethoxy-phenyl)-isoxazole;
4-(Pyridazin-4-yl)-3-(3,4,5-trimethoxy-phenyl)-isoxazole;
4-(Pyrimidin-5-yl)-3-(3,4,5-trimethoxy-phenyl)-isoxazole;
4-(Pyridin-3-yl)-3-(3,4,5-trimethoxy-phenyl)-isoxazole, hydrochloric acid salt;
4-(3-Mercapto-4-methoxy-phenyl)-3-(3,4,5-trimethoxy-phenyl)-isoxazole;
4-(3-Phosphonosulfanyl-4-methoxy-phenyl)-3-(3,4,5-trimethoxy-phenyl)-isoxazole, disodium salt;
4-(3-Acetylamino-4-methoxy-phenyl)-3-(3,4,5-trimethoxy-phenyl)-isoxazole;
4-(3-Amino-4-methoxy-phenyl)-3-(3,4,5-trimethoxy-phenyl)-isoxazole, hydrochloric acid salt;
4-(2-Hydroxy-4-methoxy-phenyl)-3-(3,4,5-trimethoxy-phenyl)-isoxazole;
4-(2-Methoxy-pyridine-5-yl)-3-(3,4,5-trimethoxy-phenyl)-isoxazole;
4-(5-Methoxy-pyridine-2-yl)-3-(3,4,5-trimethoxy-phenyl)-isoxazole;
4-(3-Carboxy-4-methoxy-phenyl)-3-(3,4,5-trimethoxy-phenyl)-isoxazole, sodium salt;
4-(3-Methoxycarbonyl-4-methoxy-phenyl)-3-(3,4,5-trimethoxy-phenyl)-isoxazole;
4-(3-Sulfooxy-4-methoxy-phenyl)-3-(3,4,5-trimethoxy-phenyl)-isoxazole, sodium salt;
4-(2-Amino-4-methoxy-phenyl)-3-(3,4,5-trimethoxy-phenyl)-isoxazole;
4-(3,4-Dimethoxy-5-phosphonooxy-phenyl)-3-(3,4,5-trimethoxy-phenyl)-isoxazole, disodium salt;
4-(2-Phosphonooxy-4-methoxy-phenyl)-3-(3,4,5-trimethoxy-phenyl)-isoxazole, disodium salt;
4-(4-Methylsulfanyl-phenyl)-3-(3,4,5-trimethoxy-phenyl)-isoxazole;
4-(3-Phosphonooxy-4-methylsulfanyl-phenyl)-3-(3,4,5-trimethoxy-phenyl)-isoxazole, disodium salt;
4-(3-Amino-4-methylsulfanyl-phenyl)-3-(3,4,5-trimethoxy-phenyl)-isoxazole;
4-(2,3-Dihydro-benzofuran-6-yl)-3-(3,4,5-trimethoxy-phenyl)-isoxazole;
4-(4-Hydroxy-phenyl)-3-(3,4,5-trimethoxy-phenyl)-isoxazole, sodium salt;
4-(4-Phosphonooxy-phenyl)-3-(3,4,5-trimethoxy-phenyl)-isoxazole, disodium salt;
4-(4-1H-Tetrazol-5-yl-phenyl)-3-(3,4,5-trimethoxy-phenyl)-isoxazole
4-[4-(1-Methyl -1H-tetrazol-5-yl)-phenyl]-3-(3,4,5-trimethoxy-phenyl)-isoxazole
4-(1-Methyl-1H-indol -5-yl)-3-(3,4,5-trimethoxy-phenyl)-isoxazole;
4-(Pyridazin-4-yl)-3-(4-methoxy-benzo[1,3]dioxol-6-yl)-isoxazole;
4-(Pyrimidin-5-yl)-3-(4-methoxy-benzo[1,3]dioxol-6-yl)-isoxazole;
4-(Pyridin-3-yl)-3-(4-methoxy-benzo[1,3]dioxol -6-yl)-isoxazole, hydrochloric acid salt;
4-(3-Mercapto-4-methoxy-phenyl)-3-(4-methoxy-benzo[1,3]dioxol-6-yl)-isoxazole;
4-(3-Phosphonosulfanyl-4-methoxy-phenyl)-3-(4-methoxy-benzo[1,3]dioxol-6-yl)-isoxazole;
4-(3-Acetylamino-4-methoxy-phenyl)-3-(4-methoxy-benzo[1,3]dioxol-6-yl)-isoxazole;
4-(3-Amino-4-methoxy-phenyl)-3-(4-methoxy-benzo[1,3]dioxol -6-yl)-isoxazole, hydrochloric acid salt;
4-(2-Hydroxy-4-methoxy-phenyl)-3-(4-methoxy-benzo[1,3]dioxol-6-yl)-isoxazole;
4-(2-Methoxy-pyridin-5-yl)-3-(4-methoxy-benzo[1,3]dioxol-6-yl)-isoxazole;
4-(5-Methoxy-pyridin-2-yl)-3-(4-methoxy-benzo[1,3]dioxol-6-yl)-isoxazole;
4-(3-Carboxy-4-methoxy-phenyl)-3-(4-methoxy-benzo[1,3]dioxol-6-yl)-isoxazole, sodium salt;
4-(3-Methoxycarbonyl-4-methoxy-phenyl)-3-(4-methoxy-benzo[1,3]dioxol-6-yl)-isoxazole;
4-(3-Sulfooxy-4-methoxy-phenyl)-3-(4-methoxy-benzo[1,3]dioxol-6-yl)-isoxazole, sodium salt;
4-(3-Amino-4-methoxy-phenyl)-3-(4-methoxy-benzo[1,3]dioxol-6-yl)-isoxazole;
4-(3,4-Dimethoxy-5-phosphonooxy-phenyl)-3-(4-methoxy-benzo[1,3]dioxol -6-yl)-isoxazole, disodium salt;
4-(2-Phosphonooxy-4-methoxy-phenyl)-3-(4-methoxy-benzo[1,3]dioxol-6-yl)-isoxazole, disodium salt;
4-(4-Methylsulfanyl-phenyl)-3-(4-methoxy-benzo[1,3]dioxol-6-yl)-isoxazole;
4-(3-Phosphonooxy-4-methylsulfanyl-phenyl)-3-(4-methoxy-benzo[1,3]dioxol-6-yl)-isoxazole, disodium salt;
4-(3-Amino-4-methylsulfanyl-phenyl)-3-(4-methoxy-benzo[1,3]dioxol-6-yl)-isoxazole;
4-(2,3-Dihydro-benzofuran-6-yl)-3-(4-methoxy-benzo[1,3]dioxol-6-yl)-isoxazole;
4-(4-Hydroxy-phenyl)-3-(4-methoxy-benzo[1,3]dioxol-6-yl)-isoxazole, sodium salt;
4-(4-Phosphonooxy-phenyl)-3-(4-methoxy-benzo[1,3]dioxol-6-yl)-isoxazole;
4-(4-1H-Tetrazol-5-yl-phenyl)-3-(4-methoxy-benzo[1,3]dioxol-6-yl)-isoxazole;
4-[4-(1-Methyl-1H-tetrazol-5-yl)-phenyl]-3-(4-methoxy-benzo[1,3]dioxol-6-yl)-isoxazole;
4-(1-Methyl-1H-indol -5-yl)-3-(4-methoxy-benzo[1,3]dioxol-6-yl)-isoxazole;
4-(3,4,5-Trimethoxy-phenyl)-3-(1-methyl -1H-indol-5-yl)-isoxazole;
4-(3,4,5-Trimethoxy-phenyl)-3-(3-phosphonooxy-4-methoxy-phenyl)-isoxazole, disodium salt;
4-(3,4,5-Trimethoxy-phenyl)-3-(N,N-dimethylamino-phenyl)-isoxazole;
4-(3,4,5-Trimethoxy-phenyl)-3-(3-amino-4-methoxy-phenyl)-isoxazole, hydrochloric acid salt;
4-(3,4,5-Trimethoxy-phenyl)-3-[3-(3-hydroxy-2S-amino-propionamido)-4-methoxy-phenyl]-isoxazole, hydrochloric acid salt;
4-(4-Methoxy-phenyl)-3-(2,4,5-trimethoxy-phenyl)-isoxazole;
4-(4-Methyl-phenyl)-3-(2,4,5-trimethoxy-phenyl)-isoxazole;
4-(4-Ethoxy-phenyl)-3-(2,4,5-trimethoxy-phenyl)-isoxazole;
4-(4-Ethyl-phenyl)-3-(2,4,5-trimethoxy-phenyl)-isoxazole;
4-(4-Propoxy-phenyl)-3-(2,4,5-trimethoxy-phenyl)-isoxazole;
4-(4-Propyl-phenyl)-3-(2,4,5-trimethoxy-phenyl)-isoxazole;
4-(4-Butoxy-phenyl)-3-(2,4,5-trimethoxy-phenyl)-isoxazole;
4-(4-Butyl-phenyl)-3-(2,4,5-trimethoxy-phenyl)-isoxazole;
4-(4-Bromo-phenyl)-3-(2,4,5-trimethoxy-phenyl)-isoxazole;
4-(4-Chloro-phenyl)-3-(2,4,5-trimethoxy-phenyl)-isoxazole;
4-(4-Fluoro-phenyl)-3-(2,4,5-trimethoxy-phenyl)-isoxazole;
4-(4-Nitro-phenyl)-3-(2,4,5-trimethoxy-phenyl)-isoxazole;
4-[4-(N,N-Dimethylamino)-phenyl]-3-(2,4,5-trimethoxy-phenyl)-isoxazole;
4-(3,4-Dimethoxy-phenyl)-3-(2,4,5-trimethoxy-phenyl)-isoxazole;
4-(3-Hydroxy-4-methoxy-phenyl)-3-(2,4,5-trimethoxy-phenyl)-isoxazole;
4-(3,4,5-Trimethoxy-phenyl)-3-(2,4,5-trimethoxy-phenyl)-isoxazole;
4-(4-Methoxy-phenyl)-3-(2,3,5-trimethoxy-phenyl)-isoxazole;
4-(4-Methyl-phenyl)-3-(2,3,5-trimethoxy-phenyl)-isoxazole;
4-(4-Ethoxy-phenyl)-3-(2,3,5-trimethoxy-phenyl)-isoxazole;
4-(4-Ethyl-phenyl)-3-(2,3,5-trimethoxy-phenyl)-isoxazole;
4-(4-Propoxy-phenyl)-3-(2,3,5-trimethoxy-phenyl)-isoxazole;
4-(4-Propyl-phenyl)-3-(2,3,5-trimethoxy-phenyl)-isoxazole;
4-(4-Butoxy-phenyl)-3-(2,3,5-trimethoxy-phenyl)-isoxazole;
4-(4-Butyl-phenyl)-3-(2,3,5-trimethoxy-phenyl)-isoxazole;
4-(4-Bromo-phenyl)-3-(2,3,5-trimethoxy-phenyl)-isoxazole;
4-(4-Chloro-phenyl)-3-(2,3,5-trimethoxy-phenyl)-isoxazole;
4-(4-Fluoro-phenyl)-3-(2,3,5-trimethoxy-phenyl)-isoxazole;
4-(4-Nitro-phenyl)-3-(2,3,5-trimethoxy-phenyl)-isoxazole;
4-[4-(N,N-Dimethylamino)-phenyl]-3-(2,3,5-trimethoxy-phenyl)-isoxazole;
4-(3,4-Dimethoxy-phenyl)-3-(2,3,5-trimethoxy-phenyl)-isoxazole;
4-(3-Hydroxy-4-methoxy-phenyl)-3-(2,3,5-trimethoxy-phenyl)-isoxazole;
4-(3,4,5-Trimethoxy-phenyl)-3-(2,3,5-trimethoxy-phenyl)-isoxazole;
4-(2,3,4,5-Tetramethoxy-phenyl)-3-(4-methoxy-phenyl)-isoxazole;
4-(2,3,4,5-Tetramethoxy-phenyl)-3-(4-methyl-phenyl)-isoxazole
4-(2,3,4,5-Tetramethoxy-phenyl)-3-(4-ethoxy-phenyl)-isoxazole;
4-(2,3,4,5-Tetramethoxy-phenyl)-3-(4-ethyl-phenyl)-isoxazole;
4-(2,3,4,5-Tetramethoxy-phenyl)-3-(4-propoxy-phenyl)-isoxazole;
4-(2,3,4,5-Tetramethoxy-phenyl)-3-(4-propyl-phenyl)-isoxazole;
4-(2,3,4,5-Tetramethoxy-phenyl)-3-(4-butoxy-phenyl)-isoxazole;
4-(2,3,4,5-Tetramethoxy-phenyl)-3-(4-butyl-phenyl)-isoxazole;
4-(2,3,4,5-Tetramethoxy-phenyl)-3-(4-bromo-phenyl)-isoxazole;
4-(2,3,4,5-Tetramethoxy-phenyl)-3-(4-chloro-phenyl)-isoxazole;
4-(2,3,4,5-Tetramethoxy-phenyl)-3-(4-fluoro-phenyl)-isoxazole;
4-(2,3,4,5-Tetramethoxy-phenyl)-3-(4-nitro-phenyl)-isoxazole
4-(2,3,4,5-Tetramethoxy-phenyl)-3-[4-(N,N,-dimethylamino)-phenyl]-isoxazole;
4-(2,3,4,5-Tetramethoxy-phenyl)-3-(3,4-dimethoxy-phenyl)-isoxazole;
4-(2,3,4,5-Tetramethoxy-phenyl)-3-(3-hydroxy-4-methoxy-phenyl)-isoxazole;
4-(2,3,4,5-Tetramethoxy-phenyl)-3-(3,4,5-trimethoxy-phenyl)-isoxazole;
4-(2,3-Dihydro-benzo[1,4]dioxin-6-yl)-3-(3,4-dimethoxy-phenyl)-isoxazole;
4-(3,4-Dimethy-phenyl)-3-(2-hydroxy-4-methoxy-5-ethyl-phenyl)-isoxazole;
4-(4-Chloro-phenyl)-3-(2-hydroxy-4-methoxy-5-ethyl-phenyl)-isoxazole;
4-(4-Methyl-phenyl)-3-(2-hydroxy-4-methoxy-5-ethyl-phenyl)-isoxazole;
4-(4-Amino-phenyl)-3-(2-hydroxy-4-methoxy-5-ethyl-phenyl)-isoxazole;
4-(4-Trifluoromethyl-phenyl)-3-(2-hydroxy-4-methoxy-5-ethyl-phenyl)-isoxazole;
4-(4-Methoxy-phenyl)-3-(2-hydroxy-4-methoxy-5-ethyl-phenyl)-isoxazole; and
4-(3,4,5-Trimethoxy-phenyl)-3-(4-bromo-phenyl)-isoxazole; or

pharmaceutically acceptable salts, solvates, clathrates, or prodrugs thereof.

In another embodiment, the invention relates to compounds selected from the group consisting of:
2-amino-N-(2-methoxy-5-[5-(3,4,5-trimethoxy-phenyl)-isoxazol-4-yl)-phenyl)acetamide hydrochloride;
2-amino-3-hydroxy-N-(2-methoxy-5-[5-(3,4,5-trimethoxy-phenyl)-isoxazol-4-yl)-phenyl)propanamide hydrochloride;
2-amino-N-(2-methoxy-5-[5-(3,4,5-trimethoxy-phenyl)-isoxazol-4-yl)-phenyl)propanamide;
2-amino-N-(2-methoxy-5-[5-(3,4,5-trimethoxy-phenyl)-isoxazol-4-yl)-phenyl)-4-(methylthio)butanamide hydrochloride;
2-amino-N-(2-methoxy-5-[5-(3,4,5-trimethoxy-phenyl)-isoxazol-4-yl)-phenyl)butanamide;
2-amino-N-(2-methoxy-5-[5-(3,4,5-trimethoxy-phenyl)-isoxazol-4-yl)-phenyl)-3-phenylpropanamide hydrochloride;
2-amino-N-(2-methoxy-5-[5-(3,4,5-trimethoxy-phenyl)-isoxazol-4-yl)-phenyl)-4-methylpentanamide hydrochloride;
2-amino-N-(2-methoxy-5-[5-(3,4,5-trimethoxy-phenyl)-isoxazol-4-yl)-phenyl)-3-(4-methoxyphenyl)propanamide hydrochloride;
1-{2-methoxy-5-[5-(3,4,5-trimethoxy-phenyl)-isoxazol-4-yl]-phenylcarbomoyl}-2-methyl-propyl-ammonium chloride;
1-{2-methoxy-5-[5-(3,4,5-trimethoxy-phenyl)-isoxazol-4-yl]-phenylcarbomoyl}-2-methyl-butyl-ammonium chloride;
2-hydroxy-1-{2-methoxy-5-[5-(3,4,5-trimethoxy-phenyl)-isoxazol-4-yl]-phenylcarbomoyl}-propyl-ammonium chloride;
2-(4-hydroxy-phenyl)-1-{2-methoxy-5-[5-(3,4,5-trimethoxy-phenyl)-isoxazol-4-yl]-phenylcarbomoyl}-ethyl-ammonium chloride;
C-{2-methoxy-5-[5-(3,4,5-trimethoxy-phenyl)-isoxazol-4-yl]-phenylcarbomoyl}-C-phenyl-methyl-ammonium chloride;
2-(1H-indol -2-yl)-1-{2-methoxy-5-[5-(3,4,5-trimethoxy-phenyl)-isoxazol-4-yl]-phenylcarbomoyl}-ethyl-ammonium chloride;
2-benzofuran-2-yl-1-{2-methoxy-5-[5-(3,4,5-trimethoxy-phenyl)-isoxazol-4-yl]-phenylcarbomoyl}-ethyl-ammonium chloride;
2-carboxyl-1-{2-methoxy-5-[5-(3,4,5-trimethoxy-phenyl)-isoxazol-4-yl]-phenylcarbomoyl}-ethyl-ammonium chloride;
3-carboxyl-1-{2-methoxy-5-[5-(3,4,5-trimethoxy-phenyl)-isoxazol-4-yl]-phenylcarbomoyl}-propyl-ammonium chloride;
3-carbamoyl-1-{2-methoxy-5-[5-(3,4,5-trimethoxy-phenyl)-isoxazol-4-yl]-phenylcarbomoyl}-propyl-ammonium chloride;
2-carbamoyl-1-{2-methoxy-5-[5-(3,4,5-trimethoxy-phenyl)-isoxazol-4-yl]-phenylcarbomoyl}-ethyl-ammonium chloride;
2-(3H-imidazol-4-yl)-1-{2-methoxy-5-[5-(3,4,5-trimethoxy-phenyl)-isoxazol-4-yl]-phenylcarbomoyl}-ethyl-ammonium chloride;
5-amino-1-{2-methoxy-5-[5-(3,4,5-trimethoxy-phenyl)-isoxazol-4-yl]-phenylcarbomoyl}-pentyl-ammonium chloride;
4-guanidino-1-{2-methoxy-5-[5-(3,4,5-trimethoxy-phenyl)-isoxazol-4-yl]-phenylcarbomoyl}-butyl-ammonium chloride;
N-{2-methoxy-5-[5-(3,4,5-trimethoxy-phenyl)-isoxazol-4-yl]-phenyl}succinamic acid;
4-{2-methoxy-5-[5-(3,4,5-trimethoxy-phenyl)-isoxazol-4-yl]-phenylcarbamoyl}-butyric acid;
2-{2-methoxy-5-[5-(3,4,5-trimethoxy-phenyl)-isoxazol-4-yl]-phenylcarbamoyl}-ethyl-ammonium chloride;
3-(2-methoxy-ethoxy)-N-{2-methoxy-5-[5-(3,4,5-trimethoxy-phenyl)-isoxazol-4-yl]-phenyl}-propionamide;
3-(2-PEG)-N-{2-methoxy-5-[5-(3,4,5-trimethoxy-phenyl)-isoxazol-4-yl]-phenyl}-propionamide;
N-(2-methoxy-5-[5-(3,4,5-trimethoxy-phenyl)-isoxazol-4-yl]-phenyl}-3-(2-methylamino-ethylamino)-propionamide;
3-PEG-N-{2-methoxy-5-[5-(3,4,5-trimethoxy-phenyl)-isoxazol-4-yl]-phenylcarbamoyl)-methyl)-propionamide;
N-(2-methoxy-5-[5-(3,4,5-trimethoxy-phenyl)-isoxazol-4-yl-phenylcarbamoyl}-methyl)-succinamic acid
{2-methoxy-5-[5-(3,4,5-trimethoxy-phenyl)-isoxazol-4-yl]-phenyl}-carbamic acid 2-methoxy-ethyl ester;
2-methoxy-5-(5-(3,4,5-trimethoxyphenyl)isoxazol-4-yl)phenylcarbamate-PEG;
3-amino-N-[4-guanadino-1-{2-methoxy-5-[5-(3,4,5-trimethoxy-phenyl)-isoxazol-4-yl]-phenylcarbamoyl}-butylcarbamoyl)-methyl]-succinamic acid;
3-amino-N-[4-guanadino-1-{2-methoxy-5-[5-(3,4,5-trimethoxy-phenyl)-isoxazol-4-yl]-phenylcarbamoyl}-butylcarbamoyl)-methyl]-succinamic acid;
2-amino-N-(2-methoxy-5-[5-(3,4,5-trimethoxy-phenyl)-isoxazol-4-yl)-phenyl)propanamide hydrochloride;
2-amino-N-(2-methoxy-5-[3-(3,4,5-trimethoxy-phenyl)-isoxazol-4-yl)-phenyl)acetamide hydrochloride;
2-amino-3-hydroxy-N-(2-methoxy-5-[3-(3,4,5-trimethoxy-phenyl)-isoxazol-4-yl)-phenyl)propanamide hydrochloride;
2-amino-N-(2-methoxy-5-[3-(3,4,5-trimethoxy-phenyl)-isoxazol-4-yl)-phenyl)propanamide;
2-amino-N-(2-methoxy-5-[3-(3,4,5-trimethoxy-phenyl)-isoxazol-4-yl)-phenyl)-4-(methylthio)butanamide hydrochloride;
2-amino-N-(2-methoxy-5-[3-(3,4,5-trimethoxy-phenyl)-isoxazol-4-yl)-phenyl)butanamide;
2-amino-N-(2-methoxy-5-[3-(3,4,5-trimethoxy-phenyl)-isoxazol-4-yl)-phenyl)-3-phenylpropanamide hydrochloride;
2-amino-N-(2-methoxy-5-[3-(3,4,5-trimethoxy-phenyl)-isoxazol-4-yl)-phenyl)-4-methylpentanamide hydrochloride;
2-amino-N-(2-methoxy-5-[3-(3,4,5-trimethoxy-phenyl)-isoxazol-4-yl)-phenyl)-3-(4-methoxyphenyl)propanamide hydrochloride;
1-{2-methoxy-5-[3-(3,4,5-trimethoxy-phenyl)-isoxazol-4-yl]-phenylcarbomoyl}-2-methyl-propyl-ammonium chloride;
1-{2-methoxy-5-[3-(3,4,5-trimethoxy-phenyl)-isoxazol-4-yl]phenylcarbomoyl}-2-methyl-butyl-ammonium chloride;
2-hydroxy-1-{2-methoxy-5-[3-(3,4,5-trimethoxy-phenyl)-isoxazol-4-yl]-phenylcarbomoyl}-propyl-ammonium chloride;
2-(4-hydroxy-phenyl)-1-(2-methoxy-5-[3-(3,4,5-trimethoxy-phenyl)-isoxazol-4-yl]-phenylcarbomoyl}-ethyl-ammonium chloride;
C-{2-methoxy-5-[3-(3,4,5-trimethoxy-phenyl)-isoxazol-4-yl]-phenylcarbomoyl}-C-phenyl-methyl-ammonium chloride;
2-(1H-indol -2-yl)-1-{2-methoxy-5-[3-(3,4,5-trimethoxy-phenyl)-isoxazol-4-yl]-phenylcarbomoyl}-ethyl-ammonium chloride;
2-benzofuran-2-yl-1-{2-methoxy-5-[3-(3,4,5-trimethoxy-phenyl)-isoxazol-4-yl]-phenylcarbomoyl}-ethyl-ammonium chloride;
2-carboxyl-1-{2-methoxy-5-[3-(3,4,5-trimethoxy-phenyl)-isoxazol-4-yl]-phenylcarbomoyl}-ethyl-ammonium chloride;
3-carboxyl-1-{2-methoxy-5-[3-(3,4,5-trimethoxy-phenyl)-isoxazol-4-yl]-phenylcarbomoyl}-propyl-ammonium chloride;
3-carboxyl-1-{2-methoxy-5-[3-(3,4,5-trimethoxy-phenyl)-isoxazol-4-yl]-phenylcarbomoyl}-propyl-ammonium chloride;
3-carbamoyl-1-{2-methoxy-5-[3-(3,4,5-trimethoxy-phenyl)-isoxazol-4-yl]-phenylcarbomoyl}-propyl-ammonium chloride;
2-carbamoyl-1-{2-methoxy-5-[3-(3,4,5-trimethoxy-phenyl)-isoxazol-4-yl]-phenylcarbomoyl}-ethyl-ammonium chloride;
2-(3H-imidazol-4-yl)-1-{2-methoxy-5-[3-(3,4,5-trimethoxy-phenyl)-isoxazol-4-yl]phenylcarbomoyl}-ethyl-ammonium chloride;
5-amino-1-{2-methoxy-5-[3-(3,4,5-trimethoxy-phenyl)-isoxazol-4-yl]-phenylcarbomoyl}-pentyl-ammonium chloride;
4-guanidino-1-{2-methoxy-5-[3-(3,4,5-trimethoxy-phenyl)-isoxazol-4-yl]-phenylcarbomoyl}-butyl-ammonium chloride;
N-{2-methoxy-5-[3-(3,4,5-trimethoxy-phenyl)-isoxazol-4-yl]-phenyl}succinamic acid;
4-{2-methoxy-5-[3-(3,4,5-trimethoxy-phenyl)-isoxazol-4-yl]-phenylcarbamoyl}-butyric acid;
2-{2-methoxy-5-[3-(3,4,5-trimethoxy-phenyl)-isoxazol-4-yl]-phenylcarbamoyl}-ethyl-ammonium chloride;
3-(2-methoxy-ethoxy)-N-{2-methoxy-5-[3-(3,4,5-trimethoxy-phenyl)-isoxazol-4-yl]-phenyl}-propionamide;
3-(2-PEG)-N-{2-methoxy-5-[3-(3,4,5-trimethoxy-phenyl)-isoxazol-4-yl]-phenyl}-propionamide;
N-{2-methoxy-5-[3-(3,4,5-trimethoxy-phenyl)-isoxazol-4-yl]-phenyl}-3-(2-methylamino-ethylamino)-propionamide;
3-PEG-N-{2-methoxy-5-[3-(3,4,5-trimethoxy-phenyl)-isoxazol-4-yl]-phenylcarbamoyl}-methyl)-propionamide;
N-{2-methoxy-5-[3-(3,4,5-trimethoxy-phenyl)-isoxazol-4-yl]-phenylcarbamoyl}-methyl)-succinamic acid;
{2-methoxy-5-[5-(3,4,5-trimethoxy-phenyl)-isoxazol-4-yl]-phenyl}-carbamic acid 2-methoxy-ethyl ester;
2-methoxy-5-(3-(3,4,5-trimethoxyphenyl)isoxazol-4-yl)phenylcarbamate-PEG;
3-amino-N-[4-guanadino-1-{2-methoxy-5-[3-(3,4,5-trimethoxy-phenyl)-isoxazol-4-yl]-phenylcarbamoyl}-butylcarbamoyl)-methyl]-succinamic acid;
3-amino-N-[4-guanadino-1-{2-methoxy-5-[3-(3,4,5-trimethoxy-phenyl)-isoxazol-4-yl]-phenylcarbamoyl}-butylcarbamoyl)-methyl]succinamic acid;
2-amino-N-(2-methoxy-5-[3-(3,4,5-trimethoxy-phenyl)-isoxazol-4-yl)-phenyl)propanamide hydrochloride;
methyl-2-(2-(2-methoxy-5-(5-(3,4,5-trimethoxyphenypisoxazol-4-yl)phenylamino)-2-oxoethylamino)acetate;
4-amino-5-(2-methoxy-5-(5-(3,4,5-trimethoxyphenypisoxazol-4-yl)phenylamino)-5-oxopentanoic acid hydrochloride;
3-amino-N-(2-methoxy-5-(5-(3,4,5-trimethoxyphenyl)isoxazol-4-yl)phenyl)propanamide hydrochloride;
3-amino-N-(2-methoxy-5-(5-(3,4,5-trimethoxyphenypisoxazol-4-yl)phenyl)-4-methylpentanamide hydrochloride;
methyl-2-(2-(2-methoxy-5-(3-(3,4,5-trimethoxyphenypisoxazol-4-yl)phenylamino)-2-oxoethylamino)acetate;
4-amino-5-(2-methoxy-5-(3-(3,4,5-trimethoxyphenypisoxazol-4-yl)phenylamino)-5-oxopentanoic acid hydrochloride;
3-amino-N-(2-methoxy-5-(3-(3,4,5-trimethoxyphenypisoxazol-4-yl)phenyl)propanamide hydrochloride; and
3-amino-N-(2-methoxy-5-(3-(3,4,5-trimethoxyphenypisoxazol-4-yl)phenyl)-4-methylpentanamide hydrochloride; or

a pharmaceutically acceptable salt, solvate, clathrate, or prodrug thereof.

In one embodiment, the invention relates to compounds of formula (XI):

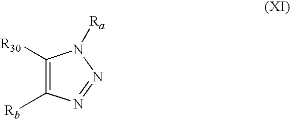

or a pharmaceutically acceptable salt, solvate, clathrate, or prodrug thereof, wherein Rₐ, R_{b}, and R₃₀ are defined as above. In another embodiment, in the compounds represented by formula (XI), Rₐ is not acridinyl.

In another embodiment, the invention relates to compounds of formula (XII):

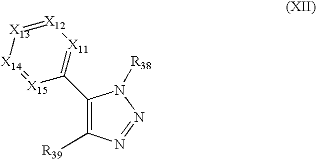

or a pharmaceutically acceptable salt, solvate, clathrate, or prodrug thereof, wherein:

one of R₃₈ or R₃₉ is —H and the other is

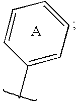

ring A is optionally substituted; and

X₁₁, X₁₂, X₁₃, X₁₄, and X₁₅ are each, independently, N or CR₃₁, provided that at least two of X₁₁, X₁₂, X₁₃, X₁₄, and X₁₅ are CR₃₁; and

R₃₁ is —H or a substituent.

In another embodiment, the invention relates to compounds of formula (XIII):

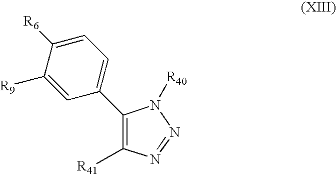

or a pharmaceutically acceptable salt, solvate, clathrate, or prodrug thereof, wherein:

one of R₄₀ or R₄₁ is —H and the other is

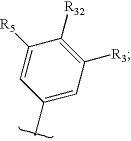

R₃, R₃₂, R₅, and R₆ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₉ is —H, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂)², —SR₇, —S(O)ₚR₂, —OS(O)ₚR₇, —S(O)ₚOR₇, —NR₈S(O)ₚR₇, or 'S(O)ₚNR₁₀R₁₁;

R₇, R₈, R₁₀, R₁₁, and p are defined as above.

In another embodiment, the invention relates to compounds represented by formulae (XIIa) through

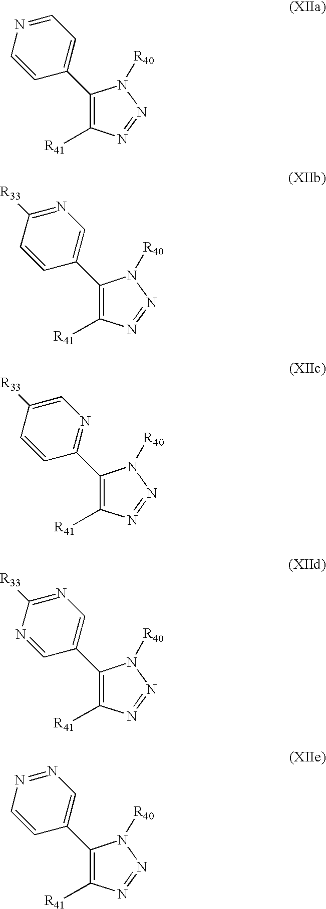

or a pharmaceutically acceptable salt, solvate, clathrate, or prodrug thereof, wherein:

R₄₀ and R₄₁ are defined as above;

R₃₃ is —H, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁; and

R₇, R₈, R₁₀, R₁₁, and p are defined as above.

In another embodiment, the invention relates to compounds of formula (XIV):

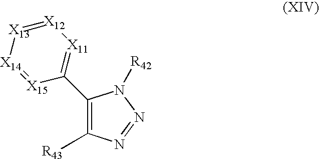

or a pharmaceutically acceptable salt, solvate, clathrate, or prodrug thereof, wherein:

one of R₄₂ or R₄₃ is —H and the other is

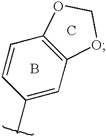

X₁₁, X₁₂, X₁₃, and X₁₄ are each, independently, N or CR₃₁, provided that at least two of X₁₁, X₁₂, X₁₃, X₁₄, and X₁₅ are CR₃₁;

Ring B is optionally substituted with one to three substituents;

Ring C is optionally substituted with one or two substituents; and

R₃₁ is defined as above.

In another embodiment, the invention relates to compounds of formula (XV):

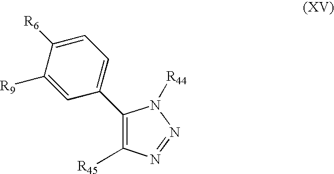

or a pharmaceutically acceptable salt, solvate, clathrate, or prodrug thereof, wherein:

one of R₄₄ or R₄₅ is —H and the other is

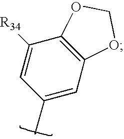

R₆ and R₉ are defined as above; and

R₃₄ is —H, an alkyl, an alkoxy, a halo, nitro, cyano, —OH, —NH₂, an alkyl amino, or a dialkyl amino.

In another embodiment, the invention relates to compounds of formulae:

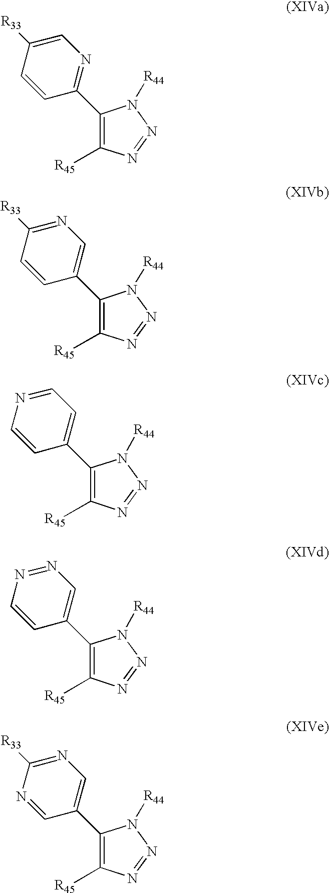

or a pharmaceutically acceptable salt, solvate, clathrate, or prodrug thereof, wherein R₄₄, R₄₅, and R₃₃ are defined as above.

In another embodiment, the invention relates to compounds of formula (XXIX):

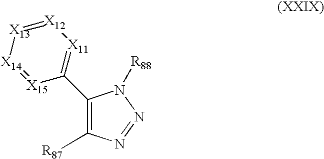

or a pharmaceutically acceptable salt, solvate, clathrate, or prodrug thereof, wherein:

one of R₈₆ or R₈₇ is —H and the other is

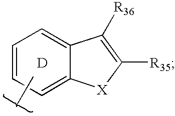

Ring D is optionally substituted one to three substituents;

X₁₁, X₁₂, X₁₃, X₁₄, and X₁₅ are each, independently, N or CR₃₁, provided that at least two of X₁₁, X₁₂, X₁₃, X₁₄, and X₁₅ are CR₃₁;

R₃₅, and R₃₆ are each, independently, —H or a substituent;

X is O or NR₅₆;

R₅₆ is —H, an optionally substituted alkyl, —C(O)R₇, —C(O)OR₇, or —C(O)NR₁₀R₁₁ and R₃₁ is defined as above.

In another embodiment, the invention relates to compounds of formula (XVI):

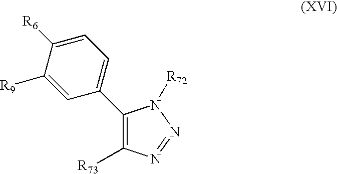

or a pharmaceutically acceptable salt, solvate, clathrate, or prodrug thereof, wherein:

one of R₇₂ or R₇₃ is —H and the other is

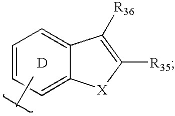

Ring D, X, R₆, R₉, R₃₅ and R₃₆ are defined as above.

In another embodiment, the invention relates to compounds of formula (XVII):

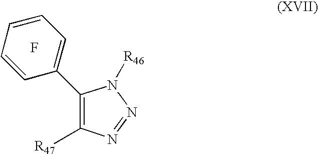

or a pharmaceutically acceptable salt, solvate, clathrate, or prodrug thereof, wherein:

one of R₄₆ or R₄₇ is —H and the other is

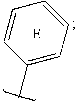

one of rings E or F is substituted with three or four substituents and the other is substituted with one or more substituents. In another embodiment, in the compounds represented by formula (XVII), when R₄₆ is —H, then Ring E is not 4-aminophenyl.

In another embodiment, the invention relates to compounds of formula (XVIII):

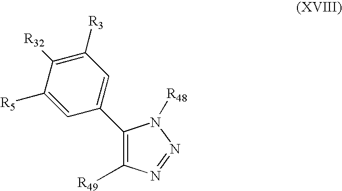

or a pharmaceutically acceptable salt, solvate, clathrate, or prodrug thereof, wherein:

one of R₄₈ or R₄₉ is —H and the other is

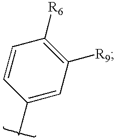

R₃, R₃₂, R₅, R₆ and R₉ are defined as above.

In another embodiment, the invention relates to compounds of formula (XIX):

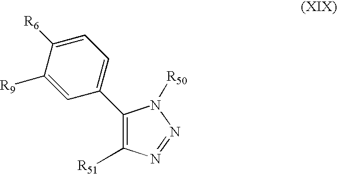

or a pharmaceutically acceptable salt, solvate, clathrate, or prodrug thereof, wherein:

one of R₅₀ or R₅₁ is —H and the other is

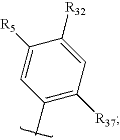

R₃₇ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁; and

R₃₂, R₅, R₆, R₇, R₈, R₉, R₁₀, R₁₁ and p are defined as above.

In another embodiment, the invention relates to compounds of formula (XX):

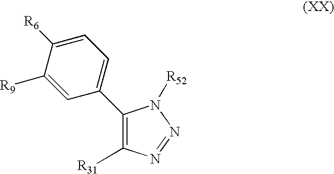

or a pharmaceutically acceptable salt, solvate, clathrate, or prodrug thereof, wherein:

one of R₅₂ or R₅₃ is —H and the other is

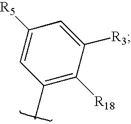

R₁₈ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(O R₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁; and

R₃, R₅, R₆, R₇, R₈, R₉, R₁₀, R₁₁ and p are defined as above.

In another embodiment, the invention relates to compounds of formula (XXI):

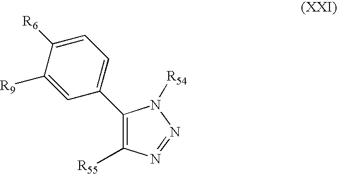

or a pharmaceutically acceptable salt, solvate, clathrate, or prodrug thereof, wherein:

one of R₅₄ or R₅₅ is —H and the other is

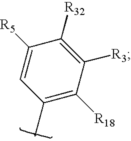

R₃, R₃₂, R₅, R₆, R₉, and R₁₈ are defined as above.

In another embodiment, the invention relates to compounds of formula (XXII):

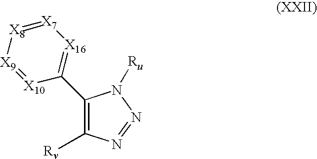

or a pharmaceutically acceptable salt, solvate, clathrate, or prodrug thereof, wherein:

one of Rᵤ or Rᵥ is —H and the other is

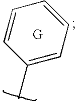

ring G is substituted with three to five substituents;

X₁₆, X₇, X₈, X₉, and X₁₀ are each, independently, N or CR₃₁, provided that at least one of X₁₆, X₇, X₈, X₉, or X₁₀ is N and at least two of X₁₆, X₇, X₈, X₉, and X₁₀ are CR₃₁; and

R₃₁ is defined as above.

In another embodiment, the invention relates to compounds of formula (XXIV):

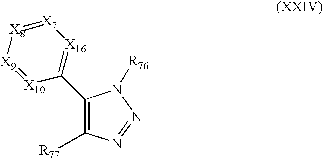

or a pharmaceutically acceptable salt, solvate, clathrate, or prodrug thereof, wherein:

one of R₇₆ or R₇₇ is —H and the other is

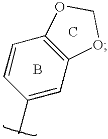

X₁₆, X₇, X₈, X₉, and X₁₀ are each, independently, N or CR₃₁, provided that at least one of X₁₆, X₇, X₈, X₉, or X₁₀ is N and at least two of X₁₆, X₇, X₈, X₉, and X₁₀ are CR₃₁;

Ring B is optionally substituted with one to three substituents;

Ring C is optionally substituted with one or two substituents; and

R₃₁ is defined as above.

In another embodiment, the invention relates to compounds of formula (XXV):

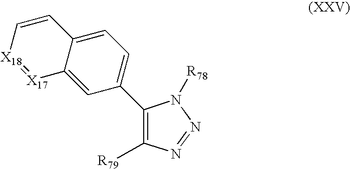

or a pharmaceutically acceptable salt, solvate, clathrate, or prodrug thereof, wherein:

one of R₇₈ or R₇₉ is —H and the other is

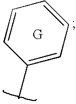

ring G is optionally substituted with one to five substituents;

ring H is optionally substituted with one to three substituents;

ring I is optionally substituted with one or two substituents;

X₁₇, and X₁₈ are each, independently, N or CR₃₁, provided that at least one X₉, or X₁₀ is N; and

R₃₁ is defined as above.

In another embodiment, the invention relates to compounds of formula (XXVI):

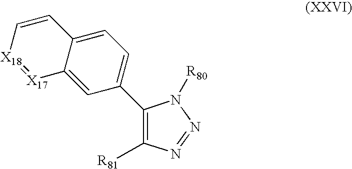

or a pharmaceutically acceptable salt, solvate, clathrate, or prodrug thereof, wherein;

one of R₈₀ or R₈₁ is —H and the other is

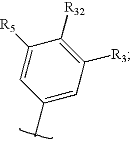

X₁₇, X₁₈, R₃, R₃₂, and R₅ are defined as above.

In another embodiment, the invention relates to compounds of formula (XXVII):

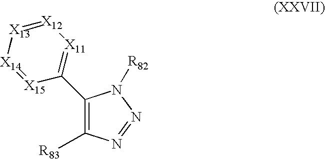

or a pharmaceutically acceptable salt, solvate, clathrate, or prodrug thereof, wherein:

one of R₈₂ or R₈₃ is —H and the other is

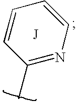

ring J is substituted with three or four substituents;

X₁₁, X₁₂, X₁₃, X₁₄, and X₁₅ are each, independently, N or CR₃₁, provided that at least two of X₁₁, X₁₂, X₁₃, X₁₄, and X₁₅ are CR₃₁; and

R₃₁ is defined as above.

In another embodiment, the invention relates to compounds of formula (XXVIII):

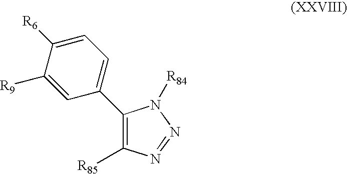

or a pharmaceutically acceptable salt, solvate, clathrate, or prodrug thereof, wherein:

one of R₈₄ or R₈₅ is —H and the other is

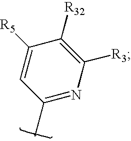

R₃, R₃₂, R₅, R₆ and R₉ are defined as above.

In one embodiment, the invention relates to compounds of formula (XIA):

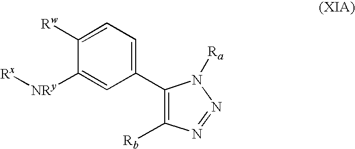

or a pharmaceutically acceptable salt, solvate, clathrate, or prodrug thereof, wherein one of Rₐ or R_{b} is —H and the other is an optionally substituted aryl or an optionally substituted heteroaryl;

R^{x} is (R^{aa})ₘ, —R^{aa}—C(O)(CH₂)ₙC(O)OH, —C(O)(CH₂)ₙC(O)OH, —C(O)YR^{z}, —C(O)NH—R^{aa}, or —(R^{aa})_{q}C(O)(Y₁);

R^{y} is —H or lower alkyl;

R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl;

R₇, for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R^{aa} is an amino acid residue or an amino acid residue analog;

Y is CH₂, O, or NH;

R^{z} is Alk-NH₂, Alk-C(O)OH, Het, or Y₁;

Alk is an optionally substituted alkylene;

Het is an optionally substituted heteroalkyl;

Y₁ is a water soluble polymer with a molecular weight less than 60,000 daltons;

n is 1, 2, 3, or 4;

m is an integer from 1 to 10; and

q is 0 or 1.

In another embodiment, in the compounds represented by formula (XIA), neither Rₐ or R_{b} is acridinyl.

In another embodiment, the invention relates to compounds of formula (XIIA):

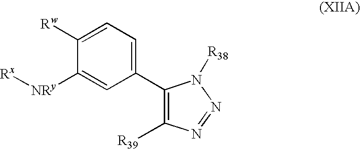

or a pharmaceutically acceptable salt, solvate, clathrate, or prodrug thereof, wherein:

one of R₃₈ or R₃₉ is —H and the other is

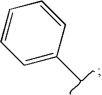

ring A is optionally substituted; and

R^{x}, R^{y}, and R^{w} are defined as above.

In another embodiment, the invention relates to compounds of formula (XIIIA):

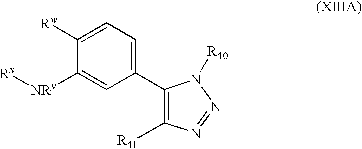

or a pharmaceutically acceptable salt, solvate, clathrate, or prodrug thereof, wherein:

one of R₄₀ or R₄₁ is —H and the other is

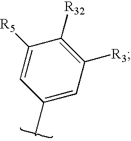

R₃, R₃₂, and R₅ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁; and

R^{x}, R^{y}, R^{w}, R₇, R₈, R₁₀, R₁₁, and p are defined as above.

In another embodiment, the invention relates to compounds of formula (XIVA):

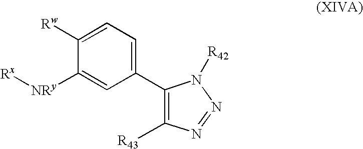

or a pharmaceutically acceptable salt, solvate, clathrate, or prodrug thereof, wherein:

one of R₄₂ or R₄₃ is —H and the other is

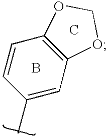

Ring B is optionally substituted with one to three substituents;

Ring C is optionally substituted with one or two substituents; and

R^{x}, R^{y}, and R^{w} are defined as above.

In another embodiment, the invention relates to compounds of formula (XVA):

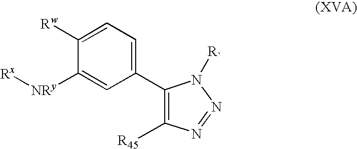

or a pharmaceutically acceptable salt, solvate, clathrate, or prodrug thereof, wherein:

one of R₄₄ or R₄₅ is —H and the other is

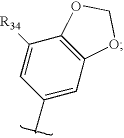

R^{x}, R^{y}, R^{w} are defined as above; and

R₃₄ is —H, an alkyl, an alkoxy, a halo, nitro, cyano, —OH, —NH₂, an alkyl amino, or a dialkyl amino.

In another embodiment, the invention relates to compounds of formula (XVIIA):

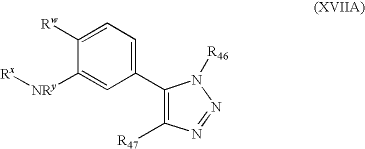

or a pharmaceutically acceptable salt, solvate, clathrate, or prodrug thereof, wherein:

one of R₄₆ or R₄₇ is —H and the other is

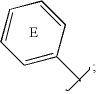

Ring E is substituted with one or more substituents.

In another embodiment, the invention relates to compounds of formula (XXIXA):

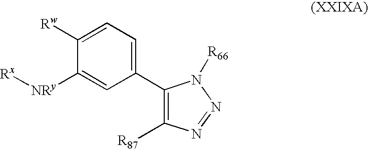

or a pharmaceutically acceptable salt, solvate, clathrate, or prodrug thereof, wherein:

one of R₈₆ or R₈₇ is —H and the other is

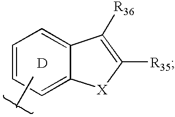

Ring D is optionally substituted one to three substituents; and

R^{x}, R^{y}, R^{w}, X, R₃₅, and R₃₆ are defined as above.

In another embodiment, the invention relates to compounds of formula (XVIIIA):

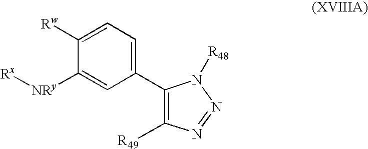

or a pharmaceutically acceptable salt, solvate, clathrate, or prodrug thereof, wherein

one of R₄₈ or R₄₉ is —H and the other is

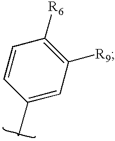

R^{x}, R^{y}, R^{w} R₆ and R₉ are defined as above.

In another embodiment, the invention relates to compounds of formula (XIXA):

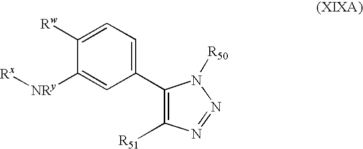

or a pharmaceutically acceptable salt, solvate, clathrate, or prodrug thereof, wherein:

one of R₅₀ or R₅₁ is —H and the other is

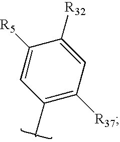

R₃₇ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)R₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁; and

R₃₂, R₅, R^{x}, R^{y}, R^{w}, R₇, R₈, R₁₀, R₁₁ and p are defined as above.

In another embodiment, the invention relates to compounds of formula (XXA):

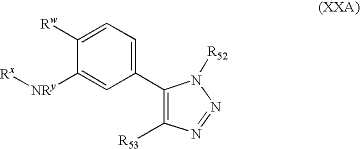

or a pharmaceutically acceptable salt, solvate, clathrate, or prodrug thereof, wherein:

one of R₅₂ or R₅₃ is —H and the other is

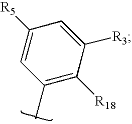

R₁₈ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁; and

R₃, R₅, R^{x}, R^{y}, R^{w}, R₇, R₈, R₉, R₁₀, R₁₁ and p are defined as above.

In another embodiment, the invention relates to compounds of formula (XXIA):

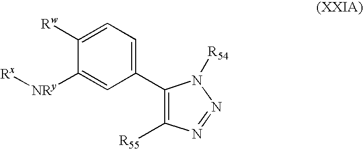

or a pharmaceutically acceptable salt, solvate, clathrate, or prodrug thereof, wherein:

one of R₅₄ or R₅₅ is —H and the other is

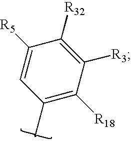

R₃, R₃₂, R₅, R^{x}, R^{y}, R^{w} and R₁₈ are defined as above.

In another embodiment, the invention relates to compounds of formula (XXVIIA):

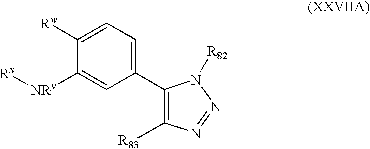

or a pharmaceutically acceptable salt, solvate, clathrate, or prodrug thereof, wherein:

one of R₈₂ or R₈₃ is —H and the other is

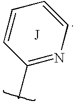

ring J is substituted with three or four substituents; and

R^{x}, R^{y}, R^{w}, and R₃₁ are defined as above.

In another embodiment, the invention relates to compounds of formula (XXVIIIA):

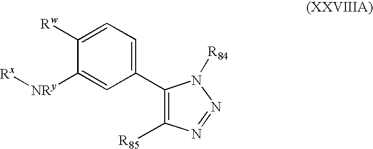

or a pharmaceutically acceptable salt, solvate, clathrate, or prodrug thereof, wherein:

one of R₈₄ or R₈₅ is —H and the other is

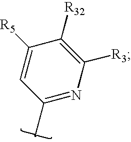

R₃, R₃₂, R₅, R^{x}, R^{y}, and R^{w} are defined as above.

In one embodiment, the invention relates to compounds of formula (XIB):

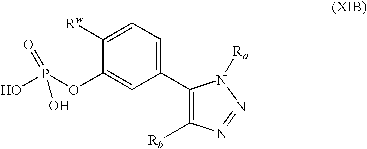

or a pharmaceutically acceptable salt, solvate, clathrate, or prodrug thereof, wherein

one of Rₐ or R_{b} is —H and the other is an optionally substituted aryl or an optionally substituted heteroaryl;

R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl;

R_{7,} for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl.

In another embodiment, in the compounds represented by formula (X113), neither Rₐ or R_{b} is acridinyl.

In another embodiment, the invention relates to compounds of formula (XIIB):

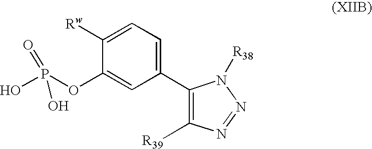

or a pharmaceutically acceptable salt, solvate, clathrate, or prodrug thereof, wherein:

one of R₃₈ or R₃₉ is —H and the other is

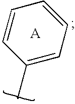

ring A is optionally substituted; and

R^{x}, R^{y}, and R^{w} are defined as above.

In another embodiment, the invention relates to compounds of formula (XIIIB):

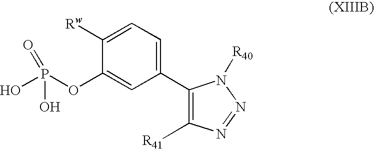

or a pharmaceutically acceptable salt, solvate, clathrate, or prodrug thereof, wherein:

one of R₄₀ or R₄₁ is —H and the other is

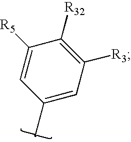

R₃, R₃₂, R₅, and R₆ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁; and

R^{x}, R^{y}, R^{w}, R₇, R₈, R₁₀, R₁₁, and p are defined as above.

In another embodiment, the invention relates to compounds of formula (XIVB):

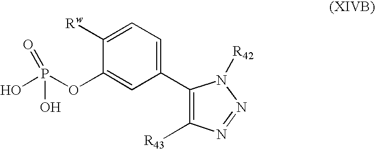

or a pharmaceutically acceptable salt, solvate, clathrate, or prodrug thereof, wherein:

one of R₄₂ or R₄₃ is —H and the other is

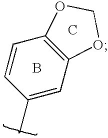

Ring B is optionally substituted with one to three substituents;

Ring C is optionally substituted with one or two substituents; and

R^{x}, R^{y}, and R^{w} are defined as above.

In another embodiment, the invention relates to compounds of formula (XVB):

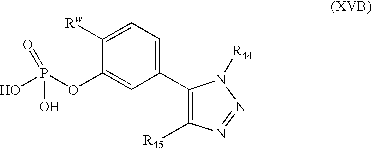

or a pharmaceutically acceptable salt, solvate, clathrate, or prodrug thereof; wherein:

one of R₄₄ or R₄₅ is —H and the other is

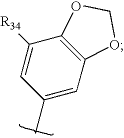

R^{x}, R^{y}, and R^{w} are defined as above; and

R₃₄ is —H, an alkyl, an alkoxy, a halo, nitro, cyano, —OH, —NH₂, an alkyl amino, or a dialkyl amino.

In another embodiment, the invention relates to compounds of formula (XVIIB):

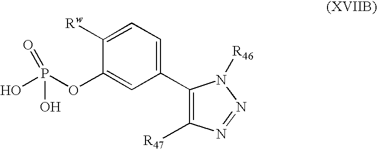

or a pharmaceutically acceptable salt, solvate, clathrate, or prodrug thereof, wherein:

one of R₄₆ or R₄₇ is —H and the other is

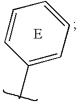

one of rings E or F is substituted with three or four substituents and the other is substituted with one or more substituents.

In another embodiment, the invention relates to compounds of formula (XXIXB):

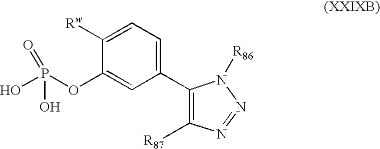

or a pharmaceutically acceptable salt, solvate, clathrate, or prodrug thereof, wherein:

one of R₈₆ or R₈₇ is —H and the other is

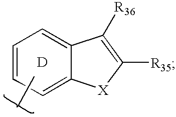

Ring D is optionally substituted one to three substituents; and

R^{x}, R^{y}, R^{w}, X, R₃₅, and R₃₆ are defined as above.

In another embodiment, the invention relates to compounds of formula (XVIIIB):

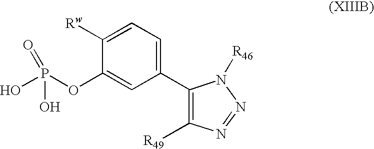

or a pharmaceutically acceptable salt, solvate, clathrate, or prodrug thereof, wherein:

one of R₄₈ or R₄₉ is —H and the other is

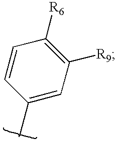

R^{x}, R^{y}, R^{w} R₆ and R₉ are defined as above.

In another embodiment, the invention relates to compounds of formula (XIXB):

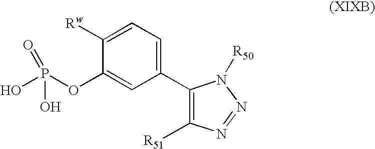

or a pharmaceutically acceptable salt, solvate, clathrate, or prodrug thereof, wherein:

one of R₅₀ or R₅₁ is —H and the other is

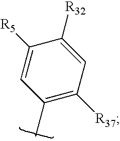

R₃₇ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁; and

R₃₂, R₅, R^{x}, R^{y}, R^{w}, R₇, R₈, R₁₀, R₁₁ and p are defined as above.

In another embodiment, the invention relates to compounds of formula (XXB):

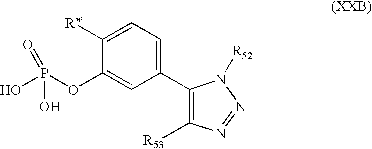

or a pharmaceutically acceptable salt, solvate, clathrate, or prodrug thereof, wherein:

one of R₅₂ or R₅₃ is —H and the other is

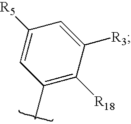

R₁₈ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁; and

R₃, R₅, R^{x}, R^{y}, R^{w}, R₇, R₈, R₉, R₁₀, R₁₁ and p are defined as above.

In another embodiment, the invention relates to compounds of formula (XXIB):

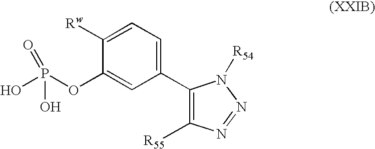

or a pharmaceutically acceptable salt, solvate, clathrate, or prodrug thereof, wherein:

one of R₅₄ or R₅₅ is —H and the other is

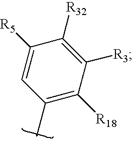

R₃, R₃₂, R₅, R^{x}, R^{y}, R^{w} and R₁₈ are defined as above.

In another embodiment, the invention relates to compounds of formula (XXVIIB):

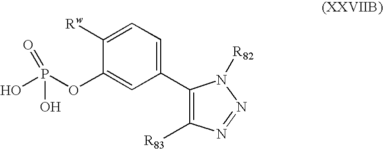

or a pharmaceutically acceptable salt, solvate, clathrate, or prodrug thereof, wherein:

one of R₈₂ or R₈₃ is —H and the other is

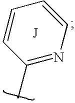

ring J is substituted with three or four substituents; and

R^{x}, R^{y}, R^{w}, and R₃₁ are defined as above.

In another embodiment, the invention relates to compounds of formula (XXVIIIB):

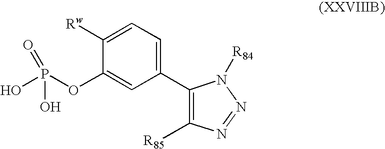

or a pharmaceutically acceptable salt, solvate, clathrate, or prodrug thereof, wherein:

one of R₈₄ or R₈₅ is —H and the other is

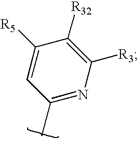

R₃, R₃₂, R₅, R^{x}, R^{y}, and R^{w} are defined as above.

In some embodiments, Rₐ and R₃₀ in formula (XI) are each independently a substituted or unsubstituted phenyl. In some embodiments, R_{b} and R₃₀ in formula (XI) are each independently a substituted or unsubstituted phenyl.

In some embodiments, Rₐ in formula (XI), (XIA), or (XIB) is —H. In some embodiments, R_{b} in formula (XI), (XIA), or (XIB) is —H.

In some embodiments, R₃ of formula (XIII), (XIIIA), (XIIIB), (XIIa), (XIIb), (XIIc), (XIId), (XIIe), (XVIII), (XVIIIA), (XVIIIB), (XX), (XXA), (XXB), (XXI), (XXIA), (XXIB), (XXVI), (XXVIII), (XVIIIA), or (XXVIIIB) is a lower alkyl, lower alkoxy, lower alkylsulfanyl, —OH, —SH, —NH₂, halo, lower dialkyl amino, lower alkyl amino, nitro, cyano, pyridinyl, carboxy, lower alkoxycarbonyl, oxazolyl, —SP(O)(OR₅₈)₂, —OP(O)(OR₅₈)₂, —OC(O)R₅₈, —OS(O)₂(OR₅₈), tetrazolyl, 1-methyl-tetrazolyl, —NHC(O)R₅₈, or —NHC(O)CH(R₅₇)NH₂, wherein R₅₈ for each occurrence is independently, —H or a lower alkyl; and R₅₇ is H or an amino acid sidechain. In some embodiments, R₃ of formula (XIII), (XIIIA), (XIIIB), (XIIa), (XIIb), (XIIc), (XIId), (XIIe), (XVIII), (XVIIIA), (XVIIIB), (XX), (XXA), (XXB), (XXI), (XXIA), (XXIB), (XXVI), (XXVIII), (XVIIIA), or (XXVIIIB) is methyl, ethyl, or methoxy; preferably, R₃ is methoxy.

In some embodiments, R₃₂ of formula (XIII), (XIIIA), (XIIIB), (XIIa), (XIIb), (XIIc), (XIId), (XIIe), (XVIII), (XVIIIA), (XVIIIB), (XX), (XXA), (XXB), (XXI), (XXIA), (XXIB), (XXVI), (XXVIII), (XVIIIA), or (XXVIIIB) is a lower alkyl, lower alkoxy, lower alkylsulfanyl, —OH, —SH, —NH₂, halo, lower dialkyl amino, lower alkyl amino, nitro, cyano, pyridinyl, carboxy, lower alkoxycarbonyl, oxazolyl, —SP(O)(OR₅₈)₂, —OP(O)(OR₅₈)₂, —OC(O)R₅₈, —OS(O)₂(OR₅₈), tetrazolyl, 1-methyl-tetrazolyl, —NHC(O)R₅₈, or —NHC(O)CH(R₅₇)NH₂, wherein R₅₈ for each occurrence is independently, —H or a lower alkyl; and R₅₇ is H or an amino acid sidechain. In some embodiments, R₃₂ of formula (XIII), (XIIIA), (XIIIB), (XIIa), (XIIb), (XIIc), (XIId), (XIIe), (XVIII), (XVIIIA), (XVIIIB), (XX), (XXA), (XXB), (XXI), (XXIA), (XXIB), (XXVI), (XXVIII), (XVIIIA), or (XXVIIIB) is methyl, ethyl, or methoxy; preferably, R₃₂ is methoxy.

In some embodiments, R₅ of formula (XIII), (XIIIA), (XIIIB), (XIIa), (XIIb), (XIIc), (XIId), (XIIe),(XVIII), (XVIIIA), (XVIIIB), (XX), (XXA), (XXB), (XXI), (XXIA), (XXIB), (XXVI), (XXVIII), (XVIIIA), or (XXVIIIB) is a lower alkyl, lower alkoxy, lower alkylsulfanyl, —OH, —SH, —NH₂, halo, lower dialkyl amino, lower alkyl amino, nitro, cyano, pyridinyl, carboxy, lower alkoxycarbonyl, oxazolyl, —SP(O)(OR₅₈)₂, —OP(O)(OP₅₈)₂, —OC(O)R₅₈, —OS(O)₂(OR₅₈), tetrazolyl, 1-methyl-tetrazolyl, —NHC(O)R₅₈, or —NHC(O)CH(R₅₇)NH₂, wherein R₅₈ for each occurrence is independently, —H or a lower alkyl; and R₅₇ is H or an amino acid sidechain. In some embodiments, R₅ of formula (XIII), (XIIIA), (XIIIB), (XIIa), (XIIb), (XIIc), (XIId), (XIIe), (XVIII), (XVIIIA), (XVIIIB), (XX), (XXA), (XXB), (XXI), (XXIA), (XXIB), (XXVI), (XXVIII), (XVIIIA), or (XXVIIIB) is methyl, ethyl, or methoxy; preferably, R₃ is methoxy.

In some embodiments, R₆ of formula (XIII), (XV), (XVI), (XVIII), (XIX), (XX), (XXI), or (XXVIII) is a lower alkyl, lower alkoxy, lower alkylsulfanyl, —OH, —SH, —NH₂, halo, lower dialkyl amino, lower alkyl amino, nitro, cyano, pyridinyl, carboxy, lower alkoxycarbonyl, oxazolyl, —SP(O)(OR₅₈)₂, —OP(O)(OR₅₈)₂, —OC(O)R₅₈, —OS(O)₂(OR₅₈), tetrazolyl, 1-methyl-tetrazolyl, —NHC(O)R₅₈, or —NHC(O)CH(R₅₇)NH₂, wherein R₅₈ for each occurrence is independently, —H or a lower alkyl; and R₅₇ is H or an amino acid sidechain. In some embodiments, R₆ of formula (XIII), (XV), (XVI), (XVIII), (XIX), (XX), (XXI), or (XXVIII) is methyl, ethyl, or methoxy; preferably, R₃ is methoxy. In some embodiments, R₆ of formula (XIII), (XV), (XVI), (XVIII), (XIX), (XX), (XXI), or (XXVIII) is a lower alkyl, lower alkoxy, lower alkylsulfanyl, —OH, —SH, —NH₂, halo, lower dialkyl amino, lower alkyl amino, nitro, pyridinyl, carboxy, lower alkoxycarbonyl, oxazolyl, —SP(O)(OR₅₈)₂, —OP(O)(OR₅₈)₂, —OC(O)R₅₈, —OS(O)₂(OR₅₈), tetrazolyl, 1-methyl-tetrazolyl, or —NHC(O)R₅₈; preferably, R₆ is methoxy, dimethyl amino, methyl amino, hydroxy, —NHC(O)CH(R₅₇)NH₂, —OS(O)₂OR₅₈, —SP(O)(OR₅₈)₂, or —OP(O)(OR₅₈)₂; more preferably, R₃ is methoxy.

In some embodiments, R₃, R₃₂, and R₅ of formula (XIII), (XIIIA), (XIIIB), (XIIa), (XIIb), (XIIc), (XIId), (XIIe), (XVIII), (XVIIIA), (XVIIIB), (XX), (XXA), (XXB), (XXI), (XXIA), (XXIB), (XXVI), (XXVIII), (XVIIIA), or (XXVIIIB) are each, independently, a lower alkyl, lower alkoxy, lower alkylsulfanyl, —OH, —SH, —NH₂, halo, lower dialkyl amino, lower alkyl amino, nitro, cyano, pyridinyl, carboxy, lower alkoxycarbonyl, oxazolyl, —SP(O)(OR₅₈)₂, —OP(O)(OR₅₈)₂, —OC(O)R₅₈, —OS(O)₂(OR₅₈), tetrazolyl, 1-methyl-tetrazolyl, —NHC(O)R₅₈, or —NHC(O)CH(R₅₇)NH₂, wherein R₅₈ for each occurrence is independently, —H or a lower alkyl; and R₅₇ is H or an amino acid sidechain. In some embodiments, R₃, R₃₂, and R₅ of formula (XIII), (XIIIA), (XIBB), (XIIa), (XIIb), (XIId), (XIIe), (XVIIIA), (XVIIIB), (XX), (XXA), (XXB), (XXI), (XXIA), (XXIB), (XXVI), (XXVIII), (XVIIIA), or (XXVIIIB) are each, independently, methyl, ethyl, or methoxy; preferably, R₃, R₃₂, and R₅ are each methoxy.

In some embodiments, R₃, R₃₂, R₅, and R₆ of formula (XIII), (XVIII), (XXI), or (XXVIII) are each, independently, a lower alkyl, lower alkoxy, lower alkylsulfanyl, —OH, —SH, —NH₂, halo, lower dialkyl amino, lower alkyl amino, nitro, cyano, pyridinyl, carboxy, lower alkoxycarbonyl, oxazolyl, —SP(O)(OR₅₈)₂, —OP(O)(OR₅₈)₂, —OC(O)R₅₈, —OS(O)₂(OR₅₈), tetrazolyl, 1-methyl-tetrazolyl, —NHC(O)R₅₈, or —NHC(O)CH(R₅₇)NH₂, wherein R₅₈ for each occurrence is independently, —H or a lower alkyl; and R₅₇ is H or an amino acid sidechain. In some embodiments, R₃, R₃₂, R₅, and R₆ of formula (XIII), (XVIII), (XXI), or (XXVIII) are each, independently, methyl, ethyl, or methoxy; preferably, R₃, R₃₂, R₅ and R₆ are methoxy. In some embodiments, R₃, R₃₂, and R₅ of formula (XIII), (XVIII), (XXI), or (XXVIII) are each, independently, methyl, ethyl, or methoxy, and R₆ of formula (XIII), (XVIII), (XXI), or (XXVIII) is a lower alkyl, lower alkoxy, lower alkylsulfanyl, —OH, —SH, —NH₂, halo, lower dialkyl amino, lower alkyl amino, nitro, pyridinyl, carboxy, lower alkoxycarbonyl, oxazolyl, —SP(O)(OR₅₈)₂, —OP(O)(OR₅₈)₂, —OC(O)R₅₈, —OS(O)₂(OR₅₈), tetrazolyl, 1-methyl-tetrazolyl, or —NHC(O)R₅₈; preferably, R₆ is methoxy, dimethyl amino, methyl amino, hydroxy, —NHC(O)CH(R₅₇)NH₂, —OS(O)₂OR₅₈, —SP(O)(OR₅₈)₂, or —OP(O)(OR₅₈)₂; preferably, R₃, R₃₂, R₅ and R₆ are methoxy.

In some embodiments, R₃₂, R₅, and R₆ of formula (XIX) are each, independently, a lower alkyl, lower alkoxy, lower alkylsulfanyl, —OH, —SH, —NH₂, halo, lower dialkyl amino, lower alkyl amino, nitro, cyano, pyridinyl, carboxy, lower alkoxycarbonyl, oxazolyl, —SP(O)(OR₅₈)₂, —OP(O)(OR₅₈)₂, —OC(O)R₅₈, —0S(O)₂(0R₅₈), tetrazolyl, 1-methyl-tetrazolyl, —NHC(O)R₅₈, or —NHC(O)CH(R₅₇)NH₂, wherein R₅₈ for each occurrence is independently, —H or a lower alkyl; and R₅₇ is H or an amino acid sidechain. In some embodiments, R₃₂, R₅, and R₆ of formula (XIX) are each, independently, methyl, ethyl, or methoxy; preferably, R₃₂, R₅ and R₆ are methoxy. In some embodiments, R₃₂ and R₅ of formula (XIX) are each, independently, methyl, ethyl, or methoxy, and R₆ of formula (XIX) is a lower alkyl, lower alkoxy, lower alkylsulfanyl, —OH, —SH, —NH₂, halo, lower dialkyl amino, lower alkyl amino, nitro, pyridinyl, carboxy, lower alkoxycarbonyl, oxazolyl, —SP(O)(OR₅₈)₂, —OP(O)(OR₅₈)₂, —OC(O)R₅₈, —OS(O)₂(OR₅₈), tetrazolyl, 1-methyl-tetrazolyl, or —NHC(O)R₅₈; preferably, R₆ is methoxy, dimethyl amino, methyl amino, hydroxy, —NHC(O)CH(R₅₇)NH₂, —OS(O)₂OR₅₈, —SP(O)(OR₅₈)₂, or —OP(O)(OR₅₈)₂; preferably, R₃₂, R₅ and R₆ are methoxy.

In some embodiments, R₃, R₅, and R₆ of formula (XX) are each, independently, a lower alkyl, lower alkoxy, lower alkylsulfanyl, —OH, —SH, —NH₂, halo, lower dialkyl amino, lower alkyl amino, nitro, cyano, pyridinyl, carboxy, lower alkoxycarbonyl, oxazolyl, —SP(O)(OR₅₈)₂, —OP(O)(OR₅₈)₂, —OC(O)R₅₈, —OS(O)₂(OR₅₈), tetrazolyl, 1-methyl-tetrazolyl, —NHC(O)R₅₈, or —NHC(O)CH(R₅₇)NH₂, wherein R₅₈ for each occurrence is independently, —H or a lower alkyl; and R₅₇ is H or an amino acid sidechain. In some embodiments, R₃, R₅, and R₆ of formula (XX) are each, independently, methyl, ethyl, or methoxy; preferably, R₃, R₅ and R₆ are methoxy. In some embodiments, R₃ and R₅ of formula (XX) are each, independently, methyl, ethyl, or methoxy, and R₆ of formula (XX) is a lower alkyl, lower alkoxy, lower alkylsulfanyl, —OH, —SH, —NH₂, halo, lower dialkyl amino, lower alkyl amino, nitro, pyridinyl, carboxy, lower alkoxycarbonyl, oxazolyl, —SP(O)(OR₅₈)₂, —OP(O)(OR₅₈)₂, —OC(O)R₅₈, —OS(O)₂(OR₅₈), tetrazolyl, 1-methyl-tetrazolyl, or —NHC(O)R₅₈; preferably, R₆ is methoxy, dimethyl amino, methyl amino, hydroxy, —NHC(O)CH(R₅₇)NH₂, —OS(O)₂OR₅₈, —SP(O)(OR₅₈)₂, or —OP(O)(OR₅₈)₂; preferably, R₃, R₅ and R₆ are methoxy.

In some embodiments, R₉ of formula (XIII), (XV), (XVI), (XVIII), (XVIIIA), (XVIIIB), (XIX), (XX), (XXI), or (XXVIII) is —H, halo, —OH, —SH, —NH₂, carboxy, —OP(O)(OR₅₈)₂, —SP(O)(OR₅₈)₂, —NHC(O)R₅₈, —NHC(O)CH(R₅₇)NH₂, —0S(O)₂(0R₅₈), lower alkoxycarbonyl, or lower alkoxy; preferably, R₉ is —H, amino, hydroxy, —NHC(O)CH(R₅₇)NH₂, or —OP(O)(OR₅₈)₂.

In some embodiments, R₃₄ of formula (XV), (XVA), (XVB), (XIVa), (XIVb), (XIVc), (XIVd) or (XIVe) is —H or a lower alkoxy.

In some embodiments, ring A of formula (XII), (XIIA), or (XIIB) is optionally substituted with one to five substituents independently selected from the group consisting of an optionally substituted alkyl, an optionally substituted alkoxy, an optionally substituted alkylsulfanyl, an optionally substituted alkylamino, an optionally substituted dialkylamino, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, an optionally substituted haloalkyl, —C(O)NR₃₄R₃₅, —NR₃₆C(O)R₃₇, halo, —OR₃₆, cyano, nitro, haloalkoxy, —C(O)R₃₆, —NR₃₄R₃₅, —SR₃₆, —C(O)OR₃₆, —OC(O)R₃₆, —NR₃₆C(O)NR₃₄R₃₅, —NR₃₆C(N—R₃₈)NR₃₄R₃₅, —OC(O)NR₃₄R₃₅, —NR₃₆C(O)OR₃₇, —OP(O)(OR₃₆)₂, —SP(O)(OR₃₆)₂, —OS(O)₂(OR₃₆), —S(O)ₚR₃₆) or —S(O)ₚNR₃₄R₃₅, wherein R₃₄ and R₃₅, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₃₄ and R₃₅ taken together with the nitrogen to which they are attached is an optionally substituted heterocyclyl or an optionally substituted heteroaryl; R₃₆ and R₃₇ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; and R₃₈ is H, an optionally substituted alkyl, —C(O)R₃₆, —C(O)OR₃₆, or an optionally substituted aralkyl.

In some embodiments, ring B of formula (XIV), (XIVA), or (XIVB) is optionally substituted with one to three substituents independently selected from the group consisting of an optionally substituted alkyl, an optionally substituted alkoxy, an optionally substituted alkylsulfanyl, an optionally substituted alkylamino, an optionally substituted dialkylamino, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, an optionally substituted haloalkyl, —C(O)NR₃₄R₃₅, —NR₃₆C(O)R₃₇, halo, —OR₃₆, cyano, nitro, haloalkoxy, —C(O)R₃₆, —NR₃₄R₃₅, —SR₃₆, —C(O)OR₃₆, —OC(O)R₃₆, —NR₃₆C(O)NR₃₄R₃₅, —NR₃₆C(N—R₃₈)NR₃₄R₃₅, —OC(O)NR₃₄R₃₅, —NR₃₆C(O)OR₃₇, —OP(O)(OR₃₆)₂, —SP(O)(OR₃₆)₂, —OS(O)₂(OR₃₆), —S(O)ₚR₃₆, or —S(O)ₚNR₃₄R₃₅.

In some embodiments, ring C of formula (XIV), (XIVA), or (XIVB) is optionally substituted with one or two substituents independently selected from the group consisting of an optionally substituted alkyl, an optionally substituted alkoxy, an optionally substituted alkylsulfanyl, an optionally substituted alkylamino, an optionally substituted dialkylamino, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, an optionally substituted haloalkyl, —C(O)NR₃₄R₃₅, —NR₃₆C(O)R₃₇, halo, —OR₃₆, cyano, nitro, haloalkoxy, —C(O)R₃₆, —NR₃₄R₃₅, —SR₃₆, —C(O)OR₃₆, —OC(O)R₃₆, —NR₃₆C(O)NR₃₄R₃₅, —NR₃₆C(N—R₃₈)NR₃₄R₃₅, —OC(O)NR₃₄R₃₅, —NR₃₆C(O)OR₃₇, —OP(O)(OR₃₆)₂, —SP(O)(OR₃₆)₂, —OS(O)₂(OR₃₆), —S(O)ₚR₃₆, —S(O)ₚNR₃₄R₃₅, ═O, ═S, ═NR₃₈.

In some embodiments, ring D of formula (XXIX), (XXIXA), (XXIXB), or (XVI) are optionally substituted with one to three substituents independently selected from the group consisting of an optionally substituted alkyl, an optionally substituted alkoxy, an optionally substituted alkylsulfanyl, an optionally substituted alkylamino, an optionally substituted dialkylamino, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, an optionally substituted haloalkyl, —C(O)NR₃₄R₃₅, —NR₃₆C(O)R₃₇, halo, —OR₃₆, cyano, nitro, haloalkoxy, —C(O)R₃₆, —NR₃₄R₃₅, —SR₃₆, —C(O)OR₃₆, —OC(O)R₃₆, —NR₃₆C(O)NR₃₄R₃₅, —NR₃₆C(N—R₃₈)NR₃₄R₃₅, —OC(O)NR₃₄R₃₅, —NR₃₆C(O)OR₃₇, —OP(O)(OR₃₆)₂, —SP(O)(OR₃₆)₂, —OS(O)₂(OR₃₆), —S(O)ₚR₃₆, or —S(O)ₚNR₃₄R₃₅. Preferably, ring D of formula (XXIX) or (XVI) is optionally substituted with one to five substituents independently selected from the group consisting of halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁; and R₃₅ and R₃₆ are independently selected from the group consisting of —H or a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁.

In some embodiments, ring E and/or ring F of formula (XVII) are optionally substituted with one to five substituents independently selected from the group consisting of an optionally substituted alkyl, an optionally substituted alkoxy, an optionally substituted alkylsulfanyl, an optionally substituted alkylamino, an optionally substituted dialkylamino, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, an optionally substituted haloalkyl, —C(O)NR₃₄R₃₅, —NR₃₆C(O)R₃₇, halo, —OR₃₆, cyano, nitro, haloalkoxy, —C(O)R₃₆, —NR₃₄R₃₅, —SR₃₆, —C(O)OR₃₆, —OC(O)R₃₆, —NR₃₆C(O)NR₃₄R₃₅, —NR₃₆C(N—R₃₈)NR₃₄R₃₅, —OC(O)NR₃₄R₃₅, —NR₃₆C(O)OR₃₇, —OP(O)(OR₃₆)₂, —SP(O)(OR₃₆)₂, —OS(O)₂(OR₃₆), —S(O)ₚR₃₆, or —S(O)₂NR₃₄R₃₅.

In some embodiments, ring E of formula (XVIIA) or (XVIIB) is optionally substituted with one to five substituents independently selected from the group consisting of an optionally substituted alkyl, an optionally substituted alkoxy, an optionally substituted alkylsulfanyl, an optionally substituted alkylamino, an optionally substituted dialkylamino, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, an optionally substituted haloalkyl, —C(O)NR₃₄R₃₅, —NR₃₆C(O)R₃₇, halo, —OR₃₆, cyano, nitro, haloalkoxy, —C(O)R₃₆, —NR₃₄R₃₅, —SR₃₆, —C(O)OR₃₆, —OC(O)R₃₆, —NR₃₆C(O)NR₃₄R₃₅, —NR₃₆C(N—R₃₈)NR₃₄R₃₅, —OC(O)NR₃₄R₃₅, —NR₃₆C(O)OR₃₇, —OP(O)(OR₃₆)₂, —S(O)(OR₃₆)₂, —OS(O)₂(OR₃₆), —S(O)ₚR₃₆, or —S(O)ₚNR₃₄R₃₅.

In some embodiments, R₃₁, for each occurrence, is independently selected from the group consisting of —H, an optionally substituted alkyl, an optionally substituted alkoxy, an optionally substituted alkylsulfanyl, an optionally substituted alkylamino, an optionally substituted dialkylamino, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, an optionally substituted haloalkyl, —C(O)NR₃₄R₃₅, —NR₃₆C(O)R₃₇, halo, —OR₃₆, cyano, nitro, haloalkoxy, —C(O)R₃₆) —NR₃₄R₃₅, —SR₃₆, —C(O)OR₃₆, —OC(O)R₃₆, —NR₃₆C(O)NR₃₄R₃₅, —NR₃₆C(N—R₃₈)NR₃₄R₃₅, —OC(O)NR₃₄R₃₅, —NR₃₆C(O)0R₃₇, —OP(O)(OR₃₆)₂, —SP(O)(OR₃₆)₂, —OS(O)₂(OR₃₆), —S(O)ₚR₃₆, or —S(O)ₚNR₃₄R₃₅. Preferably, R₃₁ is —H, a lower alkyl, a lower alkoxy, a lower alkyl sulfanyl, a amino, a lower alkyl amino, a lower dialkyl amino, hydroxy, —NHC(O)CH(R₅₇)NH₂, —OP(O)(OR₅₈)₂, halo, —SH, carboxy, —SP(O)(OR₅₈)₂, —NHC(O)R₅₈, —OS(O)₂(OR₅₈), lower alkoxycarbonyl, or lower alkoxy; preferably, R₃₁ is —H, amino, hydroxy, —NHC(O)CH(R₅₇)NH₂, or —OP(O)(OR₅₈)₂.

In some embodiments, in the compounds represented by formula (XVIIA) or (XVIIIB), R₆ is lower alkyl, lower alkoxy, lower alkylsulfanyl, —OH, —SH, —NH₂, halo, lower dialkyl amino, lower alkyl amino, nitro, cyano, pyridinyl, carboxy, lower alkoxycarbonyl, oxazolyl, —SP(O)(OR₅₈)₂, —OP(O)(OR₅₈)₂, —OC(O)R₅₈, —OS(O)₂(OR₅₈), tetrazolyl, 1-methyl-tetrazolyl, —NHC(O)R₅₈, or —NHC(O)CH(R₅₇)NH₂, wherein R₅₈ for each occurrence is independently, —H or a lower alkyl; and R₅₇ is H or an amino acid sidechain; and R₉ is —H, halo, —OH, —SH, —NH₂, carboxy, —OP(O)(OR₅₈)₂, —SP(O)(OR₅₈)₂, —NHC(O)R₅₈, —NHC(O)CH(R₅₇)NH₂, —OS(O)₂(OR₅₈), lower alkoxycarbonyl, or lower alkoxy.

In some embodiments, in the compounds represented by formula (XIXA) or (XIXB), R₃₂, R₅, and R₃₇ are each, independently, a lower alkyl, a lower alkoxy, or —OH.

In some embodiments, in the compounds represented by formula (XXA) or (XXB), R₃, R₅, and R₁₈ are each, independently, a lower alkyl, a lower alkoxy, or —OH.

In some embodiments, in the compounds represented by formula (XXIA) or (XXIB), R₃, R₃₂, R₅, and R₁₈ are each, independently, a lower alkyl, a lower alkoxy, or —OH.

In one embodiment, in formula (XVI), (XXIX), (XXIXA), or (XXIXB), X is NR₅₆; Ring D is unsubstituted; R₅₆ is —H or a lower alkyl; and R₃₅ and R₃₆ are —H.

In one embodiment, in formula (XVI), (XXIX), (XXIXA), or (XXIXB), X is O; Ring D is unsubstituted; and R₃₅ and R₃₆ are —H.

In some embodiments, in the compounds represented by formula (XIA), (XIIA), (XIIIA), (XIVA), (XVA), (XVIIA), (XVIIIA), (XIXA), (XXA), (XXIA), (XXVIIA), (XXVIIIA) or (XXIXA), R^{x} is R^{aa}, —C(O)YR^{z}, or —C(O)NH—R^{aa}. In one aspect, R^{x} is R^{aa}. In another aspect, R^{x} is —C(O)YR^{z}. R^{aa}, R^{z}, and Y are defined as for formula (IA).

In some embodiments, in the compounds represented by formula (XIA), (XIIA), (XIIIA), (XIVA), (XVA), (XVIIA), (XVIIIA), (XVIIIA), (XXA), (XXIA), (XXVIIA), (XXVIIIA) or (XXIXA), R^{x} is R^{aa} and R^{aa} is defined as for formula (IA). In one aspect, R^{aa} is glycine, serine, alanine, phenylalanine, leucine, or methionine.

In some embodiments, in the compounds represented by formula (XIA), (XIIA), (XIIIA), (XIVA), (XVA), (XVIIA), (XVIIIA), (XIXA), (XXA), (XXIA), (XXVIIA), (XXVIIIA) or (XXIXA), R^{x} is R^{aa} and R^{y} is —H, wherein R^{aa} is defined as for formula (XIA). In one aspect, R^{aa} is glycine, alanine, valine, leucine, isoleucine, serine, threonine, cysteine, methionine, phenylalanine, tyrosine, tryptophan, aspartic acid, asparagine, glutamic acid, glutamine, arginine, histidine, lysine, or proline. In another aspect, R^{aa} is glycine, serine, alanine, phenylalanine, leucine, or methionine.

In some embodiments, in the compounds represented by formula (XIA), (XIIA), (XIIIA), (XIVA), (XVA), (XVIIA), (XIXA), (XXA), (XXIA), (XXVIIA), (XXVIIIA) or (XXIXA), R^{x} is R^{aa} and R^{aa} is a D-amino acid residue or a D-amino acid residue analog. In one aspect, R^{aa} is D-alanine, D-valine, D-leucine, D-isoleucine, D-serine, D-threonine, D-cysteine, D-methionine, D-phenylalanine, D-tyrosine, D-tryptophan, D-aspartic acid, D-asparagine, D-glutamic acid, D-glutamine, D-arginine, D-histidine, D-lysine, or D-proline.

In some embodiments, in the compounds represented by formula (XIA), (XIIA), (XIIIA), (XIVA), (XVA), (XVIIA), (XVIIIA), (XIXA), (XXA), (XXIA), (XXVIIA), (XXVIIIA) or (XXIXA), R^{x} is R^{aa} and R^{aa} is an L-amino acid residue or an L-amino acid residue analog. In one aspect, R^{aa} is L-alanine, L-valine, L-leucine, L-isoleucine, L-serine, L-threonine, L-cysteine, L-methionine, L-phenylalanine, L-tyrosine, L-tryptophan, L-aspartic acid, L-asparagine, L-glutamic acid, L-glutamine, L-arginine, L-histidine, L-lysine, or L-proline.

In some embodiments, in the compounds represented by formula (XIA), (XIIA), (XIIIA), (XIVA), (XVA), (XVIIA), (XVIIIA), (XIXA), (XXA), (XXIA), (XXVIIA), (XXVIIIA) or (XXIXA), R^{x} is —C(O)YR^{z} and Y and R^{z} are defined as for formula (IA). In one aspect, Y is CH₂. In another aspect, Y is O. In another aspect, Y is NH. In one aspect, R^{z} is Y₁ and Y₁ is defined as for formula (XIA). In another aspect, R^{z} is Alk-NH₂. In another aspect, R^{z} is Alk-C(O)OH. In another aspect, R^{z} is Het. Alk and Het and defined as for formula X(IA).

In some embodiments, in the compounds represented by formula (XIA), (XIIA), (XIVA), (XVA), (XVIIA), (XVIIIA), (XIXA), (XXA), (XXIA), (XXVIIA), (XXVIIIA) or (XXIXA), m is 1, 2 or 3.

In some embodiments, in the compounds represented by formula (XIA), (XIIA), (XIIIA), (XIVA), (XVA), (XVIIA), (XVIIIA), (XIXA), (XXA), (XXIA), (XXVIIA), (XXVIIIA) or (XXIXA), Y₁ is PEG, HPMA copolymer-methacryloyl-Gly-Phe-Leu-Gly-ethylenediamine, or HPMA copolymer-methacryloyl-Gly-Phe-Leu-Gly-OH. In one aspect, Y₁ is PEG.

In some embodiments, in the compounds represented by formula (XIA), (XIIA), (XIIIA), (XIVA), (XVA), (XVIIA), (XVIIIA), (XIXA), (XXA), (XXIA), (XXVIIA), (XXVIIIA) or (XXIXA), R^{y} is —H.

In some embodiments, in the compounds represented by formula (XIA), (XIIA), (XIIIA), (XIVA), (XVA), (XVIIA), (XIXA), (XXA), (XXIA), (XXVIIA), (XXVIIIA) or (XXIXA), R^{y} is a lower alkyl.

In some embodiments, in the compounds represented by formula (XIA), (XIIA), (XIIIA), (XIVA), (XVA), (XVIIA), (XVIIA), (XIXA), (XXA), (XXIA), (XXVIIA), (XXVIIIA) or (XXIXA)), Y₁ has a molecular weight greater than 20,000 daltons. In one aspect, Y₁ has a molecular weight of less than 40,000 daltons, but greater than 25,000 daltons.

In some embodiments, in the compounds represented by formula (XIA), (XIIIA), (XIVA), (XVA), (XVIIA), (XVIIIA), (XIXA), (XXA), (XXIA), (XXVIIA), (XXVIIIA) or (XXIXA), Alk is an optionally substituted lower alkylene.

In some embodiments, in the compounds represented by formula (XIA), (XIIA), (XIIIA), (XIVA), (XVA), (XVIIA), (XVIIIA), (XIXA), (XXA), (XXIA), (XXVIIA), (XXVIIIA) or (XXIXA), Het is an optionally substituted lower heteroalkyl.

In some embodiments, in the compounds represented by formula (XIIIA), R₃, R₃₂, and R₅ are each methoxy. In one aspect, R^{x} is R^{aa}. In another aspect, R^{x} is (R^{aa})ₘ. In another aspect, R^{x} is —R^{aa}—C(O)(CH₂)ₙC(O)OH. In another aspect, R^{x} is —C(O)(CH₂)ₙC(O)OH. In another aspect, R^{x} is —C(O)YR^{z}. In another aspect, R^{x} is —C(O)NH—R^{aa}. In another aspect, R^{x} is —(R^{aa})_{q}C(O)(Y₁). R^{aa}, Y, R^{z}, Y₁, m, n, and q are defined as for formula (XIA).

In some embodiments, in the compounds represented by formula (XIIIA), R₃, R₃₂, and R₅ are each methoxy. In one aspect, R^{x} is R^{aa} and R^{w} is alkoxy. In another aspect, R^{x} is R^{aa} and R^{y} is —H. In another aspect, R^{x} is R^{aa}, R^{w} is alkoxy, and R^{y} is —H. In another aspect, R^{x} is R^{aa}, R^{w} is alkoxy, and R^{y} is —H. In another aspect, R^{x} is R^{aa}, R^{w} is methoxy, and R^{y} is —H. R^{aa} is defined as for formula (XIA).

In some embodiments, in the compounds represented by formula (XIIIB), R₃, R₃₂, and R₅ are each methoxy; and R^{w} is alkoxy. In one aspect, R^{w} is methoxy.

In some embodiments, in the compounds represented by formula (XIA or B), (XIIA or B), (XIIIA or B), (XIVA or B), (XVA or B), (XVIIA or B), (XVIIA or B), (XIXA or B), (XXA or B), (XXIA or B), (XXVIIA or B), (XXVIIIA or B), or (XXIXA or B), R^{w} is alkoxy. In one aspect, R^{w} is methoxy.

In another embodiment, the invention relates to compounds selected from the group consisting of:
1-(3,4,5-trimethoxy-phenyl)-5-(4-bromo-phenyl)-1H-[1,2,3]triazole;
1-(2-hydroxy-4-methoxy-5-ethyl-phenyl)-5-(naphthylen-2-yl)-1H-[1,2,3]triazole;
1-(3,4,5-trimethoxy-phenyl)-5-(4-methoxy-phenyl)-1H-[1,2,3]triazole;
1-(2-hydroxy-4-methoxy-5-ethyl-phenyl)-5-(4-iodo-phenyl)-1H-[1,2,3]triazole;
1-(3,4,5-trimethoxy-phenyl)-5-[4-(N,N-dimethylamino)-phenyl]-1H-[1,2,3]triazole;
1-(2-hydroxy-4-methoxy-5-ethyl-phenyl)-5-(4-bromo-phenyl)-1H-[1,2,3]triazole;
1-(2-hydroxy-4-methoxy-5-(2,3-dihydro-benzo[1,4]dioxin-6-yl)-5-(4-bromo-phenyl)-1H-[1,2,3]triazole;
1-(3,4,5-hydroxy-phenyl)-5-(4-hyoxy-phenyl)-1H-[1,2,3]triazole;
1-(3,4,5-trimethoxy-phenyl)-5-(4-iodo-phenyl)-1H-[1,2,3]triazole;
1-(3,4,5-trimethoxy-phenyl)-5-(3-fluoro-4-methoxy-phenyl)-1H-[1,2,3]triazole;
1-(3,4,5-trimethoxy-phenyl)-5-(4-nitro-phenyl)-1H-[1,2,3]triazole;
1-(3,4,5-trimethoxy-phenyl)-5-(4-amino-phenyl)-1H-[1,2,3]triazole;
1-(3,4,5-trimethoxy-phenyl)-5-(4′-methoxy-biphenyl-4-yl)-1H-[1,2,3]triazole;
1-(3,4,5-trimethoxy-phenyl)-5-[4-(pyridin-3-yl)-phenyl]-1H-[1,2,3]triazole;
1-(3,4,5-trimethoxy-phenyl)-5-[4-(pyridin-4-yl)-phenyl]-1H-[1,2,3]triazole;
1-(3,4,5-trimethoxy-phenyl)-5-[4-(pyridin-2-yl)-phenyl]-1H-[1,2,3]triazole;
1-(3,4,5-trimethoxy-phenyl)-5-(quinolin-7-yl)-1H-[1,2,3]triazole;
1-(3,4,5-trimethoxy-phenyl)-5-(pyridine-4-yl)-1H-[1,2,3]triazole;
1-(3,4,5-trimethoxy-phenyl)-5-(isoquinolin-7-yl)-1H-[1,2,3]triazole;
1-(3,4,5-trimethoxy-phenyl)-5-(1-methyl-1H-indol-5-yl)-1H-[1,2,3]triazole;
1-(benzo[1,3]dioxol-5-yl)-5-(4-methoxy-phenyl)-1H-[1,2,3]triazole;
1-(1-ethyl-1H-indol-6-yl)-5-(4-methoxy-phenyl)-1H-[1,2,3]triazole;
1-(3,4,5-trimethoxy-phenyl)-5-(4-carboxy-phenyl)-1H-[1,2,3]triazole;
1-(3,4,5-trimethoxy-phenyl)-5-(4-carbomethoxy-phenyl)-1H-[1,2,3]triazole;
1-(3,4,5-trimethoxy-phenyl)-5-[4-(oxazol-2-yl)-phenyl]-1H-[1,2,3]triazole;
1-(3,4,5-triethyl-phenyl)-5-(4-methoxy-phenyl)-1H-[1,2,3]triazole;
1-(3,4,5-triethyl-phenyl)-5-(4-iodo-phenyl)-1H-[1,2,3]triazole;
1-(3,4,5-triethyl-phenyl)-5-(3-fluoro-4-methoxy-phenyl)-1H-[1,2,3]triazole;
1-(3,4,5-triethyl-phenyl)-5-(4-nitro-phenyl)-1H -[1,2,3]triazole;
1-(3,4,5-triethyl-phenyl)-5-[4-(N,N-dimethylamino)-phenyl]-1H-[1,2,3]triazole;
1-(3,4,5-trimethyl-phenyl)-5-(4-methoxy-phenyl)-1H-[1,2,3]triazole;
1-(3,4,5-triethyl-phenyl)-5-[4-(pyridine-3-yl)-phenyl]-1H-[1,2,3]triazole;
1-(3,4,5-triethyl-phenyl)-5-[4-(pyridine-4-yl)-phenyl]-1H-[1,2,3]triazole;
1-(3,4,5-triethyl-phenyl)-5-[4-(pyridine-2-yl)-phenyl]-1H-[1,2,3]triazole;
1-(3,4,5-triethyl-phenyl)-5-(quinolin-7-yl)-1H-[1,2,3]triazole;
1-(3,4,5-triethyl-phenyl)-5-(pyridine-4-yl)-1H-[1,2,3]triazole;
1-(3,4,5-triethyl-phenyl)-5-(isoquinolin-7-yl)-1H-[1,2,3]triazole;
1-(3,4,5-triethyl-phenyl)-5-(1H-indol-5-yl)-1H-[1,2,3]triazole;
1-(benzo[1,3]dioxol-5-yl)-5-(4-methoxy-phenyl)-1H-[1,2,3]triazole;
1-(1-isopropyl-1H-indol-6-yl)-5-(4-methoxy-phenyl)-1H-[1,2,3]triazole;
1-(2,3,4-trimethoxy-phenyl)-5-(4-methoxy-phenyl)-1H-[1,2,3]triazole;
1-(3,4,5-trimethoxy-phenyl)-5-(3-hydroxy-4-methoxy-phenyl)-1H-[1,2,3]triazole;
O-ethyl-O-{2-methoxy-5-[1-(3,4,5-trimethoxy-phenyl)-1H-[1,2,3]triazol-5-yl]-phenyl}-phosphate;
1-(2-hydroxy-4-methoxy-5-ethyl-phenyl)-5-(4-methoxy-phenyl)-1H-[1,2,3]triazole;
1-(3,4,5-trimethoxy-phenyl)-5-(4-isopropyl-phenyl)-1H-[1,2,3]triazole;
1-(3,4,5-trimethoxy-phenyl)-5-(2,3-dihydro-benzo[1,4]dioxine-6-yl)-1H-[1,2,3]triazole;
1-(3,4,5-trimethoxy-phenyl)-5-(4-ethyl-phenyl)-1H-[1,2,3]triazole;
1-(3,4,5-trimethoxy-phenyl)-5-(5-methoxy-pyridine-2-yl)-1H-[1,2,3]triazole;
1-(4,5,6-trimethoxy-pyridin-2-yl)-5-(4-methoxy-phenyl)-1H-[1,2,3]triazole;
1-(3,5-dimethoxy-4-carbomethoxy-phenyl)-5-(4-methoxy-phenyl)-1H-[1,2,3]triazole;
1-(3,5-diacetoxy-phenyl)-5-(4-methoxy-phenyl)-1H-[1,2,3]triazole;
1-(3,4,5-trimethoxy-phenyl)-5-(2-methoxy-pyridine-5-yl)-1H-[1,2,3]triazole;
1-(1-methyl-5-methoxy-1H-indol-7-yl)-5-(4-methoxy-phenyl)-1H-[1,2,3]triazole;
1-(1-methyl-1H-indol-7-yl)-5-(4-methoxy-phenyl)-1H-[1,2,3]triazole;
1-(Benzo[1,3]dioxol-4-yl)-5-(4-methoxy-phenyl)-1H-[1,2,3]triazole;
1-(3,4,5-trimethoxy-phenyl)-5-(2-hydroxy-4-methoxy-phenyl)-1H-[1,2,3]triazole;
O-ethyl-O-{5-methoxy-2-[1-(3,4,5-trimethoxy-phenyl)-1H-[1,2,3]triazol-5-yl]-phenyl}-phosphate;
1-(3,4,5-trimethoxy-phenyl)-5-(pyridazin-4-yl)-1H-[1,2,3]triazole;
1-(3,4,5-trimethoxy-phenyl)-5-(pyrimidin-5-yl)-1H-[1,2,3]triazole;
1-(3,4,5-trimethoxy-phenyl)-5-(pyridin-2-yl)-1H-[1,2,3]triazole, hydrochloric acid salt;
1-(3,4,5-trimethoxy-phenyl)-5-(2-mercapto-4-methoxy-phenyl)-1H-[1,2,3]triazole;
S-{2-methoxy-5-[1-(3,4,5-trimethoxy-phenyl)-1H-[1,2,3]triazol-5-yl-phenyl}-thiophosphate, disodium salt;
1-(3,4,5-trimethoxy-phenyl)-5-(3-acetamido-4-methoxy-phenyl)-1H-[1,2,3]triazole;
1-(3,4,5-trimethoxy-phenyl)-5-(3-amino-4-methoxy-phenyl)-1H-[1,2,3]triazole, hydrochloric acid salt;
1-(3,4,5-trimethoxy-phenyl)-5-(2-hydroxy-4-methoxy-phenyl)-1H-[1,2,3]triazole;
1-(3,4,5-trimethoxy-phenyl)-5-(2-methoxy-pyridin-5-yl)-1H-[1,2,3]triazole;
1-(3,4,5-trimethoxy-phenyl)-5-(5-methoxy-pyridin-2-yl)-1H-[1,2,3]triazole;
1-(3,4,5-trimethoxy-phenyl)-5-(3-carboxy-4-methoxy-phenyl)-1H-[1,2,3]triazole, sodium salt;
1-(3,4,5-trimethoxy-phenyl)-5-(3-methoxycarbonyl-4-methoxy-phenyl)-1H-[1,2,3]triazole;
1-(3,4,5-trimethoxy-phenyl)-5-(3-sulfooxy-4-methoxy-phenyl)-1H-[1,2,3]triazole, sodium salt;
1-(3,4,5-trimethoxy-phenyl)-5-(2-amino-4-methoxy-phenyl)-1 H-[1,2,3]triazole;
1-(3,4,5-trimethoxy-phenyl)-5-(3-phosphonooxy-4,5-dimethoxy-phenyl)-1H-[1,2,3]triazole, disodium salt;
1-(3,4,5-trimethoxy-phenyl)-5-(2-phosphonooxy-4-methoxy-phenyl)-1H-[1,2,3]triazole, disodium salt;
1-(3,4,5-trimethoxy-phenyl)-5-(4-methylsulfanyl-phenyl)-1H-[1,2,3]triazole;
1-(3,4,5-trimethoxy-phenyl)-5-(3-phosphonooxy-4-methylsulfanyl-phenyl)-1H-[1,2,3]triazole, disodium salt;
1-(3,4,5-trimethoxy-phenyl)-5-(3-amino-4-methylsulfanyl-phenyl)-1H-[1,2,3]triazole;
1-(3,4,5-trimethoxy-phenyl)-5-(2,3-dihydro-benzofuran-6-yl)-1H-[1,2,3]triazole;
1-(3,4,5-trimethoxy-phenyl)-5-(4-hydroxy-phenyl)-1H-[1,2,3]triazole, sodium salt;
1-(3,4,5-trimethoxy-phenyl)-5-(4-phosphonooxy-phenyl)-1H-[1,2,3]triazole,disodium salt;
1-(3,4,5-trimethoxy-phenyl)-5-[4-(tetrazol-5-yl)-phenyl]-1H-[1,2,3]triazole;
1-(3,4,5-trimethoxy-phenyl)-5-[4-(1-methyl-tetrazol-5-yl)-phenyl]-1H-[1,2,3]triazole;
1-(3,4,5-trimethoxy-phenyl)-5-(1-methyl-1H-indol-5-yl)-1 H-[1,2,3]triazole;
1-(7-methoxy-benzo[1,3]dioxol-5-yl)-5-(pyridazin-4-yl)-1H-[1,2,3]triazole;
1-(7-methoxy-benzo[1,3]dioxol-5-yl)-5-(pyrimidin-5-yl)-1H-[1,2,3]triazole;
1-(7-methoxy-benzo[1,3]dioxol-5-yl)-5-(pyridine-3-yl)-1H-[1,2,3]triazole, hydrochloric acid salt;
1-(7-methoxy-benzo[1,3]dioxol-5-yl)-5-(3-mercapto-4-methoxy-phenyl)-1H-[1,2,3]triazole;
S-{2-methoxy-5-[1-(7-methoxy-benzo[1,3]dioxol-5-yl)-1H-[1,2,3]triazol-5-yl]-phenyl}-thiophosphate, disodium salt;
1-(7-methoxy-benzo[1,3]dioxol-5-yl)-5-(3-acetamindo-4-methoxy-phenyl)-1H-[1,2,3]triazole;
1-(7-methoxy-benzo[1,3]dioxol-5-yl)-5-(3-amino-4-methoxy-phenyl)-1H-[1,2,3]triazole, hydrochloric acid salt;
1-(7-methoxy-benzo[1,3]dioxol-5-yl)-5-(2-hydroxy-4-methoxy-phenyl)-1H-[1,2,3]triazole;
1-(7-methoxy-benzo[1,3]dioxol-5-yl)-5-(2-methoxy-pyridin-5-yl)-1H-[1,2,3]triazole;
1-(7-methoxy-benzo[1,3]dioxol-5-yl)-5-(5-methoxy-pyridin-2-yl)-1H-[1,2,3]triazole;
1-(7-methoxy-benzo[1,3]dioxol-5-yl)-5-(3-carboxy-4-methoxy-phenyl)-1H-[1,2,3]triazole;
1-(7-methoxy-benzo[1,3]dioxol-5-yl)-5-(3-methoxycarbonyl-4-methoxy-phenyl)-1H-[1,2,3]triazole;
1-(7-methoxy-benzo[1,3]dioxol-5-yl)-5-(3-sulfooxy-4-methoxy-phenyl)-1H-[1,2,3]triazole, sodium salt;
1-(7-methoxy-benzo[1,3]dioxol-5-yl)-5-(2-amino-4-methoxy-phenyl)-1H-[1,2,3]triazole;
1-(7-methoxy-benzo[1,3]dioxol-5-yl)-5-(3-phosphonyl-4,5-dimethoxy-phenyl)-1H-[1,2,3]triazole, disodium salt;
1-(7-methoxy-benzo[1,3]dioxol-5-yl)-5-(2-phosphonyl-4-methoxy-phenyl)-1H-[1,2,3]triazole, sodium salt;
1-(7-methoxy-benzo[1,3]dioxol-5-yl)-5-(4-methylsulfanyl-phenyl)-1H-[1,2,3]triazole;
1-(7-methoxy-benzo[1,3]dioxol-5-yl)-5-(3-phosphonyl-4-methylsulfanyl-phenyl)-1H-[1,2,3]triazole, disodium salt;
1-(7-methoxy-benzo[1,3]dioxol-5-yl)-5-(3-amino-4-methylsulfanyl-phenyl)-1H-[1,2,3]triazole;
1-(7-methoxy-benzo[1,3]dioxol-5-yl)-5-(2,3-dihydro-benzofuran-6-yl)-1H-[1,2,3]triazole;
1-(7-methoxy-benzo[1,3]dioxol-5-yl)-5-(4-hydroxy-phenyl)-1H-[1,2,3]triazole, sodium salt;
1-(7-methoxy-benzo[1,3]dioxol-5-yl)-5-(4-phosphonooxy-phenyl)-1H-[1,2,3]triazole, disodium salt;
1-(7-methoxy-benzo[1,3]dioxol-5-yl)-5-[1H-tetrazol-5-yl)-phenyl]-1H-[1,2,3]triazole;
1-(7-methoxy-benzo[1,3]dioxol-5-yl)-5-[1-methyl-1H-tetrazol-5-yl)-phenyl]-1H-[1,2,3]triazole;
1-(7-methoxy-benzo[1,3]dioxol-5-yl)-5-(1-methyl-1H-indol-5-yl)-1H-[1,2,3]triazole;
1-(1-methyl-1H-indol-5-yl)-5-(3,4,5-trimethoxy-phenyl)-1H-[1,2,3]triazole;
1-(3-phosphonooxy-4-methoxy-phenyl)-5-(3,4,5-trimethoxy-phenyl)-1H-[1,2,3]triazole, disodium salt;
1-[4-(N,N-dimethylamino)-phenyl]-5-(3,4,5-trimethoxy-phenyl)-1H-[1,2,3]triazole;
1-(3-amino-4-methoxy-phenyl)-5-(3,4,5-trimethoxy-phenyl)-1H-[1,2,3]triazole, hydrochloric acid salt;
2-hydroxy-1-{2-methoxy-5-[5-(3,4,5-trimethoxy-phenyl)-[1,2,3]triazol-1-yl]-phenylcarbamoyl}-ethyl-ammonium chloride;
1-(2,4,5-trimethoxy-phenyl)-5-(4-methoxy-phenyl)-1H-[1,2,3]triazole;
1-(2,4,5-trimethoxy-phenyl)-5-(4-methyl-phenyl)-1H-[1,2,3]triazole;
1-(2,4,5-trimethoxy-phenyl)-5-(4-ethoxy-phenyl)-1H-[1,2,3]triazole;
1-(2,4,5-trimethoxy-phenyl)-5-(4-ethyl-phenyl)-1H-[1,2,3]triazole;
1-(2,4,5-trimethoxy-phenyl)-5-(4-propoxy-phenyl)-1H-[1,2,3]triazole;
1-(2,4,5-trimethoxy-phenyl)-5-(4-propyl-phenyl)-1H-[1,2,3]triazole;
1-(2,4,5-trimethoxy-phenyl)-5-(4-butoxy-phenyl)-1H-[1,2,3]triazole;
1-(2,4,5-trimethoxy-phenyl)-5-(4-butyl-phenyl)-1H-[1,2,3]triazole;
1-(2,4,5-trimethoxy-phenyl)-5-(4-bromo-phenyl)-1H-[1,2,3]triazole;
1-(2,4,5-trimethoxy-phenyl)-5-(4-chloro-phenyl)-1H-[1,2,3]triazole;
1-(2,4,5-trimethoxy-phenyl)-5-(4-fluoro-phenyl)-1H-[1,2,3]triazole;
1-(2,4,5-trimethoxy-phenyl)-5-(4-nitro-phenyl)-1H-[1,2,3]triazole;
1-(2,4,5-trimethoxy-phenyl)-5-[4-(N,N-dimethylamino)-phenyl]-1H-[1,2,3]triazole;
1-(2,4,5-trimethoxy-phenyl)-5-(3,4-dimethoxy-phenyl)-1H-[1,2,3]triazole;
1-(2,4,5-trimethoxy-phenyl)-5-(3-hydroxy-4-methoxy-phenyl)-1H-[1,2,3]triazole;
1-(2,4,5-trimethoxy-phenyl)-5-(3,4,5-trimethoxy-phenyl)-1H-[1,2,3]triazole;
1-(2,3,5-trimethoxy-phenyl)-5-(4-methoxy-phenyl)-1H-[1,2,3]triazole;
1-(2,3,5-trimethoxy-phenyl)-5-(4-methyl-phenyl)-1H-[1,2,3]triazole;
1-(2,3,5-trimethoxy-phenyl)-5-(4-ethoxy-phenyl)-1H-[1,2,3]triazole;
1-(2,3,5-trimethoxy-phenyl)-5-(4-ethyl-phenyl)-1H-[1,2,3]triazole;
1-(2,3,5-trimethoxy-phenyl)-5-(4-propoxy-phenyl)-1H-[1,2,3]triazole;
1-(2,3,5-trimethoxy-phenyl)-5-(4-propyl-phenyl)-1H-[1,2,3]triazole;
1-(2,3,5-trimethoxy-phenyl)-5-(4-butoxy-phenyl)-1H-[1,2,3]triazole;
1-(2,3,5-trimethoxy-phenyl)-5-(4-butyl-phenyl)-1H-[1,2,3]triazole;
1-(2,3,5-trimethoxy-phenyl)-5-(4-bromo-phenyl)-1H-[1,2,3]triazole;
1-(2,3,5-trimethoxy-phenyl)-5-(4-chloro-phenyl)-1H-[1,2,3]triazole;
1-(2,3,5-trimethoxy-phenyl)-5-(4-fluoro-phenyl)-1H-[1,2,3]triazole;
1-(2,3,5-trimethoxy-phenyl)-5-(4-nitro-phenyl)-1H-[1,2,3]triazole;
1-(2,3,5-trimethoxy-phenyl)-5-[4-(N,N-dimethylamino)-phenyl]-1H-[1,2,3]triazole;
1-(2,3,5-trimethoxy-phenyl)-5-(3,4-dimethoxy-phenyl)-1H-[1,2,3]triazole;
1-(2,3,5-trimethoxy-phenyl)-5-(3-hydroxy-4-methoxy-phenyl)-1H-[1,2,3]triazole;
1-(2,3,5-trimethoxy-phenyl)-5-(3,4,5-trimethoxy-phenyl)-1H-[1,2,3]triazole;
1-(4-methoxy-phenyl)-5-(2,3,4,5-tetramethoxy-phenyl)-1H-[1,2,3]triazole;
1-(4-methyl-phenyl)-5-(2,3,4,5-tetramethoxy-phenyl)-1H-[1,2,3]triazole;
1-(4-ethoxy-phenyl)-5-(2,3,4,5-tetramethoxy-phenyl)-1H-[1,2,3]triazole;
1-(4-ethyl-phenyl)-5-(2,3,4,5-tetramethoxy-phenyl)-1H-[1,2,3]triazole;
1-(4-propoxy-phenyl)-5-(2,3,4,5-tetramethoxy-phenyl)-1H-[1,2,3]triazole;
1-(4-propyl-phenyl)-5-(2,3,4,5-tetramethoxy-phenyl)-1H-[1,2,3]triazole;
1-(4-butoxy-phenyl)-5-(2,3,4,5-tetramethoxy-phenyl)-1H-[1,2,3]triazole;
1-(4-butyl-phenyl)-5-(2,3,4,5-tetramethoxy-phenyl)-1H-[1,2,3]triazole;
1-(4-bromo-phenyl)-5-(2,3,4,5-tetramethoxy-phenyl)-1H-[1,2,3]triazole;
1-(4-chloro-phenyl)-5-(2,3,4,5-tetramethoxy-phenyl)-1H-[1,2,3]triazole;
1-(4-fluoro-phenyl)-5-(2,3,4,5-tetramethoxy-phenyl)-1H-[1,2,3]triazole;
1-(4-nitro-phenyl)-5-(2,3,4,5-tetramethoxy-phenyl)-1H-[1,2,3]triazole;
1-[4-(N,N-dimethylamino)-phenyl]-5-(2,3,4,5-tetramethoxy-phenyl)-1H-[1,2,3]triazole;
1-(3,4-dimethoxy-phenyl)-5-(2,3,4,5-tetramethoxy-phenyl)-1H-[1,2,3]triazole;
1-(3-hydroxy-4-methoxy-phenyl)-5-(2,3,4,5-tetramethoxy-phenyl)-1H-[1,2,3]triazole;
1-(3,4,5-trimethoxy-phenyl)-5-(2,3,4,5-tetramethoxy-phenyl)-1H-[1,2,3]triazole;
1-(3,4-trimethoxy-phenyl)-5-(2,3-dihydro-benzo[1,4]dioxin-6-yl)-1H-[1,2,3]triazole;
1-(2-hydroxy-4-methoxy-5-ethyl-phenyl)-5-(3,4-dimethyl-phenyl)-1H-[1,2,3]triazole;
1-(2-hydroxy-4-methoxy-5-ethyl-phenyl)-5-(4-chloro-phenyl)-1H-[1,2,3]triazole;
1-(2-hydroxy-4-methoxy-5-propyl-phenyl)-5-phenyl-1H-[1,2,3]triazole;
1-(2-hydroxy-4-methoxy-5-ethyl-phenyl)-5-(4-methyl-phenyl)-1H-[1,2,3]triazole;
1-(2-hydroxy-4-methoxy-5-ethyl-phenyl)-5-(4-amino-phenyl)-1H-[1,2,3]triazole;
1-(2-hydroxy-4-methoxy-5-ethyl-phenyl)-5-(4-trifluoromethyl-phenyl)-1H-[1,2,3]triazole;
1-(2-hydroxy-4-methoxy-5-ethyl-phenyl)-5-(4-methoxy-phenyl)-1H-[1,2,3]triazole;
1-(4-bromo-phenyl)-5-(3,4,5-trimethoxy-phenyl)-1H-[1,2,3]triazole;

or pharmaceutically acceptable salts, solvates, clathrates, or prodrugs thereof.

In another embodiment, the invention relates to compounds selected from the group consisting of:
2-amino-N-(2-methoxy-5-(1-(3,4,5-trimethoxyphenyl)-1H-1,2,3-triazol-5-yl)phenyl)acetamide hydrochloride;
2-amino-3-hydroxy-N-(2-methoxy-5-(1-(3,4,5-trimethoxyphenyl)-1H-1,2,3-triazol-5-yl)phenyl)propanamide hydrochloride;
2-amino-N-(2-methoxy-5-(1-(3,4,5-trimethoxyphenyl)-1H-1,2,3-triazol-5-yl)phenyl)propanamide;
2-amino-N-(2-methoxy-5-(1-(3,4,5-trimethoxyphenyl)-1H-1,2,3-triazol-5-yl)phenyl)-4-(methylthio)butanamide hydrochloride;
2-amino-N-(2-methoxy-5-(1-(3,4,5-trimethoxyphenyl)-1H-1,2,3-triazol-5-yl)phenyl)butanamide hydrochloride;
2-amino-N-(2-methoxy-5-(1-(3,4,5-trimethoxyphenyl)-1H-1,2,3-triazol-5-yl)phenyl)-3-phenylpropanamide hydrochloride;
2-amino-N-(2-methoxy-5-(1-(3,4,5-trimethoxyphenyl)-1H-1,2,3-triazol-5-yl)phenyl)-4-methylpentanamide hydrochloride;
2-amino-N-(2-methoxy-5-(1-(3,4,5-trimethoxyphenyl)-1H-1,2,3-triazol-5-yl)phenyl)-3-(4-methoxyphenyl)propanamide hydrochloride;
1-(2-methoxy-5-(1-(3,4,5-trimethoxyphenyl)-1H-1,2,3-triazol-5-yl)phenylamino)-3-methyl-1-oxobutan-2-aminium chloride;
1-(2-methoxy-5-(1-(3,4,5-trimethoxyphenyl)-1H-1,2,3-triazol-5-yl)phenylamino)-3-methyl-1-oxopentan-2-aminium chloride;
3-hydroxy-1-(2-methoxy-5-(1-(3,4,5-trimethoxyphenyl)-1H-1,2,3-triazol-5-yl)phenylamino)-1-oxobutan-2-aminium chloride;
3-(4-hydroxyphenyl)-1-(2-methoxy-5-(1-(3,4,5-trimethoxyphenyl)-1H-1,2,3-triazol-5-yl)phenylamino)-1-oxopropan-2-aminium chloride;
2-(2-methoxy-5-(1-(3,4,5-trimethoxyphenyl)-1H-1,2,3-triazol-5-yl)phenylamino)-2-oxo-1-phenylethanaminium chloride;
3-(1H-indol-2-yl)-1-(2-methoxy-5-(1-(3,4,5-trimethoxyphenyl)-1H-1,2,3-triazol-5-yl)phenylamino)-1-oxopropan-2-aminium chloride;
3-(benzofuran-2-yl)-1-(2-methoxy-5-(1-(3,4,5-trimethoxyphenyl)-1H-1,2,3-triazol-5-yl)phenylamino)-1-oxopropan-2-aminium chloride;
3-carboxy-1-(2-methoxy-5-(1-(3,4,5-trimethoxyphenyl)-1H-1,2,3-triazol-5-yl)phenylamino)-1-oxopropan-2-aminium chloride;
4-carboxy-1-(2-methoxy-5-(1-(3,4,5-trimethoxyphenyl)-1H-1,2,3-triazol-5-yl)phenylamino)-1-oxobutan-2-aminium chloride;
5-amino-1-(2-methoxy-5-(1-(3,4,5-trimethoxyphenyl)-1H-1,2,3-triazol-5-yl)phenylamino)-1,5-dioxopentan-2-aminium chloride;
4-amino-1-(2-methoxy-5-(1-(3,4,5-trimethoxyphenyl)-1H-1,2,3-triazol-5-yl)phenylamino)-1,4-dioxobutan-2-aminium chloride;
3-(1H-imidazol-5-yl)-1-(2-methoxy-5-(1-(3,4,5-trimethoxyphenyl)-1H-1,2,3-triazol-5-yl)phenylamino)-1-oxopropan-2-aminium chloride;
6-amino-1-(2-methoxy-5-(1-(3,4,5-trimethoxyphenyl)-1H-1,2,3-triazol-5-yl)phenylamino)-1-oxohexan-2-aminium chloride;
5-guanidino-1-(2-methoxy-5-(1-(3,4,5-trimethoxyphenyl)-1H-1,2,3-triazol-5-yl)phenylamino)-1-oxopentan-2-aminium chloride;
4-(2-methoxy-5-(1-(3,4,5-trimethoxyphenyl)-1H-1,2,3-triazol-5-yl)phenylamino)-4-oxobutanoic acid;
5-(2-methoxy-5-(1-(3,4,5-trimethoxyphenyl)-1H-1,2,3-triazol-5-yl)phenylamino)-5-oxopentanoic acid;
3-(2-methoxy-5-(1-(3,4,5-trimethoxyphenyl)-1H-1,2,3-triazol-5-yl)phenylamino)-3-oxopropan-1-aminium chloride;
N-(2-methoxy-5-(1-(3,4,5-trimethoxyphenyl)-1H-1,2,3-triazol-5-yl)phenyl)-3-(2-methoxyethoxy)propanamide;
3-(2-PEG)-N-(2-methoxy-5-(1-(3,4,5-trimethoxyphenyl)-1H-1,2,3-triazol-5-yl)phenyl)butyramide;
N-(2-methoxy-5-(1-(3,4,5-trimethoxyphenyl)-1H-1,2,3-triazol-5-yl)phenyl)-3-(2-(methylamino)ethylamino)propanamide;
3-PEG-N-(2-methoxy-5-(1-(3,4,5-trimethoxyphenyl)-1H-1,2,3-triazol-5-yl)phenylamino)-2-oxoethypbutyramide;
4-(2-(2-methoxy-5-(1-(3,4,5-trimethoxyphenyl)-1H-1,2,3-triazol-5-yl)phenylamino)-2-oxoethylamino)-4-oxobutanoic acid;
2-methoxyethyl 2-methoxy-5-(1-(3,4,5-trimethoxyphenyl)-1H-1,2,3-triazol-5-yl)phenylcarbamate;
PEG-2-methoxy-5-(1-(3,4,5-trimethoxyphenyl)-1H-1,2,3-triazol-5-yl)phenylcarbamate;
3-amino-4-(2-((R)-5-guanidino-1-(2-methoxy-5-(1-(3,4,5-trimethoxyphenyl)-1H-1,2,3-triazol-5-yl)phenylamino)-1-oxopentan-2-ylamino)-2-oxoethylamino)-4-oxobutanoic acid; and
2-amino-N-(2-methoxy-5-(1-(3,4,5-trimethoxyphenyl)-1H-1,2,3-triazol-5-yl)phenyl)propanamide hydrochloride;

or pharmaceutically acceptable salts, solvates, clathrates, or prodrugs thereof.

In another embodiment, the invention relates to compounds selected from the group consisting of:
4-(3,4,5-trimethoxy-phenyl)-5-(4-bromo-phenyl)-1H-[1,2,3]triazole;
4-ethyl-5-methoxy-2-(5-(naphthalen-2-yl)-1H-1,2,3-triazol-4-yl)phenol;
5-(4-methoxyphenyl)-4-(3,4,5-trimethoxyphenyl)-1H-1,2,3-triazole;
4-ethyl-2-(5-(4-iodophenyl)-1H-1,2,3-triazol-4-yl)-5-methoxyphenol;
N,N-dimethyl-4-(4-(3,4,5-trimethoxyphenyl)-1H-1,2,3-triazol-5-yl)aniline;
2-(5-(4-bromophenyl)-1H-1,2,3-triazol-4-yl)-4-ethyl-5-methoxyphenol;
2-(5-(2,3-dihydrobenzo[b][1,4]dioxin-6-yl)-1H-1,2,3-triazol-4-yl)-5-methoxy-4-propylphenol;
5-(5-(4-hydroxyphenyl)-1H-1,2,3-triazol-4-yl)benzene-1,2,3-triol;
5-(4-iodophenyl)-4-(3,4,5-trimethoxyphenyl)-1H-1,2,3-triazole;
5-(3-fluoro-4-methoxyphenyl)-4-(3,4,5-trimethoxyphenyl)-1H-1,2,3-triazole;
5-(4-nitrophenyl)-4-(3,4,5-trimethoxyphenyl)-1H-1,2,3-triazole;
4-(4-(3,4,5-trimethoxyphenyl)-1H-1,2,3-triazol-5-yl)aniline;
5-(4′-methoxybiphenyl-4-yl)-4-(3,4,5-trimethoxyphenyl)-1H-1,2,3-triazole;
3-(4-(4-(3,4,5-trimethoxyphenyl)-1H-1,2,3-triazol-5-yl)phenyl)pyridine;
4-(4-(4-(3,4,5-trimethoxyphenyl)-1H-1,2,3-triazol-5-yl)phenyl)pyridine;
2-(4-(4-(3,4,5-trimethoxyphenyl)-1H-1,2,3-triazol-5-yl)phenyl)pyridine;
7-(4-(3,4,5-trimethoxyphenyl)-1H-1,2,3-triazol-5-yl)quinoline;
4-(4-(3,4,5-trimethoxyphenyl)-1H-1,2,3-triazol-5-yl)pyridine;
7-(4-(3,4,5-trimethoxyphenyl)-1H-1,2,3-triazol-5-yl)isoquinoline;
1-methyl-5-(4-(3,4,5-trimethoxyphenyl)-1H-1,2,3-triazol-5-yl)-1H-indole;
4-(benzo[d][1,3]dioxol-5-yl)-5-(4-methoxyphenyl)-1H-1,2,3-triazole;
1-ethyl-6-(5-(4-methoxyphenyl)-1H-1,2,3-triazol-4-yl)-1H-indole;
4-(4-(3,4,5-trimethoxyphenyl)-1H-1,2,3-triazol-5-yl)benzoic acid;
methyl 4-(4-(3,4,5-trimethoxyphenyl)-1H-1,2,3-triazol-5-yl)benzoate;
2-(4-(4-(3,4,5-trimethoxyphenyl)-1H-1,2,3-triazol-5-yl)phenypoxazole;
5-(4-methoxyphenyl)-4-(3,4,5-triethylphenyl)-1H-1,2,3-triazole;
5-(4-iodophenyl)-4-(3,4,5-triethylphenyl)-1H-1,2,3-triazole;
5-(3-fluoro-4-methoxyphenyl)-4-(3,4,5-triethylphenyl)-1H-1,2,3-triazole;
5-(4-nitrophenyl)-4-(3,4,5-triethylphenyl)-1H-1,2,3-triazole;
N,N-dimethyl-4-(4-(3,4,5-triethylphenyl)-1H-1,2,3-triazol-5-yl)aniline;
5-(4-methoxyphenyl)-4-(3,4,5-trimethylphenyl)-1H-1,2,3-triazole;
3-(4-(4-(3,4,5-triethylphenyl)-1H-1,2,3-triazol-5-yl)phenyl)pyridine;
4-(4-(4-(3,4,5-triethylphenyl)-1H-1,2,3-triazol-5-yl)phenyl)pyridine;
2-(4-(4-(3,4,5-triethylphenyl)-1H-1,2,3-triazol-5-yl)phenyl)pyridine;
7-(4-(3,4,5-triethylphenyl)-1H-1,2,3-triazol-5-yl)quinoline;
4-(4-(3,4,5-triethylphenyl)-1H-1,2,3-triazol-5-yl)pyridine;
7-(4-(3,4,5-triethylphenyl)-1H-1,2,3-triazol-5-yl)isoquinoline;
5-(4-(3,4,5-triethylphenyl)-1H-1,2,3-triazol-5-yl)-1H-indole;
4-(benzo[d][1,3]dioxol-5-yl)-5-(4-methoxyphenyl)-1H-1,2,3-triazole;
1-isopropyl-6-(5-(4-methoxyphenyl)-1H-1,2,3-triazol-4-yl)-1H-indole;
5-(4-methoxyphenyl)-4-(2,3,4-trimethoxyphenyl)-1H-1,2,3-triazole;
2-methoxy-5-(4-(3,4,5-trimethoxyphenyl)-1H-1,2,3-triazol-5-yl)phenol;
ethyl 2-methoxy-5-(4-(3,4,5-trimethoxyphenyl)-1H-1,2,3-triazol-5-yl)phenyl hydrogen phosphate;
4-ethyl-2-(5-(4-methoxyphenyl)-1H-1,2,3-triazol-4-yl)-5-methoxyphenol;
5-(4-isopropylphenyl)-4-(3,4,5-trimethoxyphenyl)-1H-1,2,3-triazole;
5-(2,3-dihydrobenzo[b][1,4]dioxin-6-yl)-4-(3,4,5-trimethoxyphenyl)-1H-1,2,3-triazole;
5-(4-ethylphenyl)-4-(3,4,5-trimethoxyphenyl)-1H-1,2,3-triazole;
5-methoxy-2-(4-(3,4,5-trimethoxyphenyl)-1H-1,2,3-triazol-5-yl)pyridine;
6-(5-(4-methoxyphenyl)-1H-1,2,3-triazol-4-yl)-2,3,4-trimethoxypyridine;
methyl 2,6-dimethoxy-4-(5-(4-methoxyphenyl)-1H-1,2,3-triazol-4-yl)benzoate;
5-(5-(4-methoxyphenyl)-1H-1,2,3-triazol-4-yl)-1,3-phenylene diacetate;
2-methoxy-5-(4-(3,4,5-trimethoxyphenyl)-1H-1,2,3-triazol-5-yl)pyridine;
5-methoxy-7-(5-(4-methoxyphenyl)-1H-1,2,3-triazol-4-yl)-1-methyl-1H-indole;
1-ethyl-7-(5-(4-methoxyphenyl)-1H-1,2,3-triazol-4-yl)-1H-indole;
4-(benzo[d][1,3]dioxol-4-yl)-5-(4-methoxyphenyl)-1H-1,2,3-triazole;
5-methoxy-2-(4-(3,4,5-trimethoxyphenyl)-1H-1,2,3-triazol-5-yl)phenol;
ethyl 5-methoxy-2-(4-(3,4,5-trimethoxyphenyl)-1H-1,2,3-triazol-5-yl)phenyl hydrogen phosphate;
4-(4-(3,4,5-trimethoxyphenyl)-1H-1,2,3-triazol-5-yl)pyridazine;
5-(4-(3,4,5-trimethoxyphenyl)-1H-1,2,3-triazol-5-yl)pyrimidine;
3-(4-(3,4,5-trimethoxyphenyl)-1H-1,2,3-triazol-5-yl)pyridine hydrochloride;
2-methoxy-5-(4-(3,4,5-trimethoxyphenyl)-1H-1,2,3-triazol-5-yl)benzenethiol;
sodium S-2-methoxy-5-(4-(3,4,5-trimethoxyphenyl)-1H-1,2,3-triazol-5-yl)phenyl phosphorothioate;
N-(2-methoxy-5-(4-(3,4,5-trimethoxyphenyl)-1H-1,2,3-triazol-5-yl)phenyl)acetamide;
2-methoxy-5-(4-(3,4,5-trimethoxyphenyl)-1H-1,2,3-triazol-5-yl)benzenaminium chloride;
5-methoxy-2-(4-(3,4,5-trimethoxyphenyl)-1H-1,2,3-triazol-5-yl)phenol;
2-methoxy-5-(4-(3,4,5-trimethoxyphenyl)-1H-1,2,3-triazol-5-yl)pyridine;
5-methoxy-2-(4-(3,4,5-trimethoxyphenyl)-1H-1,2,3-triazol-5-yl)pyridine;
sodium 2-methoxy-5-(4-(3,4,5-trimethoxyphenyl)-1H-1,2,3-triazol-5-yl)benzoate;
methyl 2-methoxy-5-(4-(3,4,5-trimethoxyphenyl)-1H-1,2,3-triazol-5-yl)benzoate;
sodium 2-methoxy-5-(4-(3,4,5-trimethoxyphenyl)-1H-1,2,3-triazol-5-yl)phenyl sulfate;
5-methoxy-2-(4-(3,4,5-trimethoxyphenyl)-1H-1,2,3-triazol-5-yl)aniline;
sodium 2,3-dimethoxy-5-(4-(3,4,5-trimethoxyphenyl)-1H-1,2,3-triazol-5-yl)phenyl phosphate;
sodium 5-methoxy-2-(4-(3,4,5-trimethoxyphenyl)-1H-1,2,3-triazol-5-yl)phenyl phosphate;
5-(4-(methylthio)phenyl)-4-(3,4,5-trimethoxyphenyl)-1H-1,2,3-triazole;
sodium 2-(methylthio)-5-(4-(3,4,5-trimethoxyphenyl)-1H-1,2,3-triazol-5-yl)phenyl phosphate;
2-(methylthio)-5-(4-(3,4,5-trimethoxyphenyl)-1H-1,2,3-triazol-5-yl)aniline;
5-(2,3-dihydrobenzofuran-6-yl)-4-(3,4,5-trimethoxyphenyl)-1H-1,2,3-triazole;
sodium 4-(4-(3,4,5-trimethoxyphenyl)-1H-1,2,3-triazol-5-yl)phenolate;
monosodium monosodium(II) mono(4-(4-(3,4,5-trimethoxyphenyl)-1H-1,2,3-triazol-5-yl)phenyl phosphate);
5-(4-(4-(3,4,5-trimethoxyphenyl)-1H-1,2,3-triazol-5-yl)phenyl)-1H-tetrazole;
1-methyl-5-(4-(4-(3,4,5-trimethoxyphenyl)-1H-1,2,3-triazol-5-yl)phenyl)-1H-tetrazole;
1-methyl-5-(4-(3,4,5-trimethoxyphenyl)-1H-1,2,3-triazol-5-yl)-1H-indole;
4-(4-(7-methoxybenzo[d][1,3]dioxol-5-yl)-1H-1,2,3-triazol-5-yl)pyridazine;
5-(4-(7-methoxybenzo[d][1,3]dioxol-5-yl)-1H-1,2,3-triazol-5-yl)pyrimidine;
3-(4-(7-methoxybenzo[d][1,3]dioxol-5-yl)-1H-1,2,3-triazol-5-yl)pyridine hydrochloride;
2-methoxy-5-(4-(7-methoxybenzo[d][1,3]dioxol-5-yl)-1H-1,2,3-triazol-5-yl)benzenethiol;
sodium S-2-methoxy-5-(4-(7-methoxybenzo[d][1,3]dioxol-5-yl)-1H-1,2,3-triazol-5-yl)phenyl phosphorothioate;
N-(2-methoxy-5-(4-(7-methoxybenzo[d][1,3]dioxol-5-yl)-1H-1,2,3-triazol-5-yl)phenyl)acetamide;
2-methoxy-5-(4-(7-methoxybenzo[d][1,3]dioxol-5-yl)-1H-1,2,3-triazol-5-yl)benzenaminium chloride;
5-methoxy-2-(4-(7-methoxybenzo[d][1,3]dioxol-5-yl)-1H-1,2,3-triazol-5-yl)phenol;
2-methoxy-5-(4-(7-methoxybenzo[d][1,3]dioxol-5-yl)-1H-1,2,3-triazol-5-yl)pyridine;
5-methoxy-2-(4-(7-methoxybenzo[d][1,3]dioxol-5-yl)-1H-1,2,3-triazol-5-yl)pyridine;
sodium 2-methoxy-5-(4-(7-methoxybenzo[d][1,3]dioxol-5-yl)-1H-1,2,3-triazol-5-yl)benzoate;
methyl 2-methoxy-5-(4-(7-methoxybenzo[d][1,3]dioxol-5-yl)-1H-1,2,3-triazol-5-yl)benzoate;
sodium 2-methoxy-5-(4-(7-methoxybenzo[d][1,3]dioxol-5-yl)-1H-1,2,3-triazol-5-yl)phenyl sulfate;
5-methoxy-2-(4-(7-methoxybenzo[d][1,3]dioxol-5-yl)-1H-1,2,3-triazol-5-yl)aniline;
sodium 2,3-dimethoxy-5-(4-(7-methoxybenzo[d][1,3]dioxol-5-yl)-1H-1,2,3-triazol-5-yl)phenyl phosphate;
sodium 5-methoxy-2-(4-(7-methoxybenzo[d][1,3]dioxol-5-yl)-1H-1,2,3-triazol-5-yl)phenyl phosphate;
4-(7-methoxybenzo[d][1,3]dioxol-5-yl)-5-(4-(methylthio)phenyl)-1H-1,2,3-triazole;
sodium 5-(4-(7-methoxybenzo[d][1,3]dioxol-5-yl)-1H-1,2,3-triazol-5-yl)-2-(methylthio)phenyl phosphate;
5-(4-(7-methoxybenzo[d][1,3]dioxol-5-yl)-1H-1,2,3-triazol-5-yl)-2-(methylthio)aniline;
5-(2,3-dihydrobenzofuran-6-yl)-4-(7-methoxybenzo[d][1,3]dioxol-5-yl)-1H-1,2,3-triazole;
sodium 4-(4-(7-methoxybenzo[d][1,3]dioxol-5-yl)-1H-1-1,2,3-triazol-5-yl)phenolate;
sodium 4-(4-(7-methoxybenzo[d][1,3]dioxol-5-yl)-1H-1,2,3-triazol-5-yl)phenyl phosphate;
5-(4-(4-(7-methoxybenzo[d][1,3]dioxol-5-yl)-1 H-1,2,3-triazol-5-yl)phenyl)-1H-tetrazole;
5-(4-(4-(7-methoxybenzo[d][1,3]dioxol-5-yl)-1H-1,2,3-triazol-5-yl)phenyl)-1-methyl-1H-tetrazole;
5-(4-(7-methoxybenzo[d][1,3]dioxol-5-yl)-1H-1,2,3-triazol-5-yl)-1-methyl-1H-indole;
1-methyl-5-(5-(3,4,5-trimethoxyphenyl)-1H-1,2,3-triazol-4-yl)-1H-indole;
monosodium monosodium(II) mono(2-methoxy-5-(5-(3,4,5-trimethoxyphenyl)-1H-1,2,3-triazol-4-yl)phenyl phosphate);
N,N-dimethyl-4-(5-(3,4,5-trimethoxyphenyl)-1H-1,2,3-triazol-4-yl)aniline;
2-methoxy-5-(5-(3,4,5-trimethoxyphenyl)-1H-1,2,3-triazol-4-yl)benzenaminium chloride;
3-hydroxy-1-(2-methoxy-5-(5-(3,4,5-trimethoxyphenyl)-1H-1,2,3-triazol-4-yl)phenylamino)-1-oxopropan-2-aminium chloride;
5-(4-methoxyphenyl)-4-(2,4,5-trimethoxyphenyl)-1H-1,2,3-triazole;
5-p-tolyl-4-(2,4,5-trimethoxyphenyl)-1H-1,2,3-triazole;
5-(4-ethoxyphenyl)-4-(2,4,5-trimethoxyphenyl)-1H-1,2,3-triazole;
5-(4-ethylphenyl)-4-(2,4,5-trimethoxyphenyl)-1H-1,2,3-triazole;
5-(4-propoxyphenyl)-4-(2,4,5-trimethoxyphenyl)-1H-1,2,3-triazole;
5-(4-propylphenyl)-4-(2,4,5-trimethoxyphenyl)-1H-1,2,3-triazole;
5-(4-butoxyphenyl)-4-(2,4,5-trimethoxyphenyl)-1H-1,2,3-triazole;
5-(4-butylphenyl)-4-(2,4,5-trimethoxyphenyl)-1H-1,2,3-triazole;
5-(4-bromophenyl)-4-(2,4,5-trimethoxyphenyl)-1H-1,2,3-triazole;
5-(4-chlorophenyl)-4-(2,4,5-trimethoxyphenyl)-1H-1,2,3-triazole;
5-(4-fluorophenyl)-4-(2,4,5-trimethoxyphenyl)-1H-1,2,3-triazole;
5-(4-nitrophenyl)-4-(2,4,5-trimethoxyphenyl)-1H-1,2,3-triazole;
N,N-dimethyl-4-(4-(2,4,5-trimethoxyphenyl)-1H-1,2,3-triazol-5-yl)aniline;
5-(3,4-dimethoxyphenyl)-4-(2,4,5-trimethoxyphenyl)-1H-1,2,3-triazole;
2-methoxy-5-(4-(2,4,5-trimethoxyphenyl)-1H-1,2,3-triazol-5-yl)phenol;
4-(2,4,5-trimethoxyphenyl)-5-(3,4,5-trimethoxyphenyl)-1H-1,2,3-triazole;
5-(4-methoxyphenyl)-4-(2,3,5-trimethoxyphenyl)-1H-1,2,3-triazole;
5-p-tolyl-4-(2,3,5-trimethoxyphenyl)-1H-1,2,3-triazole;
5-(4-ethoxyphenyl)-4-(2,3,5-trimethoxyphenyl)-1H-1,2,3-triazole;
5-(4-ethylphenyl)-4-(2,3,5-trimethoxyphenyl)-1H-1,2,3-triazole;
5-(4-propoxyphenyl)-4-(2,3,5-trimethoxyphenyl)-1H-1,2,3-triazole;
5-(4-propylphenyl)-4-(2,3,5-trimethoxyphenyl)-1H-1,2,3-triazole;
5-(4-butoxyphenyl)-4-(2,3,5-trimethoxyphenyl)-1H-1,2,3-triazole;
5-(4-butylphenyl)-4-(2,3,5-trimethoxyphenyl)-1H-1,2,3-triazole;
5-(4-bromophenyl)-4-(2,3,5-trimethoxyphenyl)-1H-1,2,3-triazole;
5-(4-chlorophenyl)-4-(2,3,5-trimethoxyphenyl)-1H-1,2,3-triazole;
5-(4-fluorophenyl)-4-(2,3,5-trimethoxyphenyl)-1H-1,2,3-triazole;
5-(4-nitrophenyl)-4-(2,3,5-trimethoxyphenyl)-1H-1,2,3-triazole;
N,N-dimethyl-4-(4-(2,3,5-trimethoxyphenyl)-1H-1,2,3-triazol-5-yl)aniline;
5-(3,4-dimethoxyphenyl)-4-(2,3,5-trimethoxyphenyl)-1H-1,2,3-triazole;
2-methoxy-5-(4-(2,3,5-trimethoxyphenyl)-1H-1,2,3-triazol-5-yl)phenol;
4-(2,3,5-trimethoxyphenyl)-5-(3,4,5-trimethoxyphenyl)-1H-1,2,3-triazole;
4-(4-methoxyphenyl)-5-(2,3,4,5-tetramethoxyphenyl)-1H-1,2,3-triazole;
5-(2,3,4,5-tetramethoxyphenyl)-4-p-tolyl-1H-1,2,3-triazole;
4-(4-ethoxyphenyl)-5-(2,3,4,5-tetramethoxyphenyl)-1H-1,2,3-triazole;
4-(4-ethylphenyl)-5-(2,3,4,5-tetramethoxyphenyl)-1H-1,2,3-triazole;
4-(4-propoxyphenyl)-5-(2,3,4,5-tetramethoxyphenyl)-1H-1,2,3-triazole;
4-(4-propylphenyl)-5-(2,3,4,5-tetramethoxyphenyl)-1H-1,2,3-triazole;
4-(4-butoxyphenyl)-5-(2,3,4,5-tetramethoxyphenyl)-1H-1,2,3-triazole;
4-(4-butylphenyl)-5-(2,3,4,5-tetramethoxyphenyl)-1H-1,2,3-triazole;
4-(4-bromophenyl)-5-(2,3,4,5-tetramethoxyphenyl)-1H-1,2,3-triazole;
4-(4-chlorophenyl)-5-(2,3,4,5-tetramethoxyphenyl)-1H-1,2,3-triazole;
4-(4-fluorophenyl)-5-(2,3,4,5-tetramethoxyphenyl)-1H-1,2,3-triazole;
4-(4-nitrophenyl)-5-(2,3,4,5-tetramethoxyphenyl)-1H-1,2,3-triazole;
N,N-dimethyl-4-(5-(2,3,4,5-tetramethoxyphenyl)-1H-1,2,3-triazol-4-yl)aniline;
4-(3,4-dimethoxyphenyl)-5-(2,3,4,5-tetramethoxyphenyl)-1H-1,2,3-triazole;
2-methoxy-5-(5-(2,3,4,5-tetramethoxyphenyl)-1H-1,2,3-triazol-4-yl)phenol;
5-(2,3,4,5-tetramethoxyphenyl)-4-(3,4,5-trimethoxyphenyl)-1H-1,2,3-triazole;
5-(2,3-dihydrobenzo[b][1,4]dioxin-6-yl)-4-(3,4-dimethoxyphenyl)-1H-1,2,3-triazole;
2-(5-(3,4-dimethylphenyl)-1H-1,2,3-triazol-4-yl)-4-ethyl-5-methoxyphenol;
2-(5-(4-chlorophenyl)-1H-1,2,3-triazol-4-yl)-4-ethyl-5-methoxyphenol;
5-methoxy-2-(5-phenyl-1H-1,2,3-triazol-4-yl)-4-propylphenol;
4-ethyl-5-methoxy-2-(5-p-tolyl-1H-1,2,3-triazol-4-yl)phenol;
2-(5-(4-aminophenyl)-1H-1,2,3-triazol-4-yl)-4-ethyl-5-methoxyphenol;
4-ethyl-5-methoxy-2-(5-(4-(trifluoromethyl)phenyl)-1H-1,2,3-triazol-4-yl)phenol;
4-ethyl-5-methoxy-2-(5-(4-methoxyphenyl)-1H-1,2,3-triazol-4-yl)phenol; or
4-(4-bromophenyl)-5-(3,4,5-trimethoxyphenyl)-1H-1,2,3-triazole;

or pharmaceutically acceptable salts, solvates, clathrates, or prodrugs thereof.

In another embodiment, the invention relates to compounds selected from the group consisting of:
2-amino-N-(2-methoxy-5-(4-(3,4,5-trimethoxyphenyl)-1H-1,2,3-triazol-5-yl)phenyl)acetamide hydrochloride;
2-amino-3-hydroxy-N-(2-methoxy-5-(4-(3,4,5-trimethoxyphenyl)-1H-1,2,3-triazol-5-yl)phenyl)propanamide hydrochloride;
2-amino-N-(2-methoxy-5-(4-(3,4,5-trimethoxyphenyl)-1H-1,2,3-triazol-5-yl)phenyl)propanamide;
2-amino-N-(2-methoxy-5-(4-(3,4,5-trimethoxyphenyl)-1H-1,2,3-triazol-5-yl)phenyl)-4-(methylthio)butanamide hydrochloride;
2-amino-N-(2-methoxy-5-(4-(3,4,5-trimethoxyphenyl)-1H-1,2,3-triazol-5-yl)phenyl)butanamide hydrochloride;
2-amino-N-(2-methoxy-5-(4-(3,4,5-trimethoxyphenyl)-1H-1,2,3-triazol-5-yl)phenyl)-3-phenylpropanamide hydrochloride;
2-amino-N-(2-methoxy-5-(4-(3,4,5-trimethoxyphenyl)-1H-1,2,3-triazol-5-yl)phenyl)-4-methylpentanamide hydrochloride;
2-amino-N-(2-methoxy-5-(4-(3,4,5-trimethoxyphenyl)-1H-1-1,2,3-triazol-5-yl)phenyl)-4-methylpentanamide hydrochloride;
1-(2-methoxy-5-(4-(3,4,5-trimethoxyphenyl)-1H-1,2,3-triazol-5-yl)phenylamino)-3-methyl-1-oxobutan-2-aminium chloride;
1-(2-methoxy-5-(4-(3,4,5-trimethoxyphenyl)-1H-1,2,3-triazol-5-yl)phenylamino)-3-methyl-1-oxopentan-2-aminium chloride;
3-hydroxy-1-(2-methoxy-5-(4-(3,4,5-trimethoxyphenyl)-1H-1,2,3-triazol-5-yl)phenylamino)-1-oxobutan-2-aminium chloride;
3-(4-hydroxyphenyl)-1-(2-methoxy-5-(4-(3,4,5-trimethoxyphenyl)-1H-1,2,3-triazol-5-yl)phenylamino)-1-oxopropan-2-aminium chloride;
2-(2-methoxy-5-(4-(3,4,5-trimethoxyphenyl)-1H-1,2,3-triazol-5-yl)phenylamino)-2-oxo-1-phenylethanaminium chloride;
3-(1H-indol-2-yl)-1-(2-methoxy-5-(4-(3,4,5-trimethoxyphenyl)-1H-1,2,3-triazol-5-yl)phenylamino)-1-oxopropan-2-aminium chloride;
3-(benzofuran-2-yl)-1-(2-methoxy-5-(4-(3,4,5-trimethoxyphenyl)-1H-1,2,3-triazol-5-yl)phenylamino)-1-oxopropan-2-aminium chloride;
3-carboxy-1-(2-methoxy-5-(4-(3,4,5-trimethoxyphenyl)-1H-1,2,3-triazol-5-yl)phenylamino)-1-oxopropan-2-aminium chloride;
4-carboxy-1-(2-methoxy-5-(4-(3,4,5-trimethoxyphenyl)-1H-1,2,3-triazol-5-yl)phenylamino)-1-oxobutan-2-aminium chloride;
5-amino-1-(2-methoxy-5-(4-(3,4,5-trimethoxyphenyl)-1H-1,2,3-triazol-5-yl)phenylamino)-1,5-dioxopentan-2-aminium chloride;
4-amino-1-(2-methoxy-5-(4-(3,4,5-trimethoxyphenyl)-1H-1,2,3-triazol-5-yl)phenylamino)-1,4-dioxobutan-2-aminium chloride;
3-(1H-imidazol-5-yl)-1-(2-methoxy-5-(4-(3,4,5-trimethoxyphenyl)-1H-1,2,3-triazol-5-yl)phenylamino)-1-oxopropan-2-aminium chloride;
6-amino-1-(2-methoxy-5-(4-(3,4,5-trimethoxyphenyl)-1H-1,2,3-triazol-5-yl)phenylamino)-1-oxohexan-2-aminium chloride;
5-guanidino-1-(2-methoxy-5-(4-(3,4,5-trimethoxyphenyl)-1H-1,2,3-triazol-5-yl)phenylamino)-1-oxopentan-2-aminium chloride;
4-(2-methoxy-5-(4-(3,4,5-trimethoxyphenyl)-1H-1,2,3-triazol-5-yl)phenylamino)-4-oxobutanoic acid;
5-(2-methoxy-5-(4-(3,4,5-trimethoxyphenyl)-1H-1,2,3-triazol-5-yl)phenylamino)-5-oxopentanoic acid;
3-(2-methoxy-5-(4-(3,4,5-trimethoxyphenyl)-1H-1,2,3-triazol-5-yl)phenylamino)-3-oxopropan-1-aminium chloride;
N-(2-methoxy-5-(4-(3,4,5-trimethoxyphenyl)-1H-1,2,3-triazol-5-yl)phenyl)-3-(2-methoxyethoxy)propanamide;
3-(2-PEG)-N-(2-methoxy-5-(4-(3,4,5-trimethoxyphenyl)-1H-1,2,3-triazol-5-yl)phenyl)butyramide;
N-(2-methoxy-5-(4-(3,4,5-trimethoxyphenyl)-1H-1,2,3-triazol-5-yl)phenyl)-3-(2-(methylamino)ethylamino)propanamide;
3-PEG-N-(2-methoxy-5-(4-(3,4,5-trimethoxyphenyl)-1H-1,2,3-triazol-5-yl)phenylamino)-2-oxoethyl)butyramide;
4-(2-(2-methoxy-5-(4-(3,4,5-trimethoxyphenyl)-1H-1,2,3-triazol-5-yl)phenylamino)-2-oxoethylamino)-4-oxobutanoic acid;
2-methoxyethyl 2-methoxy-5-(4-(3,4,5-trimethoxyphenyl)-1H-1,2,3-triazol-5-yl)phenylcarbamate;
PEG-2-methoxy-5-(4-(3,4,5-trimethoxyphenyl)-1H-1,2,3-triazol-5-yl)phenylcarbamate;
3-amino-4-(2-(5-guanidino-1-(2-methoxy-5-(4-(3,4,5-trimethoxyphenyl)-1H-1,2,3-triazol-5-yl)phenylamino)-1-oxopentan-2-ylamino)-2-oxoethylamino)-4-oxobutanoic acid; or
2-amino-N-(2-methoxy-5-(4-(3,4,5-trimethoxyphenyl)-1H-1,2,3-triazol-5-yl)phenyl)propanamide hydrochloride;

or pharmaceutically acceptable salts, solvates, clathrates, or prodrugs thereof.

In one embodiment, the invention relates to compounds of formula (XXXI):

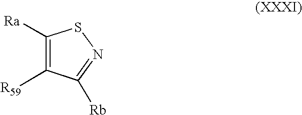

or a pharmaceutically acceptable salt, solvate, clathrate, or prodrug thereof, wherein:

one of Rₐ or R_{b} is —H and the other is an optionally substituted aryl or an optionally substituted heteroaryl; and

R₅₉ is an optionally substituted aryl or an optionally substituted heteroaryl, provided that R₅₉ is not an unsubstituted phenyl.

In another embodiment, the invention relates to compounds of formula (XXXV):

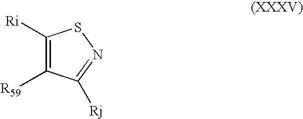

or a pharmaceutically acceptable salt, solvate, clathrate, or prodrug thereof, wherein:

one of Rᵢ or Rⱼ is —H and the other is represented by the following formula:

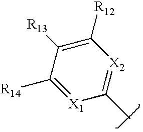

X₁ and X₂ are each, independently, CH or N;

R₁₂, R₁₃ and R₁₄ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₅₉ is defined as for formula (XXXI); and

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl; and

p is 1 or 2.

In another embodiment, the invention relates to compounds of formula (XXXVI):

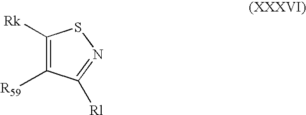

or a pharmaceutically acceptable salt, solvate, clathrate, or prodrug thereof, wherein:

one of Rₖ or Rₗ is —H and the other is represented by the following formula:

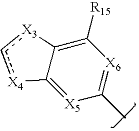

the dashed line indicates that the bond is a single bond or a double bond;

X₃ and X₄ are each, independently, CH, N, CH₂, NR₁₆, O, or S;

X₅ and X₆ are each, independently, CR₂₉ or N;

R₁₅ is H, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₁₆ is H, an alkyl, a cycloalkyl, an aralkyl, —C(O)R, wherein R is an alkyl, a cycloalkyl, or an aralkyl;

R₂₉, for each occurrence, is independently, H or a substituent; and

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently -H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₁₇, for each occurrence, is independently, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

p is 1 or 2; and

R₅₉ is defined as for formula (XXXI).

In another embodiment, the invention relates to compounds of formula (XXXVII):

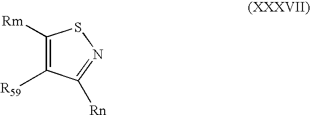

or a pharmaceutically acceptable salt, solvate, clathrate, or prodrug thereof, wherein:

one of Rₘ or Rₙ is —H and the other is represented by the following formula:

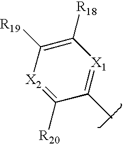

R₅₉ is defined as for formula (XXXVI);

R₁₈ and R₁₉ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₂₀ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁; and

X₁ and X₂ are are defined as for formula (XXXV).

In another embodiment, the invention relates to compounds of formula (XXXVIII):

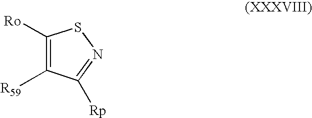

or a pharmaceutically acceptable salt, solvate, clathrate, or prodrug thereof, wherein:

one of Rₒ or Rₚ is —H and the other is represented by the following formula:

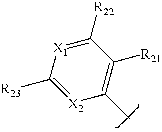

R₅₉ is defined as for formula (XXXVI);

R₂₂ and R₂₃, are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₂₁ is halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁; and

X₁ and X₂ are are defined as for formula (XXXV).

In another embodiment, the invention relates to compounds of formula (XXXIX):

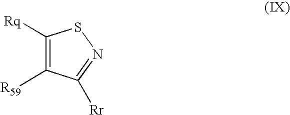

or a pharmaceutically acceptable salt, solvate, clathrate, or prodrug thereof, wherein:

one of R_{q} or Rᵣ is —H and the other is represented by the following formula:

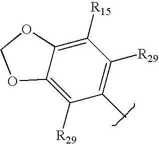

R₅₉, R₁₅, and R₂₉ are defined as for formula (XXXVI).

In another embodiment, the invention relates to compounds of formula (XL):

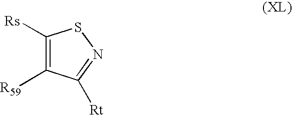

or a pharmaceutically acceptable salt, solvate, clathrate, or prodrug thereof, wherein:

one of Rs or Rt is —H and the other is represented by the following formula:

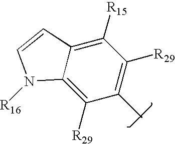

R₅₉, R₁₅, R₁₆, and R₂₉ are defined as for formula (XXXVI).

In another embodiment, the invention relates to compounds of formula (XXXIA):

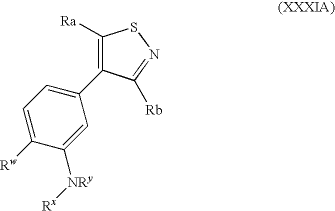

or a pharmaceutically acceptable salt, solvate, clathrate, or prodrug thereof, wherein:

one of Rₐ or R_{b} is —H and the other is an optionally substituted aryl, or an optionally substituted heteroaryl; and

R^{x} is (R^{aa})ₘ, —R^{aa}—C(O)(CH₂)ₙC(O)OH, —C(O)(CH₂)ₙC(O)OH, —C(O)YR^{z}, —C(O)NH—R^{aa}, or —(R^{aa})_{q}C(O)(Y₁);

R^{y} is —H or lower alkyl;

R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl;

R_{7,} for each occurrence, is independently -H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R^{aa} is an amino acid residue or an amino acid residue analog;

Y is CH₂, O, or NH;

R^{z} is Alk-NH₂, Alk-C(O)OH, Het, or Y₁;

Alk is an optionally substituted alkylene;

Het is an optionally substituted heteroalkyl;

Y₁ is a water soluble polymer with a molecular weight less than 60,000 daltons;

n is 1, 2, 3, or 4;

m is an integer from 1 to 10; and

q is 0 or 1.

In another embodiment, the invention relates to compounds of formula (XXXVA):

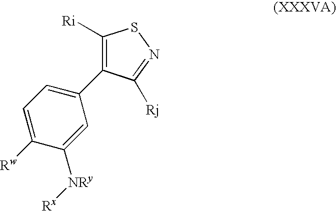

or a pharmaceutically acceptable salt, solvate, clathrate, or prodrug thereof, wherein:

one of Rᵢ or Rⱼ is —H and the other is represented by the following formula:

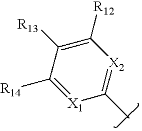

X₁ and X₂ are each, independently, CH or N;

R₁₂, R₁₃ and R₁₄ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)ₚOR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently -H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl; and

p is 1 or 2; and

R^{x}, R^{y}, and R^{w} are defined as for formula (XXXIA).

In another embodiment, this invention relates to compounds of formula (XXXVIA):

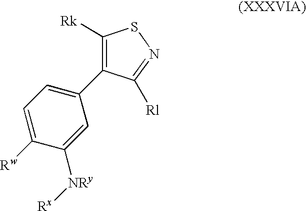

or a pharmaceutically acceptable salt, solvate, clathrate, or prodrug thereof, wherein:

one of Rₖ or Rₗ is —H and the other is represented by the following formula:

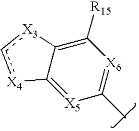

the dashed line indicates that the bond is a single bond or a double bond;

X₃ and X₄ are each, independently, CH, N, CH₂, NR₁₆, O, or S;

X₅ and X₆ are each, independently, CR₂₉ or N;

R₁₅ is H, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₂, —NR₁₀R₁₁, —C(O)R₇, —C(O)OR₇, —OC(O)R₂, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₂, —NR₈S(O)ₚR₂, —S(O)ₚNR₁₀R₁₁;

R₁₆ is H, an alkyl, a cycloalkyl, an aralkyl, —C(O)R, wherein R is an alkyl, a cycloalkyl, or an aralkyl;

R₂₉, for each occurrence, is independently, H or a substituent

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₁₇, for each occurrence, is independently, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

p is 1 or 2; and

R^{x}, R^{y}, and R^{w} are defined as for formula (XXXIA).

In another embodiment, the invention relates to compounds of formula (XXXVIIA):

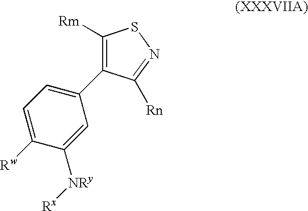

or a pharmaceutically acceptable salt, solvate, clathrate, or prodrug thereof,

wherein:

one of Rₘ or Rₙ is —H and the other is represented by the following formula:

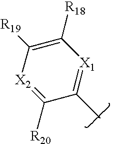

X₁ and X₂ are each, independently, CH or N;

R₁₈ and R₁₉ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₇, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₂₀ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁; and

R^{x}, R^{y}, and R^{w} are defined as for formula (XXXIA).

In another embodiment, the invention relates to compounds of formula (XXXVIIIA):

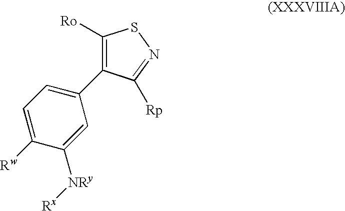

or a pharmaceutically acceptable salt, solvate, clathrate, or prodrug thereof, wherein:

one of Rₒ or Rₚ is —H and the other is represented by the following formula:

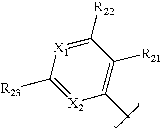

X₁ and X₂ are each, independently, CH or N;

R₂₂ and R₂₃, are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₂₁ is halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁; and

R^{x}, R^{y}, and R^{w} are defined as for formula (XXXIA).

In another embodiment, the invention relates to compounds of formula (XXXIXA):

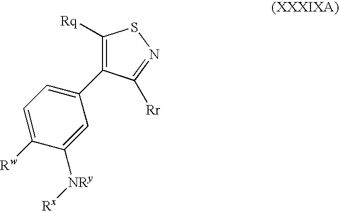

or a pharmaceutically acceptable salt, solvate, clathrate, or prodrug thereof, wherein:

one of R_{q} or Rᵣ is —H and the other is represented by the following formula:

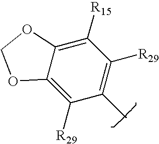

R₁₅, R₁₉, and R₂₉ are defined as for formula (XXXVIA); and

R^{x}, R^{y}, R^{w} are defined as for formula (XXXIA).

In another embodiment, the invention relates to compounds of formula (XLA):

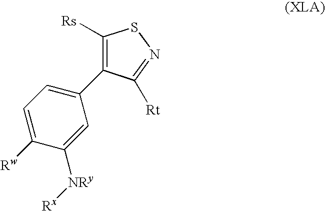

or a pharmaceutically acceptable salt, solvate, clathrate, or prodrug thereof, wherein:

one of Rₛ or Rₜ is —H and the other is represented by the following formula:

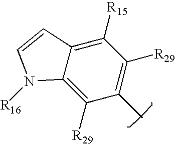

R₁₅, R₁₆, and R₂₉ are defined as for formula (XXXVIA); and

R^{x}, R^{y}, and R^{w} are defined as for formula (XXXIA).

In another embodiment, the invention relates to compounds of formula (XXXIB):

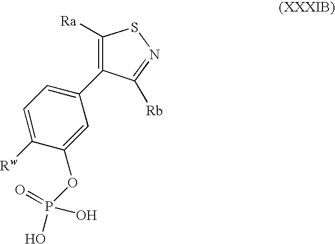

or a pharmaceutically acceptable salt, solvate, or clathrate, thereof, wherein:

R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl;

R_{7,} for each occurrence, is independently -H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

one of Rₐ or R_{b} is —H and the other is an optionally substituted aryl or an optionally substituted heteroaryl.

In another embodiment, the invention relates to compounds of formula (XXXVB):

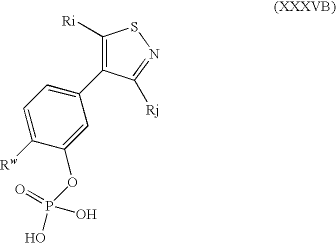

or a pharmaceutically acceptable salt, solvate, clathrate, or prodrug thereof, wherein:

one of Rᵢ or Rⱼ is —H and the other is represented by the following formula:

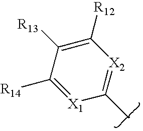

X₁ and X₂ are each, independently, CH or N;

R₁₂, R₁₃ and R₁₄ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R^{w} is defined as for formula (XXXIB);

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently -H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀and R₁₁, taken together with the nitrogen to which they are attached, form an optionally substituted heterocyclyl or an optionally substituted heteroaryl; and

p is 1 or 2.

In another embodiment, the invention relates to compounds of formula (XXXVIB):

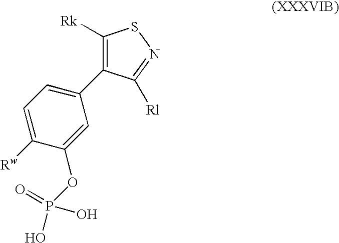

or a pharmaceutically acceptable salt, solvate, clathrate, or prodrug thereof, wherein:

one of Rₖ or Rₗ is —H and the other is represented by the following formula:

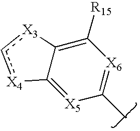

the dashed line indicates that the bond is a single bond or a double bond;

X₃ and X₄ are each, independently, CH, N, CH₂, NR₁₆, O, or S;

X₅ and X₆ are each, independently, CR₂₉ or N;

R₁₅ is H, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₁₆ is H, an alkyl, a cycloalkyl, an aralkyl, —C(O)R, wherein R is an alkyl, a cycloalkyl, or an aralkyl;

R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;

R₁₇, for each occurrence, is independently, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

p is 1 or 2;

R^{w} is defined as for formula (XXXIB); and

R₂₉, for each occurrence, is independently, H or a substituent.

In another embodiment, the invention relates to compounds of formula (XXXVIIB):

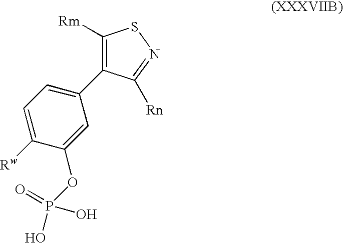

or a pharmaceutically acceptable salt, solvate, clathrate, or prodrug thereof, wherein:

one of Rₘ or Rₙ is —H and the other is represented by the following formula:

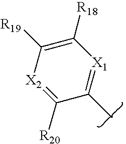

X₁ and X₂ are each, independently, CH or N;

R^{w} is defined as for formula (XXXIB);

R₁₈ and R₁₉ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁; and

R₂₀ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —0P(O)(0R₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁.

In another embodiment, the invention relates to compounds of formula (XXXVIIIB):

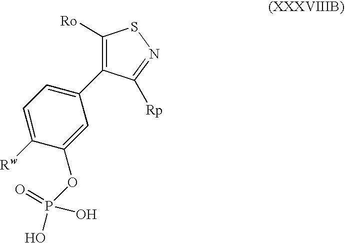

or a pharmaceutically acceptable salt, solvate, clathrate, or prodrug thereof, wherein:

one of Rₒ or Rₚ is —H and the other is represented by the following formula:

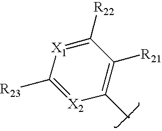

X₁ and X₂ are each, independently, CH or N;

R₂₂ and R₂₃, are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;

R₂₁ is halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁; and

R^{w} is defined as for formula (XXXIB).

In another embodiment, the invention relates to compounds of formula (XXXIXB):

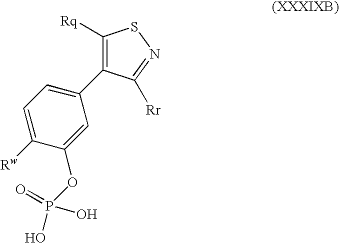

or a pharmaceutically acceptable salt, solvate, clathrate, or prodrug thereof, wherein:

one of R_{q} or Rᵣ is —H and the other is represented by the following formula:

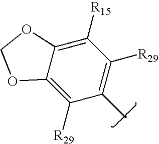

R^{w} is defined as for formula (XXXIB); and

R₁₅ and R₁₉ are defined as for formula (XXXVIB).

In another embodiment, the invention relates to compounds of formula (XLB):

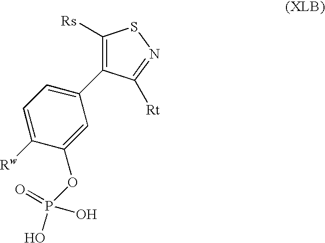

or a pharmaceutically acceptable salt, solvate, clathrate, or prodrug thereof, wherein:

one of Rₛ or Rₜ is —H and the other is represented by the following formula:

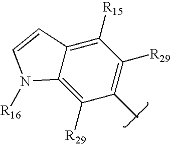

R^{w} is defined as for formula (XXXIB); and

R₁₅, R₁₆, and R₂₉ are defined as for formula (XXXVIB).

In some embodiments, in the compounds represented by formula (XXXI), (XXXIA), or (XXXIB), one of Rₐ or R_{b} is —H and the other is an optionally substituted phenyl. In one aspect of this embodiment, the phenyl group represented by Rₐ or R_{b} is unsubstituted. In another aspect of this embodiment, the phenyl group represented by Rₐ or R_{b} is substituted with from one to five substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the phenyl group represented by Rₐ or R_{b} is substituted with from one to five substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the phenyl group represented by Rₐ or R_{b} is substituted with from one to three substituents. More preferably, the phenyl group represented by Rₐ or R_{b} is substituted with three substituents.

In some embodiments, in the compounds represented by formula (XXXI), (XXXIA), or (XXXIB), one of Rₐ or R_{b} is —H and the other is an optionally substituted pyridinyl. In one aspect of this embodiment, the pyridinyl group represented by Rₐ or R_{b} is unsubstituted. In another aspect of this embodiment, the pyridinyl group represented by Rₐ or R_{b} is substituted with one or more substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the pyridinyl group represented by Rₐ or R_{b} is substituted with one or more substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the pyridinyl group represented by Rₐ or R_{b} is substituted with from one to three substituents. More preferably, the pyridinyl group represented by Rₐ or R_{b} is substituted with three substituents.

In some embodiments, in the compounds represented by formula (XXXI), (XXXIA), or (XXXIB), one of Rₐ or R_{b} is —H and the other is an optionally substituted benzo[1,3]dioxolyl. In one aspect of this embodiment, the benzo[1,3]dioxolyl group represented by Rₐ or R_{b} is unsubstituted. In another aspect of this embodiment, the benzo[1,3]dioxolyl group represented by Rₐ or R_{b} is substituted with one or more substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁, wherein R₇, R₈, R₁₀, R₁₁, and p are defined as above. In another aspect of this embodiment, the benzo[1,3]dioxolyl group represented by Rₐ or R_{b} is substituted with one or more substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the benzo[1,3]dioxolyl group represented by Rₐ or R_{b} is substituted with from one to three substituents. More preferably, the benzo[1,3]dioxolyl group represented by Rₐ or R_{b} is substituted with one substituent.

In some embodiments, in the compounds represented by formula (XXXI), (XXXIA), or (XXXIB), one of Rₐ or R_{b} is —H and the other is an optionally substituted 1H-indolyl. In one aspect of this embodiment, the 1 H-indolyl group represented by Rₐ or R_{b} is unsubstituted. In another aspect of this embodiment, the 1H-indolyl group represented by Rₐ or R_{b} is substituted with one or more substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or -8(O)ₚNR₁₀R₁₁, wherein R₇, R₈, R₁₀, R₁₁, and p are defined as above. In another aspect of this embodiment, the 1H-indolyl group represented by Rₐ or R_{b} is substituted with one or more substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the 1H-indolyl group represented by Rₐ or R_{b} is substituted with from one to three substituents. More preferably, the 1H-indolyl group represented by Rₐ or R_{b} is substituted with one substituent.

In some embodiments, in the compounds represented by formula (XXXI), (XXXIA), or (XXXIB), one of Rₐ or R_{b} is —H and the other is an optionally substituted pyridinyl. In one aspect of this embodiment, the pyridinyl group represented by Rₐ or R_{b} is unsubstituted. In another aspect of this embodiment, the pyridinyl group represented by Rₐ or R_{b} is substituted with one or more substituents independently selected from a halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₂, —NR₈S(O)ₚR₇, or —S(O)ₚNR₁₀R₁₁, wherein R₇, R₈, R₁₀, R₁₁, and p are defined as above. In another aspect of this embodiment, the pyridinyl group represented by Rₐ or R_{b} is substituted with one or more substituents, independently, selected from an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. Preferably, the pyridinyl group represented by Rₐ or R_{b} is substituted with from one to three substituents. More preferably, the pyridinyl group represented by Rₐ or R_{b} is substituted with three substituents.

In some embodiments, in the compounds represented by formula (XXXI) or (XXXV), R₅₉ is a substituted phenyl. In another aspect of this embodiment, the phenyl group represented by R₅₉ is substituted with from one to five groups independently selected from alkoxy, halo, alkyl, haloalkyl, haloalkoxy, nitro, cyano, oxazolyl, 1H-tetrazolyl, 1-methyl-1H-tetrazolyl, —OR₂₄, —SR_{N}, —C(O)R₂₄, —C(O)OR₂₄, —OC(O)R₂₄, —C(O)NR₂₅R₂₆, —NR₂₄C(O)R₂₇, —NR₂₄C(O)OR₂₇, —OC(O)NR₂₅R₂₆, guanidino, amino, alkyl amino, dialkylamino, —NR₂₄S(O)ₚR₂₈, —S(O)ₚR₂₈, —S(O)ₚ0R₂₇, —OS(O)ₚR₂₈, —OS(O)ₚOR₂₇, —OP(O)(OR₂₇)₂, or —SP(O)(OR₂₇)₂, wherein:

p is defined as above;

R₂₄ and R₂₇, for each occurrence are, independently, H, an alkyl, or a cycloalkyl;

R₂₅ and R₂₆, for each occurrence are, independently, H, an alkyl, or a cycloalkyl; or R₂₅ and R₂₆, together with the nitrogen to which they are attached are a heterocyclyl or a heteroaryl; and

R₂₈, for each occurrence, is an alkyl or a cycloalkyl.

In one aspect of this embodiment, the phenyl group represented by R₅₉ is substituted with from one to three substituents. Preferably, the phenyl represented by R₅₉ is substituted with one substituent.

In some embodiments, in compounds represented by formulas (XXXI) or (XXXV), R₅₉ is a substituted phenyl, an optionally substituted 2,3-dihydro-benzo[1,4]dioxinyl, an optionally substituted benzo[1,3]dioxolyl, an optionally substituted biphenyl, an optionally substituted 4-pyridinyl-phenyl, an optionally substituted quinolinyl, an optionally substituted isoquinolinyl, an optionally substituted 1H-indolyl, an optionally substituted pyridinyl, an optionally substituted oxazolyl, an optionally substituted isoxazolyl, an optionally substituted thiazolyl, an optionally substituted isothiazolyl, an optionally substituted imidazolyl, an optionally substituted pyrrolyl, an optionally substituted pyrazolyl, an optionally substituted furanyl, an optionally substituted thiophenyl, an optionally substituted thiadiazolyl, an optionally substituted oxadiazolyl, an optionally substituted chromanyl, an optionally substituted isochromanyl, an optionally substituted pyridazinyl, an optionally substituted pyrimidinyl, an optionally substituted pyrazinyl, an optionally substituted benzothiophenyl, an optionally substituted 2,3-dihydro-benzothiophenyl, an optionally substituted benzofuranyl, an optionally substituted 2,3-dihydro-benzofuranyl, an optionally substituted 1H-benzoimidazolyl, an optionally substituted benzothiazolyl, an optionally substituted benzooxazolyl, an optionally substituted 1H-benzotriazolyl, an optionally substituted 1H-indazolyl, an optionally substituted 9H-purinyl, an optionally substituted pyrrolopyrimidinyl, an optionally substituted pyrrolopyrazinyl, an optionally substituted pyrrolopyridazinyl, an optionally substituted imidazopyrazinyl, or an optionally substituted imidazolpyridazinyl.

In some embodiments, in the compounds represented by formula (XXXI) or (XXXV), R₅₉ is an optionally substituted pyridinyl. In one aspect of this embodiment, the pyridinyl group represented by R₅₉ is unsubstituted. In another aspect of this embodiment, the pyridinyl group represented by R₅₉ is substituted with one or more substituents independently selected from alkoxy, halo, alkyl, haloalkyl, haloalkoxy, nitro, cyano, oxazolyl, 1H-tetrazolyl, 1-methyl-1H-tetrazolyl, —OR₂₄, —SR₂₄, —C(O)R₂₄, —C(O)OR₂₄, —OC(O)R₂₄, —C(O)NR₂₅R₂₆, —NR₂₄C(O)R₂₇, —NR₂₄C(O)OR₂₇, —OC(O)NR₂₅R₂₆, guanidino, amino, alkyl amino, dialkylamino, —NR₂₄S(O)ₚR₂₈, —S(O)ₚR₂₈, —S(O)ₚOR₂₇, —OS(O)ₚR₂₈, —OS(O)ₚOR₂₇, —OP(O)(OR₂₇)₂, or —SP(O)(OR₂₇)₂, wherein R₂₄, R₂₅, R₂₆, R₂₇, R₂₈ and p are defined as above. In one aspect of this embodiment, the pyridinyl group represented by R₅₉ is substituted with from one to three substituents. Preferably, the pyridinyl represented by R₅₉ is substituted with one substituent.

In some embodiments, in the compounds represented by formula (XXXI) or (XXXV), R₅₉ is an optionally substituted 2,3-dihydro-benzo[1,4]dioxinyl, an optionally substituted biphenyl, an optionally substituted pyridinyl-phenyl, an optionally substituted pyridinyl, an optionally substituted quinolinyl, an optionally substituted isoquinolinyl, an optionally substituted 1H-indolyl, an optionally substituted oxazolyl, an optionally substituted benzo[1,3]dioxolyl, an optionally substituted pyridazinyl, an optionally substituted pyrimidinyl, or an optionally substituted benzofuranyl. In one aspect of this embodiment, R₅₉ is unsubstituted. In another aspect of this embodiment, R₅₉ is substituted with one or more substituents independently selected from alkoxy, halo, alkyl, haloalkyl, haloalkoxy, nitro, cyano, oxazolyl, 1H-tetrazolyl, 1-methyl-1H-tetrazolyl, —OR₂₄, —SR_{N}, —C(O)R₂₄, —C(O)OR₂₄, —OC(O)R₂₄, —C(O)NR₂₅R₂₆, —NR₂₄C(O)R₂₇, —NR₂₄C(O)OR₂₂, —OC(O)NR₂₅R₂₆, guanidino, amino, alkyl amino, dialkylamino, —NR₂₄S(O)ₚR₂₈, —S(O)ₚR₂₈, —S(O)ₚOR₂₇, —OS(O)ₚR₂₈, —OS(O)ₚOR₂₇, —OP(O)(OR₂₇)₂, or —SP(O)(OR₂₇)₂, wherein R₂₄, R₂₅, R₂₆, R₂₇, R₂₈ and p are defined as above. In one aspect of this embodiment, R₅₉ is substituted with from one to three substituents. Preferably, R₅₉ is substituted with one substituent.

In some embodiments, in the compounds represented by formula (XXXVI), (XXXVII), (XXXVIII), (XXXIX), or (XL), R₅₉ is an optionally substituted phenyl. In one aspect of this embodiment, the phenyl group represented by R₅₉ is unsubstituted. In another aspect of this embodiment, the phenyl group represented by R₅₉ is substituted with from one to five groups independently selected from alkoxy, halo, alkyl, haloalkyl, haloalkoxy, nitro, cyano, oxazolyl, 1H-tetrazolyl, 1-methyl-1H-tetrazolyl, —OR₂₄, —SR₂₄, —C(O)R₂₄, —C(O)OR₂₄, —OC(O)R₂₄, —C(O)NR₂₅R₂₆, —NR₂₄C(O)R₂₇, —NR₂₄C(O)OR₂₇, —OC(O)NR₂₅R₂₆, guanidino, amino, alkyl amino, dialkylamino, —NR₂₄S(O)ₚR₂₈, —S(O)ₚR₂₈, —S(O)ₚOR₂₇, —OS(O)ₚR₂₈, —OS(O)ₚOR₂₂, —OP(O)(OR₂₂)₂, or —SP(O)(OR₂₇)₂, wherein R₂₄, R₂₅, R₂₆, R₂₇, R₂₈ and p are defined as above. In one aspect of this embodiment, the phenyl group represented by R₅₉ is substituted with from one to three substituents. Preferably, the phenyl represented by R₅₉ is substituted with one substituent.

In some embodiments, in the compounds represented by formula (XXXVI), (XXXVII), (XXXVIII), (XXXIX), or (XL), R₅₉ is an optionally substituted pyridinyl. In one aspect of this embodiment, the pyridinyl group represented by R₅₉ is unsubstituted. In another aspect of this embodiment, the pyridinyl group represented by R₅₉ is substituted with one or more substituents independently selected from alkoxy, halo, alkyl, haloalkyl, haloalkoxy, nitro, cyano, oxazolyl, 1H-tetrazolyl, 1-methyl-1H-tetrazolyl, —OR₂₄, —SR_{N}, —C(O)R₂₄, —C(O)OR₂₄, —OC(O)R₂₄, —C(O)NR₂₅R₂₆, —NR₂₄C(O)R₂₇, —NR₂₄C(O)OR₂₇, —OC(O)NR₂₅R₂₆, guanidino, amino, alkyl amino, dialkylamino, —NR₂₄S(O)ₚR₂₈, —S(O)ₚR₂₈, —S(O)ₚOR₂₇, —OS(O)ₚR₂₈, —0S(O)ₚ0R₂₇, —OP(O)(OR₂₇)₂, or —SP(O)(OR₂₇)₂, wherein R₂₄, R₂₅, R₂₆, R₂₇, R₂₈ and p are defined as above. In one aspect of this embodiment, the pyridinyl group represented by R₅₉ is substituted with from one to three substituents. Preferably, the pyridinyl represented by R₅₉ is substituted with one substituent.

In some embodiments, in the compounds represented by formula (XXXVI), (XXXVII), (XXXVIII), (XXXIX), or (XL), R₅₉ is an optionally substituted 2,3-dihydro-benzo[1,4]dioxinyl, an optionally substituted biphenyl, an optionally substituted pyridinyl-phenyl, an optionally substituted pyridinyl, an optionally substituted quinolinyl, an optionally substituted isoquinolinyl, an optionally substituted 1H-indolyl, an optionally substituted oxazolyl, an optionally substituted benzo[1,3]dioxolyl, an optionally substituted pyridazinyl, an optionally substituted pyrimidinyl, or an optionally substituted benzofuranyl. In one aspect of this embodiment, R₅₉ is unsubstituted. In another aspect of this embodiment, R₅₉ is substituted with one or more substituents independently selected from alkoxy, halo, alkyl, haloalkyl, haloalkoxy, nitro, cyano, oxazolyl, 1H-tetrazolyl, 1-methyl-1H-tetrazolyl, —OR₂₄, —SR₂₄, —C(O)R₂₄, —C(O)OR₂₄, —OC(O)R₂₄, —C(O)NR₂₅R₂₆, —NR₂₄C(O)R₂₇, NR₂₄C(O)OR₂₇, —OC(O)NR₂₅R₂₆, guanidino, amino, alkyl amino, dialkylamino, —NR₂₄S(O)ₚR₂₈, —S(O)ₚR₂₈, —S(O)ₚOR₂₇, —OS(O)ₚR₂₈, —OS(O)ₚ0R₂₇, —OP(O)(OR₂₇)₂, or —SP(O)(OR₂₇)₂, wherein R₂₄, R₂₅, R₂₆, R₂₇, R₂₈ and p are defined as above. In one aspect of this embodiment, R₅₉ is substituted with from one to three substituents. Preferably, R₅₉ is substituted with one substituent.

In some embodiments, in the compounds represented by formulas (XXXV), (XXXVA), or (XXXVB), R₁₂, R₁₃, and R₁₄ are each, independently, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl. In one aspect of this embodiment, R₁₂, R₁₃, and R₁₄ are each, independently, an alkoxy. In another aspect of this embodiment, R₁₂, R₁₃, and R₁₄ are each methoxy.

In some embodiments, in the compounds represented by formulas (XXXV), (XXXVA), (XXXVB), (XXXVII), (XXXVIIA), (XXXVIIB), (XXXVIII), (XXXVIIIA), or (XXXVIIIB), X₁ and X₂ are CH.

In some embodiments, in the compounds represented by formulas (XXXV), (XXXVA), (XXXVB), (XXXVII), (XXXVIIA), (XXXVIIB), (XXXVIIB, (XXXVIIIA), or (XXXVIIIB), X₁ and X₂ are N.

In some embodiments, in the compounds represented by formula by formulas (XXXV), (XXXVA), (XXXVB), (XXXVII), (XXXVIIA), (XXXVIIB), (XXXVIII), (XXXVIIIA), or (XXXVIIIB), X₁ is N and X₂ is CH.

In some embodiments, in the compounds represented by by formulas (XXXV), (XXXVA), (XXXVB), (XXXVII), (XXXVIIA), (XXXVIIB), (XXXVIII), (XXXVIIIA), or (XXXVIIIB), X₁ is CH and X₂ is N.

In some embodiments, in the compounds represented by formulas (XXXVI), (XXXVIA), or (XXXVIB), X₃ and X₄ are O and X₅ and X₆ are CH. In one aspect of this embodiment, X₃ and X₄ are O; X₅ and X₆ are CH; and R₁₅ is an alkoxy, such as methoxy.

In some embodiments, in the compounds represented by formulas (XXXVI), (XXXVIA), or (XXXVIB), X₃ is CH; X₄ are NR₁₆; and X₅ and X₆ are CH. In one aspect of this embodiment, X₃ is CH; X₄ are NR₁₆; X₅ and X₆ are CH; and R₁₆ is H. In one aspect of this embodiment, X₃ is CH; X₄ are NR₁₆; X₅ and X₆ are CH; and R₁₆ is a lower alkyl.

In some embodiments, in the compounds represented by formulas (XXXVI), (XXXVIA), (XXXVIB), (XXXIX), (XXXIA), (XXXIXB), (XL), (XLA), or (XLB), R₁₅ is H, alkoxy, halo, alkyl, haloalkyl, haloalkoxy, nitro, cyano, —SR₂₄, —C(O)R₂₄, —C(O)OR₂₄, —OC(O)R₂₄, —C(O)NR₂₅R₂₆, —NR₂₄C(O)R₂₇, —NR₂₄C(O)OR₂₇, —OC(O)NR₂₅R₂₆, guanidino, amino, alkylamino, dialkylamino, —NR₂₄S(O)ₚR₂₈, —S(O)ₚR₂₈, —S(O)ₚOR₂₇, —OS(O)ₚR₂₈, —OS(O)ₚOR₂₇, —OP(O)(OR₂₇)₂, or —SP(O)(OR₂₇)₂; wherein R₂₄, R₂₅, R₂₆, R₂₇, R₂₈, and p are defined as above.

In some embodiments, in the compounds represented by formulas (XXXIX), (XXXIXA), (XXXIXB), (XL), (XLA), or (XLB), R₁₅ is H, alkoxy, halo, alkyl, haloalkyl, haloalkoxy, nitro, cyano, —SR₂₄, —C(O)R₂₄, —C(O)0R₂₄, —OC(O)R₂₄, —C(O)NR₂₅R₂₆, —NR₂₄C(O)R₂₇, —NR₂₄C(O)OR₂₇, —OC(O)NR₂₅R₂₆, guanidino, amino, alkylamino, dialkylamino, —NR₂₄S(O)ₚR₂₈, —S(O)ₚR₂₈, —S(O)ₚOR₂₇, —OS(O)ₚR₂₈, —OS(O)ₚOR₂₇, —OP(O)(OR₂₇)₂, or —SP(O)(OR₂₇)₂; and R₂₉, for each occurrence, is independently, H, alkoxy, halo, alkyl, haloalkyl, haloalkoxy, nitro, cyano, —OR₂₄, —SR₂₄, —C(O)R₂₄, —C(O)OR₂₄, —OC(O)R₂₄, —C(O)NR₂₅R₂₆, —NR₂₄C(O)R₂₇, —NR₂₄C(O)OR₂₇, —OC(O)NR₂₅R₂₆, guanidino, amino, alkyl amino, dialkylamino, —NR₂₄S(O)ₚR₂₈, —S(O)ₚR₂₈, —S(O)ₚOR₂₇, —OS(O)ₚR₂₈, —OS(O)ₚ0R₂₇, —OP(O)(OR₂₇)₂, or —SP(O)(OR₂₇)₂; wherein R₂₄, R₂₅, R₂₆, R₂₇, R₂₈, and p are defined as above.

In some embodiments, in the compounds represented by formulas (XXXVII), (XXXVIIA), or (XXXVIIB), R₁₈ and R₁₉ are each, independently, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl; and R₂₀ is an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, or an alkyl ester; wherein R₇ is defined as above.

In some embodiments, in the compounds represented by formula (XXXI), (XXXIA), or (XXXIB), one of Rₐ or R_{b} is —H and the other is a substituted phenyl represented by the following structural formula:

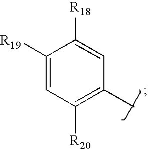

and R₁₈ and R_{1 9} are each, independently, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl; and R₂₀ is an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, or an alkyl ester; wherein R₇ is defined as above and “}” represents the point of attachment of the phenyl ring to the isothiazole ring.

In some embodiments, in the compounds represented by formula (XXXVIII), (XXXVIIIA), or (XXXVIIIB), R₂₂ and R₂₃ are each, independently, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl; and R₂₁ is an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, or an alkyl ester, wherein R₇ is defined as above.

In some embodiments, in the compounds represented by formula (XXXI), (XXXIA), or (XXXIB), one of Rₐ or R_{b} is —H and the other is a substituted phenyl represented by the following structural formula:

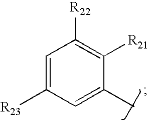

and R₂₂ and R₂₃ are each, independently, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl; and R₂₁ is an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, or an alkyl ester, wherein R₇ is defined as above and “}” represents the point of attachment of the phenyl ring to the isothiazole ring.

In some embodiments, in the compounds represented by formula (XXXI), (XXXIA), or (XXXIB), one of Rₐ or R_{b} is —H and the other is a substituted phenyl. In one aspect, the substituents for Rₐ or R_{b} are independently selected from the group consisting of halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, and —S(O)ₚNR₁₀R₁₁;

R₇ and R₈, for each occurrence, are, independently, -H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;

R₁₀ and R₁₁, for each occurrence, are independently -H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl; and

p is 1 or 2.

In some embodiments, in the compounds represented by formula (XXXI), (XXXIA), or (XXXIB), one of Rₐ or R_{b} is —H and the other is an optionally substituted heteroaryl. In one aspect, the optionally substituted heteroaryl is selected from the group consisting of an optionally substituted 2,3-dihydro-benzo[1,4]dioxinyl, an optionally substituted benzo[1,3]dioxolyl, an optionally substituted quinolinyl, an optionally substituted isoquinolinyl, an optionally substituted 1H-indolyl, an optionally substituted pyridinyl, an optionally substituted oxazolyl, an optionally substituted isoxazolyl, an optionally substituted thiazolyl, an optionally substituted isothiazolyl, an optionally substituted imidazolyl, an optionally substituted pyrazolyl, an optionally substituted furanyl, an optionally substituted thiophenyl, an optionally substituted thiadiazolyl, an optionally substituted oxadiazolyl, an optionally substituted chromanyl, an optionally substituted isochromanyl, an optionally substituted pyridazinyl, an optionally substituted pyrimidinyl, an optionally substituted pyrazinyl, an optionally substituted benzothiophenyl, an optionally substituted 2,3-dihydro-benzothiophenyl, an optionally substituted benzofuranyl, an optionally substituted 2,3-dihydro-benzofuranyl, an optionally substituted 1H-benzoimidazolyl, an optionally substituted benzothiazolyl, an optionally substituted benzooxazolyl, an optionally substituted 1H-benzotriazolyl, an optionally substituted 1H-indazolyl, an optionally substituted 9H-purinyl, an optionally substituted pyrrolopyrimidinyl, an optionally substituted pyrrolopyrazinyl, an optionally substituted pyrrolopyridazinyl, an optionally substituted imidazopyrazinyl, and an optionally substituted imidazolpyridazinyl.

In some embodiments, in the compounds represented by formula (XXXIA), (XXXVA), (XXXVIA), (XXXVIIA), or (XXXVIIIA), R^{x} is R^{aa}, —C(O)YR^{z}, or —C(O)NH—R^{aa}. In one aspect, R^{x} is R^{aa}. In another aspect, R^{x} is —C(O)YR^{z}. R^{aa}, R^{z}, and Y are defined as for formula (XXXIA).

In some embodiments, in the compounds represented by formula (XXXIA), (XXXVA), (XXXVIA), (XXXVIIA), or (XXXVIIIA), R^{x} is R^{aa} and R^{aa} is defined as for formula (XXXIA). In one aspect, R^{aa} is glycine, serine, alanine, phenylalanine, leucine, or methionine.

In some embodiments, in the compounds represented by formula (XXXIA), (XXXVA), (XXXVIA), (XXXVIIA), or (XXXVIIIA), R^{x} is R^{aa} and R^{y} is —H, wherein R^{aa} is defined as for formula (XXXIA). In one aspect, R^{aa} is glycine, alanine, valine, leucine, isoleucine, serine, threonine, cysteine, methionine, phenylalanine, tyrosine, tryptophan, aspartic acid, asparagine, glutamic acid, glutamine, arginine, histidine, lysine, or proline. In another aspect, R^{aa} is glycine, serine, alanine, phenylalanine, leucine, or methionine.

In some embodiments, in the compounds represented by formula (XXXIA), (XXXVA), (XXXVIA), (XXXVIIA), or (XXXVIIIA), R^{x} is —C(O)YR^{z} and Y and R^{z} are defined as for formula (XXXIA). In one aspect, Y is CH₂. In another aspect, Y is O. In another aspect, Y is NH. In one aspect, R^{z} is Y₁ and Y₁ is defined as for formula (XXXIA). In another aspect, R^{z} is Alk-NH₂. In another aspect, R^{z} is Alk-C(O)OH. In another aspect, R^{z} is Het. Alk and Het and defined as for formula (XXXIA).

In some embodiments, in the compounds represented by formula (XXXIA), (XXXVA), (XXXVIA), (XXXVIIA), or (XXXVIIIA), m is 1, 2 or 3.

In some embodiments, in the compounds represented by formula (XXXIA), (XXXVA), (XXXVIA), (XXXVIIA), or (XXXVIIIA), Y₁ is PEG, HPMA copolymer-methacryloyl-Gly-Phe-Leu-Gly-ethylenediamine, or HPMA copolymer-methacryloyl-Gly-Phe-Leu-Gly-OH. In one aspect, Y₁ is PEG.

In some embodiments, in the compounds represented by formula (XXXIA), (XXXVA), (XXXVIA), (XXXVIIA), or (XXXVIIIA), R^{y} is —H.

In some embodiments, in the compounds represented by formula (XXXIA), (XXXVA), (XXXVIA), (XXXVIIA), or (XXXVIIIA), R^{y} is a lower alkyl.

In some embodiments, in the compounds represented by formula (XXXIA), (XXXVA), (XXXVIA), (XXXVIIA), or (XXXVIIIA), Y₁ has a molecular weight greater than 20,000 daltons. In one aspect, Y₁ has a molecular weight of less than 40,000 daltons, but greater than 25,000 daltons.

In some embodiments, in the compounds represented by formula (XXXIA), (XXXVA), (XXXVIA), (XXXVIIA), or (XXXVIIIA), Alk is an optionally substituted lower alkylene.

In some embodiments, in the compounds represented by formula (XXXIA), (XXXVA), (XXXVIA), (XXXVIIA), or (XXXVIIIA), Het is an optionally substituted lower heteroalkyl.

In some embodiments, in the compounds represented by formula (XXXVA), X₁ and X₂ are CH and R₁₂, R₁₃, and R₁₄ are each methoxy. In one aspect, R^{x} is R^{aa}. In another aspect, R^{x} is (R^{aa})ₘ. In another aspect, R^{x} is —R^{aa}—C(O)(CH₂)ₙC(O)OH. In another aspect, R^{x} is —C(O)(CH₂)ₙC(O)OH. In another aspect, R^{x} is —C(O)YR^{z}. In another aspect, R^{x} is —C(O)NH—R^{aa}. In another aspect, R^{x} is —(R^{aa})_{q}C(O)(Y₁). R^{aa}, Y, R^{z}, Y₁, m, n, and q are defined as for formula (XXXIA).

In some embodiments, in the compounds represented by formula (XXXVA), X₁ and X₂ are CH and R₁₂, R₁₃, and R₁₄ are each methoxy. In one aspect, R^{x} is R^{aa} and R^{w} is alkoxy. In another aspect, R^{x} is R^{aa} and R^{y} is —H. In another aspect, R^{x} is R^{aa}, R^{w} is alkoxy, and R^{y} is —H. In another aspect, R^{x} is R^{aa}, R^{w} is alkoxy, and R^{y} is —H. In another aspect, R^{x} is R^{aa}, R^{w} is methoxy, and R^{y} is —H. R^{aa} is defined as for formula (XXXIA).

In some embodiments, in the compounds represented by formula (XXXVB), X₁ and X₂ are CH; R₁₂, R₁₃, and R₁₄ are each methoxy; and R^{w} is alkoxy. In one aspect, R^{w} is methoxy.

In some embodiments, in the compounds represented by formula (XXXIA or B), (XXXVA or B), (XXXVIA or B), (XXXVIIA or B), (XXXVIIIA or B), (XXXIXA or B), or (XLA or B), R^{w} is alkoxy. In one aspect, R^{w} is methoxy.

In some embodiments, in the compounds represented by formula (XXXI), (XXXIA), or (XXXIB), Rₐ is —H. In some embodiments, in the compounds represented by formulas (XXXI), (XXXIA), or (XXXIB), R_{b} is —H. In some embodiments, in the compounds represented by formula (XXXV), (XXXVA), or (XXXVB), Rᵢ is —H. In some embodiments, in the compounds represented by formulas (XXXV), (XXXVA), or (XXXVB), Rⱼ is —H. In some embodiments, in the compounds represented by formula (XXXVI), (XXXVIA), or (XXXVIB), Rₖ is —H. In some embodiments, in the compounds represented by formulas (XXXVI), (XXXVIA), or (XXXVIB), Rₗ is —H. In some embodiments, in the compounds represented by formula (XXXVII), (XXXVIIA), or (XXXVIIB), Rₘ is —H. In some embodiments, in the compounds represented by formulas (XXXVII), (XXXVIIA), or (XXXVIIB), Rₙ is —H. In some embodiments, in the compounds represented by formula (XXXVIII), (XXXVIIIA), or (XXXVIIIB), Rₒ is —H. In some embodiments, in the compounds represented by formulas (XXXVIII), (XXXVIIIA), or (XXXVIIIB), Rₚ is —H.

In another embodiment, the invention relates to compounds selected from the group consisting of:
4-(4-Bromo-phenyl)-5-(3,4,5-trimethoxy-phenyl)-isothiazole;
4-(4-Bromo-phenyl)-5-(3,4,5-trimethoxy-phenyl)-isothiazole;
4-(4-methoxyphenyl)-5-(3,4,5-trimethoxyphenypisothiazole;
4-(4-Iodo-phenyl)-5-(2-hydroxy-4-methoxy-5-ethyl-phenyl)-isothiazole;
4-(4-Bromo-phenyl)-5-(2-hydroxy-4-methoxy-5-ethyl-phenyl)-isothiazole;
4-(2,3-Dihydro-benzo[1,4]di-oxin-6-yl)-5-(2-hydroxy-4-methoxy-5-propyl-phenyl)-isothiazole;
4-(4-hydroxy-phenyl)-5-(3,4,5-trihydroxy-phenyl)-isothiazole;
4-(4-Iodo-phenyl)-5-(3,4,5-trimethoxy-phenyl)-isothiazole;
4-(3-Fluoro-4-methoxy-phenyl)-5-(3,4,5-trimethoxy-phenyl)-isothiazole;
4-(4-Nitro-phenyl)-5-(3,4,5-trimethoxy-phenyl)-isothiazole;
4-(4-Amino-phenyl)-5-(3,4,5-trimethoxy-phenyl)-isothiazole;
4-(4′-Methoxy-biphenyl-4-yl)-5-(3,4,5-trimethoxy-phenyl)-isothiazole;
4-[4-(pyridine-3-yl)-phenyl]-5-(3,4,5-trimethoxy-phenyl)-isothiazole;
4-[4-(pyridine-4-yl)-phenyl]-5-(3,4,5-trimethoxy-phenyl)-isothiazole;
4-[4-(pyridine-2-yl)-phenyl]-5-(3,4,5-trimethoxy-phenyl)-isothiazole;
4-(Quinolin-7-yl)-5-(3,4,5-trimethoxy-phenyl)-isothiazole;
4-(Pyridin-4-yl)-5-(3,4,5-trimethoxy-phenyl)-isothiazole;
4-(Isoquinolin-7-yl)-5-(3,4,5-trimethoxy-phenyl)-isothiazole;
4-(1-Methyl-1H-indol-5-yl)-5-(3,4,5-trimethoxy-phenyl)-isothiazole;
4-(4-Methoxy-phenyl)-5-(benzo[1,3]dioxol-5-yl)-isothiazole;
4-(4-Methoxy-phenyl)-5-(1-ethyl-1H-indol-6-yl)-isothiazole;
4-(4-Carboxy-phenyl)-5-(3,4,5-trimethoxy-phenyl)-isothiazole;
4-(4-Methoxycarbonyl-phenyl)-5-(3,4,5-trimethoxy-phenyl)-isothiazole;
4-[4-(Oxazol-2-yl)-phenyl]-5-(3,4,5-trimethoxy-phenyl)-isothiazole;
4-(4-Methoxy-phenyl)-5-(3,4,5-triethyl-phenyl)-isothiazole;
4-(4-Iodo-phenyl)-5-(3,4,5-triethyl-phenyl)-isothiazole;
4-(3-Fluoro-4-methoxy-phenyl)-5-(3,4,5-triethyl-phenyl)-isothiazole;
4-(4-Nitro-phenyl)-5-(3,4,5-triethyl-phenyl)-isothiazole;
4-(4-N,N-dimethylamino-phenyl)-5-(3,4,5-triethyl-phenyl)-isothiazole;
4-(4-Methoxy-phenyl)-5-(3,4,5-trimethyl-phenyl)-isothiazole;
4-[4-(Pyridin-3-yl)-phenyl]-5-(3,4,5-triethyl-phenyl)-isothiazole;
4-[4-(Pyridin-4-yl)-phenyl]-5-(3,4,5-triethyl-phenyl)-isothiazole;
4-[4-(Pyridin-2-yl)-phenyl]-5-(3,4,5-triethyl-phenyl)-isothiazole;
4-(Quinolin-7-yl)-5-(3,4,5-triethyl-phenyl)-isothiazole;
4-(Pyridin-4-yl)-5-(3,4,5-triethyl-phenyl)-isothiazole;
4-(Isoquinolin-7-yl)-5-(3,4,5-triethyl-phenyl)-isothiazole;
4-(1H-Indol-5-yl)-5-(3,4,5-triethyl-phenyl)-isothiazole;
4-(4-Methoxy-phenyl)-5-(benzo[1,3]dioxol-5-yl)-isothiazole;
4-(4-Methoxy-phenyl)-5-[1-isopropyl-1H-indol-6-yl)-isothiazole;
4-(4-Methoxy-phenyl)-5-(2,3,4-trimethoxy-phenyl)-isothiazole;
4-(3-Hydroxy-4-methoxy-phenyl)-5-(3,4,5-trimethoxy-phenyl)-isothiazole;
4-[3-(Ethyl-hydroxy-phosphoryloxy)-4-methoxy-phenyl]-5-(3,4,5-trimethoxy-phenyl)-isothiazole;
4-(4-Methoxy-phenyl)-5-(2-hydroxy-4-methoxy-5-ethyl-phenyl)-isothiazole;
4-(4-Isopropyl-phenyl)-5-(3,4,5-trimethoxy-phenyl)-isothiazole;
4-(2,3-Dihydro-benzo[1,4]dioxin-6-yl)-5-(3,4,5-trimethoxy-phenyl)-isothiazole;
4-(4-Ethyl-phenyl)-5-(3,4,5-trimethoxy-phenyl)-isothiazole;
4-(5-Methoxy-pyridin-2-yl)-5-(3,4,5-trimethoxy-phenyl)-isothiazole;
4-(4-Methoxy-phenyl)-5-(2,3,4-trimethoxy-pyridin-6-yl)-isothiazole;
4-(4-Methoxy-phenyl)-5-(3,5-dimethoxy-4-methoxycarbonyl-phenyl)-isothiazole;
4-(4-Methoxy-phenyl)-5-(3,5-diacetoxy-phenyl)-isothiazole;
4-(2-Methoxy-pyridin-5-yl)-5-(3,4,5-trimethoxy-phenyl)-isothiazole;
4-(4-Methoxy-phenyl)-5-(1-methyl-5-methoxy-1H-indol-7-yl)-isothiazole;
4-(4-Methoxy-phenyl)-5-(1-ethyl-1H-indol-7-yl)-isothiazole;
4-(4-Methoxy-phenyl)-5-(benzo[1,3]dioxol-4-yl)-isothiazole;
4-(2-Hydroxy-4-methoxy-phenyl)-5-(3,4,5-trimethoxy)-isothiazole;
4-[2-(Ethyl-hydroxy-phosphoryloxy)-4-methoxy-phenyl]-5-(3,4,5-trimethoxy-phenyl)-isothiazole;
4-(Pyridazin-4-yl)-5-(3,4,5-trimethoxy-phenyl)-isothiazole;
4-(Pyrimidin-5-yl)-5-(3,4,5-trimethoxy-phenyl)-isothiazole;
4-(Pyridin-3-yl)-5-(3,4,5-trimethoxy-phenyl)-isothiazole, hydrochloric acid salt;
4-(3-Mercapto-4-methoxy-phenyl)-5-(3,4,5-trimethoxy-phenyl)-isothiazole;
4-(3-Phosphonosulfanyl-4-methoxy-phenyl)-5-(3,4,5-trimethoxy-phenyl)-isothiazole, disodium salt;
4-(3-Acetylamino-4-methoxy-phenyl)-5-(3,4,5-trimethoxy-phenyl)-isothiazole;
2-methoxy-5-[5-(3,4,5-trimethoxy-phenyl)-isothiozol-4-yl)-phenylamine;
4-(2-Hydroxy-4-methoxy-phenyl)-5-(3,4,5-trimethoxy-phenyl)-isothiazole;
4-(2-Methoxy-pyridine-5-yl)-5-(3,4,5-trimethoxy-phenyl)-isothiazole;
4-(5-Methoxy-pyridine-2-yl)-5-(3,4,5-trimethoxy-phenyl)-isothiazole;
4-(3-Carboxy-4-methoxy-phenyl)-5-(3,4,5-trimethoxy-phenyl)-isothiazole, sodium salt;
4-(3-Methoxycarbonyl-4-methoxy-phenyl)-5-(3,4,5-trimethoxy-phenyl)-isothiazole;
4-(3-Sulfooxy-4-methoxy-phenyl)-5-(3,4,5-trimethoxy-phenyl)-isothiazole, sodium salt;
4-(2-Amino-4-methoxy-phenyl)-5-(3,4,5-trimethoxy-phenyl)-isothiazole;
4-(3,4-Dimethoxy-5-phosphonooxy-phenyl)-5-(3,4,5-trimethoxy-phenyl)-isothiazole, disodium salt;
4-(2-Phosphonooxy-4-methoxy-phenyl)-5-(3,4,5-trimethoxy-phenyl)-isothiazole, disodium salt;
4-(4-Methylsulfanyl-phenyl)-5-(3,4,5-trimethoxy-phenyl)-isothiazole
4-(3-Phosphonooxy-4-methylsulfanyl-phenyl)-5-(3,4,5-trimethoxy-phenyl)-isothiazole, disodium salt;
4-(3-Amino-4-methylsulfanyl-phenyl)-5-(3,4,5-trimethoxy-phenyl)-isothiazole;
4-(2,3-Dihydro-benzofuran-6-yl)-5-(3,4,5-trimethoxy-phenyl)-isothiazole;
4-(4-Hydroxy-phenyl)-5-(3,4,5-trimethoxy-phenyl)-isothiazole, sodium salt;
4-(4-Phosphonooxy-phenyl)-5-(3,4,5-trimethoxy-phenyl)-isothiazole, disodium salt;
4-(4-1H-Tetrazol-5-yl-phenyl)-5-(3,4,5-trimethoxy-phenyl)-isothiazole;
4-[4-(1-Methyl-1H-tetrazol-5-yl)-phenyl]-5-(3,4,5-trimethoxy-phenyl)-isothiazole;
4-(1-Methyl-1H-indol-5-yl)-5-(3,4,5-trimethoxy-phenyl)-isothiazole;
4-(Pyridazin-4-yl)-5-(4-methoxy-benzo[1,3]dioxol-6-yl)-isothiazole;
4-(Pyrimidin-5-yl)-5-(4-methoxy-benzo[1,3]dioxol-6-yl)-isothiazole;
4-(Pyridin-3-yl)-5-(4-methoxy-benzo[1,3]dioxol-6-yl)-isothiazole hydrochloric acid salt;
4-(3-Mercapto-4-methoxy-phenyl)-5-(4-methoxy-benzo[1,3]dioxol-6-yl)-isothiazole;
4-(3-Phosphonosulfanyl-4-methoxy-phenyl)-5-(4-methoxy-benzo[1,3]dioxol-6-yl)-isothiazole;
4-(3-Acetylamino-4-methoxy-phenyl)-5-(4-methoxy-benzo[1,3]dioxol-6-yl)-isothiazole;
4-(3-Amino-4-methoxy-phenyl)-5-(4-methoxy-benzo[1,3]dioxol-6-yl)-isothiazole, hydrochloric acid salt;
4-(2-Hydroxy-4-methoxy-phenyl)-5-(4-methoxy-benzo[1,3]dioxol-6-yl)-isothiazole;
4-(2-Methoxy-pyridin-5-yl)-5-(4-methoxy-benzo[1,3]dioxol-6-yl)-isothiazole;
4-(5-Methoxy-pyridin-2-yl)-5-(4-methoxy-benzo[1,3]dioxol-6-yl)-isothiazole;
4-(3-Carboxy-4-methoxy-phenyl)-5-(4-methoxy-benzo[1,3]dioxol-6-yl)-isothiazole, sodium salt;
4-(3-Methoxycarbonyl-4-methoxy-phenyl)-5-(4-methoxy-benzo[1,3]dioxol-6-yl)-isothiazole;
4-(3-Sulfooxy-4-methoxy-phenyl)-5-(4-methoxy-benzo[1,3]dioxol-6-yl)-isothiazole, sodium salt;
4-(3-Amino-4-methoxy-phenyl)-5-(4-methoxy-benzo[1,3]dioxol-6-yl)-isothiazole;
4-(3,4-Dimethoxy-5 phosphonooxy-phenyl)-5-(4-methoxy-benzo[1,3]dioxol-6-yl)-isothiazole, disodium salt;
4-(2-Phosphonooxy-4-methoxy-phenyl)-5-(4-methoxy-benzo[1,3]dioxol-6-yl)-isothiazole, disodium salt;
4-(4-Methylsulfanyl-phenyl)-5-(4-methoxy-benzo[1,3]dioxol-6-yl)-isothiazole;
4-(3-Phosphonooxy-4-methylsulfanyl-phenyl)-5-(4-methoxy-benzo[1,3]dioxol-6-yl)-isothiazole, disodium salt;
4-(3-Amino-4-methylsulfanyl-phenyl)-5-(4-methoxy-benzo[1,3]dioxol-6-yl)-isothiazole;
4-(2,3-Dihydro-benzofuran-6-yl)-5-(4-methoxy-benzo[1,3]dioxol-6-yl)-isothiazole;
4-(4-Hydroxy-phenyl)-5-(4-methoxy-benzo[1,3]dioxol-6-yl)-isothiazole, sodium salt;
4-(4-Phosphonooxy-phenyl)-5-(4-methoxy-benzo[1,3]dioxol-6-yl)-isothiazole;
4-(4-1H-Tetrazol-5-yl-phenyl)-5-(4-methoxy-benzo[1,3]dioxol-6-yl)-isothiazole;
4-[4-(1-Methyl-1H-tetrazol-5-yl)-phenyl]-5-(4-methoxy-benzo[1,3]dioxol-6-yl)-isothiazole;
4-(1-Methyl-1H-indo1-5-yl)-5-(4-methoxy-benzo[1,3]dioxol-6-yl)-isothiazole;
4-(3,4,5-Trimethoxy-phenyl)-5-(1-methyl-1H-indol-5-yl)-isothiazole;
4-(3,4,5-Trimethoxy-phenyl)-5-(3-phosphonooxy-4-methoxy-phenyl) isothiazole, disodium salt;
4-(3,4,5-Trimethoxy-phenyl)-5-(N,N-dimethylamino-phenyl)-isothiazole;
4-(3,4,5-Trimethoxy-phenyl)-5-(3-amino-4-methoxy-phenyl)-isothiazole, hydrochloric acid salt;
4-(3,4,5-Trimethoxy-phenyl)-5-[3-(3-hydroxy-2S-amino-propionamido)-4-methoxy-phenyl]-isothiazole, hydrochloric acid salt;
4-(4-Methoxy-phenyl)-5-(2,4,5-trimethoxy-phenyl)-isothiazole;
4-(4-Methyl-phenyl)-5-(2,4,5-trimethoxy-phenyl)-isothiazole;
4-(4-Ethoxy-phenyl)-5-(2,4,5-trimethoxy-phenyl)-isothiazole;
4-(4-Ethyl-phenyl)-5-(2,4,5-trimethoxy-phenyl)-isothiazole;
4-(4-Propoxy-phenyl)-5-(2,4,5-trimethoxy-phenyl)-isothiazole;
4-(4-Propyl-phenyl)-5-(2,4,5-trimethoxy-phenyl)-isothiazole;
4-(4-Butoxy-phenyl)-5-(2,4,5-trimethoxy-phenyl)-isothiazole;
4-(4-Butyl-phenyl)-5-(2,4,5-trimethoxy-phenyl)-isothiazole;
4-(4-Bromo-phenyl)-5-(2,4,5-trimethoxy-phenyl)-isothiazole;
4-(4-Chloro-phenyl)-5-(2,4,5-trimethoxy-phenyl)-isothiazole;
4-(4-Fluoro-phenyl)-5-(2,4,5-trimethoxy-phenyl)-isothiazole;
4-(4-Nitro-phenyl)-5-(2,4,5-trimethoxy-phenyl)-isothiazole
4-[4-(N,N-Dimethylamino)-phenyl]-5-(2,4,5-trimethoxy-phenyl)-isothiazole;
4-(3,4-Dimethoxy-phenyl)-5-(2,4,5-trimethoxy-phenyl)-isothiazole;
4-(3-Hydroxy-4-methoxy-phenyl)-5-(2,4,5-trimethoxy-phenyl)-isothiazole;
4-(3,4,5-Trimethoxy-phenyl)-5-(2,4,5-trimethoxy-phenyl)-isothiazole;
4-(4-Methoxy-phenyl)-5-(2,3,5-trimethoxy-phenyl)-isothiazole;
4-(4-Methyl-phenyl)-5-(2,3,5-trimethoxy-phenyl)-isothiazole;
4-(4-Ethoxy-phenyl)-5-(2,3,5-trimethoxy-phenyl)-isothiazole;
4-(4-Ethyl-phenyl)-5-(2,3,5-trimethoxy-phenyl)-isothiazole;
4-(4-Propoxy-phenyl)-5-(2,3,5-trimethoxy-phenyl)-isothiazole;
4-(4-Propyl-phenyl)-5-(2,3,5-trimethoxy-phenyl)-isothiazole;
4-(4-Butoxy-phenyl)-5-(2,3,5-trimethoxy-phenyl)-isothiazole;
4-(4-Butyl-phenyl)-5-(2,3,5-trimethoxy-phenyl)-isothiazole;
4-(4-Bromo-phenyl)-5-(2,3,5-trimethoxy-phenyl)-isothiazole;
4-(4-Chloro-phenyl)-5-(2,3,5-trimethoxy-phenyl)-isothiazole;
4-(4-Fluoro-phenyl)-5-(2,3,5-trimethoxy-phenyl)-isothiazole;
4-(4-Nitro-phenyl)-5-(2,3,5-trimethoxy-phenyl)-isothiazole;
4-[4-(N,N-Dimethylamino)-phenyl]-5-(2,3,5-trimethoxy-phenyl)-isothiazole;
4-(3,4-Dimethoxy-phenyl)-5-(2,3,5-trimethoxy-phenyl)-isothiazole;
4-(3-Hydroxy-4-methoxy-phenyl)-5-(2,3,5-trimethoxy-phenyl)-isothiazole;
4-(3,4,5-Trimethoxy-phenyl)-5-(2,3,5-trimethoxy-phenyl)-isothiazole;
4-(2,3,4,5-Tetramethoxy-phenyl)-5-(4-methoxy-phenyl)-isothiazole;
4-(2,3,4,5-Tetramethoxy-phenyl)-5-(4-methyl-phenyl)-isothiazole;
4-(2,3,4,5-Tetramethoxy-phenyl)-5-(4-ethoxy-phenyl)-isothiazole;
4-(2,3,4,5-Tetramethoxy-phenyl)-5-(4-ethyl-phenyl)-isothiazole;
4-(2,3,4,5-Tetramethoxy-phenyl)-5-(4-propoxy-phenyl)-isothiazole;

4-(2,3,4,5-Tetramethoxy-phenyl)-5-(4-propyl-phenyl)-isothiazole;
4-(2,3,4,5-Tetramethoxy-phenyl)-5-(4-butoxy-phenyl)-isothiazole;
4-(2,3,4,5-Tetramethoxy-phenyl)-5-(4-butyl-phenyl)-isothiazole;
4-(2,3,4,5-Tetramethoxy-phenyl)-5-(4-bromo-phenyl)-isothiazole;
4-(2,3,4,5-Tetramethoxy-phenyl)-5-(4-chloro-phenyl)-isothiazole;
4-(2,3,4,5-Tetramethoxy-phenyl)-5-(4-fluoro-phenyl)-isothiazole;
4-(2,3,4,5-Tetramethoxy-phenyl)-5-(4-nitro-phenyl)-isothiazole;
4-(2,3,4,5-Tetramethoxy-phenyl)-5-[4-(N,N,-dimethylamino)-phenyl]-isothiazole;
4-(2,3,4,5-Tetramethoxy-phenyl)-5-(3,4-dimethoxy-phenyl)-isothiazole;
4-(2,3,4,5-Tetramethoxy-phenyl)-5-(3-hydroxy-4-methoxy-phenyl)-isothiazole;
4-(2,3,4,5-Tetramethoxy-phenyl)-5-(3,4,5-trimethoxy-phenyl)-isothiazole;
4-(2,3-Dihydro-benzo[1,4]dioxin-6-yl)-5-(3,4-dimethoxy-phenyl)-isothiazole;
4-(3,4-Dimethy-phenyl)-5-(2-hydroxy-4-methoxy-5-ethyl-phenyl)-isothiazole;
4-(4-Chloro-phenyl)-5-(2-hydroxy-4-methoxy-5-ethyl-phenyl)-isothiazole;
4-(4-Methyl-phenyl)-5-(2-hydroxy-4-methoxy-5-ethyl-phenyl)-isothiazole;
4-(4-Amino-phenyl)-5-(2-hydroxy-4-methoxy-5-ethyl-phenyl)-isothiazole;
4-(4-Trifluoromethyl-phenyl)-5-(2-hydroxy-4-methoxy-5-ethyl-phenyl)-isothiazole;
4-(4-Methoxy-phenyl)-5-(2-hydroxy-4-methoxy-5-ethyl-phenyl)-isothiazole; and
4-(3,4,5-Trimethoxy-phenyl)-5-(4-bromo-phenyl)-isothiazole;

or pharmaceutically acceptable salts, solvates, clathrates, or prodrugs thereof.

In another embodiment, the invention relates to compounds selected from the group consisting of:
4-(4-Bromo-phenyl)-3-(3,4,5-trimethoxy-phenyl)-isothiazole;
4-(Naphthalen-2-yl)-3-(2-hydroxy-4-methoxy-5-ethyl-phenyl)-isothiazole;
4-(4-methoxyphenyl)-3-(3,4,5-trimethoxyphenyl)isothiazole;
4-(4-Iodo-phenyl)-3-(2-hydroxy-4-methoxy-5-ethyl-phenyl)-isothiazole;
4-(4-Bromo-phenyl)-3-(2-hydroxy-4-methoxy-5-ethyl-phenyl)-isothiazole;
4-(2,3-Dihydro-benzo[1,4]di-oxin-6-yl)-3-(2-hydroxy-4-methoxy-5-propyl-phenyl)-isothiazole;
4-(4-hydroxy-phenyl)-3-(3,4,5-trihydroxy-phenyl)-isothiazole;
4-(4-Iodo-phenyl)-3-(3,4,5-trimethoxy-phenyl)-isothiazole;
4-(3-Fluoro-4-methoxy-phenyl)-3-(3,4,5-trimethoxy-phenyl)-isothiazole;
4-(4-Nitro-phenyl)-3-(3,4,5-trimethoxy-phenyl)-isothiazole;
4-(4-Amino-phenyl)-3-(3,4,5-trimethoxy-phenyl)-isothiazole;
4-(4′-Methoxy-biphenyl-4-yl)-3-(3,4,5-trimethoxy-phenyl)-isothiazole;
4-[4-(pyridine-3-yl)-phenyl]-3-(3,4,5-trimethoxy-phenyl)-isothiazole;
4-[4-(pyridine-4-yl)-phenyl]-3-(3,4,5-trimethoxy-phenyl)-isothiazole;
4-[4-(pyridine-2-yl)-phenyl]-3-(3,4,5-trimethoxy-phenyl)-isothiazole;
4-(Quinolin-7-yl)-3-(3,4,5-trimethoxy-phenyl)-isothiazole;
4-(Pyridin-4-yl)-3-(3,4,5-trimethoxy-phenyl)-isothiazole;
4-(Isoquinolin-7-yl)-3-(3,4,5-trimethoxy-phenyl)-isothiazole;
4-(1-Methyl-1H-indol-5-yl)-3-(3,4,5-trimethoxy-phenyl)-isothiazole;
4-(4-Methoxy-phenyl)-3-(benzo[1,3]dioxol-5-yl)-isothiazole;
4-(4-Methoxy-phenyl)-3-(1-ethyl-1H-indol-6-yl)-isothiazole;
4-(4-Carboxy-phenyl)-3-(3,4,5-trimethoxy-phenyl)-isothiazole;
4-(4-Methoxycarbonyl-phenyl)-3-(3,4,5-trimethoxy-phenyl)-isothiazole;
4-[4-(Oxazol-2-yl)-phenyl]-3-(3,4,5-trimethoxy-phenyl)-isothiazole;
4-(4-Methoxy-phenyl)-3-(3,4,5-triethyl-phenyl)-isothiazole;
4-(4-Iodo-phenyl)-3-(3,4,5-triethyl-phenyl)-isothiazole;
4-(3-Fluoro-4-methoxy-phenyl)-3-(3,4,5-triethyl-phenyl)-isothiazole;
4-(4-Nitro-phenyl)-3-(3,4,5-triethyl-phenyl)-isothiazole;
4-(4-N,N-dimethylamino-phenyl)-3-(3,4,5-triethyl-phenyl)-isothiazole;
4-(4-Methoxy-phenyl)-3-(3,4,5-trimethyl-phenyl)-isothiazole;
4-[4-(Pyridin-3-yl)-phenyl]-3-(3,4,5-triethyl-phenyl)-isothiazole;
4-[4-(Pyridin-4-yl)-phenyl]-3-(3,4,5-triethyl-phenyl)-isothiazole;
4-[4-(Pyridin-2-yl)-phenyl]-3-(3,4,5-triethyl-phenyl)-isothiazole;
4-(Quinolin-7-yl)-3-(3,4,5-triethyl-phenyl)-isothiazole;
4-(Pyridin-4-yl)-3-(3,4,5-triethyl-phenyl)-isothiazole;
4-(Isoquinolin-7-yl)-3-(3,4,5-triethyl-phenyl)-isothiazole;
4-(1H-Indol-5-yl)-3-(3,4,5-triethyl-phenyl)-isothiazole;
4-(4-Methoxy-phenyl)-3-(benzo[1,3]dioxol-5-yl)-isothiazole;
4-(4-Methoxy-phenyl)-3-[1-isopropyl-1H-indol-6-yl)-isothiazole;
4-(4-Methoxy-phenyl)-3-(2,3,4-trimethoxy-phenyl)-isothiazole;
4-(3-Hydroxy-4-methoxy-phenyl)-3-(3,4,5-trimethoxy-phenyl)-isothiazole;
4-[3-(Ethyl-hydroxy-phosphoryloxy)-4-methoxy-phenyl]-3-(3,4,5-trimethoxy-phenyl)-isothiazole;
4-(4-Methoxy-phenyl)-3-(2-hydroxy-4-methoxy-5-ethyl-phenyl)-isothiazole;
4-(4-Isopropyl-phenyl)-3-(3,4,5-trimethoxy-phenyl)-isothiazole;
4-(2,3-Dihydro-benzo[1,4]dioxin-6-yl)-3-(3,4,5-trimethoxy-phenyl)-isothiazole;
4-(4-Ethyl-phenyl)-3-(3,4,5-trimethoxy-phenyl)-isothiazole;
4-(5-Methoxy-pyridin-2-yl)-3-(3,4,5-trimethoxy-phenyl)-isothiazole;
4-(4-Methoxy-phenyl)-3-(2,3,4-trimethoxy-pyridin-6-yl)-isothiazole;
4-(4-Methoxy-phenyl)-3-(3,5-dimethoxy-4-methoxycarbonyl-phenyl)-isothiazole;
4-(4-Methoxy-phenyl)-3-(3,5-diacetoxy-phenyl)-isothiazole;
4-(2-Methoxy-pyridin-5-yl)-3-(3,4,5-trimethoxy-phenyl)-isothiazole;
4-(4-Methoxy-phenyl)-3-(1-methyl-5-methoxy-1H-indol-7-yl)-isothiazole;
4-(4-Methoxy-phenyl)-3-(1-ethyl-1H-indol-7-yl)-isothiazole;
4-(4-Methoxy-phenyl)-3-(benzo[1,3]dioxol-4-yl)-isothiazole;
4-(2-Hydroxy-4-methoxy-phenyl)-3-(3,4,5-trimethoxy)-isothiazole;
4-[2-(Ethyl-hydroxy-phosphoryloxy)-4-methoxy-phenyl]-3-(3,4,5-trimethoxy-phenyl)-isothiazole;
4-(Pyridazin-4-yl)-3-(3,4,5-trimethoxy-phenyl)-isothiazole;
4-(Pyrimidin-5-yl)-3-(3,4,5-trimethoxy-phenyl)-isothiazole;
4-(Pyridin-3-yl)-3-(3,4,5-trimethoxy-phenyl)-isothiazole, hydrochloric acid salt;
4-(3-Mercapto-4-methoxy-phenyl)-3-(3,4,5-trimethoxy-phenyl)-isothiazole;
4-(3-Phosphonosulfanyl-4-methoxy-phenyl)-3-(3,4,5-trimethoxy-phenyl)-isothiazole, disodium salt;
4-(3-Acetylamino-4-methoxy-phenyl)-3-(3,4,5-trimethoxy-phenyl)-isothiazole;
4-(3-Amino-4-methoxy-phenyl)-3-(3,4,5-trimethoxy-phenyl)-isothiazole;
4-(2-Hydroxy-4-methoxy-phenyl)-3-(3,4,5-trimethoxy-phenyl)-isothiazole;
4-(2-Methoxy-pyridine-5-yl)-3-(3,4,5-trimethoxy-phenyl)-isothiazole;
4-(5-Methoxy-pyridine-2-yl)-3-(3,4,5-trimethoxy-phenyl)-isothiazole;
4-(3-Carboxy-4-methoxy-phenyl)-3-(3,4,5-trimethoxy-phenyl)-isothiazole, sodium salt;
4-(3-Methoxycarbonyl-4-methoxy-phenyl)-3-(3,4,5-trimethoxy-phenyl)-isothiazole;
4-(3-Sulfooxy-4-methoxy-phenyl)-3-(3,4,5-trimethoxy-phenyl)-isothiazole, sodium salt;
4-(2-Amino-4-methoxy-phenyl)-3-(3,4,5-trimethoxy-phenyl)-isothiazole;
4-(3,4-Dimethoxy-5-phosphonooxy-phenyl)-3-(3,4,5-trimethoxy-phenyl)-isothiazole, disodium salt;
4-(2-Phosphonooxy-4-methoxy-phenyl)-3-(3,4,5-trimethoxy-phenyl)-isothiazole, disodium salt;
4-(4-Methylsulfanyl-phenyl)-3-(3,4,5-trimethoxy-phenyl)-isothiazole;
4-(3-Phosphonooxy-4-methylsulfanyl-phenyl)-3-(3,4,5-trimethoxy-phenyl)-isothiazole, disodium salt;
4-(3-Amino-4-methylsulfanyl-phenyl)-3-(3,4,5-trimethoxy-phenyl)-isothiazole;
4-(2,3-Dihydro-benzofuran-6-yl)-3-(3,4,5-trimethoxy-phenyl)-isothiazole;
4-(4-Hydroxy-phenyl)-3-(3,4,5-trimethoxy-phenyl)-isothiazole, sodium salt;
4-(4-Phosphonooxy-phenyl)-3-(3,4,5-trimethoxy-phenyl)-isothiazole, disodium salt;
4-(4-1H-Tetrazol-5-yl-phenyl)-3-(3,4,5-trimethoxy-phenyl)-isothiazole;
4-[4-(1-Methyl-1H-tetrazol-5-yl)-phenyl]-3-(3,4,5-trimethoxy-phenyl)-isothiazole;
4-(1-Methyl-1H-indol-5-yl)-3-(3,4,5-trimethoxy-phenyl)-isothiazole;
4-(Pyridazin-4-yl)-3-(4-methoxy-benzo[1,3]dioxol-6-yl)-isothiazole;
4-(Pyrimidin-5-yl)-3-(4-methoxy-benzo[1,3]dioxol-6-yl)-isothiazole;
4-(Pyridin-3-yl)-3-(4-methoxy-benzo[1,3]dioxol-6-yl)-isothiazole, hydrochloric acid salt;
4-(3-Mercapto-4-methoxy-phenyl)-3-(4-methoxy-benzo[1,3]dioxol-6-yl)-isothiazole;
4-(3-Phosphonosulfanyl-4-methoxy-phenyl)-3-(4-methoxy-benzo[1,3]dioxol-6-yl)-isothiazole;
4-(3-Acetylamino-4-methoxy-phenyl)-3-(4-methoxy-benzo[1,3]dioxol-6-yl)-isothiazole;
4-(3-Amino-4-methoxy-phenyl)-3-(4-methoxy-benzo[1,3]dioxol-6-yl)-isothiazole, hydrochloric acid salt;
4-(2-Hydroxy-4-methoxy-phenyl)-3-(4-methoxy-benzo[1,3]dioxol-6-yl)-isothiazole;
4-(2-Methoxy-pyridin-5-yl)-3-(4-methoxy-benzo[1,3]dioxol-6-yl)-isothiazole;
4-(5-Methoxy-pyridin-2-yl)-3-(4-methoxy-benzo[1,3]dioxol-6-yl)-isothiazole;
4-(3-Carboxy-4-methoxy-phenyl)-3-(4-methoxy-benzo[1,3]dioxol-6-yl)-isothiazole, sodium salt;
4-(3-Methoxycarbonyl-4-methoxy-phenyl)-3-(4-methoxy-benzo[1,3]dioxol-6-yl)-isothiazole;
4-(3-Sulfooxy-4-methoxy-phenyl)-3-(4-methoxy-benzo[1,3]dioxol-6-yl)-isothiazole, sodium salt;
4-(3-Amino-4-methoxy-phenyl)-3-(4-methoxy-benzo[1,3]dioxol-6-yl)-isothiazole;
4-(3,4-Dimethoxy-5 phosphonooxy-phenyl)-3-(4-methoxy-benzo[1,3]dioxol-6-yl)-isothiazole, disodium salt;
4-(2-Phosphonooxy-4-methoxy-phenyl)-3-(4-methoxy-benzo[1,3]dioxol-6-yl)-isothiazole, disodium salt;
4-(4-Methylsulfanyl-phenyl)-3-(4-methoxy-benzo[1,3]dioxol-6-yl)-isothiazole;
4-(3-Phosphonooxy-4-methylsulfanyl-phenyl)-3-(4-methoxy-benzo[1,3]dioxol-6-yl)-isothiazole, disodium salt;
4-(3-Amino-4-methylsulfanyl-phenyl)-3-(4-methoxy-benzo[1,3]dioxol-6-yl)-isothiazole;
4-(2,3-Dihydro-benzofuran-6-yl)-3-(4-methoxy-benzo[1,3]dioxol-6-yl)-isothiazole;
4-(4-Hydroxy-phenyl)-3-(4-methoxy-benzo[1,3]dioxol-6-yl)-isothiazole, sodium salt;
4-(4-Phosphonooxy-phenyl)-3-(4-methoxy-benzo[1,3]dioxol-6-yl)-isothiazole;
4-(4-1H-Tetrazol-5-yl-phenyl)-3-(4-methoxy-benzo[1,3]dioxol-6-yl)-isothiazole;
4-[4-(1-Methyl-1H-tetrazol-5-yl)-phenyl]-3-(4-methoxy-benzo[1,3]dioxol-6-yl)-isothiazole;
4-(1-Methyl-1H-indo1-5-yl)-3-(4-methoxy-benzo[1,3]dioxol-6-yl)-isothiazole;

4-(3,4,5-Trimethoxy-phenyl)-3-(1-methyl-1H-indol-5-yl)-isothiazole;
4-(3,4,5-Trimethoxy-phenyl)-3-(3-phosphonooxy-4-methoxy-phenyl)-isothiazole, disodium salt;
4-(3,4,5-Trimethoxy-phenyl)-3-(N,N-dimethylamino-phenyl)-isothiazole;
4-(3,4,5-Trimethoxy-phenyl)-3-(3-amino-4-methoxy-phenyl)-isothiazole, hydrochloric acid salt;
4-(3,4,5-Trimethoxy-phenyl)-343-(3-hydroxy-2S-amino-propionamido)-4-methoxy-phenyl]-isothiazole, hydrochloric acid salt;
4-(4-Methoxy-phenyl)-3-(2,4,5-trimethoxy-phenyl)-isothiazole;
4-(4-Methyl-phenyl)-3-(2,4,5-trimethoxy-phenyl)-isothiazole;
4-(4-Ethoxy-phenyl)-3-(2,4,5-trimethoxy-phenyl)-isothiazole;
4-(4-Ethyl-phenyl)-3-(2,4,5-trimethoxy-phenyl)-isothiazole;
4-(4-Propoxy-phenyl)-3-(2,4,5-trimethoxy-phenyl)-isothiazole;
4-(4-Propyl-phenyl)-3-(2,4,5-trimethoxy-phenyl)-isothiazole;
4-(4-Butoxy-phenyl)-3-(2,4,5-trimethoxy-phenyl)-isothiazole;
4-(4-Butyl-phenyl)-3-(2,4,5-trimethoxy-phenyl)-isothiazole;
4-(4-Bromo-phenyl)-3-(2,4,5-trimethoxy-phenyl)-isothiazole;
4-(4-Chloro-phenyl)-3-(2,4,5-trimethoxy-phenyl)-isothiazole;
4-(4-Fluoro-phenyl)-3-(2,4,5-trimethoxy-phenyl)-isothiazole;
4-(4-Nitro-phenyl)-3-(2,4,5-trimethoxy-phenyl)-isothiazole;
4-[4-(N,N-Dimethylamino)-phenyl]-3-(2,4,5-trimethoxy-phenyl)-isothiazole;
4-(3,4-Dimethoxy-phenyl)-3-(2,4,5-trimethoxy-phenyl)-isothiazole;
4-(3-Hydroxy-4-methoxy-phenyl)-3-(2,4,5-trimethoxy-phenyl)-isothiazole;
4-(3,4,5-Trimethoxy-phenyl)-3-(2,4,5-trimethoxy-phenyl)-isothiazole;
4-(4-Methoxy-phenyl)-3-(2,3,5-trimethoxy-phenyl)-isothiazole;
4-(4-Methyl-phenyl)-3-(2,3,5-trimethoxy-phenyl)-isothiazole;
4-(4-Ethoxy-phenyl)-3-(2,3,5-trimethoxy-phenyl)-isothiazole;
4-(4-Ethyl-phenyl)-3-(2,3,5-trimethoxy-phenyl)-isothiazole;
4-(4-Propoxy-phenyl)-3-(2,3,5-trimethoxy-phenyl)-isothiazole;
4-(4-Propyl-phenyl)-3-(2,3,5-trimethoxy-phenyl)-isothiazole;
4-(4-Butoxy-phenyl)-3-(2,3,5-trimethoxy-phenyl)-isothiazole;
4-(4-Butyl-phenyl)-3-(2,3,5-trimethoxy-phenyl)-isothiazole;
4-(4-Bromo-phenyl)-3-(2,3,5-trimethoxy-phenyl)-isothiazole;
4-(4-Chloro-phenyl)-3-(2,3,5-trimethoxy-phenyl)-isothiazole;
4-(4-Fluoro-phenyl)-3-(2,3,5-trimethoxy-phenyl)-isothiazole;
4-(4-Nitro-phenyl)-3-(2,3,5-trimethoxy-phenyl)-isothiazole;
4-[4-(N,N-Dimethylamino)-phenyl]-3-(2,3,5-trimethoxy-phenyl)-isothiazole;
4-(3,4-Dimethoxy-phenyl)-3-(2,3,5-trimethoxy-phenyl)-isothiazole;
4-(3-Hydroxy-4-methoxy-phenyl)-3-(2,3,5-trimethoxy-phenyl)-isothiazole;
4-(3,4,5-Trimethoxy-phenyl)-3-(2,3,5-trimethoxy-phenyl)-isothiazole;
4-(2,3,4,5-Tetramethoxy-phenyl)-3-(4-methoxy-phenyl)-isothiazole;
4-(2,3,4,5-Tetramethoxy-phenyl)-3-(4-methyl-phenyl)-isothiazole;
4-(2,3,4,5-Tetramethoxy-phenyl)-3-(4-ethoxy-phenyl)-isothiazole;
4-(2,3,4,5-Tetramethoxy-phenyl)-3-(4-ethyl-phenyl)-isothiazole;
4-(2,3,4,5-Tetramethoxy-phenyl)-3-(4-propoxy-phenyl)-isothiazole;
4-(2,3,4,5-Tetramethoxy-phenyl)-3-(4-propyl-phenyl)-isothiazole;
4-(2,3,4,5-Tetramethoxy-phenyl)-3-(4-butoxy-phenyl)-isothiazole;
4-(2,3,4,5-Tetramethoxy-phenyl)-3-(4-butyl-phenyl)-isothiazole;
4-(2,3,4,5-Tetramethoxy-phenyl)-3-(4-bromo-phenyl)-isothiazole;
4-(2,3,4,5-Tetramethoxy-phenyl)-3-(4-chloro-phenyl)-isothiazole;
4-(2,3,4,5-Tetramethoxy-phenyl)-3-(4-fluoro-phenyl)-isothiazole;
4-(2,3,4,5-Tetramethoxy-phenyl)-3-(4-nitro-phenyl)-isothiazole;
4-(2,3,4,5-Tetramethoxy-phenyl)-3-[4-(N,N,-dimethylamino)-phenyl]-isothiazole;
4-(2,3,4,5-Tetramethoxy-phenyl)-3-(3,4-dimethoxy-phenyl)-isothiazole;
4-(2,3,4,5-Tetramethoxy-phenyl)-3-(3-hydroxy-4-methoxy-phenyl)-isothiazole;
4-(2,3,4,5-Tetramethoxy-phenyl)-3-(3,4,5-trimethoxy-phenyl)-isothiazole;
4-(2,3-Dihydro-benzo[1,4]dioxin-6-yl)-3-(3,4-dimethoxy-phenyl)-isothiazole;
4-(3,4-Dimethy-phenyl)-3-(2-hydroxy-4-methoxy-5-ethyl-phenyl)-isothiazole;
4-(4-Chloro-phenyl)-3-(2-hydroxy-4-methoxy-5-ethyl-phenyl)-isothiazole;
4-(4-Methyl-phenyl)-3-(2-hydroxy-4-methoxy-5-ethyl-phenyl)-isothiazole;
4-(4-Amino-phenyl)-3-(2-hydroxy-4-methoxy-5-ethyl-phenyl)-isothiazole;
4-(4-Trifluoromethyl-phenyl)-3-(2-hydroxy-4-methoxy-5-ethyl-phenyl)-isothiazole;
4-(4-Methoxy-phenyl)-3-(2-hydroxy-4-methoxy-5-ethyl-phenyl)-isothiazole; and
4-(3,4,5-Trimethoxy-phenyl)-3-(4-bromo-phenyl)-isothiazole;

or pharmaceutically acceptable salts, solvates, clathrates, or prodrugs thereof.

In another embodiment, the invention relates to compounds selected from the group consisting of:
2-amino-N-(2-methoxy-5-[5-(3,4,5-trimethoxy-phenyl)-isothiazol-4-yl)-phenyl)acetamide hydrochloride;
2-amino-3-hydroxy-N-(2-methoxy-5-[5-(3,4,5-trimethoxy-phenyl)-isothiazol-4-yl)-phenyl)propanamide hydrochloride;
2-amino-N-(2-methoxy-5-[5-(3,4,5-trimethoxy-phenyl)-isothiazol -4-yl)-phenyl)propanamide;
2-amino-N-(2-methoxy-5-[5-(3,4,5-trimethoxy-phenyl)-isothiazol -4-yl)-phenyl)-4-(methylthio)butanamide hydrochloride;
2-ammo-N-(2-methoxy-5-[5-(3,4,5-trimethoxy-phenyl)-isothiazol -4-yl)-phenyl)butanamide;
2-amino-N-(2-methoxy-5-[5-(3,4,5-trimethoxy-phenyl)-isothiazol -4-yl)-phenyl)-3-phenylpropanamide hydrochloride;
2-amino-N-(2-methoxy-5-[5-(3,4,5-trimethoxy-phenyl)-isothiazol -4-yl)-phenyl)-4-methylpentanamide hydrochloride;
2-amino-N-(2-methoxy-5-[5-(3,4,5-trimethoxy-phenyl)-isothiazol -4-yl)-phenyl)-3-(4-methoxyphenyl)propanamide hydrochloride;
1-{2-methoxy-5-[5-(3,4,5-trimethoxy-phenyl)-isothiazol-4-yl]-phenylcarbomoyl}-2-methyl-propyl-ammonium chloride;
1-{2-methoxy-5-[5-(3,4,5-trimethoxy-phenyl)-isothiazol -4-yl]-phenylcarbomoyl}-2-methyl-butyl-ammonium chloride;
2-hydroxy-1-{2-methoxy-5-[5-(3,4,5-trimethoxy-phenyl)-isothiazol -4-yl]-phenylcarbomoyl}-propyl-ammonium chloride;
2-(4-hydroxy-phenyl)-1-{2-methoxy-5-[5-(3,4,5-trimethoxy-phenyl)-isothiazol-4-yl]-phenylcarbomoyl}-ethyl-ammonium chloride;
C-{2-methoxy-5-[5-(3,4,5-trimethoxy-phenyl)-isothiazol-4-yl]-phenylcarbomoyl}-C-phenyl-methyl-ammonium chloride;
2-(1H-indol-2-yl)-1-{2-methoxy-5-[5-(3,4,5-trimethoxy-phenyl)-isothiazol-4-yl]-phenylcarbomoyl}-ethyl-ammonium chloride;
2-benzofuran-2-yl-1-{2-methoxy-545-(3,4,5-trimethoxy-phenyl)-isothiazol-4-yl]-phenylcarbomoyl}-ethyl-ammonium chloride;
2-carboxyl-1-{2-methoxy-5-[5-(3,4,5-trimethoxy-phenyl)-isothiazol-4-yl]-phenylcarbomoyl}-ethyl-ammonium chloride;
3-carboxyl-1-{2-methoxy-5-[5-(3,4,5-trimethoxy-phenyl)-isothiazol-4-yl]-phenylcarbomoyl}-propyl-ammonium chloride;
3-carbamoyl-1-{2-methoxy-5-[5-(3,4,5-trimethoxy-phenyl)-isothiazol-4-yl]-phenylcarbomoyl}-propyl-ammonium chloride;
2-carbamoyl-1-{2-methoxy-5-[5-(3,4,5-trimethoxy-phenyl)-isothiazol-4-yl]-phenylcarbomoyl}-ethyl-ammonium chloride;
2-(3H-imidazol-4-yl)-1-{2-methoxy-5-[5-(3,4,5-trimethoxy-phenyl)-isothiazol-4-yl]-phenylcarbomoyl}-ethyl-ammonium chloride;
5-amino-1-{2-methoxy-5-[5-(3,4,5-trimethoxy-phenyl)-isothiazol-4-yl]-phenylcarbomoyl}-pentyl-ammonium chloride;
4-guanidino-1-{2-methoxy-5-[5-(3,4,5-trimethoxy-phenyl)-isothiazol-4-yl]-phenylcarbomoyl}-butyl-ammonium chloride;
N-{2-methoxy-5-[5-(3,4,5-trimethoxy-phenyl)-isothiazol-4-yl]-phenyl}succinamic acid;
4-{2-methoxy-5-[5-(3,4,5-trimethoxy-phenyl)-isothiazol-4-yl]-phenylcarbamoyl}-butyric acid;
2-{2-methoxy-5-[5-(3,4,5-trimethoxy-phenyl)-isothiazol-4-yl]-phenylcarbamoyl}-ethyl-ammonium chloride;
3-(2-methoxy-ethoxy)-N-{2-methoxy-5-[5-(3,4,5-trimethoxy-phenyl)-isothiazol-4-yl]-phenyl}-propionamide;
3-(2-PEG)-N-{2-methoxy-5-[5-(3,4,5-trimethoxy-phenyl)-isothiazol-4-yl]-phenyl}-propionamide;
N-{2-methoxy-5-[5-(3,4,5-trimethoxy-phenyl)-isothiazol-4-yl]-phenyl}-3-(2-methylamino-ethylamino)-propionamide;
3-PEG-N-{2-methoxy-5-[5-(3,4,5-trimethoxy-phenyl)-isothiazol-4-yl]-phenylcarbamoyl}-methyl)-propionamide;
N-{2-methoxy-5-[5-(3,4,5-trimethoxy-phenyl)-isothiazol-4-yl]-phenylcarbamoyl}-methyl)-succinamic acid;
{2-methoxy-5-[5-(3,4,5-trimethoxy-phenyl)-isothiazol-4-yl]-phenyl}-carbamic acid 2-methoxy-ethyl ester;
2-methoxy-5-(5-(3,4,5-trimethoxyphenyl)isothiazol-4-yl)phenylcarbamate-PEG;
3-amino-N-[4-guanadino-1-{2-methoxy-5-[5-(3,4,5-trimethoxy-phenyl)-isothiazol-4-yl]-phenylcarbamoyl}-butylcarbamoyl)-methyl]-succinamic acid;
2-amino-N-(2-methoxy-5-[5-(3,4,5-trimethoxy-phenyl)-isothiazol-4-yl)-phenyl)propanamide hydrochloride;
2-amino-N-(2-methoxy-5-[3-(3,4,5-trimethoxy-phenyl)-isothiazol -4-yl)-phenyl)acetamide hydrochloride;
2-amino-3-hydroxy-N-(2-methoxy-5-[3-(3,4,5-trimethoxy-phenyl)-isothiazol-4-yl)-phenyl)propanamide hydrochloride;
2-amino-N-(2-methoxy-5-[3-(3,4,5-trimethoxy-phenyl)-isothiazol-4-yl)-phenyl)propanamide;
2-amino-N-(2-methoxy-5-[3-(3,4,5-trimethoxy-phenyl)-isothiazol-4-yl)-phenyl)-4-(methylthio)butanamide hydrochloride;
2-amino-N-(2-methoxy-5-[3-(3,4,5-trimethoxy-phenyl)-isothiazol-4-yl)-phenyl)butanamide;
2-amino-N-(2-methoxy-5-[3-(3,4,5-trimethoxy-phenyl)-isothiazol-4-yl)-phenyl)-3-phenylpropanamide hydrochloride;
2-amino-N-(2-methoxy-5-[3-(3,4,5-trimethoxy-phenyl)-isothiazol-4-yl)-phenyl)-4-methylpentanamide hydrochloride;
2-amino-N-(2-methoxy-5-[3-(3,4,5-trimethoxy-phenyl)-isothiazol-4-yl)-phenyl)-3-(4-methoxyphenyl) propanamide hydrochloride;
1-{2-methoxy-5-[3-(3,4,5-trimethoxy-phenyl)-isothiazol-4-yl]-phenylcarbomoyl}-2-methyl-propyl-ammonium chloride;
1-{2-methoxy-5-[3-(3,4,5-trimethoxy-phenyl)-isothiazol-4-yl]-phenylcarbomoyl}-2-methyl-butyl-ammonium chloride;
2-hydroxy-1-{2-methoxy-5-[3-(3,4,5-trimethoxy-phenyl)-isothiazol-4-yl]-phenylcarbomoyl}-propyl-ammonium chloride;
2-(4-hydroxy-phenyl)-1-{2-methoxy-5-[3-(3,4,5-trimethoxy-phenyl)-isothiazol-4-yl]-phenylcarbomoyl}-ethyl-ammonium chloride;
C-{2-methoxy-5-[3-(3,4,5-trimethoxy-phenyl)-isothiazol-4-yl]-phenylcarbomoyl}-C-phenyl-methyl-ammonium chloride;
2-(1H-indol-2-yl)-1-{2-methoxy-5-[3-(3,4,5-trimethoxy-phenyl)-isothiazol-4-yl]-phenylcarbomoyl}-ethyl-ammonium chloride;
2-benzofuran-2-yl-1-{2-methoxy-5-[3-(3,4,5-trimethoxy-phenyl)-isothiazol-4-yl]-phenylcarbomoyl}-ethyl-ammonium chloride;
2-carboxyl-1-{2-methoxy-5-[3-(3,4,5-trimethoxy-phenyl)-isothiazol-4-yl]-phenylcarbomoyl}-ethyl-ammonium chloride;
3-carboxyl-1-{2-methoxy-5-[3-(3,4,5-trimethoxy-phenyl)-isothiazol-4-yl]-phenylcarbomoyl}-propyl-ammonium chloride;
3-carbamoyl-1-{2-methoxy-5-[3-(3,4,5-trimethoxy-phenyl)-isothiazol-4-yl]-phenylcarbomoyl}-propyl-ammonium chloride;
2-carbamoyl-1-{2-methoxy-5-[3-(3,4,5-trimethoxy-phenyl)-isothiazol -4-yl]-phenylcarbomoyl}-ethyl-ammonium chloride;
2-(3H-imidazol-4-yl)-1-{2-methoxy-5-[3-(3,4,5-trimethoxy-phenyl)-isothiazol-4-yl]-phenylcarbomoyl}-ethyl-ammonium chloride;
5-amino-1-{2-methoxy-5-[3-(3,4,5-trimethoxy-phenyl)-isothiazol-4-yl]-phenylcarbomoyl}-pentyl-ammonium chloride;
4-guanidino-1-{2-methoxy-5-[3-(3,4,5-trimethoxy-phenyl)-isothiazol-4-yl]-phenylcarbomoyl}-butyl-ammonium chloride;
N-{2-methoxy-5-[3-(3,4,5-trimethoxy-phenyl)-isothiazol-4-yl]-phenyl}succinamic acid;
4-{2-methoxy-5-[3-(3,4,5-trimethoxy-phenyl)-isothiazol-4-yl]-phenylcarbamoyl}-butyric acid;
2-{2-methoxy-5-[3-(3,4,5-trimethoxy-phenyl)-isothiazol-4-yl]-phenylcarbamoyl}-ethyl-ammonium chloride;
3-(2-methoxy-ethoxy)-N-{2-methoxy-5-[3-(3,4,5-trimethoxy-phenyl)-isothiazol-4-yl]-phenyl}-propionamide;
3-(2-PEG)-N-{2-methoxy-5-[3-(3,4,5-trimethoxy-phenyl)-isothiazol-4-yl]-phenyl}-propionamide;
N-{2-methoxy-5-[3-(3,4,5-trimethoxy-phenyl)-isothiazol-4-yl]-phenyl}-3-(2-methylamino-ethylamino)-propionamide;
3-PEG-N-{2-methoxy-5-[3-(3,4,5-trimethoxy-phenyl)-isothiazol-4-yl]-phenylcarbamoyl}-methyl)-propionamide;
N-{2-methoxy-5-[3-(3,4,5-trimethoxy-phenyl)-isothiazol-4-yl]-phenylcarbamoyl}-methyl)-succinamic acid;
{2-methoxy-5-[3-(3,4,5-trimethoxy-phenyl)-isothiazol-4-yl]-phenyl}-carbamic acid 2-methoxy-ethyl ester;
2-methoxy-5-(3-(3,4,5-trimethoxyphenyl)isothiazol-4-yl)phenylcarbamate-PEG;
3-amino-N-[4-guanadino-1-{2-methoxy-5-[3-(3,4,5-trimethoxy-phenyl)-isothiazol-4-yl]-phenylcarbamoyl}-butylcarbamoyl)-methyl]-succinamic acid;
2-amino-N-(2-methoxy-5-[3-(3,4,5-trimethoxy-phenyl)-isothiazol-4-yl)-phenyl)propanamide hydrochloride;
methyl 2-(2-(2-methoxy-5-(5-(3,4,5-trimethoxyphenyl)isothiazol-4-yl)phenylamino)-2-oxoethylamino)acetate;
4-amino-5-(2-methoxy-5-(5-(3,4,5-trimethoxyphenyl)isothiazol-4-yl)phenylamino)-5-oxopentanoic acid hydrochloride;
3-amino-N-(2-methoxy-5-(5-(3,4,5-trimethoxyphenyl)isothiazol-4-yl)phenyl)propanamide hydrochloride;
3-amino-N-(2-methoxy-5-(5-(3,4,5-trimethoxyphenyl)isothiazol-4-yl)phenyl)-4-methylpentanamide hydrochloride;
methyl 2-(2-(2-methoxy-5-(3-(3,4,5-trimethoxyphenyl)isothiazol-4-yl)phenylamino)-2-oxoethylamino)acetate;
4-amino-5-(2-methoxy-5-(3-(3,4,5-trimethoxyphenyl) isothiazol-4-yl)phenylamino)-5-oxopentanoic acid hydrochloride;
3-amino-N-(2-methoxy-5-(3-(3,4,5-trimethoxyphenyl)isothiazol-4-yl)phenyl)propanamide hydrochloride; and
3-amino-N-(2-methoxy-5-(3-(3,4,5-trimethoxyphenyl)isothiazol-4-yl)phenyl)-4-methylpentanamide hydrochloride;

or pharmaceutically acceptable salts, solvates, clathrates, or prodrugs thereof.

All of the features, specific embodiments and particular substituents disclosed herein may be combined in any combination. Each feature, embodiment or substituent disclosed in this specification may be replaced by an alternative feature, embodiment or substituent serving the same, equivalent, or similar purpose. In the case of chemical compounds, specific values for variables (e.g., values shown in the exemplary compounds disclosed herein) in any chemical formula disclosed herein can be combined in any combination resulting in a stable structure. Furthermore, specific values (whether preferred or not) for substituents in one type of chemical structure may be combined with values for other substituents (whether preferred or not) in the same or different type of chemical structure. Thus, unless expressly stated otherwise, each feature, embodiment or substituent disclosed is only an example of a generic series of equivalent or similar features, embodiments or substituents.

In another embodiment, the invention relates to pharmaceutical compositions that comprise a compound of any one of formulas (I)-(XXIX), (XXXI), (XXXV)-(XL), (IA)-(XXIA), (XXVIIA)-(XXIXA), (XXXIA), (XXXVA)-(XLA), (IB)-(XXIB), (XXVIIB)-(XXIXB), (XXXIB), (XXXVB)-(XLB), or of Table 1, or a pharmaceutically acceptable salt, solvate, clathrate, or prodrug thereof, as an active ingredient, and a pharmaceutically acceptable carrier or vehicle. The compositions are useful for treating or inhibiting angiogenesis.

### Exemplary Compounds of the Invention

Exemplary compounds of the invention are depicted in Table 1 below.

TABLE 1

| Compound | |
|---|---|
| No. | Chemical Name |
| 1 | 4-(4-Bromo-phenyl)-5-(3,4,5-trimethoxy-phenyl)-isoxazole |
| 2 | 4-(4-Bromo-phenyl)-5-(3,4,5-trimethoxy-phenyl)-isoxazole |
| 3 | 4-(4-Methoxy-phenyl)-5-(3,4,5-trimethoxy-phenyl)-isoxazole |
| 4 | 4-(4-Iodo-phenyl)-5-(2-hydroxy-4-methoxy-5-ethyl-phenyl)-isoxazole |
| 5 | 4-Phenyl-5-(2-hydroxy-4-methoxy-5-propyl-phenyl)-isoxazole |
| 6 | 4-(4-Bromo-phenyl)-5-(2-hydroxy-4-methoxy-5-ethyl-phenyl)-isoxazole |
| 7 | 4-(2,3-Dihydro-benzo[1,4]di-oxin-6-yl)-5-(2-hydroxy-4- |
| | methoxy-5-propyl-phenyl)-isoxazole |
| 8 | 4-(4-hydroxy-phenyl)-5-(3,4,5-trihydroxy-phenyl)-isoxazole |
| 9 | 4-(4-Iodo-phenyl)-5-(3,4,5-trimethoxy-phenyl)-isoxazole |
| 10 | 4-(3-Fluoro-4-methoxy-phenyl)-5-(3,4,5-trimethoxy-phenyl)-isoxazole |
| 11 | 4-(4-Nitro-phenyl)-5-(3,4,5-trimethoxy-phenyl)-isoxazole |
| 12 | 4-(4-Amino-phenyl)5-(3,4,5-trimethoxy-phenyl)-isoxazole |
| 13 | 4-(4′-Methoxy-biphenyl-4-yl)-5-(3,4,5-trimethoxy-phenyl)-isoxazole |
| 14 | 4-[4-(pyridine-3-yl)-phenyl]-5-(3,4,5-trimethoxy-phenyl)-isoxazole |
| 15 | 4-[4-(pyridine-4-yl)-phenyl]-5-(3,4,5-trimethoxy-phenyl)-isoxazole |
| 16 | 4-[4-(pyridine-2-yl)-phenyl]-5-(3,4,5-trimethoxy-phenyl)-isoxazole |
| 17 | 4-(Quinolin-7-yl)-5-(3,4,5-trimethoxy-phenyl)-isoxazole |
| 18 | 4-(pyridin-4-yl)-5-(3,4,5-trimethoxy-phenyl)-isoxazole |
| 19 | 4-(Isoquinolin-7-yl)-5-(3,4,5-trimethoxy-phenyl)-isoxazole |
| 20 | 4-(1-Methyl-1H-indol-5-yl)-5-(3,4,5-trimethoxy-phenyl)-isoxazole |
| 21 | 4-(4-Methoxy-phenyl)-5-(benzo[1,3]dioxol-5-yl)-isoxazole |
| 22 | 4-(4-Methoxy-phenyl)-5-(1-ethyl-1H-indol-6-yl)-isoxazole |
| 23 | 4-(4-Carboxy-phenyl)-5-(3,4,5-trimethoxy-phenyl)-isoxazole |
| 24 | 4-(4-Methoxycarbonyl-phenyl)-5-(3,4,5-trimethoxy-phenyl)-isoxazole |
| 25 | 4-[4-(Oxazol-2-yl)-phenyl]-5-(3,4,5-trimethoxy-phenyl)-isoxazole |
| 26 | 4-(4-Methoxy-phenyl)-5-(3,4,5-triethyl-phenyl)-isoxazole |
| 27 | 4-(4-Iodo-phenyl)-5-(3,4,5-triethyl-phenyl)-isoxazole |
| 28 | 4-(3-Fluoro-4-methoxy-phenyl)-5-(3,4,5-triethyl-phenyl)-isoxazole |
| 29 | 4-(4-Nitro-phenyl)-5-(3,4,5-triethyl-phenyl)-isoxazole |
| 30 | 4-(4-N,N-dimethylamino-phenyl)-5-(3,4,5-triethyl-phenyl)-isoxazole |
| 31 | 4-(4-Methoxy-phenyl)-5-(3,4,5-trimethyl-phenyl)-isoxazole |
| 32 | 4-[4-(Pyridin-3-yl)-phenyl]-5-(3,4,5-triethyl-phenyl)-isoxazole |
| 33 | 4-[4-(Pyridin-4-yl)-phenyl]-5-(3,4,5-triethyl-phenyl)-isoxazole |
| 34 | 4-[4-(Pyridin-2-yl)-phenyl]-5-(3,4,5-triethyl-phenyl)-isoxazole |
| 35 | 4-(Quinolin-7-yl)-5-(3,4,5-triethyl-phenyl)-isoxazole |
| 36 | 4-(Pyridin-4-yl)-5-(3,4,5-triethyl-phenyl)-isoxazole |
| 37 | 4-(Isoquinolin-7-yl)-5-(3,4,5-triethyl-phenyl)-isoxazole |
| 38 | 4-(1H-Indol-5-yl)-5-(3,4,5-triethyl-phenyl)-isoxazole |
| 39 | 4-(4-Methoxy-phenyl)-5-(benzo[1,3]dioxol-5-yl)-isoxazole |
| 40 | 4-(4-Methoxy-phenyl)-5-[1-isopropyl-1H-indol-6-yl)-isoxazole |
| 41 | 4-(4-Methoxy-phenyl)-5-(2,3,4-trimethoxy-phenyl)-isoxazole |
| 42 | 4-(3-Hydroxy-4-methoxy-phenyl)-5-(3,4,5-trimethoxy-phenyl)-isoxazole |
| 43 | 4-[3-(Ethyl-hydroxy-phosphoryloxy)-4-methoxy-phenyl]-5-(3,4,5-trimethoxy- |
| | phenyl)-isoxazole |
| 44 | 4-(4-Methoxy-phenyl)-5-(2-hydroxy-4-methoxy-5-ethyl-phenyl)-isoxazole |
| 45 | 4-(4-Isopropyl-phenyl)-5-(3,4,5-trimethoxy-phenyl)-isoxazole |
| 46 | 4-(2,3-Dihydro-benzo[1,4]dioxin-6-yl)-5-(3,4,5-trimethoxy-phenyl)-isoxazole |
| 47 | 4-(4-Ethyl-phenyl)-5-(3,4,5-trimethoxy-phenyl)-isoxazole |
| 48 | 4-(5-Methoxy-pyridin-2-yl)-5-(3,4,5-trimethoxy-phenyl)-isoxazole |
| 49 | 4-(4-Methoxy-phenyl)-5-(2,3,4-trimethoxy-pyridin-6-yl)-isoxazole |
| 50 | 4-(4-Methoxy-phenyl)-5-(3,5-dimethoxy-4-methoxycarbonyl-phenyl)-isoxazole |
| 51 | 4-(4-Methoxy-phenyl)-5-(3,5-diacetoxy-phenyl)-isoxazole |
| 52 | 4-(2-Methoxy-pyridin-5-yl)-5-(3,4,5-trimethoxy-phenyl)-isoxazole |
| 53 | 4-(4-Methoxy-phenyl)-5-(1-methyl-5-methoxy-1H-indol-7-yl)-isoxazole |
| 54 | 4-(4-Methoxy-phenyl)-5-(1-ethyl-1H-indol-7-yl)-isoxazole |
| 55 | 4-(4-Methoxy-phenyl)-5-(benzo[1,3]dioxol-4-yl)-isoxazole |
| 56 | 4-(2-Hydroxy-4-methoxy-phenyl)-5-(3,4,5-trimethoxy)-isoxazole |
| 57 | 4-[2-(Ethyl-hydroxy-phosphoryloxy)-4-methoxy-phenyl]-5-(3,4,5-trimethoxy- |
| | phenyl)-isoxazole |
| 58 | 4-(Pyridazin-4-yl)-5-(3,4,5-trimethoxy-phenyl)-isoxazole |
| 59 | 4-(Pyrimidin-5-yl)-5-(3,4,5-trimethoxy-phenyl)-isoxazole |
| 60 | 4-(Pyridin-3-yl)-5-(3,4,5-trimethoxy-phenyl)-isoxazole, hydrochloric acid salt |
| 61 | 4-(3-Mercapto-4-methoxy-phenyl)-5-(3,4,5-trimethoxy-phenyl)-isoxazole |
| 62 | 4-(3-Phosphonosulfanyl-4-methoxy-phenyl)-5-(3,4,5-trimethoxy-phenyl)- |
| | isoxazole, disodium salt |
| 63 | 4-(3-Acetylamino-4-methoxy-phenyl)-5-(3,4,5-trimethoxy-phenyl)-isoxazole |
| 64 | 2-methoxy-5-[5-(3,4,5-trimethoxy-phenyl)-isoxazol-4-yl)-phenylamine |
| | hydrochloride |
| 65 | 4-(2-Hydroxy-4-methoxy-phenyl)-5-(3,4,5-trimethoxy-phenyl)-isoxazole |
| 66 | 4-(2-Methoxy-pyridine-5-yl)-5-(3,4,5-trimethoxy-phenyl)-isoxazole |
| 67 | 4-(5-Methoxy-pyridine-2-yl)-5-(3,4,5-trimethoxy-phenyl)-isoxazole |
| 68 | 4-(3-Carboxy-4-methoxy-phenyl)-5-(3,4,5-trimethoxy-phenyl)-isoxazole, |
| | sodium salt |
| 69 | 4-(3-Methoxycarbonyl-4-methoxy-phenyl)-5-(3,4,5- |
| | trimethoxy-phenyl)-isoxazole |
| 70 | 4-(3-Sulfooxy-4-methoxy-phenyl)-5-(3,4,5-trimethoxy-phenyl)-isoxazole, |
| | sodium salt |
| 71 | 4-(2-Amino-4-methoxy-phenyl)-5-(3,4,5-trimethoxy-phenyl)-isoxazole |
| 72 | 4-(3,4-Dimethoxy-5-phosphonooxy-phenyl)-5-(3,4,5-trimethoxy-phenyl)- |
| | isoxazole, disodium salt |
| 73 | 4-(2-Phosphonooxy-4-methoxy-phenyl)-5-(3,4,5-trimethoxy-phenyl)-isoxazole, |
| | disodium salt |
| 74 | 4-(4-Methylsulfanyl-phenyl)-5-(3,4,5-trimethoxy-phenyl)-isoxazole |
| 75 | 4-(3-Phosphonooxy-4-methylsulfanyl-phenyl)-5-(3,4,5-trimethoxy-phenyl)- |
| | isoxazole, disodium salt |
| 76 | 4-(3-Amino-4-methylsulfanyl-phenyl)-5-(3,4,5-trimethoxy-phenyl)-isoxazole |
| 77 | 4-(2,3-Dihydro-benzofuran-6-yl)-5-(3,4,5-trimethoxy-phenyl)-isoxazole |
| 78 | 4-(4-Hydroxy-phenyl)-5-(3,4,5-trimethoxy-phenyl)-isoxazole, sodium salt |
| 79 | 4-(4-Phosphonooxy-phenyl)-5-(3,4,5-trimethoxy-phenyl)-isoxazole, disodium |
| | salt |
| 80 | 4-(4-1H-Tetrazol-5-yl-phenyl)-5-(3,4,5-trimethoxy-phenyl)-isoxazole |
| 81 | 4-[4-(1-Methyl-1H-tetrazol-5-yl)-phenyl]-5-(3,4,5-trimethoxy-phenyl)-isoxazole |
| 82 | 4-(1-Methyl-1H-indol-5-yl)-5-(3,4,5-trimethoxy-phenyl)-isoxazole |
| 83 | 4-(Pyridazin-4-yl)-5-(4-methoxy-benzo[1,3]dioxol-6-yl)-isoxazole |
| 84 | 4-(Pyrimidin-5-yl)-5-(4-methoxy-benzo[1,3]dioxol-6-yl)-isoxazole |
| 85 | 4-(Pyridin-3-yl)-5-(4-methoxy-benzo[1,3]dioxol-6-yl)-isoxazole, hydrochloric |
| | acid salt |
| 86 | 4-(3-Mercapto-4-methoxy-phenyl)-5-(4-methoxy-benzo[1,3]dioxol-6-yl)- |
| | isoxazole |
| 87 | 4-(3-Phosphonosulfanyl-4-methoxy-phenyl)-5-(4- |
| | methoxy-benzo[1,3]dioxol-6-yl)-isoxazole |
| 88 | 4-(3-Acetylamino-4-methoxy-phenyl)-5-(4- |
| | methoxy-benzo[1,3]dioxol-6-yl)-isoxazole |
| 89 | 4-(3-Amino-4-methoxy-phenyl)-5-(4-methoxy-benzo[1,3]dioxol-6-yl)- |
| | isoxazole, hydrochloric acid salt |
| 90 | 4-(2-Hydroxy-4-methoxy-phenyl)-5-(4-methoxy-benzo[1,3]dioxol-6-yl)- |
| | isoxazole |
| 91 | 4-(2-Methoxy-pyridin-5-yl)-5-(4-methoxy-benzo[1,3]dioxol-6-yl)-isoxazole |
| 92 | 4-(5-Methoxy-pyridin-2-yl)-5-(4-methoxy-benzo[1,3]dioxol-6-yl)-isoxazole |
| 93 | 4-(3-Carboxy-4-methoxy-phenyl)-5-(4-methoxy-benzo[1,3]dioxol-6-yl)- |
| | isoxazole, sodium salt |
| 94 | 4-(3-Methoxycarbonyl-4-methoxy-phenyl)-5-(4- |
| | methoxy-benzo[1,3]dioxol-6-yl)-isoxazole |
| 95 | 4-(3-Sulfooxy-4-methoxy-phenyl)-5-(4-methoxy-benzo[1,3]dioxol-6-yl)- |
| | isoxazole, sodium salt |
| 96 | 4-(3-Amino-4-methoxy-phenyl)-5-(4-methoxy-benzo[1,3]dioxol-6-yl)- |
| | isoxazole |
| 97 | 4-(3,4-Dimethoxy-5 phosphonooxy-phenyl)-5-(4- |
| | methoxy-benzo[1,3]dioxol-6-yl)-isoxazole, disodium salt |
| 98 | 4-(2-Phosphonooxy-4-methoxy-phenyl)-5-(4- |
| | methoxy-benzo[1,3]dioxol-6-yl)-isoxazole, disodium salt |
| 99 | 4-(4-Methylsulfanyl-phenyl)-5-(4-methoxy-benzo[1,3]dioxol-6-yl)-isoxazole |
| 100 | 4-(3-Phosphonooxy-4-methylsulfanyl-phenyl)-5-(4- |
| | methoxy-benzo[1,3]dioxol-6-yl)-isoxazole, disodium salt |
| 101 | 4-(3-Amino-4-methylsulfanyl-phenyl)-5-(4-methoxy-benzo[1,3]dioxol-6-yl)- |
| | isoxazole |
| 102 | 4-(2,3-Dihydro-benzofuran-6-yl)-5-(4-methoxy-benzo[1,3]dioxol-6-yl)- |
| | isoxazole |
| 103 | 4-(4-Hydroxy-phenyl)-5-(4-methoxy-benzo[1,3]dioxol-6-yl)-isoxazole, sodium |
| | salt |
| 104 | 4-(4-Phosphonooxy-phenyl)-5-(4-methoxy-benzo[1,3]dioxol-6-yl)-isoxazole |
| 105 | 4-(4-1H-Tetrazol-5-yl-phenyl)-5-(4-methoxy-benzo[1,3]dioxol-6-yl)-isoxazole |
| 106 | 4-[4-(1-Methyl-1H-tetrazol-5-yl)-phenyl]-5-(4-methoxy-benzo[1,3]dioxol-6-yl)- |
| | isoxazole |
| 107 | 4-(1-Methyl-1H-indol-5-yl)-5-(4-methoxy-benzo[1,3]dioxol-6-yl)-isoxazole |
| 108 | 4-(3,4,5-Trimethoxy-phenyl)-5-(1-methyl-1H-indol-5-yl)-isoxazole |
| 109 | 4-(3,4,5-Trimethoxy-phenyl)-5-(3-phosphonooxy-4-methoxy-phenyl)-isoxazole, |
| | disodium salt |
| 110 | 4-(3,4,5-Trimethoxy-phenyl)-5-(N,N-dimethylamino-phenyl)-isoxazole |
| 111 | 4-(3,4,5-Trimethoxy-phenyl)-5-(3-amino-4-methoxy-phenyl)-isoxazole, |
| | hydrochloric acid salt |
| 112 | 4-(3,4,5-Trimethoxy-phenyl)-5-[3-(3-hydroxy-2S-amino- |
| | propionamido)-4-methoxy-phenyl]-isoxazole, hydrochloric acid salt |
| 113 | 4-(4-Methoxy-phenyl)-5-(2,4,5-trimethoxy-phenyl)-isoxazole |
| 114 | 4-(4-Methyl-phenyl)-5-(2,4,5-trimethoxy-phenyl)-isoxazole |
| 115 | 4-(4-Ethoxy-phenyl)-5-(2,4,5-trimethoxy-phenyl)-isoxazole |
| 116 | 4-(4-Ethyl-phenyl)-5-(2,4,5-trimethoxy-phenyl)-isoxazole |
| 117 | 4-(4-Propoxy-phenyl)-5-(2,4,5-trimethoxy-phenyl)-isoxazole |
| 118 | 4-(4-Propyl-phenyl)-5-(2,4,5-trimethoxy-phenyl)-isoxazole |
| 119 | 4-(4-Butoxy-phenyl)-5-(2,4,5-trimethoxy-phenyl)-isoxazole |
| 120 | 4-(4-Butyl-phenyl)-5-(2,4,5-trimethoxy-phenyl)-isoxazole |
| 121 | 4-(4-Bromo-phenyl)-5-(2,4,5-trimethoxy-phenyl)-isoxazole |
| 122 | 4-(4-Chloro-phenyl)-5-(2,4,5-trimethoxy-phenyl)-isoxazole |
| 123 | 4-(4-Fluoro-phenyl)-5-(2,4,5-trimethoxy-phenyl)-isoxazole |
| 124 | 4-(4-Nitro-phenyl)-5-(2,4,5-trimethoxy-phenyl)-isoxazole |
| 125 | 4-[4-(N,N-Dimethylamino)-phenyl]-5-(2,4,5-trimethoxy-phenyl)-isoxazole |
| 126 | 4-(3,4-Dimethoxy-phenyl)-5-(2,4,5-trimethoxy-phenyl)-isoxazole |
| 127 | 4-(3-Hydroxy-4-methoxy-phenyl)-5-(2,4,5-trimethoxy-phenyl)-isoxazole |
| 128 | 4-(3,4,5-Trimethoxy-phenyl)-5-(2,4,5-trimethoxy-phenyl)-isoxazole |
| 129 | 4-(4-Methoxy-phenyl)-5-(2,3,5-trimethoxy-phenyl)-isoxazole |
| 130 | 4-(4-Methyl-phenyl)-5-(2,3,5-trimethoxy-phenyl)-isoxazole |
| 131 | 4-(4-Ethoxy-phenyl)-5-(2,3,5-trimethoxy-phenyl)-isoxazole |
| 132 | 4-(4-Ethyl-phenyl)-5-(2,3,5-trimethoxy-phenyl)-isoxazole |
| 133 | 4-(4-Propoxy-phenyl)-5-(2,3,5-trimethoxy-phenyl)-isoxazole |
| 134 | 4-(4-Propyl-phenyl)-5-(2,3,5-trimethoxy-phenyl)-isoxazole |
| 135 | 4-(4-Butoxy-phenyl)-5-(2,3,5-trimethoxy-phenyl)-isoxazole |
| 136 | 4-(4-Butyl-phenyl)-5-(2,3,5-trimethoxy-phenyl)-isoxazole |
| 137 | 4-(4-Bromo-phenyl)-5-(2,3,5-trimethoxy-phenyl)-isoxazole |
| 138 | 4-(4-Chloro-phenyl)-5-(2,3,5-trimethoxy-phenyl)-isoxazole |
| 139 | 4-(4-Fluoro-phenyl)-5-(2,3,5-trimethoxy-phenyl)-isoxazole |
| 140 | 4-(4-Nitro-phenyl)-5-(2,3,5-trimethoxy-phenyl)-isoxazole |
| 141 | 4-[4-(N,N-Dimethylamino)-phenyl]-5-(2,3,5-trimethoxy-phenyl)-isoxazole |
| 142 | 4-(3,4-Dimethoxy-phenyl)-5-(2,3,5-trimethoxy-phenyl)-isoxazole |
| 143 | 4-(3-Hydroxy-4-methoxy-phenyl)-5-(2,3,5-trimethoxy-phenyl)-isoxazole |
| 144 | 4-(3,4,5-Trimethoxy-phenyl)-5-(2,3,5-trimethoxy-phenyl)-isoxazole |
| 145 | 4-(2,3,4,5-Tetramethoxy-phenyl)-5-(4-methoxy-phenyl)-isoxazole |
| 146 | 4-(2,3,4,5-Tetramethoxy-phenyl)-5-(4-methyl-phenyl)-isoxazole |
| 147 | 4-(2,3,4,5-Tetramethoxy-phenyl)-5-(4-ethoxy-phenyl)-isoxazole |
| 148 | 4-(2,3,4,5-Tetramethoxy-phenyl)-5-(4-ethyl-phenyl)-isoxazole |
| 149 | 4-(2,3,4,5-Tetramethoxy-phenyl)-5-(4-propoxy-phenyl)-isoxazole |
| 150 | 4-(2,3,4,5-Tetramethoxy-phenyl)-5-(4-propyl-phenyl)-isoxazole |
| 151 | 4-(2,3,4,5-Tetramethoxy-phenyl)-5-(4-butoxy-phenyl)-isoxazole |
| 152 | 4-(2,3,4,5-Tetramethoxy-phenyl)-5-(4-butyl-phenyl)-isoxazole |
| 153 | 4-(2,3,4,5-Tetramethoxy-phenyl)-5-(4-bromo-phenyl)-isoxazole |
| 154 | 4-(2,3,4,5-Tetramethoxy-phenyl)-5-(4-chloro-phenyl)-isoxazole |
| 155 | 4-(2,3,4,5-Tetramethoxy-phenyl)-5-(4-fluoro-phenyl)-isoxazole |
| 156 | 4-(2,3,4,5-Tetramethoxy-phenyl)-5-(4-nitro-phenyl)-isoxazole |
| 157 | 4-(2,3,4,5-Tetramethoxy-phenyl)-5-[4-(N,N,-dimethylamino)-phenyl]- |
| | isoxazole |
| 158 | 4-(2,3,4,5-Tetramethoxy-phenyl)-5-(3,4-dimethoxy-phenyl)-isoxazole |
| 159 | 4-(2,3,4,5-Tetramethoxy-phenyl)-5-(3-hydroxy-4-methoxy-phenyl)-isoxazole |
| 160 | 4-(2,3,4,5-Tetramethoxy-phenyl)-5-(3,4,5-trimethoxy-phenyl)-isoxazole |
| 161 | 4-(2,3-Dihydro-benzo[1,4]dioxin-6-yl)-5-(3,4-dimethoxy-phenyl)-isoxazole |
| 162 | 4-(3,4-Dimethy-phenyl)-5-(2-hydroxy-4-methoxy-5-ethyl-phenyl)-isoxazole |
| 163 | 4-(4-Chloro-phenyl)-5-(2-hydroxy-4-methoxy-5-ethyl-phenyl)-isoxazole |
| 164 | 4-(4-Methyl-phenyl)-5-(2-hydroxy-4-methoxy-5-ethyl-phenyl)-isoxazole |
| 165 | 4-(4-Amino-phenyl)-5-(2-hydroxy-4-methoxy-5-ethyl-phenyl)-isoxazole |
| 166 | 4-(4-Trifluoromethyl-phenyl)-5-(2-hydroxy-4-methoxy-5- |
| | ethyl-phenyl)-isoxazole |
| 167 | 4-(4-Methoxy-phenyl)-5-(2-hydroxy-4-methoxy-5-ethyl-phenyl)-isoxazole |
| 168 | 4-(3,4,5-Trimethoxy-phenyl)-5-(4-bromo-phenyl)-isoxazole |
| 169 | 2-amino-N-(2-methoxy-5-[5-(3,4,5-trimethoxy-phenyl)- |
| | isoxazol-4-yl)-phenyl)acetamide hydrochloride |
| 170 | 2-amino-3-hydroxy-N-(2-methoxy-5-[5-(3,4,5-trimethoxy-phenyl)-isoxazol-4-yl)- |
| | phenyl)propanamide hydrochloride |
| 171 | 2-amino-N-(2-methoxy-5-[5-(3,4,5-trimethoxy-phenyl)-isoxazol-4-yl)- |
| | phenyl)propanamide |
| 172 | 2-amino-N-(2-methoxy-5-[5-(3,4,5-trimethoxy-phenyl)-isoxazol-4-yl)-phenyl)-4- |
| | (methylthio)butanamide hydrochloride |
| 173 | 2-amino-N-(2-methoxy-5-[5-(3,4,5-trimethoxy-phenyl)-isoxazol-4-yl)-phenyl)butanamide |
| 174 | 2-amino-N-(2-methoxy-5-[5-(3,4,5-trimethoxy-phenyl)-isoxazol-4-yl)-phenyl)-3- |
| | phenylpropanamide hydrochloride |
| 175 | 2-amino-N-(2-methoxy-5-[5-(3,4,5-trimethoxy-phenyl)-isoxazol-4-yl)-phenyl)-4- |
| | methylpentanamide hydrochloride |
| 176 | 2-amino-N-(2-methoxy-5-[5-(3,4,5-trimethoxy-phenyl)-isoxazol-4-yl)-phenyl)-3- |
| | (4-methoxyphenyl)propanamide hydrochloride |
| 177 | 2-methoxy-5-[5-(3,4,5-trimethoxy-phenyl)-isoxazol-4-yl)-phenyl dihydrogen |
| | phosphate |
| 178 | Sodium 2-methoxy-5-[5-(3,4,5-trimethoxy-phenyl)-isoxazol-4-yl)-phenyl |
| | phosphate |
| 179 | 1-{2-methoxy-5-[5-(3,4,5-trimethoxy-phenyl)-isoxazol-4-yl]phenylcarbomoyl}-2- |
| | methyl-propyl-ammonium chloride |
| 180 | 1-{2-methoxy-5-[5-(3,4,5-trimethoxy-phenyl)-isoxazol-4-yl]-phenylcarbomoyl}- |
| | 2-methyl-butyl-ammonium chloride |
| 181 | 2-hydroxy-1-{2-methoxy-5-[5-(3,4,5-trimethoxy-phenyl)-isoxazol-4-yl]- |
| | phenylcarbomoyl}-propyl-ammonium chloride |
| 182 | 2-(4-hydroxy-phenyl)-1-{2-methoxy-5-[5-(3,4,5-trimethoxy-phenyl)-isoxazol-4- |
| | yl]-phenylcarbomoyl}-ethyl-ammonium chloride |
| 183 | C-{2-methoxy-5-[5-(3,4,5-trimethoxy-phenyl)-isoxazol-4-yl]-phenylcarbomoyl}- |
| | C-phenyl-methyl-ammonium chloride |
| 184 | 2-(1H-indol-2-yl)-1-{2-methoxy-5-[5-(3,4,5-trimethoxy-phenyl)-isoxazol-4-yl]-phenylcarbomoyl}- |
| | ethyl-ammonium chloride |
| 185 | 2-benzofuran-2-yl-1-{2-methoxy-5-[5-(3,4,5-trimethoxy-phenyl)-isoxazol-4-yl]- |
| | phenylcarbomoyl}-ethyl-ammonium chloride |
| 186 | 2-carboxyl-1-{2-methoxy-5-[5-(3,4,5-trimethoxy-phenyl)-isoxazol-4-yl]- |
| | phenylcarbomoyl}-ethyl-ammonium chloride |
| 187 | 3-carboxyl-1-{2-methoxy-5-[5-(3,4,5-trimethoxy-phenyl)-isoxazol-4-yl]- |
| | phenylcarbomoyl}-propyl-ammonium chloride |
| 188 | 3-carbamoyl-1-{2-methoxy-5-[5-(3,4,5-trimethoxy-phenyl)-isoxazol-4-yl]- |
| | phenylcarbomoyl}-propyl-ammonium chloride |
| 189 | 2-carbamoyl-1-{2-methoxy-5-[5-(3,4,5-trimethoxy-phenyl)-isoxazol-4-yl]- |
| | phenylcarbomoyl}-ethyl-ammonium chloride |
| 190 | 2-(3H-imidazol-4-yl)-1-{2-methoxy-5-[5-(3,4,5-trimethoxy-phenyl)-isoxazol-4-yl]- |
| | phenylcarbomoyl}-ethyl-ammonium chloride |
| 191 | 5-amino-1-{2-methoxy-5-[5-(3,4,5-trimethoxy-phenyl)-isoxazol-4-yl]-phenylcarbomoyl}- |
| | pentyl-ammonium chloride |
| 192 | 4-guanidino-1-{2-methoxy-5-[5-(3,4,5-trimethoxy-phenyl)-isoxazol-4-yl]- |
| | phenylcarbomoyl}-butyl-ammonium chloride |
| 193 | N-{2-methoxy-5-[5-(3,4,5-trimethoxy-phenyl)-isoxazol-4-yl]-phenyl}succinamic |
| | acid |
| 194 | 4-{2-methoxy-5-[5-(3,4,5-trimethoxy-phenyl)-isoxazol-4-yl]-phenylcarbamoyl}- |
| | butyric acid |
| 195 | 2-{2-methoxy-5-[5-(3,4,5-trimethoxy-phenyl)-isoxazol-4-yl]-phenylcarbamoyl}- |
| | ethyl-ammonium chloride |
| 196 | 3-(2-methoxy-ethoxy)-N-{2-methoxy-5-[5-(3,4,5- |
| | trimethoxy-phenyl)-isoxazol-4-yl]-phenyl}-propionamide |
| 197 | 3-(2-PEG)-N-{2-methoxy-5-[5-(3,4,5-trimethoxy-phenyl)-isoxazol-4-yl]-phenyl}- |
| | propionamide |
| 198 | N-{2-methoxy-5-[5-(3,4,5-trimethoxy-phenyl)-isoxazol-4-yl]-phenyl}-3-(2-methylamino- |
| | ethylamino)-propionamide |
| 199 | 3-PEG-N-{2-methoxy-5-[5-(3,4,5-trimethoxy-phenyl)-isoxazol-4-yl]- |
| | phenylcarbamoyl}-methyl)-propionamide |
| 200 | N-{2-methoxy-5-[5-(3,4,5-trimethoxy-phenyl)-isoxazol-4-yl]-phenylcarbamoyl}- |
| | methyl)-succinamic acid |
| 201 | {2-methoxy-5-[5-(3,4,5-trimethoxy-phenyl)-isoxazol-4-yl]-phenyl}-carbamic |
| | acid 2-methoxy-ethyl ester |
| 202 | 2-methoxy-5-(5-(3,4,5-trimethoxyphenyl)isoxazol-4-yl)phenylcarbamate-PEG |
| 203 | 3-amino-N-[4-guanadino-1-{2-methoxy-5-[5-(3,4,5-trimethoxy-phenyl)-isoxazol- |
| | 4-yl]-phenylcarbamoyl}-butylcarbamoyl)-methyl]-succinamic acid |
| 204 | 2-amino-N-(2-methoxy-5-[5-(3,4,5-trimethoxy-phenyl)-isoxazol-4-yl)-phenyl)propanamide |
| | hydrochloride |
| 205 | 2-amino-N-(2-methoxy-5-[3-(3,4,5-trimethoxy-phenyl)-isoxazol-4-yl)-phenyl)acetamide |
| | hydrochloride |
| 206 | 2-amino-3-hydroxy-N-(2-methoxy-5-[3-(3,4,5- |
| | trimethoxy-phenyl)-isoxazol-4-yl)-phenyl)propanamide hydrochloride |
| 207 | 2-amino-N-(2-methoxy-5-[3-(3,4,5-trimethoxy-phenyl)- |
| | isoxazol-4-yl)-phenyl)propanamide |
| 208 | 2-amino-N-(2-methoxy-5-[3-(3,4,5-trimethoxy-phenyl)-isoxazol-4-yl)-phenyl)-4- |
| | (methylthio)butanamide hydrochloride |
| 209 | 2-amino-N-(2-methoxy-5-[3-(3,4,5-trimethoxy-phenyl)-isoxazol-4-yl)-phenyl)butanamide |
| 210 | 2-amino-N-(2-methoxy-5-[3-(3,4,5-trimethoxy-phenyl)-isoxazol-4-yl)-phenyl)-3- |
| | phenylpropanamide hydrochloride |
| 211 | 2-amino-N-(2-methoxy-5-[3(3,4,5-trimethoxy-phenyl)-isoxazol-4-yl)-phenyl)-4- |
| | methylpentanamide hydrochloride |
| 212 | 2-amino-N-(2-methoxy-5-[3-(3,4,5-trimethoxy-phenyl)-isoxazol-4-yl)-phenyl)-3- |
| | (4-methoxyphenyl)propanamide hydrochloride |
| 213 | 2-methoxy-5-[3-(3,4,5-trimethoxy-phenyl)-isoxazol-4-yl)-phenyl dihydrogen |
| | phosphate |
| 214 | Sodium 2-methoxy-5-[3-(3,4,5-trimethoxy-phenyl)-isoxazol-4-yl)-phenyl |
| | phosphate |
| 215 | 1-{2-methoxy-3-[5-(3,4,5-trimethoxy-phenyl)-isoxazol-4-yl]-phenylcarbomoyl}- |
| | 2-methyl-propyl-ammonium chloride |
| 216 | 1-{2-methoxy-5-[3-(3,4,5-trimethoxy-phenyl)-isoxazol-4-yl]-phenylcarbomoyl}- |
| | 2-methyl-butyl-ammonium chloride |
| 217 | 2-hydroxy-1-{2-methoxy-5-[3-(3,4,5-trimethoxy-phenyl)-isoxazol-4-yl]- |
| | phenylcarbomoyl}-propyl-ammonium chloride |
| 218 | 2-(4-hydroxy-phenyl)-1-{2-methoxy-5-[3-(3,4,5-trimethoxy-phenyl)-isoxazol-4- |
| | yl]-phenylcarbomoyl}-ethyl-ammonium chloride |
| 219 | C-{2-methoxy-5-[3-(3,4,5-trimethoxy-phenyl)-isoxazol-4-yl]phenylcarbomoyl}- |
| | C-phenyl-methyl-ammonium chloride |
| 220 | 2-(1H-indol-2-yl)-1-{2-methoxy-5-[3-(3,4,5-trimethoxy-phenyl)- |
| | isoxazol-4-yl]-phenylcarbomoyl}-ethyl-ammonium chloride |
| 221 | 2-benzofuran-2-yl-1-{2-methoxy-5-[3-(3,4,5-trimethoxy-phenyl)-isoxazol-4-yl]- |
| | phenylcarbomoyl}-ethyl-ammonium chloride |
| 222 | 2-carboxyl-1-{2-methoxy-5-[3-(3,4,5-trimethoxy-phenyl)-isoxazol-4-yl]- |
| | phenylcarbomoyl}-ethyl-ammonium chloride |
| 223 | 3-carboxyl-1-{2-methoxy-5-[3-(3,4,5-trimethoxy-phenyl)-isoxazol-4-yl]- |
| | phenylcarbomoyl}-propyl-ammonium chloride |
| 224 | 3-carbamoyl-1-{2-methoxy-5-[3-(3,4,5-trimethoxy-phenyl)-isoxazol-4-yl]- |
| | phenylcarbomoyl}-propyl-ammonium chloride |
| 225 | 2-carbamoyl-1-{2-methoxy-5-[3-(3,4,5-trimethoxy-phenyl)-isoxazol-4-yl]- |
| | phenylcarbomoyl}-ethyl-ammonium chloride |
| 226 | 2-(3H-imidazol-4-yl)-1-{2-methoxy-5-[3-(3,4,5-trimethoxy-phenyl)-isoxazol-4-yl]- |
| | phenylcarbomoyl}-ethyl-ammonium chloride |
| 227 | 5-amino-1-{2-methoxy-5-[3-(3,4,5-trimethoxy-phenyl)-isoxazol-4-yl]- |
| | phenylcarbomoyl}-pentyl-ammonium chloride |
| 228 | 4-guanidino-1-{2-methoxy-5-[3-(3,4,5-trimethoxy-phenyl)-isoxazol-4-yl]- |
| | phenylcarbomoyl}-butyl-ammonium chloride |
| 229 | N-{2-methoxy-5-[3-(3,4,5- |
| | trimethoxy-phenyl)-isoxazol-4-yl]-phenyl}succinamic acid |
| 230 | 4-{2-methoxy-5-[3-(3,4,5-trimethoxy-phenyl)-isoxazol-4-yl]-phenylcarbamoyl}- |
| | butyric acid |
| 231 | 2-{2-methoxy-5-[3-(3,4,5-trimethoxy-phenyl)-isoxazol-4-yl]-phenylcarbamoyl}- |
| | ethyl-ammonium chloride |
| 232 | 3-(2-methoxy-ethoxy)-N-{2-methoxy-5-[3-(3,4,5- |
| | trimethoxy-phenyl)-isoxazol-4-yl]-phenyl}-propionamide |
| 233 | 3-(2-PEG)-N-{2-methoxy-5-[3-(3,4,5-trimethoxy-phenyl)-isoxazol-4-yl]-phenyl}- |
| | propionamide |
| 234 | N-{2-methoxy-5-[3-(3,4,5-trimethoxy-phenyl)-isoxazol-4-yl]-phenyl}-3-(2-methylamino- |
| | ethylamino)-propionamide |
| 235 | 3-PEG-N-{2-methoxy-5-[3-(3,4,5-trimethoxy-phenyl)-isoxazol-4-yl]phenylcarbamoyl}- |
| | methyl)-propionamide |
| 236 | N-{2-methoxy-5-[3-(3,4,5-trimethoxy-phenyl)-isoxazol-4-yl]-phenylcarbamoyl}- |
| | methyl)-succinamic acid |
| 237 | {2-methoxy-5-[3-(3,4,5-trimethoxy-phenyl)-isoxazol-4-yl]-phenyl}-carbamic |
| | acid 2-methoxy-ethyl ester |
| 238 | 2-methoxy-5-(3-(3,4,5-trimethoxyphenyl)isoxazol-4-yl)phenylcarbamate-PEG |
| 239 | 3-amino-N-[4-guanadino-1-{2-methoxy-5-[3-(3,4,5-trimethoxy-phenyl)isoxazol- |
| | 4-yl]-phenylcarbamoyl}-butylcarbamoyl)-methyl]-succinamic acid |
| 240 | 2-amino-N-(2-methoxy-5-[3-(3,4,5-trimethoxy-phenyl)-isoxazol-4-yl)-phenyl)propanamide |
| | hydrochloride |
| 241 | methyl2-(2-(2-methoxy-5-(5-(3,4,5-trimethoxyphenyl)isoxazol-4-yl)phenylamino)- |
| | 2-oxoethylamino)acetate |
| 242 | 4-amino-5-(2-methoxy-5-(5-(3,4,5-trimethoxyphenyl)isoxazol-4-yl)phenylamino)- |
| | 5-oxopentanoic acid hydrochloride |
| 243 | 3-amino-N-(2-methoxy-5-(5-(3,4,5-trimethoxyphenyl)isoxazol-4-yl)phenyl)propanamide |
| | hydrochloride |
| 244 | 3-amino-N-(2-methoxy-5-(5-(3,4,5-trimethoxyphenyl)isoxazol-4-yl)phenyl)-4- |
| | methylpentanamide hydrochloride |
| 245 | methyl2-(2-(2-methoxy-5-(3-(3,4,5-trimethoxyphenyl)isoxazol- |
| | 4-yl)phenylamino)-2-oxoethylamino)acetate |
| 246 | 4-amino-5-(2-methoxy-5-(3-(3,4,5-trimethoxyphenyl)isoxazol- |
| | 4-yl)phenylamino)-5-oxopentanoic acid hydrochloride |
| 247 | 3-amino-N-(2-methoxy-5-(3-(3,4,5-trimethoxyphenyl)isoxazol-4-yl)phenyl)propanamide |
| | hydrochloride |
| 248 | 3-amino-N-(2-methoxy-5-(3-(3,4,5-trimethoxyphenyl)isoxazol-4-yl)phenyl)- |
| | 4-methylpentanamide hydrochloride |
| 249 | 2-methoxy-5-(5-(3,4,5-trimethoxyphenyl)isoxazol-4-yl)aniline |
| 250 | 2-methoxy-5-(3-(3,4,5-trimethoxyphenyl)isoxazol-4-yl)aniline |
| 1b | 4-(4-Bromo-phenyl)-3-(3,4,5-trimethoxy-phenyl)-isoxazole |
| 2b | 4-(Naphthalen-2-yl)-3-(2-hydroxy-4-methoxy-5-ethyl-phenyl)-isoxazole |
| 3b | 4-(4-Methoxy-phenyl)-3-(3,4,5-trimethoxy-phenyl)-isoxazole |
| 4b | 4-(4-Iodo-phenyl)-3-(2-hydroxy-4-methoxy-5-ethyl-phenyl)-isoxazole |
| 5b | 4-Phenyl-3-(2-hydroxy-4- |
| | methoxy-5-propyl-phenyl)-isoxazole |
| 6b | 4-(4-Bromo-phenyl)-3-(2-hydroxy-4-methoxy-5-ethyl-phenyl)-isoxazole |
| 7b | 4-(2,3-Dihydro-benzo[1,4]di-oxin-6-yl)-3-(2-hydroxy-4-methoxy-5-propyl-phenyl)- |
| | isoxazole |
| 8b | 4-(4-hydroxy-phenyl)-3-(3,4,5-trihydroxy-phenyl)-isoxazole |
| 9b | 4-(4-Iodo-phenyl)-3-(3,4,5-trimethoxy-phenyl)-isoxazole |
| 10b | 4-(3-Fluoro-4-methoxy-phenyl)-3-(3,4,5-trimethoxy-phenyl)-isoxazole |
| 11b | 4-(4-Nitro-phenyl)-3-(3,4,5-trimethoxy-phenyl)-isoxazole |
| 12b | 4-(4-Amino-phenyl)-3-(3,4,5-trimethoxy-phenyl)-isoxazole |
| 13b | 4-(4′-Methoxy-biphenyl-4-yl)-3-(3,4,5-trimethoxy-phenyl)-isoxazole |
| 14b | 4-[4-(pyridine-3-yl)-phenyl]-3-(3,4,5-trimethoxy-phenyl)-isoxazole |
| 15b | 4-[4-(pyridine-4-yl)-phenyl]-3-(3,4,5-trimethoxy-phenyl)-isoxazole |
| 16b | 4-[4-(pyridine-2-yl)-phenyl]-3-(3,4,5-trimethoxy-phenyl)-isoxazole |
| 17b | 4-(Quinolin-7-yl)-3-(3,4,5-trimethoxy-phenyl)-isoxazole |
| 18b | 4-(Pyridin-4-yl)-3-(3,4,5-trimethoxy-phenyl)-isoxazole |
| 19b | 4-(Isoquinolin-7-yl)-3-(3,4,5-trimethoxy-phenyl)-isoxazole |
| 20b | 4-(1-Methyl-1H-indol-5-yl)-3-(3,4,5-trimethoxy-phenyl)-isoxazole |
| 21b | 4-(4-Methoxy-phenyl)-3-(benzo[1,3]dioxol-5-yl)-isoxazole |
| 22b | 4-(4-Methoxy-phenyl)-3-(1-ethyl-1H-indol-6-yl)-isoxazole |
| 23b | 4-(4-Carboxy-phenyl)-3-(3,4,5-trimethoxy-phenyl)-isoxazole |
| 24b | 4-(4-Methoxycarbonyl-phenyl)-3-(3,4,5-trimethoxy-phenyl)-isoxazole |
| 25b | 4-[4-(Oxazol-2-yl)-phenyl]-3-(3,4,5-trimethoxy-phenyl)-isoxazole |
| 26b | 4-(4-Methoxy-phenyl)-3-(3,4,5-triethyl-phenyl)-isoxazole |
| 27b | 4-(4-Iodo-phenyl)-3-(3,4,5-triethyl-phenyl)-isoxazole |
| 28b | 4-(3-Fluoro-4-methoxy-phenyl)-3-(3,4,5-triethyl-phenyl)-isoxazole |
| 29b | 4-(4-Nitro-phenyl)-3-(3,4,5-triethyl-phenyl)-isoxazole |
| 30b | 4-(4-N,N-dimethylamino-phenyl)-3-(3,4,5-triethyl-phenyl)-isoxazole |
| 31b | 4-(4-Methoxy-phenyl)-3-(3,4,5-trimethyl-phenyl)-isoxazole |
| 32b | 4-[4-(Pyridin-3-yl)-phenyl]-3-(3,4,5-triethyl-phenyl)-isoxazole |
| 33b | 4-[4-(Pyridin-4-yl)-phenyl]-3-(3,4,5-triethyl-phenyl)-isoxazole |
| 34b | 4-[4-(Pyridin-2-yl)-phenyl]-3-(3,4,5-triethyl-phenyl)-isoxazole |
| 35b | 4-(Quinolin-7-yl)-3-(3,4,5-triethyl-phenyl)-isoxazole |
| 36b | 4-(Pyridin-4-yl)-3-(3,4,5-triethyl-phenyl)-isoxazole |
| 37b | 4-(Isoquinolin-7-yl)-3-(3,4,5-triethyl-phenyl)-isoxazole |
| 38b | 4-(1H-Indol-5-yl)-3-(3,4,5-triethyl-phenyl)-isoxazole |
| 39b | 4-(4-Methoxy-phenyl)-3-(benzo[1,3]dioxol-5-yl)-isoxazole |
| 40b | 4-(4-Methoxy-phenyl)-3-[1-isopropyl-1H-indol-6-yl)-isoxazole |
| 41b | 4-(4-Methoxy-phenyl)-3-(2,3,4-trimethoxy-phenyl)-isoxazole |
| 42b | 4-(3-Hydroxy-4-methoxy-phenyl)-3-(3,4,5-trimethoxy-phenyl)-isoxazole |
| 43b | 4-[3-(Ethyl-hydroxy-phosphoryloxy)-4-methoxy-phenyl]-3-(3,4,5-trimethoxy- |
| | phenyl)-isoxazole |
| 44b | 4-(4-Methoxy-phenyl)-3-(2-hydroxy-4-methoxy-5-ethyl-phenyl)-isoxazole; |
| 45b | 4-(4-Isopropyl-phenyl)-3-(3,4,5-trimethoxy-phenyl)-isoxazole |
| 46b | 4-(2,3-Dihydro-benzo[1,4]dioxin-6-yl)-3-(3,4,5-trimethoxy-phenyl)-isoxazole |
| 47b | 4-(4-Ethyl-phenyl)-3-(3,4,5-trimethoxy-phenyl)-isoxazole |
| 48b | 4-(5-Methoxy-pyridin-2-yl)-3-(3,4,5-trimethoxy-phenyl)-isoxazole |
| 49b | 4-(4-Methoxy-phenyl)-3-(2,3,4-trimethoxy-pyridin-6-yl)-isoxazole |
| 50b | 4-(4-Methoxy-phenyl)-3-(3,5-dimethoxy-4-methoxycarbonyl-phenyl)-isoxazole |
| 51b | 4-(4-Methoxy-phenyl)-3-(3,5-diacetoxy-phenyl)-isoxazole |
| 52b | 4-(2-Methoxy-pyridin-5-yl)-3-(3,4,5-trimethoxy-phenyl)-isoxazole |
| 53b | 4-(4-Methoxy-phenyl)-3-(1-methyl-5-methoxy-1H-indol-7-yl)-isoxazole |
| 54b | 4-(4-Methoxy-phenyl)-3-(1-ethyl-1H-indol-7-yl)-isoxazole |
| 55b | 4-(4-Methoxy-phenyl)-3-(benzo[1,3]dioxol-4-yl)-isoxazole |
| 56b | 4-(2-Hydroxy-4-methoxy-phenyl)-3-(3,4,5-trimethoxy)-isoxazole |
| 57b | 4-[2-(Ethyl-hydroxy-phosphoryloxy)-4-methoxy-phenyl]-3-(3,4,5-trimethoxy- |
| | phenyl)-isoxazole |
| 58b | 4-(Pyridazin-4-yl)-3-(3,4,5-trimethoxy-phenyl)-isoxazole |
| 59b | 4-(Pyrimidin-5-yl)-3-(3,4,5-trimethoxy-phenyl)-isoxazole |
| 60b | 4-(Pyridin-3-yl)-3-(3,4,5-trimethoxy-phenyl)-isoxazole, hydrochloric acid salt |
| 61b | 4-(3-Mercapto-4-methoxy-phenyl)-3-(3,4,5-trimethoxy-phenyl)-isoxazole |
| 62b | 4-(3-Phosphonosulfanyl-4-methoxy-phenyl)-3-(3,4,5-trimethoxy-phenyl)- |
| | isoxazole, disodium salt |
| 63b | 4-(3-Acetylamino-4-methoxy-phenyl)-3-(3,4,5-trimethoxy-phenyl)-isoxazole |
| 64b | 4-(3-Amino-4-methoxy-phenyl)-3-(3,4,5-trimethoxy-phenyl)-isoxazole, |
| | hydrochloric acid salt |
| 65b | 4-(2-Hydroxy-4-methoxy-phenyl)-3-(3,4,5-trimethoxy-phenyl)-isoxazole |
| 66b | 4-(2-Methoxy-pyridine-5-yl)-3-(3,4,5-trimethoxy-phenyl)-isoxazole |
| 67b | 4-(5-Methoxy-pyridine-2-yl)-3-(3,4,5-trimethoxy-phenyl)-isoxazole |
| 68b | 4-(3-Carboxy-4-methoxy-phenyl)-3-(3,4,5-trimethoxy-phenyl)-isoxazole, |
| | sodium salt |
| 69b | 4-(3-Methoxycarbonyl-4-methoxy-phenyl)-3-(3,4,5- |
| | trimethoxy-phenyl)-isoxazole |
| 70b | 4-(3-Sulfooxy-4-methoxy-phenyl)-3-(3,4,5-trimethoxy-phenyl)-isoxazole, |
| | sodium salt |
| 71b | 4-(2-Amino-4-methoxy-phenyl)-3-(3,4,5-trimethoxy-phenyl)-isoxazole |
| 72b | 4-(3,4-Dimethoxy-5-phosphonooxy-phenyl)-3-(3,4,5-trimethoxy-phenyl)- |
| | isoxazole, disodium salt |
| 73b | 4-(2-Phosphonooxy-4-methoxy-phenyl)-3-(3,4,5-trimethoxy-phenyl)-isoxazole, |
| | disodium salt |
| 74b | 4-(4-Methylsulfanyl-phenyl)-3-(3,4,5-trimethoxy-phenyl)-isoxazole |
| 75b | 4-(3-Phosphonooxy-4-methylsulfanyl-phenyl)-3-(3,4,5-trimethoxy-phenyl)- |
| | isoxazole, disodium salt |
| 76b | 4-(3-Amino-4-methylsulfanyl-phenyl)-3-(3,4,5-trimethoxy-phenyl)-isoxazole |
| 77b | 4-(2,3-Dihydro-benzofuran-6-yl)-3-(3,4,5-trimethoxy-phenyl)-isoxazole |
| 78b | 4-(4-Hydroxy-phenyl)-3-(3,4,5-trimethoxy-phenyl)-isoxazole, sodium salt |
| 79b | 4-(4-Phosphonooxy-phenyl)-3-(3,4,5-trimethoxy-phenyl)-isoxazole, disodium |
| | salt |
| 80b | 4-(4-1H-Tetrazol-5-yl-phenyl)-3-(3,4,5-trimethoxy-phenyl)-isoxazole |
| 81b | 4-[4-(1-Methyl-1H-tetrazol-5-yl)-phenyl]-3-(3,4,5-trimethoxy-phenyl)-isoxazole |
| 82b | 4-(1-Methyl-1H-indol-5-yl)-3-(3,4,5-trimethoxy-phenyl)-isoxazole |
| 83b | 4-(Pyridazin-4-yl)-3-(4-methoxy-benzo[1,3]dioxol-6-yl)-isoxazole |
| 84b | 4-(Pyrimidin-5-yl)-3-(4-methoxy-benzo[1,3]dioxol-6-yl)-isoxazole |
| 85b | 4-(Pyridin-3-yl)-3-(4-methoxy-benzo[1,3]dioxol-6-yl)-isoxazole, hydrochloric |
| | acid salt |
| 86b | 4-(3-Mercapto-4-methoxy-phenyl)-3-(4-methoxy-benzo[1,3]dioxol-6-yl)- |
| | isoxazole |
| 87b | 4-(3-Phosphonosulfanyl-4-methoxy-phenyl)-3-(4- |
| | methoxy-benzo[1,3]dioxol-6-yl)-isoxazole |
| 88b | 4-(3-Acetylamino-4-methoxy-phenyl)-3-(4- |
| | methoxy-benzo[1,3]dioxol-6-yl)-isoxazole |
| 89b | 4-(3-Amino-4-methoxy-phenyl)-3-(4-methoxy-benzo[1,3]dioxol-6-yl)- |
| | isoxazole, hydrochloric acid salt |
| 90b | 4-(2-Hydroxy-4-methoxy-phenyl)-3-(4-methoxy-benzo[1,3]dioxol-6-yl)- |
| | isoxazole |
| 91b | 4-(2-Methoxy-pyridin-5-yl)-3-(4-methoxy-benzo[1,3]dioxol-6-yl)-isoxazole |
| 92b | 4-(5-Methoxy-pyridin-2-yl)-3-(4-methoxy-benzo[1,3]dioxol-6-yl)-isoxazole |
| 93b | 4-(3-Carboxy-4-methoxy-phenyl)-3-(4-methoxy-benzo[1,3]dioxol-6-yl)- |
| | isoxazole, sodium salt |
| 94b | 4-(3-Methoxycarbonyl-4-methoxy-phenyl)-3-(4- |
| | methoxy-benzo[1,3]dioxol-6-yl)-isoxazole |
| 95b | 4-(3-Sulfooxy-4-methoxy-phenyl)-3-(4-methoxy-benzo[1,3]dioxol-6-yl)- |
| | isoxazole, sodium salt |
| 96b | 4-(3-Amino-4-methoxy-phenyl)-3-(4-methoxy-benzo[1,3]dioxol-6-yl)- |
| | isoxazole |
| 97b | 4-(3,4-Dimethoxy-5 phosphonooxy-phenyl)-3-(4- |
| | methoxy-benzo[1,3]dioxol-6-yl)-isoxazole, disodium salt |
| 98b | 4-(2-Phosphonooxy-4-methoxy-phenyl)-3-(4- |
| | methoxy-benzo[1,3]dioxol-6-yl)-isoxazole, disodium salt |
| 99b | 4-(4-Methylsulfanyl-phenyl)-3-(4-methoxy-benzo[1,3]dioxol-6-yl)-isoxazole |
| 100b | 4-(3-Phosphonooxy-4-methylsulfanyl-phenyl)-3-(4- |
| | methoxy-benzo[1,3]dioxol-6-yl)-isoxazole, disodium salt |
| 101b | 4-(3-Amino-4-methylsulfanyl-phenyl)-3-(4-methoxy-benzo[1,3]dioxol-6-yl)- |
| | isoxazole |
| 102b | 4-(2,3-Dihydro-benzofuran-6-yl)-3-(4-methoxy-benzo[1,3]dioxol-6-yl)- |
| | isoxazole |
| 103b | 4-(4-Hydroxy-phenyl)-3-(4-methoxy-benzo[1,3]dioxol-6-yl)-isoxazole, sodium |
| | salt |
| 104b | 4-(4-Phosphonooxy-phenyl)-3-(4-methoxy-benzo[1,3]dioxol-6-yl)-isoxazole |
| 105b | 4-(4-1H-Tetrazol-5-yl-phenyl)-3-(4-methoxy-benzo[1,3]dioxol-6-yl)-isoxazole |
| 106b | 4-[4-(1-Methyl-1H-tetrazol-5-yl)-phenyl]-3-(4-methoxy-benzo[1,3]dioxol-6-yl)- |
| | isoxazole |
| 107b | 4-(1-Methyl-1H-indol-5-yl)-3-(4-methoxy-benzo[1,3]dioxol-6-yl)-isoxazole |
| 108b | 4-(3,4,5-Trimethoxy-phenyl)-3-(1-methyl-1H-indol-5-yl)-isoxazole |
| 109b | 4-(3,4,5-Trimethoxy-phenyl)-3-(3-phosphonooxy-4-methoxy-phenyl)-isoxazole, |
| | disodium salt |
| 110b | 4-(3,4,5-Trimethoxy-phenyl)-3-(N,N-dimethylamino-phenyl)-isoxazole |
| 111b | 4-(3,4,5-Trimethoxy-phenyl)-3-(3-amino-4-methoxy-phenyl)-isoxazole, |
| | hydrochloric acid salt |
| 112b | 4-(3,4,5-Trimethoxy-phenyl)-3-[3-(3-hydroxy-2S-amino- |
| | propionamido)-4-methoxy-phenyl]-isoxazole, hydrochloric acid salt |
| 113b | 4-(4-Methoxy-phenyl)-3-(2,4,5-trimethoxy-phenyl)-isoxazole |
| 114b | 4-(4-Methyl-phenyl)-3-(2,4,5-trimethoxy-phenyl)-isoxazole |
| 115b | 4-(4-Ethoxy-phenyl)-3-(2,4,5-trimethoxy-phenyl)-isoxazole |
| 116b | 4-(4-Ethyl-phenyl)-3-(2,4,5-trimethoxy-phenyl)-isoxazole |
| 117b | 4-(4-Propoxy-phenyl)-3-(2,4,5-trimethoxy-phenyl)-isoxazole |
| 118b | 4-(4-Propyl-phenyl)-3-(2,4,5-trimethoxy-phenyl)-isoxazole |
| 119b | 4-(4-Butoxy-phenyl)-3-(2,4,5-trimethoxy-phenyl)-isoxazole |
| 120b | 4-(4-Butyl-phenyl)-3-(2,4,5-trimethoxy-phenyl)-isoxazole |
| 121b | 4-(4-Bromo-phenyl)-3-(2,4,5-trimethoxy-phenyl)-isoxazole |
| 122b | 4-(4-Chloro-phenyl)-3-(2,4,5-trimethoxy-phenyl)-isoxazole |
| 123b | 4-(4-Fluoro-phenyl)-3-(2,4,5-trimethoxy-phenyl)-isoxazole |
| 124b | 4-(4-Nitro-phenyl)-3-(2,4,5-trimethoxy-phenyl)-isoxazole |
| 125b | 4-[4-(N,N-Dimethylamino)-phenyl]-3-(2,4,5-trimethoxy-phenyl)-isoxazole |
| 126b | 4-(3,4-Dimethoxy-phenyl)-3-(2,4,5-trimethoxy-phenyl)-isoxazole |
| 127b | 4-(3-Hydroxy-4-methoxy-phenyl)-3-(2,4,5-trimethoxy-phenyl)-isoxazole |
| 128b | 4-(3,4,5-Trimethoxy-phenyl)-3-(2,4,5-trimethoxy-phenyl)-isoxazole |
| 129b | 4-(4-Methoxy-phenyl)-3-(2,3,5-trimethoxy-phenyl)-isoxazole |
| 130b | 4-(4-Methyl-phenyl)-3-(2,3,5-trimethoxy-phenyl)-isoxazole |
| 131b | 4-(4-Ethoxy-phenyl)-3-(2,3,5-trimethoxy-phenyl)-isoxazole |
| 132b | 4-(4-Ethyl-phenyl)-3-(2,3,5-trimethoxy-phenyl)-isoxazole |
| 133b | 4-(4-Propoxy-phenyl)-3-(2,3,5-trimethoxy-phenyl)-isoxazole |
| 134b | 4-(4-Propyl-phenyl)-3-(2,3,5-trimethoxy-phenyl)-isoxazole |
| 135b | 4-(4-Butoxy-phenyl)-3-(2,3,5-trimethoxy-phenyl)-isoxazole |
| 136b | 4-(4-Butyl-phenyl)-3-(2,3,5-trimethoxy-phenyl)-isoxazole |
| 137b | 4-(4-Bromo-phenyl)-3-(2,3,5-trimethoxy-phenyl)-isoxazole |
| 138b | 4-(4-Chloro-phenyl)-3-(2,3,5-trimethoxy-phenyl)-isoxazole |
| 139b | 4-(4-Fluoro-phenyl)-3-(2,3,5-trimethoxy-phenyl)-isoxazole |
| 140b | 4-(4-Nitro-phenyl)-3-(2,3,5-trimethoxy-phenyl)-isoxazole |
| 141b | 4-[4-(N,N-Dimethylamino)-phenyl]-3-(2,3,5-trimethoxy-phenyl)-isoxazole |
| 142b | 4-(3,4-Dimethoxy-phenyl)-3-(2,3,5-trimethoxy-phenyl)-isoxazole |
| 143b | 4-(3-Hydroxy-4-methoxy-phenyl)-3-(2,3,5-trimethoxy-phenyl)-isoxazole |
| 144b | 4-(3,4,5-Trimethoxy-phenyl)-3-(2,3,5-trimethoxy-phenyl)-isoxazole |
| 145b | 4-(2,3,4,5-Tetramethoxy-phenyl)-3-(4-methoxy-phenyl)-isoxazole |
| 146b | 4-(2,3,4,5-Tetramethoxy-phenyl)-3-(4-methyl-phenyl)-isoxazole |
| 147b | 4-(2,3,4,5-Tetramethoxy-phenyl)-3-(4-ethoxy-phenyl)-isoxazole |
| 148b | 4-(2,3,4,5-Tetramethoxy-phenyl)-3-(4-ethyl-phenyl)-isoxazole |
| 149b | 4-(2,3,4,5-Tetramethoxy-phenyl)-3-(4-propoxy-phenyl)-isoxazole |
| 150b | 4-(2,3,4,5-Tetramethoxy-phenyl)-3-(4-propyl-phenyl)-isoxazole |
| 151b | 4-(2,3,4,5-Tetramethoxy-phenyl)-3-(4-butoxy-phenyl)-isoxazole |
| 152b | 4-(2,3,4,5-Tetramethoxy-phenyl)-3-(4-butyl-phenyl)-isoxazole |
| 153b | 4-(2,3,4,5-Tetramethoxy-phenyl)-3-(4-bromo-phenyl)-isoxazole |
| 154b | 4-(2,3,4,5-Tetramethoxy-phenyl)-3-(4-chloro-phenyl)-isoxazole |
| 155b | 4-(2,3,4,5-Tetramethoxy-phenyl)-3-(4-fluoro-phenyl)-isoxazole |
| 156b | 4-(2,3,4,5-Tetramethoxy-phenyl)-3-(4-nitro-phenyl)-isoxazole |
| 157b | 4-(2,3,4,5-Tetramethoxy-phenyl)-3-[4-(N,N,-dimethylamino)-phenyl]- |
| | isoxazole |
| 158b | 4-(2,3,4,5-Tetramethoxy-phenyl)-3-(3,4-dimethoxy-phenyl)-isoxazole |
| 159b | 4-(2,3,4,5-Tetramethoxy-phenyl)-3-(3-hydroxy-4-methoxy-phenyl)-isoxazole |
| 160b | 4-(2,3,4,5-Tetramethoxy-phenyl)-3-(3,4,5-trimethoxy-phenyl)-isoxazole |
| 161b | 4-(2,3-Dihydro-benzo[1,4]dioxin-6-yl)-3-(3,4-dimethoxy-phenyl)-isoxazole |
| 162b | 4-(3,4-Dimethy-phenyl)-3-(2-hydroxy-4-methoxy-5-ethyl-phenyl)-isoxazole |
| 163b | 4-(4-Chloro-phenyl)-3-(2-hydroxy-4-methoxy-5-ethyl-phenyl)-isoxazole |
| 164b | 4-(4-Methyl-phenyl)-3-(2-hydroxy-4-methoxy-5-ethyl-phenyl)-isoxazole |
| 165b | 4-(4-Amino-phenyl)-3-(2-hydroxy-4-methoxy-5-ethyl-phenyl)-isoxazole |
| 166b | 4-(4-Trifluoromethyl-phenyl)-3-(2-hydroxy-4-methoxy-5- |
| | ethyl-phenyl)-isoxazole |
| 167b | 4-(4-Methoxy-phenyl)-3-(2-hydroxy-4-methoxy-5-ethyl-phenyl)-isoxazole |
| 168b | 4-(3,4,5-Trimethoxy-phenyl)-3-(4-bromo-phenyl)-isoxazole |
| 1c | 1-(3,4,5-trimethoxy-phenyl)-5-(4-bromo-phenyl)-1H-[1,2,3]triazole |
| 2c | 1-(2-hydroxy-4-methoxy-5-ethyl-phenyl)-5-(naphthylen-2-yl)-1H-[1,2,3]triazole |
| 3c | 1-(3,4,5-trimethoxy-phenyl)-5-(4-methoxy-phenyl)-1H-[1,2,3]triazole |
| 4c | 1-(2-hydroxy-4-methoxy-5-ethyl-phenyl)-5-(4-iodo-phenyl)-1H-[1,2,3]triazole |
| 5c | 1-(3,4,5-trimethoxy-phenyl)-5-[4-(N,N-dimethylamino)-phenyl]-1H-[1,2,3]triazole |
| 6c | 1-(2-hydroxy-4-methoxy-5-ethyl-phenyl)-5-(4-bromo-phenyl)-1H-[1,2,3]triazole |
| 7c | 1-(2-hydroxy-4-methoxy-5-(2,3-dihydro-benzo[1,4]dioxin-6-yl)-5-(4-bromo-phenyl)- |
| | 1H-[1,2,3]triazole |
| 8c | 1-(3,4,5-hydroxy-phenyl)-5-(4-hyoxy-phenyl)-1H-[1,2,3]triazole |
| 9c | 1-(3,4,5-trimethoxy-phenyl)-5-(4-iodo-phenyl)-1H-[1,2,3]triazole |
| 10c | 1-(3,4,5-trimethoxy-phenyl)-5-(3-fluoro-4-methoxy-phenyl)-1H-[1,2,3]triazole |
| 11c | 1-(3,4,5-trimethoxy-phenyl)-5-(4-nitro-phenyl)-1H-[1,2,3]triazole |
| 12c | 1-(3,4,5-trimethoxy-phenyl)-5-(4-amino-phenyl)-1H-[1,2,3]triazole |
| 13c | 1-(3,4,5-trimethoxy-phenyl)-5-(4′-methoxy-biphenyl-4-yl)-1H-[1,2,3]triazole |
| 14c | 1-(3,4,5-trimethoxy-phenyl)-5-[4-(pyridin-3-yl)-phenyl]-1H-[1,2,3]triazole |
| 15c | 1-(3,4,5-trimethoxy-phenyl)-5-[4-(pyridin-4-yl)-phenyl]-1H-[1,2,3]triazole |
| 16c | 1-(3,4,5-trimethoxy-phenyl)-5-[4-(pyridin-2-yl)-phenyl]-1H-[1,2,3]triazole |
| 17c | 1-(3,4,5-trimethoxy-phenyl)-5-(quinolin-7-yl)-1H-[1,2,3]triazole |
| 18c | 1-(3,4,5-trimethoxy-phenyl)-5-(pyridine-4-yl)-1H-[1,2,3]triazole |
| 19c | 1-(3,4,5-trimethoxy-phenyl)-5-(isoquinolin-7-yl)-1H-[1,2,3]triazole |
| 20c | 1-(3,4,5-trimethoxy-phenyl)-5-(1-methyl-1H-indol-5-yl)-1H-[1,2,3]triazole |
| 21c | 1-(benzo[1,3]dioxol-5-yl)-5-(4-methoxy-phenyl)-1H-[1,2,3]triazole |
| 22c | 1-(1-ethyl-1H-indol-6-yl)-5-(4-methoxy-phenyl)-1H-[1,2,3]triazole |
| 23c | 1-(3,4,5-trimethoxy-phenyl)-5-(4-carboxy-phenyl)-1H-[1,2,3]triazole |
| 24c | 1-(3,4,5-trimethoxy-phenyl)-5-(4-carbomethoxy-phenyl)-1H-[1,2,3]triazole |
| 25c | 1-(3,4,5-trimethoxy-phenyl)-5-[4-(oxazol-2-yl)-phenyl]-1H-[1,2,3]triazole |
| 26c | 1-(3,4,5-triethyl-phenyl)-5-(4-methoxy-phenyl)-1H-[1,2,3]triazole |
| 27c | 1-(3,4,5-triethyl-phenyl)-5-(4-iodo-phenyl)-1H-[1,2,3]triazole |
| 28c | 1-(3,4,5-triethyl-phenyl)-5-(3-fluoro-4-methoxy-phenyl)-1H-[1,2,3]triazole |
| 29c | 1-(3,4,5-triethyl-phenyl)-5-(4-nitro-phenyl)-1H-[1,2,3]triazole |
| 30c | 1-(3,4,5-triethyl-phenyl)-5-[4-(N,N-dimethylamino)-phenyl]-1H-[1,2,3]triazole |
| 31c | 1-(3,4,5-trimethyl-phenyl)-5-(4-methoxy-phenyl)-1H-[1,2,3]triazole |
| 32c | 1-(3,4,5-triethyl-phenyl)-5-[4-(pyridine-3-yl)-phenyl]-1H-[1,2,3]triazole |
| 33c | 1-(3,4,5-triethyl-phenyl)-5-[4-(pyridine-4-yl)-phenyl]-1H-[1,2,3]triazole |
| 34c | 1-(3,4,5-triethyl-phenyl)-5-[4-(pyridine-2-yl)-phenyl]-1H-[1,2,3]triazole |
| 35c | 1-(3,4,5-triethyl-phenyl)-5-(quinolin-7-yl)-1H-[1,2,3]triazole |
| 36c | 1-(3,4,5-triethyl-phenyl)-5-(pyridine-4-yl)-1H-[1,2,3]triazole |
| 37c | 1-(3,4,5-triethyl-phenyl)-5-(isoquinolin-7-yl)-1H-[1,2,3]triazole |
| 38c | 1-(3,4,5-triethyl-phenyl)-5-(1H-indol-5-yl)-1H-[1,2,3]triazole |
| 39c | 1-(benzo[1,3]dioxol-5-yl)-5-(4-methoxy-phenyl)-1H-[1,2,3]triazole |
| 40c | 1-(1-isopropyl-1H-indol-6-yl)-5-(4-methoxy-phenyl)-1H-[1,2,3]triazole |
| 41c | 1-(2,3,4-trimethoxy-phenyl)-5-(4-methoxy-phenyl)-1H-[1,2,3]triazole |
| 42c | 1-(3,4,5-trimethoxy-phenyl)-5-(3-hydroxy-4-methoxy-phenyl)-1H-[1,2,3]triazole |
| 43c | O-ethyl-O-{2-methoxy-5-[1-(3,4,5-trimethoxy-phenyl)-1H-[1,2,3]triazol-5-yl]-phenyl}- |
| | phosphate |
| 44c | 1-(2-hydroxy-4-methoxy-5-ethyl-phenyl)-5-(4-methoxy-phenyl)-1H-[1,2,3]triazole |
| 45c | 1-(3,4,5-trimethoxy-phenyl)-5-(4-isopropyl-phenyl)-1H-[1,2,3]triazole |
| 46c | 1-(3,4,5-trimethoxy-phenyl)-5-(2,3-dihydro-benzo[1,4]dioxine-6-yl)-1H-[1,2,3]triazole |
| 47c | 1-(3,4,5-trimethoxy-phenyl)-5-(4-ethyl-phenyl)-1H-[1,2,3]triazole |
| 48c | 1-(3,4,5-trimethoxy-phenyl)-5-(5-methoxy-pyridine-2-yl)-1H-[1,2,3]triazole |
| 49c | 1-(4,5,6-trimethoxy-pyridin-2-yl)-5-(4-methoxy-phenyl)-1H-[1,2,3]triazole |
| 50c | 1-(3,5-dimethoxy-4-carbomethoxy-phenyl)-5-(4-methoxy-phenyl)-1H-[1,2,3]triazole |
| 51c | 1-(3,5-diacetoxy-phenyl)-5-(4-methoxy-phenyl)-1H-[1,2,3]triazole |
| 52c | 1-(3,4,5-trimethoxy-phenyl)-5-(2-methoxy-pyridine-5-yl)-1H-[1,2,3]triazole |
| 53c | 1-(1-methyl-5-methoxy-1H-indol-7-yl)-5-(4-methoxy-phenyl)-1H-[1,2,3]triazole |
| 54c | 1-(1-methyl-1H-indol-7-yl)-5-(4-methoxy-phenyl)-1H-[1,2,3]triazole |
| 55c | 1-(Benzo[1,3]dioxol-4-yl)-5-(4-methoxy-phenyl)-1H-[1,2,3]triazole |
| 56c | 1-(3,4,5-trimethoxy-phenyl)-5-(2-hydroxy-4-methoxy-phenyl)-1H-[1,2,3]triazole |
| 57c | O-ethyl-O-{5-methoxy-2-[1-(3,4,5-trimethoxy-phenyl)-1H-[1,2,3]triazol-5-yl]-phenyl}- |
| | phosphate |
| 58c | 1-(3,4,5-trimethoxy-phenyl)-5-(pyridazin-4-yl)-1H-[1,2,3]triazole |
| 59c | 1-(3,4,5-trimethoxy-phenyl)-5-(pyrimidin-5-yl)-1H-[1,2,3]triazole |
| 60c | 1-(3,4,5-trimethoxy-phenyl)-5-(pyridin-2-yl)-1H-[1,2,3]triazole, hydrochloric |
| | acid salt |
| 61c | 1-(3,4,5-trimethoxy-phenyl)-5-(2-mercapto-4-methoxy-phenyl)-1H-[1,2,3]triazole |
| 62c | S-{2-methoxy-5-[1-(3,4,5-trimethoxy-phenyl)-1H-[1,2,3]triazol-5-yl]-phenyl}-thiophosphate, |
| | disodium salt |
| 63c | 1-(3,4,5-trimethoxy-phenyl)-5-(3-acetamido-4-methoxy-phenyl)-1H-[1,2,3]triazole |
| 64c | 1-(3,4,5-trimethoxy-phenyl)-5-(3-amino-4-methoxy-phenyl)-1H-[1,2,3]triazole, |
| | hydrochloric acid salt |
| 65c | 1-(3,4,5-trimethoxy-phenyl)-5-(2-hydroxy-4-methoxy-phenyl)-1H-[1,2,3]triazole |
| 66c | 1-(3,4,5-trimethoxy-phenyl)-5-(2-methoxy-pyridin-5-yl)-1H-[1,2,3]triazole |
| 67c | 1-(3,4,5-trimethoxy-phenyl)-5-(5-methoxy-pyridin-2-yl)-1H-[1,2,3]triazole |
| 68c | 1-(3,4,5-trimethoxy-phenyl)-5-(3-carboxy-4-methoxy-phenyl)-1H-[1,2,3]triazole, |
| | sodium salt |
| 69c | 1-(3,4,5-trimethoxy-phenyl)-5-(3-methoxycarbonyl-4-methoxy-phenyl)-1H-[1,2, |
| | 3]triazole |
| 70c | 1-(3,4,5-trimethoxy-phenyl)-5-(3-sulfooxy-4-methoxy-phenyl)-1H-[1,2,3]triazole, |
| | sodium salt |
| 71c | 1-(3,4,5-trimethoxy-phenyl)-5-(2-amino-4-methoxy-phenyl)-1H-[1,2,3]triazole |
| 72c | 1-(3,4,5-trimethoxy-phenyl)-5-(3-phosphonooxy-4,5-dimethoxy-phenyl)-1H-[1,2, |
| | 3]triazole, disodium salt |
| 73c | 1-(3,4,5-trimethoxy-phenyl)-5-(2-phosphonooxy-4-methoxy-phenyl)-1H-[1,2,3]triazole, |
| | disodium salt |
| 74c | 1-(3,4,5-trimethoxy-phenyl)-5-(4-methylsulfanyl-phenyl)-1H-[1,2,3]triazole |
| 75c | 1-(3,4,5-trimethoxy-phenyl)-5-(3-phosphonooxy-4-methylsulfanyl-phenyl)-1H- |
| | [1,2,3]triazole, disodium salt |
| 76c | 1-(3,4,5-trimethoxy-phenyl)-5-(3-amino-4-methylsulfanyl-phenyl)-1H-[1,2,3]triazole |
| 77c | 1-(3,4,5-trimethoxy-phenyl)-5-(2,3-dihydro-benzofuran-6-yl)-1H-[1,2,3]triazole |
| 78c | 1-(3,4,5-trimethoxy-phenyl)-5-(4-hydroxy-phenyl)-1H-[1,2,3]triazole, sodium |
| | salt |
| 79c | 1-(3,4,5-trimethoxy-phenyl)-5-(4-phosphonooxy-phenyl)-1H-[1,2,3]triazole, |
| | disodium salt |
| 80c | 1-(3,4,5-trimethoxy-phenyl)-5-[4-(tetrazol-5-yl)-phenyl]-1H-[1,2,3]triazole |
| 81c | 1-(3,4,5-trimethoxy-phenyl)-5-[4-(1-methyl-tetrazol-5-yl)-phenyl]-1H-[1,2,3]triazole |
| 82c | 1-(3,4,5-trimethoxy-phenyl)-5-(1-methyl-1H-indol-5-yl)-1H-[1,2,3]triazole |
| 83c | 1-(7-methoxy-benzo[1,3]dioxol-5-yl)-5-(pyridazin-4-yl)-1H-[1,2,3]triazole |
| 84c | 1-(7-methoxy-benzo[1,3]dioxol-5-yl)-5-(pyrimidin-5-yl)-1H-[1,2,3]triazole |
| 85c | 1-(7-methoxy-benzo[1,3]dioxol-5-yl)-5-(pyridine-3-yl)-1H-[1,2,3]triazole, |
| | hydrochloric acid salt |
| 86c | 1-(7-methoxy-benzo[1,3]dioxol-5-yl)-5-(3-mercapto-4-methoxy-phenyl)-1H-[1,2, |
| | 3]triazole |
| 87c | S-{2-methoxy-5-[1-(7-methoxy-benzo[1,3]dioxol-5-yl)-1H-[1,2,3]triazol-5-yl]-phenyl}- |
| | thiophosphate, disodium salt |
| 88c | 1-(7-methoxy-benzo[1,3]dioxol-5-yl)-5-(3-acetamindo-4-methoxy-phenyl)-1H-[1, |
| | 2,3]triazole |
| 89c | 1-(7-methoxy-benzo[1,3]dioxol-5-yl)-5-(3-amino-4-methoxy-phenyl)-1H-[1,2,3]triazole, |
| | hydrochloric acid salt |
| 90c | 1-(7-methoxy-benzo[1,3]dioxol-5-yl)-5-(2-hydroxy-4-methoxy-phenyl)-1H-[1,2, |
| | 3]triazole |
| 91c | 1-(7-methoxy-benzo[1,3]dioxol-5-yl)-5-(2-methoxy-pyridin-5-yl)-1H-[1,2,3]triazole |
| 92c | 1-(7-methoxy-benzo[1,3]dioxol-5-yl)-5-(5-methoxy-pyridin-2-yl)-1H-[1,2,3]triazole |
| 93c | 1-(7-methoxy-benzo[1,3]dioxol-5-yl)-5-(3-carboxy-4-methoxy-phenyl)-1H-[1,2, |
| | 3]triazole |
| 94c | 1-(7-methoxy-benzo[1,3]dioxol-5-yl)-5-(3-methoxycarbonyl-4-methoxy-phenyl)- |
| | 1H-[1,2,3]triazole |
| 95c | 1-(7-methoxy-benzo[1,3]dioxol-5-yl)-5-(3-sulfooxy-4-methoxy-phenyl)-1H-[1,2, |
| | 3]triazole, sodium salt |
| 96c | 1-(7-methoxy-benzo[1,3]dioxol-5-yl)-5-(2-amino-4-methoxy-phenyl)-1H-[1,2,3]triazole |
| 97c | 1-(7-methoxy-benzo[1,3]dioxol-5-yl)-5-(3-phosphonyl-4,5-dimethoxy-phenyl)-1H- |
| | [1,2,3]triazole, disodium salt |
| 98c | 1-(7-methoxy-benzo[1,3]dioxol-5-yl)-5-(2-phosphonyl-4-methoxy-phenyl)-1H- |
| | [1,2,3]triazole, sodium salt |
| 99c | 1-(7-methoxy-benzo[1,3]dioxol-5-yl)-5-(4-methylsulfanyl-phenyl)-1H-[1,2,3]triazole |
| 100c | 1-(7-methoxy-benzo[1,3]dioxol-5-yl)-5-(3-phosphonyl-4-methylsulfanyl-phenyl)- |
| | 1H-[1,2,3]triazole, disodium salt |
| 101c | 1-(7-methoxy-benzo[1,3]dioxol-5-yl)-5-(3-amino-4-methylsulfanyl-phenyl)-1H- |
| | [1,2,3]triazole |
| 102c | 1-(7-methoxy-benzo[1,3]dioxol-5-yl)-5-(2,3-dihydro-benzofuran-6-yl)-1H-[1,2,3]triazole |
| 103c | 1-(7-methoxy-benzo[1,3]dioxol-5-yl)-5-(4-hydroxy-phenyl)-1H-[1,2,3]triazole, |
| | sodium salt |
| 104c | 1-(7-methoxy-benzo[1,3]dioxol-5-yl)-5-(4-phosphonooxy-phenyl)-1H-[1,2,3]triazole, |
| | disodium salt |
| 105c | 1-(7-methoxy-benzo[1,3]dioxol-5-yl)-5-[1H-tetrazol-5-yl)-phenyl]-1H-[1,2,3]triazole |
| 106c | 1-(7-methoxy-benzo[1,3]dioxol-5-yl)-5-[1-methyl-1H-tetrazol-5-yl)-phenyl]-1H- |
| | [1,2,3]triazole |
| 107c | 1-(7-methoxy-benzo[1,3]dioxol-5-yl)-5-(1-methyl-1H-indol-5-yl)-1H-[1,2,3]triazole |
| 108c | 1-(1-methyl-1H-indol-5-yl)-5-(3,4,5-trimethoxy-phenyl)-1H-[1,2,3]triazole |
| 109c | 1-(3-phosphonooxy-4-methoxy-phenyl)-5-(3,4,5-trimethoxy-phenyl)-1H-[1,2,3]triazole, |
| | disodium salt |
| 110c | 1-[4-(N,N-dimethylamino)-phenyl]-5-(3,4,5-trimethoxy-phenyl)-1H-[1,2,3]triazole |
| 111c | 1-(3-amino-4-methoxy-phenyl)-5-(3,4,5-trimethoxy-phenyl)-1H-[1,2,3]triazole, |
| | hydrochloric acid salt |
| 112c | 2-hydroxy-1-{2-methoxy-5-[5-(3,4,5-trimethoxy-phenyl)-[1,2,3]triazol-1-yl]-phenylcarbamoyl}- |
| | ethyl-ammonium chloride |
| 113c | 1-(2,4,5-trimethoxy-phenyl)-5-(4-methoxy-phenyl)-1H-[1,2,3]triazole |
| 114c | 1-(2,4,5-trimethoxy-phenyl)-5-(4-methyl-phenyl)-1H-[1,2,3]triazole |
| 115c | 1-(2,4,5-trimethoxy-phenyl)-5-(4-ethoxy-phenyl)-1H-[1,2,3]triazole |
| 116c | 1-(2,4,5-trimethoxy-phenyl)-5-(4-ethyl-phenyl)-1H-[1,2,3]triazole |
| 117c | 1-(2,4,5-trimethoxy-phenyl)-5-(4-propoxy-phenyl)-1H-[1,2,3]triazole |
| 118c | 1-(2,4,5-trimethoxy-phenyl)-5-(4-propyl-phenyl)-1H-[1,2,3]triazole |
| 119c | 1-(2,4,5-trimethoxy-phenyl)-5-(4-butoxy-phenyl)-1H-[1,2,3]triazole |
| 120c | 1-(2,4,5-trimethoxy-phenyl)-5-(4-butyl-phenyl)-1H-[1,2,3]triazole |
| 121c | 1-(2,4,5-trimethoxy-phenyl)-5-(4-bromo-phenyl)-1H-[1,2,3]triazole |
| 122c | 1-(2,4,5-trimethoxy-phenyl)-5-(4-chloro-phenyl)-1H-[1,2,3]triazole |
| 123c | 1-(2,4,5-trimethoxy-phenyl)-5-(4-fluoro-phenyl)-1H-[1,2,3]triazole |
| 124c | 1-(2,4,5-trimethoxy-phenyl)-5-(4-nitro-phenyl)-1H-[1,2,3]triazole |
| 125c | 1-(2,4,5-trimethoxy-phenyl)-5-[4-(N,N-dimethylamino)-phenyl]-1H-[1,2,3]triazole |
| 126c | 1-(2,4,5-trimethoxy-phenyl)-5-(3,4-dimethoxy-phenyl)-1H-[1,2,3]triazole |
| 127c | 1-(2,4,5-trimethoxy-phenyl)-5-(3-hydroxy-4-methoxy-phenyl)-1H-[1,2,3]triazole |
| 128c | 1-(2,4,5-trimethoxy-phenyl)-5-(3,4,5-trimethoxy-phenyl)-1H-[1,2,3]triazole |
| 129c | 1-(2,3,5-trimethoxy-phenyl)-5-(4-methoxy-phenyl)-1H-[1,2,3]triazole |
| 130c | 1-(2,3,5-trimethoxy-phenyl)-5-(4-methyl-phenyl)-1H-[1,2,3]triazole |
| 131c | 1-(2,3,5-trimethoxy-phenyl)-5-(4-ethoxy-phenyl)-1H-[1,2,3]triazole |
| 132c | 1-(2,3,5-trimethoxy-phenyl)-5-(4-ethyl-phenyl)-1H-[1,2,3]triazole |
| 133c | 1-(2,3,5-trimethoxy-phenyl)-5-(4-propoxy-phenyl)-1H-[1,2,3]triazole |
| 134c | 1-(2,3,5-trimethoxy-phenyl)-5-(4-propyl-phenyl)-1H-[1,2,3]triazole |
| 135c | 1-(2,3,5-trimethoxy-phenyl)-5-(4-butoxy-phenyl)-1H-[1,2,3]triazole |
| 136c | 1-(2,3,5-trimethoxy-phenyl)-5-(4-butyl-phenyl)-1H-[1,2,3]triazole |
| 137c | 1-(2,3,5-trimethoxy-phenyl)-5-(4-bromo-phenyl)-1H-[1,2,3]triazole |
| 138c | 1-(2,3,5-trimethoxy-phenyl)-5-(4-chloro-phenyl)-1H-[1,2,3]triazole |
| 139c | 1-(2,3,5-trimethoxy-phenyl)-5-(4-fluoro-phenyl)-1H-[1,2,3]triazole |
| 140c | 1-(2,3,5-trimethoxy-phenyl)-5-(4-nitro-phenyl)-1H-[1,2,3]triazole |
| 141c | 1-(2,3,5-trimethoxy-phenyl)-5-[4-(N,N-dimethylamino)-phenyl]-1H-[1,2,3]triazole |
| 142c | 1-(2,3,5-trimethoxy-phenyl)-5-(3,4-dimethoxy-phenyl)-1H-[1,2,3]triazole |
| 143c | 1-(2,3,5-trimethoxy-phenyl)-5-(3-hydroxy-4-methoxy-phenyl)-1H-[1,2,3]triazole |
| 144c | 1-(2,3,5-trimethoxy-phenyl)-5-(3,4,5-trimethoxy-phenyl)-1H-[1,2,3]triazole |
| 145c | 1-(4-methoxy-phenyl)-5-(2,3,4,5-tetramethoxy-phenyl)-1H-[1,2,3]triazole |
| 146c | 1-(4-methyl-phenyl)-5-(2,3,4,5-tetramethoxy-phenyl)-1H-[1,2,3]triazole |
| 147c | 1-(4-ethoxy-phenyl)-5-(2,3,4,5-tetramethoxy-phenyl)-1H-[1,2,3]triazole |
| 148c | 1-(4-ethyl-phenyl)-5-(2,3,4,5-tetramethoxy-phenyl)-1H-[1,2,3]triazole |
| 149c | 1-(4-propoxy-phenyl)-5-(2,3,4,5-tetramethoxy-phenyl)-1H-[1,2,3]triazole |
| 150c | 1-(4-propyl-phenyl)-5-(2,3,4,5-tetramethoxy-phenyl)-1H-[1,2,3]triazole |
| 151c | 1-(4-butoxy-phenyl)-5-(2,3,4,5-tetramethoxy-phenyl)-1H-[1,2,3]triazole |
| 152c | 1-(4-butyl-phenyl)-5-(2,3,4,5-tetramethoxy-phenyl)-1H-[1,2,3]triazole |
| 153c | 1-(4-bromo-phenyl)-5-(2,3,4,5-tetramethoxy-phenyl)-1H-[1,2,3]triazole |
| 154c | 1-(4-chloro-phenyl)-5-(2,3,4,5-tetramethoxy-phenyl)-1H-[1,2,3]triazole |
| 155c | 1-(4-fluoro-phenyl)-5-(2,3,4,5-tetramethoxy-phenyl)-1H-[1,2,3]triazole |
| 156c | 1-(4-nitro-phenyl)-5-(2,3,4,5-tetramethoxy-phenyl)-1H-[1,2,3]triazole |
| 157c | 1-[4-(N,N-dimethylamino)-phenyl]-5-(2,3,4,5-tetramethoxy-phenyl)-1H-[1,2,3]triazole |
| 158c | 1-(3,4-dimethoxy-phenyl)-5-(2,3,4,5-tetramethoxy-phenyl)-1H-[1,2,3]triazole |
| 159c | 1-(3-hydroxy-4-methoxy-phenyl)-5-(2,3,4,5-tetramethoxy-phenyl)-1H-[1,2,3]triazole |
| 160c | 1-(3,4,5-trimethoxy-phenyl)-5-(2,3,4,5-tetramethoxy-phenyl)-1H-[1,2,3]triazole |
| 161c | 1-(3,4-trimethoxy-phenyl)-5-(2,3-dihydro-benzo[1,4]dioxin-6-yl)-1H-[1,2,3]triazole |
| 162c | 1-(2-hydroxy-4-methoxy-5-ethyl-phenyl)-5-(3,4-dimethyl-phenyl)-1H-[1,2,3]triazole |
| 163c | 1-(2-hydroxy-4-methoxy-5-ethyl-phenyl)-5-(4-chloro-phenyl)-1H-[1,2,3]triazole |
| 164c | 1-(2-hydroxy-4-methoxy-5-propyl-phenyl)-5-phenyl-1H-[1,2,3]triazole |
| 165c | 1-(2-hydroxy-4-methoxy-5-ethyl-phenyl)-5-(4-methyl-phenyl)-1H-[1,2,3]triazole |
| 166c | 1-(2-hydroxy-4-methoxy-5-ethyl-phenyl)-5-(4-amino-phenyl)-1H-[1,2,3]triazole |
| 167c | 1-(2-hydroxy-4-methoxy-5-ethyl-phenyl)-5-(4-trifluoromethyl-phenyl)-1H-[1,2,3]triazole |
| 168c | 1-(2-hydroxy-4-methoxy-5-ethyl-phenyl)-5-(4-methoxy-phenyl)-1H-[1,2,3]triazole |
| 169c | 1-(4-bromo-phenyl)-5-(3,4,5-trimethoxy-phenyl)-1H-[1,2,3]triazole |
| 170c | 2-amino-N-(2-methoxy-5-(1-(3,4,5-trimethoxyphenyl)-1H-1,2,3-triazol-5-yl)phenyl)acetamide |
| | hydrochloride |
| 171c | 2-amino-3-hydroxy-N-(2-methoxy-5-(1-(3,4,5-trimethoxyphenyl)-1H-1,2,3-triazol- |
| | 5-yl)phenyl)propanamide hydrochloride |
| 172c | 2-amino-N-(2-methoxy-5-(1-(3,4,5-trimethoxyphenyl)-1H-1,2,3-triazol-5-yl)phenyl)propanamide |
| 173c | 2-amino-N-(2-methoxy-5-(1-(3,4,5-trimethoxyphenyl)-1H-1,2,3-triazol-5-yl)phenyl)- |
| | 4-(methylthio)butanamide hydrochloride |
| 174c | 2-amino-N-(2-methoxy-5-(1-(3,4,5-trimethoxyphenyl)-1H-1,2,3-triazol-5-yl)phenyl)butanamide |
| | hydrochloride |
| 175c | 2-amino-N-(2-methoxy-5-(1-(3,4,5-trimethoxyphenyl)-1H-1,2,3-triazol-5-yl)phenyl)- |
| | 3-phenylpropanamide hydrochloride |
| 176c | 2-amino-N-(2-methoxy-5-(1-(3,4,5-trimethoxyphenyl)-1H-1,2,3-triazol-5-yl)phenyl)- |
| | 4-methylpentanamide hydrochloride |
| 177c | 2-amino-N-(2-methoxy-5-(1-(3,4,5-trimethoxyphenyl)-1H-1,2,3-triazol-5-yl)phenyl)- |
| | 3-(4-methoxyphenyl)propanamide |
| | hydrochloride |
| 178c | 2-methoxy-5-(1-(3,4,5-trimethoxyphenyl)-1H-1,2,3-triazol-5-yl)phenyl |
| | dihydrogen phosphate |
| 179c | sodium 2-methoxy-5-(1-(3,4,5-trimethoxyphenyl)-1H-1,2,3-triazol-5-yl)phenyl |
| | phosphate |
| 180c | 1-(2-methoxy-5-(1-(3,4,5-trimethoxyphenyl)-1H-1,2,3-triazol-5-yl)phenylamino)- |
| | 3-methyl-1-oxobutan-2-aminium chloride |
| 181c | 1-(2-methoxy-5-(1-(3,4,5-trimethoxyphenyl)-1H-1,2,3-triazol-5-yl)phenylamino)- |
| | 3-methyl-1-oxopentan-2-aminium chloride |
| 182c | 3-hydroxy-1-(2-methoxy-5-(1-(3,4,5-trimethoxyphenyl)-1H-1,2,3-triazol-5-yl)phenylamino)- |
| | 1-oxobutan-2-aminium chloride |
| 183c | 3-(4-hydroxyphenyl)-1-(2-methoxy-5-(1-(3,4,5-trimethoxyphenyl)-1H-1,2,3-triazol- |
| | 5-yl)phenylamino)-1-oxopropan-2-aminium chloride |
| 184c | 2-(2-methoxy-5-(1-(3,4,5-trimethoxyphenyl)-1H-1,2,3-triazol-5-yl)phenylamino)- |
| | 2-oxo-1-phenylethanaminium chloride |
| 185c | 3-(1H-indol-2-yl)-1-(2-methoxy-5-(1-(3,4,5-trimethoxyphenyl)-1H-1,2,3-triazol- |
| | 5-yl)phenylamino)-1-oxopropan-2-aminium chloride |
| 186c | 3-(benzofuran-2-yl)-1-(2-methoxy-5-(1-(3,4,5-trimethoxyphenyl)-1H-1,2,3-triazol- |
| | 5-yl)phenylamino)-1-oxopropan-2-aminium chloride |
| 187c | 3-carboxy-1-(2-methoxy-5-(1-(3,4,5-trimethoxyphenyl)-1H-1,2,3-triazol-5-yl)phenylamino)- |
| | 1-oxopropan-2-aminium chloride |
| 188c | 4-carboxy-1-(2-methoxy-5-(1-(3,4,5-trimethoxyphenyl)-1H-1,2,3-triazol-5-yl)phenylamino)- |
| | 1-oxobutan-2-aminium chloride |
| 189c | 5-amino-1-(2-methoxy-5-(1-(3,4,5-trimethoxyphenyl)-1H-1,2,3-triazol-5-yl)phenylamino)- |
| | 1,5-dioxopentan-2-aminium chloride |
| 190c | 4-amino-1-(2-methoxy-5-(1-(3,4,5-trimethoxyphenyl)-1H-1,2,3-triazol-5-yl)phenylamino)- |
| | 1,4-dioxobutan-2-aminium chloride |
| 191c | 3-(1H-imidazol-5-yl)-1-(2-methoxy-5-(1-(3,4,5-trimethoxyphenyl)-1H-1,2,3-triazol- |
| | 5-yl)phenylamino)-1-oxopropan-2-aminium chloride |
| 192c | 6-amino-1-(2-methoxy-5-(1-(3,4,5-trimethoxyphenyl)-1H-1,2,3-triazol-5-yl)phenylamino)- |
| | 1-oxohexan-2-aminium chloride |
| 193c | 5-guanidino-1-(2-methoxy-5-(1-(3,4,5-trimethoxyphenyl)-1H-1,2,3-triazol-5-yl)phenylamino)- |
| | 1-oxopentan-2-aminium chloride |
| 194c | 4-(2-methoxy-5-(1-(3,4,5-trimethoxyphenyl)-1H-1,2,3-triazol-5-yl)phenylamino)- |
| | 4-oxobutanoic acid |
| 195c | 5-(2-methoxy-5-(1-(3,4,5-trimethoxyphenyl)-1H-1,2,3-triazol-5-yl)phenylamino)- |
| | 5-oxopentanoic acid |
| 196c | 3-(2-methoxy-5-(1-(3,4,5-trimethoxyphenyl)-1H-1,2,3-triazol-5-yl)phenylamino)- |
| | 3-oxopropan-1-aminium chloride |
| 197c | N-(2-methoxy-5-(1-(3,4,5-trimethoxyphenyl)-1H-1,2,3-triazol-5-yl)phenyl)-3-(2- |
| | methoxyethoxy)propanamide |
| 198c | 3-(2-PEG)-N-(2-methoxy-5-(1-(3,4,5-trimethoxyphenyl)-1H-1,2,3-triazol-5-yl)phenyl)butyramide |
| 199c | N-(2-methoxy-5-(1-(3,4,5-trimethoxyphenyl)-1H-1,2,3-triazol-5-yl)phenyl)-3-(2- |
| | (methylamino)ethylamino)propanamide |
| 200c | 3-PEG-N-(2-methoxy-5-(1-(3,4,5-trimethoxyphenyl)-1H-1,2,3-triazol-5-yl)phenylamino)- |
| | 2-oxoethyl)butyramide |
| 201c | 4-(2-(2-methoxy-5-(1-(3,4,5-trimethoxyphenyl)-1H-1,2,3-triazol-5-yl)phenylamino)- |
| | 2-oxoethylamino)-4-oxobutanoic acid |
| 202c | 2-methoxyethyl |
| | 2-methoxy-5-(1-(3,4,5-trimethoxyphenyl)-1H-1,2,3-triazol-5-yl)phenylcarbamate |
| 203c | PEG-2-methoxy-5-(1-(3,4,5-trimethoxyphenyl)-1H-1,2,3-triazol-5-yl)phenylcarbamate |
| 204c | 3-amino-4-(5-guanidino-1-(2-methoxy-5-(1-(3,4,5-trimethoxyphenyl)-1H-1,2,3-triazol- |
| | 5-yl)phenylamino)-1-oxopentan-2-ylamino)-2-oxoethylamino)-4-oxobutanoic |
| | acid |
| 205c | 2-amino-N-(2-methoxy-5-(1-(3,4,5-trimethoxyphenyl)-1H-1,2,3-triazol-5-yl)phenyl)propanamide |
| | hydrochloride |
| 1d | 4-(3,4,5-trimethoxy-phenyl)-5-(4-bromo-phenyl)-1H-[1,2,3]triazole |
| 2d | 4-ethyl-5-methoxy-2-(5-(naphthalen-2-yl)-1H-1,2,3-triazol-4-yl)phenol |
| 3d | 5-(4-methoxyphenyl)-4-(3,4,5-trimethoxyphenyl)-1H-1,2,3-triazole |
| 4d | 4-ethyl-2-(5-(4-iodophenyl)-1H-1,2,3-triazol-4-yl)-5-methoxyphenol |
| 5d | N,N-dimethyl-4-(4-(3,4,5-trimethoxyphenyl)-1H-1,2,3-triazol-5-yl)aniline |
| 6d | 2-(5-(4-bromophenyl)-1H-1,2,3-triazol-4-yl)-4-ethyl-5-methoxyphenol |
| 7d | 2-(5-(2,3-dihydrobenzo[b][1,4]dioxin-6-yl)-1H-1,2,3-triazol-4-yl)-5-methoxy-4- |
| | propylphenol |
| 8d | 5-(5-(4-hydroxyphenyl)-1H-1,2,3-triazol-4-yl)benzene-1,2,3-triol |
| 9d | 5-(4-iodophenyl)-4-(3,4,5-trimethoxyphenyl)-1H-1,2,3-triazole |
| 10d | 5-(3-fluoro-4-methoxyphenyl)-4-(3,4,5-trimethoxyphenyl)-1H-1,2,3-triazole |
| 11d | 5-(4-nitrophenyl)-4-(3,4,5-trimethoxyphenyl)-1H-1,2,3-triazole |
| 12d | 4-(4-(3,4,5-trimethoxyphenyl)-1H-1,2,3-triazol-5-yl)aniline |
| 13d | 5-(4′-methoxybiphenyl-4-yl)-4-(3,4,5-trimethoxyphenyl)-1H-1,2,3-triazole |
| 14d | 3-(4-(4-(3,4,5-trimethoxyphenyl)-1H-1,2,3-triazol-5-yl)phenyl)pyridine |
| 15d | 4-(4-(4-(3,4,5-trimethoxyphenyl)-1H-1,2,3-triazol-5-yl)phenyl)pyridine |
| 16d | 2-(4-(4-(3,4,5-trimethoxyphenyl)-1H-1,2,3-triazol-5-yl)phenyl)pyridine |
| 17d | 7-(4-(3,4,5-trimethoxyphenyl)-1H-1,2,3-triazol-5-yl)quinoline |
| 18d | 4-(4-(3,4,5-trimethoxyphenyl)-1H-1,2,3-triazol-5-yl)pyridine |
| 19d | 7-(4-(3,4,5-trimethoxyphenyl)-1H-1,2,3-triazol-5-yl)isoquinoline |
| 20d | 1-methyl-5-(4-(3,4,5-trimethoxyphenyl)-1H-1,2,3-triazol-5-yl)-1H-indole |
| 21d | 4-(benzo[d][1,3]dioxol-5-yl)-5-(4-methoxyphenyl)-1H-1,2,3-triazole |
| 22d | 1-ethyl-6-(5-(4-methoxyphenyl)-1H-1,2,3-triazol-4-yl)-1H-indole |
| 23d | 4-(4-(3,4,5-trimethoxyphenyl)-1H-1,2,3-triazol-5-yl)benzoic acid |
| 24d | methyl 4-(4-(3,4,5-trimethoxyphenyl)-1H-1,2,3-triazol-5-yl)benzoate |
| 25d | 2-(4-(4-(3,4,5-trimethoxyphenyl)-1H-1,2,3-triazol-5-yl)phenyl)oxazole |
| 26d | 5-(4-methoxyphenyl)-4-(3,4,5-triethylphenyl)-1H-1,2,3-triazole |
| 27d | 5-(4-iodophenyl)-4-(3,4,5-triethylphenyl)-1H-1,2,3-triazole |
| 28d | 5-(3-fluoro-4-methoxyphenyl)-4-(3,4,5-triethylphenyl)-1H-1,2,3-triazole |
| 29d | 5-(4-nitrophenyl)-4-(3,4,5-triethylphenyl)-1H-1,2,3-triazole |
| 30d | N,N-dimethyl-4-(4-(3,4,5-triethylphenyl)-1H-1,2,3-triazol-5-yl)aniline |
| 31d | 5-(4-methoxyphenyl)-4-(3,4,5-trimethylphenyl)-1H-1,2,3-triazole |
| 32d | 3-(4-(4-(3,4,5-triethylphenyl)-1H-1,2,3-triazol-5-yl)phenyl)pyridine |
| 33d | 4-(4-(4-(3,4,5-triethylphenyl)-1H-1,2,3-triazol-5-yl)phenyl)pyridine |
| 34d | 2-(4-(4-(3,4,5-triethylphenyl)-1H-1,2,3-triazol-5-yl)phenyl)pyridine |
| 35d | 7-(4-(3,4,5-triethylphenyl)-1H-1,2,3-triazol-5-yl)quinoline |
| 36d | 4-(4-(3,4,5-triethylphenyl)-1H-1,2,3-triazol-5-yl)pyridine |
| 37d | 7-(4-(3,4,5-triethylphenyl)-1H-1,2,3-triazol-5-yl)isoquinoline |
| 38d | 5-(4-(3,4,5-triethylphenyl)-1H-1,2,3-triazol-5-yl)-1H-mdole |
| 39d | 4-(benzo[d][1,3]dioxol-5-yl)-5-(4-methoxyphenyl)-1H-1,2,3-triazole |
| 40d | 1-isopropyl-6-(5-(4-methoxyphenyl)-1H-1,2,3-triazol-4-yl)-1H-indole |
| 41d | 5-(4-methoxyphenyl)-4-(2,3,4-trimethoxyphenyl)-1H-1,2,3-triazole |
| 42d | 2-methoxy-5-(4-(3,4,5-trimethoxyphenyl)-1H-1,2,3-triazol-5-yl)phenol |
| 43d | ethyl 2-methoxy-5-(4-(3,4,5-trimethoxyphenyl)-1H-1,2,3-triazol-5-yl)phenyl |
| | hydrogen phosphate |
| 44d | 4-ethyl-2-(5-(4-methoxyphenyl)-1H-1,2,3-triazol-4-yl)-5-methoxyphenol |
| 45d | 5-(4-isopropylphenyl)-4-(3,4,5-trimethoxyphenyl)-1H-1,2,3-triazole |
| 46d | 5-(2,3-dihydrobenzo[b][1,4]dioxin-6-yl)-4-(3,4,5-trimethoxyphenyl)-1H-1,2,3-triazole |
| 47d | 5-(4-ethylphenyl)-4-(3,4,5-trimethoxyphenyl)-1H-1,2,3-triazole |
| 48d | 5-methoxy-2-(4-(3,4,5-trimethoxyphenyl)-1H-1,2,3-triazol-5-yl)pyridine |
| 49d | 6-(5-(4-methoxyphenyl)-1H-1,2,3-triazol-4-yl)-2,3,4-trimethoxypyridine |
| 50d | methyl 2,6-dimethoxy-4-(5-(4-methoxyphenyl)-1H-1,2,3-triazol-4-yl)benzoate |
| 51d | 5-(5-(4-methoxyphenyl)-1H-1,2,3-triazol-4-yl)-1,3-phenylene diacetate |
| 52d | 2-methoxy-5-(4-(3,4,5-trimethoxyphenyl)-1H-1,2,3-triazol-5-yl)pyridine |
| 53d | 5-methoxy-7-(5-(4-methoxyphenyl)-1H-1,2,3-triazol-4-yl)-1-methyl-1H-indole |
| 54d | 1-ethyl-7-(5-(4-methoxyphenyl)-1H-1,2,3-triazol-4-yl)-1H-indole |
| 55d | 4-(benzo[d][1,3]dioxol-4-yl)-5-(4-methoxyphenyl)-1H-1,2,3-triazole |
| 56d | 5-methoxy-2-(4-(3,4,5-trimethoxyphenyl)-1H-1,2,3-triazol-5-yl)phenol |
| 57d | ethyl 5-methoxy-2-(4-(3,4,5-trimethoxyphenyl)-1H-1,2,3-triazol-5-yl)phenyl |
| | hydrogen phosphate |
| 58d | 4-(4-(3,4,5-trimethoxyphenyl)-1H-1,2,3-triazol-5-yl)pyridazine |
| 59d | 5-(4-(3,4,5-trimethoxyphenyl)-1H-1,2,3-triazol-5-yl)pyrimidine |
| 60d | 3-(4-(3,4,5-trimethoxyphenyl)-1H-1,2,3-triazol-5-yl)pyridine hydrochloride |
| 61d | 2-methoxy-5-(4-(3,4,5-trimethoxyphenyl)-1H-1,2,3-triazol-5-yl)benzenethiol |
| 62d | sodium S-2-methoxy-5-(4-(3,4,5-trimethoxyphenyl)-1H-1,2,3-triazol-5-yl)phenyl |
| | phosphorothioate |
| 63d | N-(2-methoxy-5-(4-(3,4,5-trimethoxyphenyl)-1H-1,2,3-triazol-5-yl)phenyl)acetamide |
| 64d | 2-methoxy-5-(4-(3,4,5-trimethoxyphenyl)-1H-1,2,3-triazol-5-yl)benzenaminium |
| | chloride |
| 65d | 5-methoxy-2-(4-(3,4,5-trimethoxyphenyl)-1H-1,2,3-triazol-5-yl)phenol |
| 66d | 2-methoxy-5-(4-(3,4,5-trimethoxyphenyl)-1H-1,2,3-triazol-5-yl)pyridine |
| 67d | 5-methoxy-2-(4-(3,4,5-trimethoxyphenyl)-1H-1,2,3-triazol-5-yl)pyridine |
| 68d | sodium 2-methoxy-5-(4-(3,4,5-trimethoxyphenyl)-1H-1,2,3-triazol-5-yl)benzoate |
| 69d | methyl 2-methoxy-5-(4-(3,4,5-trimethoxyphenyl)-1H-1,2,3-triazol-5-yl)benzoate |
| 70d | sodium 2-methoxy-5-(4-(3,4,5-trimethoxyphenyl)-1H-1,2,3-triazol-5-yl)phenyl |
| | sulfate |
| 71d | 5-methoxy-2-(4-(3,4,5-trimethoxyphenyl)-1H-1,2,3-triazol-5-yl)aniline |
| 72d | sodium |
| | 2,3-dimethoxy-5-(4-(3,4,5-trimethoxyphenyl)-1H-1,2,3-triazol-5-yl)phenyl |
| | phosphate |
| 73d | sodium 5-methoxy-2-(4-(3,4,5-trimethoxyphenyl)-1H-1,2,3-triazol-5-yl)phenyl |
| | phosphate |
| 74d | 5-(4-(methylthio)phenyl)-4-(3,4,5-trimethoxyphenyl)-1H-1,2,3-triazole |
| 75d | sodium |
| | 2-(methylthio)-5-(4-(3,4,5-trimethoxyphenyl)-1H-1,2,3-triazol-5-yl)phenyl |
| | phosphate |
| 76d | 2-(methylthio)-5-(4-(3,4,5-trimethoxyphenyl)-1H-1,2,3-triazol-5-yl)aniline |
| 77d | 5-(2,3-dihydrobenzofuran-6-yl)-4-(3,4,5-trimethoxyphenyl)-1H-1,2,3-triazole |
| 78d | sodium 4-(4-(3,4,5-trimethoxyphenyl)-1H-1,2,3-triazol-5-yl)phenolate |
| 79d | monosodium monosodium(II) |
| | mono(4-(4-(3,4,5-trimethoxyphenyl)-1H-1,2,3-triazol-5-yl)phenyl phosphate) |
| 80d | 5-(4-(4-(3,4,5-trimethoxyphenyl)-1H-1,2,3-triazol-5-yl)phenyl)-1H-tetrazole |
| 81d | 1-methyl-5-(4-(4-(3,4,5-trimethoxyphenyl)-1H-1,2,3-triazol-5-yl)phenyl)-1H-tetrazole |
| 82d | 1-methyl-5-(4-(3,4,5-trimethoxyphenyl)-1H-1,2,3-triazol-5-yl)-1H-indole |
| 83d | 4-(4-(7-methoxybenzo[d][1,3]dioxol-5-yl)-1H-1,2,3-triazol-5-yl)pyridazine |
| 84d | 5-(4-(7-methoxybenzo[d][1,3]dioxol-5-yl)-1H-1,2,3-triazol-5-yl)pyrimidine |
| 85d | 3-(4-(7-methoxybenzo[d][1,3]dioxol-5-yl)-1H-1,2,3-triazol-5-yl)pyridine |
| | hydrochloride |
| 86d | 2-methoxy-5-(4-(7-methoxybenzo[d][1,3]dioxol-5-yl)-1H-1,2,3-triazol-5-yl)benzenethiol |
| 87d | sodium |
| | S-2-methoxy-5-(4-(7-methoxybenzo[d][1,3]dioxol-5-yl)-1H-1,2,3-triazol-5-yl)phenyl |
| | phosphorothioate |
| 88d | N-(2-methoxy-5-(4-(7-methoxybenzo[d][1,3]dioxol-5-yl)-1H-1,2,3-triazol-5-yl)phenyl)acetamide |
| 89d | 2-methoxy-5-(4-(7-methoxybenzo[d][1,3]dioxol-5-yl)-1H-1,2,3-triazol-5-yl)benzenaminium |
| | chloride |
| 90d | 5-methoxy-2-(4-(7-methoxybenzo[d][1,3]dioxol-5-yl)-1H-1,2,3-triazol-5-yl)phenol |
| 91d | 2-methoxy-5-(4-(7-methoxybenzo[d][1,3]dioxol-5-yl)-1H-1,2,3-triazol-5-yl)pyridine |
| 92d | 5-methoxy-2-(4-(7-methoxybenzo[d][1,3]dioxol-5-yl)-1H-1,2,3-triazol-5-yl)pyridine |
| 93d | sodium |
| | 2-methoxy-5-(4-(7-methoxybenzo[d][1,3]dioxol-5-yl)-1H-1,2,3-triazol-5-yl)benzoate |
| 94d | methyl |
| | 2-methoxy-5-(4-(7-methoxybenzo[d][1,3]dioxol-5-yl)-1H-1,2,3-triazol-5-yl)benzoate |
| 95d | sodium |
| | 2-methoxy-5-(4-(7-methoxybenzo[d][1,3]dioxol-5-yl)-1H-1,2,3-triazol-5-yl)phenyl |
| | sulfate |
| 96d | 5-methoxy-2-(4-(7-methoxybenzo[d][1,3]dioxol-5-yl)-1H-1,2,3-triazol-5-yl)aniline |
| 97d | sodium |
| | 2,3-dimethoxy-5-(4-(7-methoxybenzo[d][1,3]dioxol-5-yl)-1H-1,2,3-triazol-5-yl)phenyl |
| | phosphate |
| 98d | sodium |
| | 5-methoxy-2-(4-(7-methoxybenzo[d][1,3]dioxol-5-yl)-1H-1,2,3-triazol-5-yl)phenyl |
| | phosphate |
| 99d | 4-(7-methoxybenzo[d][1,3]dioxol-5-yl)-5-(4-(methylthio)phenyl)-1H-1,2,3-triazole |
| 100d | sodium |
| | 5-(4-(7-methoxybenzo[d][1,3]dioxol-5-yl)-1H-1,2,3-triazol-5-yl)-2-(methylthio)phenyl |
| | phosphate |
| 101d | 5-(4-(7-methoxybenzo[d][1,3]dioxol-5-yl)-1H-1,2,3-triazol-5-yl)-2-(methylthio)aniline |
| 102d | 5-(2,3-dihydrobenzofuran-6-yl)-4-(7-methoxybenzo[d][1,3]dioxol-5-yl)-1H-1,2, |
| | 3-triazole |
| 103d | sodium |
| | 4-(4-(7-methoxybenzo[d][1,3]dioxol-5-yl)-1H-1,2,3-triazol-5-yl)phenolate |
| 104d | sodium 4-(4-(7-methoxybenzo[d][1,3]dioxol-5-yl)-1H-1,2,3-triazol-5-yl)phenyl |
| | phosphate |
| 105d | 5-(4-(4-(7-methoxybenzo[d][1,3]dioxol-5-yl)-1H-1,2,3-triazol-5-yl)phenyl)-1H-tetrazole |
| 106d | 5-(4-(4-(7-methoxybenzo[d][1,3]dioxol-5-yl)-1H-1,2,3-triazol-5-yl)phenyl)-1-methyl- |
| | 1H-tetrazole |
| 107d | 5-(4-(7-methoxybenzo[d][1,3]dioxol-5-yl)-1H-1,2,3-triazol-5-yl)-1-methyl-1H-indole |
| 108d | 1-methyl-5-(5-(3,4,5-trimethoxyphenyl)-1H-1,2,3-triazol-4-yl)-1H-indole |
| 109d | monosodium monosodium(II) |
| | mono(2-methoxy-5-(5-(3,4,5-trimethoxyphenyl)-1H-1,2,3-triazol-4-yl)phenyl |
| | phosphate) |
| 110d | N,N-dimethyl-4-(5-(3,4,5-trimethoxyphenyl)-1H-1,2,3-triazol-4-yl)aniline |
| 111d | 2-methoxy-5-(5-(3,4,5-trimethoxyphenyl)-1H-1,2,3-triazol-4-yl)benzenaminium |
| | chloride |
| 112d | 3-hydroxy-1-(2-methoxy-5-(5-(3,4,5-trimethoxyphenyl)-1H-1,2,3-triazol-4-yl)phenylamino)- |
| | 1-oxopropan-2-aminium chloride |
| 113d | 5-(4-methoxyphenyl)-4-(2,4,5-trimethoxyphenyl)-1H-1,2,3-triazole |
| 114d | 5-p-tolyl-4-(2,4,5-trimethoxyphenyl)-1H-1,2,3-triazole |
| 115d | 5-(4-ethoxyphenyl)-4-(2,4,5-trimethoxyphenyl)-1H-1,2,3-triazole |
| 116d | 5-(4-ethylphenyl)-4-(2,4,5-trimethoxyphenyl)-1H-1,2,3-triazole |
| 117d | 5-(4-propoxyphenyl)-4-(2,4,5-trimethoxyphenyl)-1H-1,2,3-triazole |
| 118d | 5-(4-propylphenyl)-4-(2,4,5-trimethoxyphenyl)-1H-1,2,3-triazole |
| 119d | 5-(4-butoxyphenyl)-4-(2,4,5-trimethoxyphenyl)-1H-1,2,3-triazole |
| 120d | 5-(4-butylphenyl)-4-(2,4,5-trimethoxyphenyl)-1H-1,2,3-triazole |
| 121d | 5-(4-bromophenyl)-4-(2,4,5-trimethoxyphenyl)-1H-1,2,3-triazole |
| 122d | 5-(4-chlorophenyl)-4-(2,4,5-trimethoxyphenyl)-1H-1,2,3-triazole |
| 123d | 5-(4-fluorophenyl)-4-(2,4,5-trimethoxyphenyl)-1H-1,2,3-triazole |
| 124d | 5-(4-nitrophenyl)-4-(2,4,5-trimethoxyphenyl)-1H-1,2,3-triazole |
| 125d | N,N-dimethyl-4-(4-(2,4,5-trimethoxyphenyl)-1H-1,2,3-triazol-5-yl)aniline |
| 126d | 5-(3,4-dimethoxyphenyl)-4-(2,4,5-trimethoxyphenyl)-1H-1,2,3-triazole |
| 127d | 2-methoxy-5-(4-(2,4,5-trimethoxyphenyl)-1H-1,2,3-triazol-5-yl)phenol |
| 128d | 4-(2,4,5-trimethoxyphenyl)-5-(3,4,5-trimethoxyphenyl)-1H-1,2,3-triazole |
| 129d | 5-(4-methoxyphenyl)-4-(2,3,5-trimethoxyphenyl)-1H-1,2,3-triazole |
| 130d | 5-p-tolyl-4-(2,3,5-trimethoxyphenyl)-1H-1,2,3-triazole |
| 131d | 5-(4-ethoxyphenyl)-4-(2,3,5-trimethoxyphenyl)-1H-1,2,3-triazole |
| 132d | 5-(4-ethylphenyl)-4-(2,3,5-trimethoxyphenyl)-1H-1,2,3-triazole |
| 133d | 5-(4-propoxyphenyl)-4-(2,3,5-trimethoxyphenyl)-1H-1,2,3-triazole |
| 134d | 5-(4-propylphenyl)-4-(2,3,5-trimethoxyphenyl)-1H-1,2,3-triazole |
| 135d | 5-(4-butoxyphenyl)-4-(2,3,5-trimethoxyphenyl)-1H-1,2,3-triazole |
| 136d | 5-(4-butylphenyl)-4-(2,3,5-trimethoxyphenyl)-1H-1,2,3-triazole |
| 137d | 5-(4-bromophenyl)-4-(2,3,5-trimethoxyphenyl)-1H-1,2,3-triazole |
| 138d | 5-(4-chlorophenyl)-4-(2,3,5-trimethoxyphenyl)-1H-1,2,3-triazole |
| 139d | 5-(4-fluorophenyl)-4-(2,3,5-trimethoxyphenyl)-1H-1,2,3-triazole |
| 140d | 5-(4-nitrophenyl)-4-(2,3,5-trimethoxyphenyl)-1H-1,2,3-triazole |
| 141d | N,N-dimethyl-4-(4-(2,3,5-trimethoxyphenyl)-1H-1,2,3-triazol-5-yl)aniline |
| 142d | 5-(3,4-dimethoxyphenyl)-4-(2,3,5-trimethoxyphenyl)-1H-1,2,3-triazole |
| 143d | 2-methoxy-5-(4-(2,3,5-trimethoxyphenyl)-1H-1,2,3-triazol-5-yl)phenol |
| 144d | 4-(2,3,5-trimethoxyphenyl)-5-(3,4,5-trimethoxyphenyl)-1H-1,2,3-triazole |
| 145d | 4-(4-methoxyphenyl)-5-(2,3,4,5-tetramethoxyphenyl)-1H-1,2,3-triazole |
| 146d | 5-(2,3,4,5-tetramethoxyphenyl)-4-p-tolyl-1H-1,2,3-triazole |
| 147d | 4-(4-ethoxyphenyl)-5-(2,3,4,5-tetramethoxyphenyl)-1H-1,2,3-triazole |
| 148d | 4-(4-ethylphenyl)-5-(2,3,4,5-tetramethoxyphenyl)-1H-1,2,3-triazole |
| 149d | 4-(4-propoxyphenyl)-5-(2,3,4,5-tetramethoxyphenyl)-1H-1,2,3-triazole |
| 150d | 4-(4-propylphenyl)-5-(2,3,4,5-tetramethoxyphenyl)-1H-1,2,3-triazole |
| 151d | 4-(4-butoxyphenyl)-5-(2,3,4,5-tetramethoxyphenyl)-1H-1,2,3-triazole |
| 152d | 4-(4-butylphenyl)-5-(2,3,4,5-tetramethoxyphenyl)-1H-1,2,3-triazole |
| 153d | 4-(4-bromophenyl)-5-(2,3,4,5-tetramethoxyphenyl)-1H-1,2,3-triazole |
| 154d | 4-(4-chlorophenyl)-5-(2,3,4,5-tetramethoxyphenyl)-1H-1,2,3-triazole |
| 155d | 4-(4-fluorophenyl)-5-(2,3,4,5-tetramethoxyphenyl)-1H-1,2,3-triazole |
| 156d | 4-(4-nitrophenyl)-5-(2,3,4,5-tetramethoxyphenyl)-1H-1,2,3-triazole |
| 157d | N,N-dimethyl-4-(5-(2,3,4,5-tetramethoxyphenyl)-1H-1,2,3-triazol-4-yl)aniline |
| 158d | 4-(3,4-dimethoxyphenyl)-5-(2,3,4,5-tetramethoxyphenyl)-1H-1,2,3-triazole |
| 159d | 2-methoxy-5-(5-(2,3,4,5-tetramethoxyphenyl)-1H-1,2,3-triazol-4-yl)phenol |
| 160d | 5-(2,3,4,5-tetramethoxyphenyl)-4-(3,4,5-trimethoxyphenyl)-1H-1,2,3-triazole |
| 161d | 5-(2,3-dihydrobenzo[b][1,4]dioxin-6-yl)-4-(3,4-dimethoxyphenyl)-1H-1,2,3-triazole |
| 162d | 2-(5-(3,4-dimethylphenyl)-1H-1,2,3-triazol-4-yl)-4-ethyl-5-methoxyphenol |
| 163d | 2-(5-(4-chlorophenyl)-1H-1,2,3-triazol-4-yl)-4-ethyl-5-methoxyphenol |
| 164d | 5-methoxy-2-(5-phenyl-1H-1,2,3-triazol-4-yl)-4-propylphenol |
| 165d | 4-ethyl-5-methoxy-2-(5-p-tolyl-1H-1,2,3-triazol-4-yl)phenol |
| 166d | 2-(5-(4-aminophenyl)-1H-1,2,3-triazol-4-yl)-4-ethyl-5-methoxyphenol |
| 167d | 4-ethyl-5-methoxy-2-(5-(4-(trifluoromethyl)phenyl)-1H-1,2,3-triazol-4-yl)phenol |
| 168d | 4-ethyl-5-methoxy-2-(5-(4-methoxyphenyl)-1H-1,2,3-triazol-4-yl)phenol |
| 169d | 4-(4-bromophenyl)-5-(3,4,5-trimethoxyphenyl)-1H-1,2,3-triazole |
| 170d | 2-amino-N-(2-methoxy-5-(4-(3,4,5-trimethoxyphenyl)-1H-1,2,3-triazol-5-yl)phenyl)acetamide |
| | hydrochloride |
| 171d | 2-amino-3-hydroxy-N-(2-methoxy-5-(4-(3,4,5-trimethoxyphenyl)-1H-1,2,3-triazol- |
| | 5-yl)phenyl)propanamide hydrochloride |
| 172d | 2-amino-N-(2-methoxy-5-(4-(3,4,5-trimethoxyphenyl)-1H-1,2,3-triazol-5-yl)phenyl)propanamide |
| 173d | 2-amino-N-(2-methoxy-5-(4-(3,4,5-trimethoxyphenyl)-1H-1,2,3-triazol-5-yl)phenyl)- |
| | 4-(methylthio)butanamide hydrochloride |
| 174d | 2-amino-N-(2-methoxy-5-(4-(3,4,5-trimethoxyphenyl)-1H-1,2,3-triazol-5-yl)phenyl)butanamide |
| | hydrochloride |
| 175d | 2-amino-N-(2-methoxy-5-(4-(3,4,5-trimethoxyphenyl)-1H-1,2,3-triazol-5-yl)phenyl)- |
| | 3-phenylpropanamide hydrochloride |
| 176d | 2-amino-N-(2-methoxy-5-(4-(3,4,5-trimethoxyphenyl)-1H-1,2,3-triazol-5-yl)phenyl)- |
| | 4-methylpentanamide hydrochloride |
| 177d | 2-amino-N-(2-methoxy-5-(4-(3,4,5-trimethoxyphenyl)-1H-1,2,3-triazol-5-yl)phenyl)- |
| | 4-methylpentanamide hydrochloride |
| 178d | 2-methoxy-5-(4-(3,4,5-trimethoxyphenyl)-1H-1,2,3-triazol-5-yl)phenyl |
| | dihydrogen phosphate |
| 179d | sodium 2-methoxy-5-(4-(3,4,5-trimethoxyphenyl)-1H-1,2,3-triazol-5-yl)phenyl |
| | phosphate |
| 180d | 1-(2-methoxy-5-(4-(3,4,5-trimethoxyphenyl)-1H-1,2,3-triazol-5-yl)phenylamino)- |
| | 3-methyl-1-oxobutan-2-aminium chloride |
| 181d | 1-(2-methoxy-5-(4-(3,4,5-trimethoxyphenyl)-1H-1,2,3-triazol-5-yl)phenylamino)- |
| | 3-methyl-1-oxopentan-2-aminium chloride |
| 182d | 3-hydroxy-1-(2-methoxy-5-(4-(3,4,5-trimethoxyphenyl)-1H-1,2,3-triazol-5-yl)phenylamino)- |
| | 1-oxobutan-2-aminium chloride |
| 183d | 3-(4-hydroxyphenyl)-1-(2-methoxy-5-(4-(3,4,5-trimethoxyphenyl)-1H-1,2,3-triazol- |
| | 5-yl)phenylamino)-1-oxopropan-2-aminium chloride |
| 184d | 2-(2-methoxy-5-(4-(3,4,5-trimethoxyphenyl)-1H-1,2,3-triazol-5-yl)phenylamino)- |
| | 2-oxo-1-phenylethanaminium chloride |
| 185d | 3-(1H-indol-2-yl)-1-(2-methoxy-5-(4-(3,4,5-trimethoxyphenyl)-1H-1,2,3-triazol- |
| | 5-yl)phenylamino)-1-oxopropan-2-aminium chloride |
| 186d | 3-(benzofuran-2-yl)-1-(2-methoxy-5-(4-(3,4,5-trimethoxyphenyl)-1H-1,2,3-triazol- |
| | 5-yl)phenylamino)-1-oxopropan-2-aminium chloride |
| 187d | 3-carboxy-1-(2-methoxy-5-(4-(3,4,5-trimethoxyphenyl)-1H-1,2,3-triazol-5-yl)phenylamino)- |
| | 1-oxopropan-2-aminium chloride |
| 188d | 4-carboxy-1-(2-methoxy-5-(4-(3,4,5-trimethoxyphenyl)-1H-1,2,3-triazol-5-yl)phenylamino)- |
| | 1-oxobutan-2-aminium chloride |
| 189d | 5-amino-1-(2-methoxy-5-(4-(3,4,5-trimethoxyphenyl)-1H-1,2,3-triazol-5-yl)phenylamino)- |
| | 1,5-dioxopentan-2-aminium chloride |
| 190d | 4-amino-1-(2-methoxy-5-(4-(3,4,5-trimethoxyphenyl)-1H-1,2,3-triazol-5-yl)phenylamino)- |
| | 1,4-dioxobutan-2-aminium chloride |
| 191d | 3-(1H-imidazol-5-yl)-1-(2-methoxy-5-(4-(3,4,5-trimethoxyphenyl)-1H-1,2,3-triazol- |
| | 5-yl)phenylamino)-1-oxopropan-2-aminium chloride |
| 192d | 6-amino-1-(2-methoxy-5-(4-(3,4,5-trimethoxyphenyl)-1H-1,2,3-triazol-5-yl)phenylamino)- |
| | 1-oxohexan-2-aminium chloride |
| 193d | 5-guanidino-1-(2-methoxy-5-(4-(3,4,5-trimethoxyphenyl)-1H-1,2,3-triazol-5-yl)phenylamino)- |
| | 1-oxopentan-2-aminium chloride |
| 194d | 4-(2-methoxy-5-(4-(3,4,5-trimethoxyphenyl)-1H-1,2,3-triazol-5-yl)phenylamino)- |
| | 4-oxobutanoic acid |
| 195d | 5-(2-methoxy-5-(4-(3,4,5-trimethoxyphenyl)-1H-1,2,3-triazol-5-yl)phenylamino)- |
| | 5-oxopentanoic acid |
| 196d | 3-(2-methoxy-5-(4-(3,4,5-trimethoxyphenyl)-1H-1,2,3-triazol-5-yl)phenylamino)- |
| | 3-oxopropan-1-aminium chloride |
| 197d | N-(2-methoxy-5-(4-(3,4,5-trimethoxyphenyl)-1H-1,2,3-triazol-5-yl)phenyl)-3-(2- |
| | methoxyethoxy)propanamide |
| 198d | 3-(2-PEG)-N-(2-methoxy-5-(4-(3,4,5-trimethoxyphenyl)-1H-1,2,3-triazol-5-yl)phenyl)butyramide |
| 199d | N-(2-methoxy-5-(4-(3,4,5-trimethoxyphenyl)-1H-1,2,3-triazol-5-yl)phenyl)-3-(2- |
| | (methylamino)ethylamino)propanamide |
| 200d | 3-PEG-N-(2-methoxy-5-(4-(3,4,5-trimethoxyphenyl)-1H-1,2,3-triazol-5-yl)phenylamino)- |
| | 2-oxoethyl)butyramide |
| 201d | 4-(2-(2-methoxy-5-(4-(3,4,5-trimethoxyphenyl)-1H-1,2,3-triazol-5-yl)phenylamino)- |
| | 2-oxoethylamino)-4-oxobutanoic acid |
| 202d | 2-methoxyethyl |
| | 2-methoxy-5-(4-(3,4,5-trimethoxyphenyl)-1H-1,2,3-triazol-5-yl)phenylcarbamate |
| 203d | PEG-2-methoxy-5-(4-(3,4,5-trimethoxyphenyl)-1H-1,2,3-triazol-5-yl)phenylcarbamate |
| 204d | 3-amino-4-(2-(5-guanidino-1-(2-methoxy-5-(4-(3,4,5-trimethoxyphenyl)-1H-1,2, |
| | 3-triazol-5-yl)phenylamino)-1-oxopentan-2-ylamino)-2-oxoethylamino)-4-oxobutanoic |
| | acid |
| 205d | 2-amino-N-(2-methoxy-5-(4-(3,4,5-trimethoxyphenyl)-1H-1,2,3-triazol-5-yl)phenyl)propanamide |
| | hydrochloride |
| 1e | 4-(4-Bromo-phenyl)-5-(3,4,5-trimethoxy-phenyl)-isothiazole |
| 2e | 4-(4-Bromo-phenyl)-5-(3,4,5-trimethoxy-phenyl)-isothiazole |
| 3e | 4-(4-methoxyphenyl)-5-(3,4,5-trimethoxyphenyl)isothiazole |
| 4e | 4-(4-Iodo-phenyl)-5-(2-hydroxy-4-methoxy-5-ethyl-phenyl)-isothiazole |
| 6e | 4-(4-Bromo-phenyl)-5-(2-hydroxy-4-methoxy-5-ethyl-phenyl)-isothiazole |
| 7e | 4-(2,3-Dihydro-benzo[1,4]di-oxin-6-yl)-5-(2-hydroxy-4- |
| | methoxy-5-propyl-phenyl)-isothiazole |
| 8e | 4-(4-hydroxy-phenyl)-5-(3,4,5-trihydroxy-phenyl)-isothiazole |
| 9e | 4-(4-Iodo-phenyl)-5-(3,4,5-trimethoxy-phenyl)-isothiazole |
| 10e | 4-(3-Fluoro-4-methoxy-phenyl)-5-(3,4,5-trimethoxy-phenyl)-isothiazole |
| 11e | 4-(4-Nitro-phenyl)-5-(3,4,5-trimethoxy-phenyl)-isothiazole |
| 12e | 4-(4-Amino-phenyl)-5-(3,4,5-trimethoxy-phenyl)-isothiazole |
| 13e | 4-(4′-Methoxy-biphenyl-4-yl)-5-(3,4,5-trimethoxy-phenyl)-isothiazole |
| 14e | 4-[4-(pyridine-3-yl)-phenyl]-5-(3,4,5-trimethoxy-phenyl)-isothiazole |
| 15e | 4-[4-(pyridine-4-yl)-phenyl]-5-(3,4,5-trimethoxy-phenyl)-isothiazole |
| 16e | 4-[4-(pyridine-2-yl)-phenyl]-5-(3,4,5-trimethoxy-phenyl)-isothiazole |
| 17e | 4-(Quinolin-7-yl)-5-(3,4,5-trimethoxy-phenyl)-isothiazole |
| 18e | 4-(Pyridin-4-yl)-5-(3,4,5-trimethoxy-phenyl)-isothiazole |
| 19e | 4-(Isoquinolin-7-yl)-5-(3,4,5-trimethoxy-phenyl)-isothiazole |
| 20e | 4-(1-Methyl-1H-indol-5-yl)-5-(3,4,5-trimethoxy-phenyl)-isothiazole |
| 21e | 4-(4-Methoxy-phenyl)-5-(benzo[1,3]dioxol-5-yl)-isothiazole |
| 22e | 4-(4-Methoxy-phenyl)-5-(1-ethyl-1H-indol-6-yl)-isothiazole |
| 23e | 4-(4-Carboxy-phenyl)-5-(3,4,5-trimethoxy-phenyl)-isothiazole |
| 24e | 4-(4-Methoxycarbonyl-phenyl)-5-(3,4,5-trimethoxy-phenyl)-isothiazole |
| 25e | 4-[4-(Oxazol-2-yl)-phenyl]-5-(3,4,5-trimethoxy-phenyl)-isothiazole |
| 26e | 4-(4-Methoxy-phenyl)-5-(3,4,5-triethyl-phenyl)-isothiazole |
| 27e | 4-(4-Iodo-phenyl)-5-(3,4,5-triethyl-phenyl)-isothiazole |
| 28e | 4-(3-Fluoro-4-methoxy-phenyl)-5-(3,4,5-triethyl-phenyl)-isothiazole |
| 29e | 4-(4-Nitro-phenyl)-5-(3,4,5-triethyl-phenyl)-isothiazole |
| 30e | 4-(4-N,N-dimethylamino-phenyl)-5-(3,4,5-triethyl-phenyl)-isothiazole |
| 31e | 4-(4-Methoxy-phenyl)-5-(3,4,5-trimethyl-phenyl)-isothiazole |
| 32e | 4-[4-(Pyridin-3-yl)-phenyl]-5-(3,4,5-triethyl-phenyl)-isothiazole |
| 33e | 4-[4-(Pyridin-4-yl)-phenyl]-5-(3,4,5-triethyl-phenyl)-isothiazole |
| 34e | 4-[4-(Pyridin-2-yl)-phenyl]-5-(3,4,5-triethyl-phenyl)-isothiazole |
| 35e | 4-(Quinolin-7-yl)-5-(3,4,5-triethyl-phenyl)-isothiazole |
| 36e | 4-(Pyridin-4-yl)-5-(3,4,5-triethyl-phenyl)-isothiazole |
| 37e | 4-(Isoquinolin-7-yl)-5-(3,4,5-triethyl-phenyl)-isothiazole |
| 38e | 4-(1H-Indol-5-yl)-5-(3,4,5-triethyl-phenyl)-isothiazole |
| 39e | 4-(4-Methoxy-phenyl)-5-(benzo[1,3]dioxol-5-yl)-isothiazole |
| 40e | 4-(4-Methoxy-phenyl)-5-[1-isopropyl-1H-indol-6-yl)-isothiazole |
| 41e | 4-(4-Methoxy-phenyl)-5-(2,3,4-trimethoxy-phenyl)-isothiazole |
| 42e | 4-(3-Hydroxy-4-methoxy-phenyl)-5-(3,4,5-trimethoxy-phenyl)-isothiazole |
| 43e | 4-[3-(Ethyl-hydroxy-phosphoryloxy)-4-methoxy-phenyl]-5-(3,4,5-trimethoxy- |
| | phenyl)-isothiazole |
| 44e | 4-(4-Methoxy-phenyl)-5-(2-hydroxy-4-methoxy-5-ethyl-phenyl)-isothiazole |
| 45e | 4-(4-Isopropyl-phenyl)-5-(3,4,5-trimethoxy-phenyl)-isothiazole |
| 46e | 4-(2,3-Dihydro-benzo[1,4]dioxin-6-yl)-5-(3,4,5-trimethoxy-phenyl)-isothiazole |
| | 4-(4-Ethyl-phenyl)-5-(3,4,5-trimethoxy-phenyl)-isothiazole |
| 48e | 4-(5-Methoxy-pyridin-2-yl)-5-(3,4,5-trimethoxy-phenyl)-isothiazole |
| 49e | 4-(4-Methoxy-phenyl)-5-(2,3,4-trimethoxy-pyridin-6-yl)-isothiazole |
| 50e | 4-(4-Methoxy-phenyl)-5-(3,5-dimethoxy-4-methoxycarbonyl-phenyl)- |
| | isothiazole |
| 51e | 4-(4-Methoxy-phenyl)-5-(3,5-diacetoxy-phenyl)-isothiazole |
| 52e | 4-(2-Methoxy-pyridin-5-yl)-5-(3,4,5-trimethoxy-phenyl)-isothiazole |
| 53e | 4-(4-Methoxy-phenyl)-5-(1-methyl-5-methoxy-1H-indol-7-yl)-isothiazole |
| 54e | 4-(4-Methoxy-phenyl)-5-(1-ethyl-1H-indol-7-yl)-isothiazole |
| 55e | 4-(4-Methoxy-phenyl)-5-(benzo[1,3]dioxol-4-yl)-isothiazole |
| 56e | 4-(2-Hydroxy-4-methoxy-phenyl)-5-(3,4,5-trimethoxy)-isothiazole |
| 57e | 4-[2-(Ethyl-hydroxy-phosphoryloxy)-4-methoxy-phenyl]-5-(3,4,5-trimethoxy- |
| | phenyl)-isothiazole |
| 58e | 4-(Pyridazin-4-yl)-5-(3,4,5-trimethoxy-phenyl)-isothiazole |
| 59e | 4-(Pyrimidin-5-yl)-5-(3,4,5-trimethoxy-phenyl)-isothiazole |
| 60e | 4-(Pyridin-3-yl)-5-(3,4,5-trimethoxy-phenyl)-isothiazole, hydrochloric acid salt |
| 61e | 4-(3-Mercapto-4-methoxy-phenyl)-5-(3,4,5-trimethoxy-phenyl)-isothiazole |
| 62e | 4-(3-Phosphonosulfanyl-4-methoxy-phenyl)-5-(3,4,5-trimethoxy-phenyl)- |
| | isothiazole, disodium salt |
| 63e | 4-(3-Acetylamino-4-methoxy-phenyl)-5-(3,4,5-trimethoxy-phenyl)-isothiazole |
| 64e | 2-methoxy-5-[5-(3,4,5-trimethoxy-phenyl)- |
| | isothiozol-4-yl)-phenylamine |
| 65e | 4-(2-Hydroxy-4-methoxy-phenyl)-5-(3,4,5-trimethoxy-phenyl)-isothiazole |
| 66e | 4-(2-Methoxy-pyridine-5-yl)-5-(3,4,5-trimethoxy-phenyl)-isothiazole |
| 67e | 4-(5-Methoxy-pyridine-2-yl)-5-(3,4,5-trimethoxy-phenyl)-isothiazole |
| 68e | 4-(3-Carboxy-4-methoxy-phenyl)-5-(3,4,5-trimethoxy-phenyl)-isothiazole, |
| | sodium salt |
| 69e | 4-(3-Methoxycarbonyl-4-methoxy-phenyl)-5-(3,4,5-trimethoxy-phenyl)- |
| | isothiazole |
| 70e | 4-(3-Sulfooxy-4-methoxy-phenyl)-5-(3,4,5-trimethoxy-phenyl)-isothiazole, |
| | sodium salt |
| 71e | 4-(2-Amino-4-methoxy-phenyl)-5-(3,4,5-trimethoxy-phenyl)-isothiazole |
| 72e | 4-(3,4-Dimethoxy-5-phosphonooxy-phenyl)-5-(3,4,5-trimethoxy-phenyl)- |
| | isothiazole, disodium salt |
| 73e | 4-(2-Phosphonooxy-4-methoxy-phenyl)-5-(3,4,5-trimethoxy-phenyl)- |
| | isothiazole, disodium salt |
| 74e | 4-(4-Methylsulfanyl-phenyl)-5-(3,4,5-trimethoxy-phenyl)-isothiazole |
| 75e | 4-(3-Phosphonooxy-4-methylsulfanyl-phenyl)-5-(3,4,5-trimethoxy-phenyl)- |
| | isothiazole, disodium salt |
| 76e | 4-(3-Amino-4-methylsulfanyl-phenyl)-5-(3,4,5-trimethoxy-phenyl)-isothiazole |
| 77e | 4-(2,3-Dihydro-benzofuran-6-yl)-5-(3,4,5-trimethoxy-phenyl)-isothiazole |
| 78e | 4-(4-Hydroxy-phenyl)-5-(3,4,5-trimethoxy-phenyl)-isothiazole, sodium salt |
| 79e | 4-(4-Phosphonooxy-phenyl)-5-(3,4,5-trimethoxy-phenyl)-isothiazole, disodium |
| | salt |
| 80e | 4-(4-1H-Tetrazol-5-yl-phenyl)-5-(3,4,5-trimethoxy-phenyl)-isothiazole |
| 81e | 4-[4-(1-Methyl-1H-tetrazol-5-yl)-phenyl]-5-(3,4,5-trimethoxy-phenyl)- |
| | isothiazole |
| 82e | 4-(1-Methyl-1H-indol-5-yl)-5-(3,4,5-trimethoxy-phenyl)-isothiazole |
| 83e | 4-(Pyridazin-4-yl)-5-(4-methoxy-benzo[1,3]dioxol-6-yl)-isothiazole |
| 84e | 4-(Pyrimidin-5-yl)-5-(4-methoxy-benzo[1,3]dioxol-6-yl)-isothiazole |
| 85e | 4-(Pyridin-3-yl)-5-(4-methoxy-benzo[1,3]dioxol-6-yl)-isothiazole hydrochloric |
| | acid salt |
| 86e | 4-(3-Mercapto-4-methoxy-phenyl)-5-(4-methoxy-benzo[1,3]dioxol-6-yl)- |
| | isothiazole |
| 87e | 4-(3-Phosphonosulfanyl-4-methoxy-phenyl)-5-(4- |
| | methoxy-benzo[1,3]dioxol-6-yl)-isothiazole |
| 88e | 4-(3-Acetylamino-4-methoxy-phenyl)-5-(4-methoxy-benzo[1,3]dioxol-6-yl)- |
| | isothiazole |
| 89e | 4-(3-Amino-4-methoxy-phenyl)-5-(4-methoxy-benzo[1,3]dioxol-6-yl)- |
| | isothiazole, hydrochloric acid salt |
| 90e | 4-(2-Hydroxy-4-methoxy-phenyl)-5-(4-methoxy-benzo[1,3]dioxol-6-yl)- |
| | isothiazole |
| 91e | 4-(2-Methoxy-pyridin-5-yl)-5-(4-methoxy-benzo[1,3]dioxol-6-yl)-isothiazole |
| 92e | 4-(5-Methoxy-pyridin-2-yl)-5-(4-methoxy-benzo[1,3]dioxol-6-yl)-isothiazole |
| 93e | 4-(3-Carboxy-4-methoxy-phenyl)-5-(4-methoxy-benzo[1,3]dioxol-6-yl)- |
| | isothiazole, sodium salt |
| 94e | 4-(3-Methoxycarbonyl-4-methoxy-phenyl)-5-(4- |
| | methoxy-benzo[1,3]dioxol-6-yl)-isothiazole |
| 95e | 4-(3-Sulfooxy-4-methoxy-phenyl)-5-(4-methoxy-benzo[1,3]dioxol-6-yl)- |
| | isothiazole, sodium salt |
| 96e | 4-(3-Amino-4-methoxy-phenyl)-5-(4-methoxy-benzo[1,3]dioxol-6-yl)- |
| | isothiazole |
| 97e | 4-(3,4-Dimethoxy-5 phosphonooxy-phenyl)-5-(4- |
| | methoxy-benzo[1,3]dioxol-6-yl)-isothiazole, disodium salt |
| 98e | 4-(2-Phosphonooxy-4-methoxy-phenyl)-5-(4-methoxy-benzo[1,3]dioxol-6-yl)- |
| | isothiazole, disodium salt |
| 99e | 4-(4-Methylsulfanyl-phenyl)-5-(4-methoxy-benzo[1,3]dioxol-6-yl)-isothiazole |
| 100e | 4-(3-Phosphonooxy-4-methylsulfanyl-phenyl)-5-(4- |
| | methoxy-benzo[1,3]dioxol-6-yl)-isothiazole, disodium salt |
| 101e | 4-(3-Amino-4-methylsulfanyl-phenyl)-5-(4-methoxy-benzo[1,3]dioxol-6-yl)- |
| | isothiazole |
| 102e | 4-(2,3-Dihydro-benzofuran-6-yl)-5-(4-methoxy-benzo[1,3]dioxol-6-yl)- |
| | isothiazole |
| 103e | 4-(4-Hydroxy-phenyl)-5-(4-methoxy-benzo[1,3]dioxol-6-yl)-isothiazole, sodium |
| | salt |
| 104e | 4-(4-Phosphonooxy-phenyl)-5-(4-methoxy-benzo[1,3]dioxol-6-yl)-isothiazole |
| 105e | 4-(4-1H-Tetrazol-5-yl-phenyl)-5-(4-methoxy-benzo[1,3]dioxol-6-yl)-isothiazole |
| 106e | 4-[4-(1-Methyl-1H-tetrazol-5-yl)-phenyl]-5-(4-methoxy-benzo[1,3]dioxol-6-yl)- |
| | isothiazole |
| 107e | 4-(1-Methyl-1H-indol-5-yl)-5-(4-methoxy-benzo[1,3]dioxol-6-yl)-isothiazole |
| 108e | 4-(3,4,5-Trimethoxy-phenyl)-5-(1-methyl-1H-indol-5-yl)-isothiazole |
| 109e | 4-(3,4,5-Trimethoxy-phenyl)-5-(3-phosphonooxy-4-methoxy-phenyl)isothiazole, |
| | disodium salt |
| 110e | 4-(3,4,5-Trimethoxy-phenyl)-5-(N,N-dimethylamino-phenyl)-isothiazole |
| 111e | 4-(3,4,5-Trimethoxy-phenyl)-5-(3-amino-4-methoxy-phenyl)-isothiazole, |
| | hydrochloric acid salt |
| 112e | 4-(3,4,5-Trimethoxy-phenyl)-5-[3-(3-hydroxy-2S-amino- |
| | propionamido)-4-methoxy-phenyl]-isothiazole, hydrochloric acid salt |
| 113e | 4-(4-Methoxy-phenyl)-5-(2,4,5-trimethoxy-phenyl)-isothiazole |
| 114e | 4-(4-Methyl-phenyl)-5-(2,4,5-trimethoxy-phenyl)-isothiazole |
| 115e | 4-(4-Ethoxy-phenyl)-5-(2,4,5-trimethoxy-phenyl)-isothiazole |
| 116e | 4-(4-Ethyl-phenyl)-5-(2,4,5-trimethoxy-phenyl)-isothiazole |
| 117e | 4-(4-Propoxy-phenyl)-5-(2,4,5-trimethoxy-phenyl)-isothiazole |
| 118e | 4-(4-Propyl-phenyl)-5-(2,4,5-trimethoxy-phenyl)-isothiazole |
| 119e | 4-(4-Butoxy-phenyl)-5-(2,4,5-trimethoxy-phenyl)-isothiazole |
| 120e | 4-(4-Butyl-phenyl)-5-(2,4,5-trimethoxy-phenyl)-isothiazole |
| 121e | 4-(4-Bromo-phenyl)-5-(2,4,5-trimethoxy-phenyl)-isothiazole |
| 122e | 4-(4-Chloro-phenyl)-5-(2,4,5-trimethoxy-phenyl)-isothiazole |
| 123e | 4-(4-Fluoro-phenyl)-5-(2,4,5-trimethoxy-phenyl)-isothiazole |
| 124e | 4-(4-Nitro-phenyl)-5-(2,4,5-trimethoxy-phenyl)-isothiazole |
| 125e | 4-[4-(N,N-Dimethylamino)-phenyl]-5-(2,4,5-trimethoxy-phenyl)-isothiazole |
| 126e | 4-(3,4-Dimethoxy-phenyl)-5-(2,4,5-trimethoxy-phenyl)-isothiazole |
| 127e | 4-(3-Hydroxy-4-methoxy-phenyl)-5-(2,4,5-trimethoxy-phenyl)-isothiazole |
| 128e | 4-(3,4,5-Trimethoxy-phenyl)-5-(2,4,5-trimethoxy-phenyl)-isothiazole |
| 129e | 4-(4-Methoxy-phenyl)-5-(2,3,5-trimethoxy-phenyl)-isothiazole |
| 130e | 4-(4-Methyl-phenyl)-5-(2,3,5-trimethoxy-phenyl)-isothiazole |
| 131e | 4-(4-Ethoxy-phenyl)-5-(2,3,5-trimethoxy-phenyl)-isothiazole |
| 132e | 4-(4-Ethyl-phenyl)-5-(2,3,5-trimethoxy-phenyl)-isothiazole |
| 133e | 4-(4-Propoxy-phenyl)-5-(2,3,5-trimethoxy-phenyl)-isothiazole |
| 134e | 4-(4-Propyl-phenyl)-5-(2,3,5-trimethoxy-phenyl)-isothiazole |
| 135e | 4-(4-Butoxy-phenyl)-5-(2,3,5-trimethoxy-phenyl)-isothiazole |
| 136e | 4-(4-Butyl-phenyl)-5-(2,3,5-trimethoxy-phenyl)-isothiazole |
| 137e | 4-(4-Bromo-phenyl)-5-(2,3,5-trimethoxy-phenyl)-isothiazole |
| 138e | 4-(4-Chloro-phenyl)-5-(2,3,5-trimethoxy-phenyl)-isothiazole |
| 139e | 4-(4-Fluoro-phenyl)-5-(2,3,5-trimethoxy-phenyl)-isothiazole |
| 140e | 4-(4-Nitro-phenyl)-5-(2,3,5-trimethoxy-phenyl)-isothiazole |
| 141e | 4-[4-(N,N-Dimethylamino)-phenyl]-5-(2,3,5-trimethoxy-phenyl)-isothiazole |
| 142e | 4-(3,4-Dimethoxy-phenyl)-5-(2,3,5-trimethoxy-phenyl)-isothiazole |
| 143e | 4-(3-Hydroxy-4-methoxy-phenyl)-5-(2,3,5-trimethoxy-phenyl)-isothiazole |
| 144e | 4-(3,4,5-Trimethoxy-phenyl)-5-(2,3,5-trimethoxy-phenyl)-isothiazole |
| 145e | 4-(2,3,4,5-Tetramethoxy-phenyl)-5-(4-methoxy-phenyl)-isothiazole |
| 146e | 4-(2,3,4,5-Tetramethoxy-phenyl)-5-(4-methyl-phenyl)-isothiazole |
| 147e | 4-(2,3,4,5-Tetramethoxy-phenyl)-5-(4-ethoxy-phenyl)-isothiazole |
| 148e | 4-(2,3,4,5-Tetramethoxy-phenyl)-5-(4-ethyl-phenyl)-isothiazole |
| 149e | 4-(2,3,4,5-Tetramethoxy-phenyl)-5-(4-propoxy-phenyl)-isothiazole |
| 150e | 4-(2,3,4,5-Tetramethoxy-phenyl)-5-(4-propyl-phenyl)-isothiazole |
| 151e | 4-(2,3,4,5-Tetramethoxy-phenyl)-5-(4-butoxy-phenyl)-isothiazole |
| 152e | 4-(2,3,4,5-Tetramethoxy-phenyl)-5-(4-butyl-phenyl)-isothiazole |
| 153e | 4-(2,3,4,5-Tetramethoxy-phenyl)-5-(4-bromo-phenyl)-isothiazole |
| 154e | 4-(2,3,4,5-Tetramethoxy-phenyl)-5-(4-chloro-phenyl)-isothiazole |
| 155e | 4-(2,3,4,5-Tetramethoxy-phenyl)-5-(4-fluoro-phenyl)-isothiazole |
| 156e | 4-(2,3,4,5-Tetramethoxy-phenyl)-5-(4-nitro-phenyl)-isothiazole |
| 157e | 4-(2,3,4,5-Tetramethoxy-phenyl)-5-[4-(N,N,-dimethylamino)-phenyl]- |
| | isothiazole |
| 158e | 4-(2,3,4,5-Tetramethoxy-phenyl)-5-(3,4-dimethoxy-phenyl)-isothiazole |
| 159e | 4-(2,3,4,5-Tetramethoxy-phenyl)-5-(3-hydroxy-4-methoxy-phenyl)-isothiazole |
| 160e | 4-(2,3,4,5-Tetramethoxy-phenyl)-5-(3,4,5-trimethoxy-phenyl)-isothiazole |
| 161e | 4-(2,3-Dihydro-benzo[1,4]dioxin-6-yl)-5-(3,4-dimethoxy-phenyl)-isothiazole |
| 162e | 4-(3,4-Dimethy-phenyl)-5-(2-hydroxy-4-methoxy-5-ethyl-phenyl)-isothiazole |
| 163e | 4-(4-Chloro-phenyl)-5-(2-hydroxy-4-methoxy-5-ethyl-phenyl)-isothiazole |
| 164e | 4-(4-Methyl-phenyl)-5-(2-hydroxy-4-methoxy-5-ethyl-phenyl)-isothiazole |
| 165e | 4-(4-Amino-phenyl)-5-(2-hydroxy-4-methoxy-5-ethyl-phenyl)-isothiazole |
| 166e | 4-(4-Trifluoromethyl-phenyl)-5-(2-hydroxy-4-methoxy-5-ethyl-phenyl)- |
| | isothiazole |
| 167e | 4-(4-Methoxy-phenyl)-5-(2-hydroxy-4-methoxy-5-ethyl-phenyl)-isothiazole |
| 168e | 4-(3,4,5-Trimethoxy-phenyl)-5-(4-bromo-phenyl)-isothiazole |
| 169e | 4-(4-Bromo-phenyl)-3-(3,4,5-trimethoxy-phenyl)-isothiazole |
| 170e | 4-(Naphthalen-2-yl)-3-(2-hydroxy-4-methoxy-5-ethyl-phenyl)-isothiazole |
| 171e | 4-(4-methoxyphenyl)-3-(3,4,5-trimethoxyphenyl)isothiazole |
| 172e | 4-(4-Iodo-phenyl)-3-(2-hydroxy-4-methoxy-5-ethyl-phenyl)-isothiazole |
| 174e | 4-(4-Bromo-phenyl)-3-(2-hydroxy-4-methoxy-5-ethyl-phenyl)-isothiazole |
| 175e | 4-(2,3-Dihydro-benzo[1,4]di-oxin-6-yl)-3-(2-hydroxy-4- |
| | methoxy-5-propyl-phenyl)-isothiazole |
| 176e | 4-(4-hydroxy-phenyl)-3-(3,4,5-trihydroxy-phenyl)-isothiazole |
| 177e | 4-(4-Iodo-phenyl)-3-(3,4,5-trimethoxy-phenyl)-isothiazole |
| 178e | 4-(3-Fluoro-4-methoxy-phenyl)-3-(3,4,5-trimethoxy-phenyl)-isothiazole |
| 179e | 4-(4-Nitro-phenyl)-3-(3,4,5-trimethoxy-phenyl)-isothiazole |
| 180e | 4-(4-Amino-phenyl)-3-(3,4,5-trimethoxy-phenyl)-isothiazole |
| 181e | 4-(4′-Methoxy-biphenyl-4-yl)-3-(3,4,5-trimethoxy-phenyl)-isothiazole |
| 182e | 4-[4-(pyridine-3-yl)-phenyl]-3-(3,4,5-trimethoxy-phenyl)-isothiazole |
| 183e | 4-[4-(pyridine-4-yl)-phenyl]-3-(3,4,5-trimethoxy-phenyl)-isothiazole |
| 184e | 4-[4-(pyridine-2-yl)-phenyl]-3-(3,4,5-trimethoxy-phenyl)-isothiazole |
| 185e | 4-(Quinolin-7-yl)-3-(3,4,5-trimethoxy-phenyl)-isothiazole |
| 186e | 4-(Pyridin-4-yl)-3-(3,4,5-trimethoxy-phenyl)-isothiazole |
| 187e | 4-(Isoquinolin-7-yl)-3-(3,4,5-trimethoxy-phenyl)-isothiazole |
| 188e | 4-(1-Methyl-1H-indol-5-yl)-3-(3,4,5-trimethoxy-phenyl)-isothiazole |
| 189e | 4-(4-Methoxy-phenyl)-3-(benzo[1,3]dioxol-5-yl)-isothiazole |
| 190e | 4-(4-Methoxy-phenyl)-3-(1-ethyl-1H-indol-6-yl)-isothiazole |
| 191e | 4-(4-Carboxy-phenyl)-3-(3,4,5-trimethoxy-phenyl)-isothiazole |
| 192e | 4-(4-Methoxycarbonyl-phenyl)-3-(3,4,5-trimethoxy-phenyl)-isothiazole |
| 193e | 4-[4-(Oxazol-2-yl)-phenyl]-3-(3,4,5-trimethoxy-phenyl)-isothiazole |
| 194e | 4-(4-Methoxy-phenyl)-3-(3,4,5-triethyl-phenyl)-isothiazole |
| 195e | 4-(4-Iodo-phenyl)-3-(3,4,5-triethyl-phenyl)-isothiazole |
| 196e | 4-(3-Fluoro-4-methoxy-phenyl)-3-(3,4,5-triethyl-phenyl)-isothiazole |
| 197e | 4-(4-Nitro-phenyl)-3-(3,4,5-triethyl-phenyl)-isothiazole |
| 198e | 4-(4-N,N-dimethylamino-phenyl)-3-(3,4,5-triethyl-phenyl)-isothiazole |
| 199e | 4-(4-Methoxy-phenyl)-3-(3,4,5-trimethyl-phenyl)-isothiazole |
| 200e | 4-[4-(Pyridin-3-yl)-phenyl]-3-(3,4,5-triethyl-phenyl)-isothiazole |
| 201e | 4-[4-(Pyridin-4-yl)-phenyl]-3-(3,4,5-triethyl-phenyl)-isothiazole |
| 202e | 4-[4-(Pyridin-2-yl)-phenyl]-3-(3,4,5-triethyl-phenyl)-isothiazole |
| 203e | 4-(Quinolin-7-yl)-3-(3,4,5-triethyl-phenyl)-isothiazole |
| 204e | 4-(Pyridin-4-yl)-3-(3,4,5-triethyl-phenyl)-isothiazole |
| 205e | 4-(Isoquinolin-7-yl)-3-(3,4,5-triethyl-phenyl)-isothiazole |
| 206e | 4-(1H-Indol-5-yl)-3-(3,4,5-triethyl-phenyl)-isothiazole |
| 207e | 4-(4-Methoxy-phenyl)-3-(benzo[1,3]dioxol-5-yl)-isothiazole |
| 208e | 4-(4-Methoxy-phenyl)-3-[1-isopropyl-1H-indol-6-yl)-isothiazole |
| 209e | 4-(4-Methoxy-phenyl)-3-(2,3,4-trimethoxy-phenyl)-isothiazole |
| 210e | 4-(3-Hydroxy-4-methoxy-phenyl)-3-(3,4,5-trimethoxy-phenyl)-isothiazole |
| 211e | 4-[3-(Ethyl-hydroxy-phosphoryloxy)-4-methoxy-phenyl]-3-(3,4,5-trimethoxy- |
| | phenyl)-isothiazole |
| 212e | 4-(4-Methoxy-phenyl)-3-(2-hydroxy-4-methoxy-5-ethyl-phenyl)-isothiazole |
| 213e | 4-(4-Isopropyl-phenyl)-3-(3,4,5-trimethoxy-phenyl)-isothiazole |
| 214e | 4-(2,3-Dihydro-benzo[1,4]dioxin-6-yl)-3-(3,4,5-trimethoxy-phenyl)-isothiazole |
| 215e | 4-(4-Ethyl-phenyl)-3-(3,4,5-trimethoxy-phenyl)-isothiazole |
| 216e | 4-(5-Methoxy-pyridin-2-yl)-3-(3,4,5-trimethoxy-phenyl)-isothiazole |
| 217e | 4-(4-Methoxy-phenyl)-3-(2,3,4-trimethoxy-pyridin-6-yl)-isothiazole |
| 218e | 4-(4-Methoxy-phenyl)-3-(3,5-dimethoxy-4-methoxycarbonyl-phenyl)- |
| | isothiazole |
| 219e | 4-(4-Methoxy-phenyl)-3-(3,5-diacetoxy-phenyl)-isothiazole |
| 220e | 4-(2-Methoxy-pyridin-5-yl)-3-(3,4,5-trimethoxy-phenyl)-isothiazole |
| 221e | 4-(4-Methoxy-phenyl)-3-(1-methyl-5-methoxy-1H-indol-7-yl)-isothiazole |
| 222e | 4-(4-Methoxy-phenyl)-3-(1-ethyl-1H-indol-7-yl)-isothiazole |
| 223e | 4-(4-Methoxy-phenyl)-3-(benzo[1,3]dioxol-4-yl)-isothiazole |
| 224e | 4-(2-Hydroxy-4-methoxy-phenyl)-3-(3,4,5-trimethoxy)-isothiazole |
| 225e | 4-[2-(Ethyl-hydroxy-phosphoryloxy)-4-methoxy-phenyl]-3-(3,4,5-trimethoxy- |
| | phenyl)-isothiazole |
| 226e | 4-(Pyridazin-4-yl)-3-(3,4,5-trimethoxy-phenyl)-isothiazole |
| 227e | 4-(Pyrimidin-5-yl)-3-(3,4,5-trimethoxy-phenyl)-isothiazole |
| 228e | 4-(Pyridin-3-yl)-3-(3,4,5-trimethoxy-phenyl)-isothiazole, hydrochloric acid salt |
| 229e | 4-(3-Mercapto-4-methoxy-phenyl)-3-(3,4,5-trimethoxy-phenyl)-isothiazole |
| 230e | 4-(3-Phosphonosulfanyl-4-methoxy-phenyl)-3-(3,4,5-trimethoxy-phenyl)- |
| | isothiazole, disodium salt |
| 231e | 4-(3-Acetylamino-4-methoxy-phenyl)-3-(3,4,5-trimethoxy-phenyl)-isothiazole |
| 232e | 4-(3-Amino-4-methoxy-phenyl)-3-(3,4,5-trimethoxy-phenyl)-isothiazole |
| 233e | 4-(2-Hydroxy-4-methoxy-phenyl)-3-(3,4,5-trimethoxy-phenyl)-isothiazole |
| 234e | 4-(2-Methoxy-pyridine-5-yl)-3-(3,4,5-trimethoxy-phenyl)-isothiazole |
| 235e | 4-(5-Methoxy-pyridine-2-yl)-3-(3,4,5-trimethoxy-phenyl)-isothiazole |
| 236e | 4-(3-Carboxy-4-methoxy-phenyl)-3-(3,4,5-trimethoxy-phenyl)-isothiazole, |
| | sodium salt |
| 237e | 4-(3-Methoxycarbonyl-4-methoxy-phenyl)-3-(3,4,5-trimethoxy-phenyl)- |
| | isothiazole |
| 238e | 4-(3-Sulfooxy-4-methoxy-phenyl)-3-(3,4,5-trimethoxy-phenyl)-isothiazole, |
| | sodium salt |
| 239e | 4-(2-Amino-4-methoxy-phenyl)-3-(3,4,5-trimethoxy-phenyl)-isothiazole |
| 240e | 4-(3,4-Dimethoxy-5-phosphonooxy-phenyl)-3-(3,4,5-trimethoxy-phenyl)- |
| | isothiazole, disodium salt |
| 241e | 4-(2-Phosphonooxy-4-methoxy-phenyl)-3-(3,4,5-trimethoxy-phenyl)- |
| | isothiazole, disodium salt |
| 242e | 4-(4-Methylsulfanyl-phenyl)-3-(3,4,5-trimethoxy-phenyl)-isothiazole |
| 243e | 4-(3-Phosphonooxy-4-methylsulfanyl-phenyl)-3-(3,4,5-trimethoxy-phenyl)- |
| | isothiazole, disodium salt |
| 244e | 4-(3-Amino-4-methylsulfanyl-phenyl)-3-(3,4,5-trimethoxy-phenyl)-isothiazole |
| 245e | 4-(2,3-Dihydro-benzofuran-6-yl)-3-(3,4,5-trimethoxy-phenyl)-isothiazole |
| 246e | 4-(4-Hydroxy-phenyl)-3-(3,4,5-trimethoxy-phenyl)-isothiazole, sodium salt |
| 247e | 4-(4-Phosphonooxy-phenyl)-3-(3,4,5-trimethoxy-phenyl)-isothiazole, disodium |
| | salt |
| 248e | 4-(4-1H-Tetrazol-5-yl-phenyl)-3-(3,4,5-trimethoxy-phenyl)-isothiazole |
| 249e | 4-[4-(1-Methyl-1H-tetrazol-5-yl)-phenyl]-3-(3,4,5-trimethoxy-phenyl)- |
| | isothiazole |
| 250e | 4-(1-Methyl-1H-indol-5-yl)-3-(3,4,5-trimethoxy-phenyl)-isothiazole |
| 251e | 4-(Pyridazin-4-yl)-3-(4-methoxy-benzo[1,3]dioxol-6-yl)-isothiazole |
| 252e | 4-(Pyrimidin-5-yl)-3-(4-methoxy-benzo[1,3]dioxol-6-yl)-isothiazole |
| 253e | 4-(Pyridin-3-yl)-3-(4-methoxy-benzo[1,3]dioxol-6-yl)-isothiazole, hydrochloric |
| | acid salt |
| 254e | 4-(3-Mercapto-4-methoxy-phenyl)-3-(4-methoxy-benzo[1,3]dioxol-6-yl)- |
| | isothiazole |
| 255e | 4-(3-Phosphonosulfanyl-4-methoxy-phenyl)-3-(4- |
| | methoxy-benzo[1,3]dioxol-6-yl)-isothiazole |
| 256e | 4-(3-Acetylamino-4-methoxy-phenyl)-3-(4-methoxy-benzo[1,3]dioxol-6-yl)- |
| | isothiazole |
| 257e | 4-(3-Amino-4-methoxy-phenyl)-3-(4-methoxy-benzo[1,3]dioxol-6-yl)- |
| | isothiazole, hydrochloric acid salt |
| 258e | 4-(2-Hydroxy-4-methoxy-phenyl)-3-(4-methoxy-benzo[1,3]dioxol-6-yl)- |
| | isothiazole |
| 259e | 4-(2-Methoxy-pyridin-5-yl)-3-(4-methoxy-benzo[1,3]dioxol-6-yl)-isothiazole |
| 260e | 4-(5-Methoxy-pyridin-2-yl)-3-(4-methoxy-benzo[1,3]dioxol-6-yl)-isothiazole |
| 261e | 4-(3-Carboxy-4-methoxy-phenyl)-3-(4-methoxy-benzo[1,3]dioxol-6-yl)- |
| | isothiazole, sodium salt |
| 262e | 4-(3-Methoxycarbonyl-4-methoxy-phenyl)-3-(4- |
| | methoxy-benzo[1,3]dioxol-6-yl)-isothiazole |
| 263e | 4-(3-Sulfooxy-4-methoxy-phenyl)-3-(4-methoxy-benzo[1,3]dioxol-6-yl)- |
| | isothiazole, sodium salt |
| 264e | 4-(3-Amino-4-methoxy-phenyl)-3-(4-methoxy-benzo[1,3]dioxol-6-yl)- |
| | isothiazole |
| 265e | 4-(3,4-Dimethoxy-5 phosphonooxy-phenyl)-3-(4- |
| | methoxy-benzo[1,3]dioxol-6-yl)-isothiazole, disodium salt |
| 266e | 4-(2-Phosphonooxy-4-methoxy-phenyl)-3-(4-methoxy-benzo[1,3]dioxol-6-yl)- |
| | isothiazole, disodium salt |
| 267e | 4-(4-Methylsulfanyl-phenyl)-3-(4-methoxy-benzo[1,3]dioxol-6-yl)-isothiazole |
| 268e | 4-(3-Phosphonooxy-4-methylsulfanyl-phenyl)-3-(4- |
| | methoxy-benzo[1,3]dioxol-6-yl)-isothiazole, disodium salt |
| 269e | 4-(3-Amino-4-methylsulfanyl-phenyl)-3-(4-methoxy-benzo[1,3]dioxol-6-yl)- |
| | isothiazole |
| 270e | 4-(2,3-Dihydro-benzofuran-6-yl)-3-(4-methoxy-benzo[1,3]dioxol-6-yl)- |
| | isothiazole |
| 271e | 4-(4-Hydroxy-phenyl)-3-(4-methoxy-benzo[1,3]dioxol-6-yl)-isothiazole, sodium |
| | salt |
| 272e | 4-(4-Phosphonooxy-phenyl)-3-(4-methoxy-benzo[1,3]dioxol-6-yl)-isothiazole |
| 273e | 4-(4-1H-Tetrazol-5-yl-phenyl)-3-(4-methoxy-benzo[1,3]dioxol-6-yl)-isothiazole |
| 274e | 4-[4-(1-Methyl-1H-tetrazol-5-yl)-phenyl]-3-(4-methoxy-benzo[1,3]dioxol-6-yl)- |
| | isothiazole |
| 275e | 4-(1-Methyl-1H-indol-5-yl)-3-(4-methoxy-benzo[1,3]dioxol-6-yl)-isothiazole |
| 276e | 4-(3,4,5-Trimethoxy-phenyl)-3-(1-methyl-1H-indol-5-yl)-isothiazole |
| 277e | 4-(3,4,5-Trimethoxy-phenyl)-3-(3-phosphonooxy-4-methoxy-phenyl)- |
| | isothiazole, disodium salt |
| 278e | 4-(3,4,5-Trimethoxy-phenyl)-3-(N,N-dimethylamino-phenyl)-isothiazole |
| 279e | 4-(3,4,5-Trimethoxy-phenyl)-3-(3-amino-4-methoxy-phenyl)-isothiazole, |
| | hydrochloric acid salt |
| 280e | 4-(3,4,5-Trimethoxy-phenyl)-3-[3-(3-hydroxy-2S-amino- |
| | propionamido)-4-methoxy-phenyl]-isothiazole, hydrochloric acid salt |
| 281e | 4-(4-Methoxy-phenyl)-3-(2,4,5-trimethoxy-phenyl)-isothiazole |
| 282e | 4-(4-Methyl-phenyl)-3-(2,4,5-trimethoxy-phenyl)-isothiazole |
| 283e | 4-(4-Ethoxy-phenyl)-3-(2,4,5-trimethoxy-phenyl)-isothiazole |
| 284e | 4-(4-Ethyl-phenyl)-3-(2,4,5-trimethoxy-phenyl)-isothiazole |
| 285e | 4-(4-Propoxy-phenyl)-3-(2,4,5-trimethoxy-phenyl)-isothiazole |
| 286e | 4-(4-Propyl-phenyl)-3-(2,4,5-trimethoxy-phenyl)-isothiazole |
| 287e | 4-(4-Butoxy-phenyl)-3-(2,4,5-trimethoxy-phenyl)-isothiazole |
| 288e | 4-(4-Butyl-phenyl)-3-(2,4,5-trimethoxy-phenyl)-isothiazole |
| 289e | 4-(4-Bromo-phenyl)-3-(2,4,5-trimethoxy-phenyl)-isothiazole |
| 290e | 4-(4-Chloro-phenyl)-3-(2,4,5-trimethoxy-phenyl)-isothiazole |
| 291e | 4-(4-Fluoro-phenyl)-3-(2,4,5-trimethoxy-phenyl)-isothiazole |
| 292e | 4-(4-Nitro-phenyl)-3-(2,4,5-trimethoxy-phenyl)-isothiazole |
| 293e | 4-[4-(N,N-Dimethylamino)-phenyl]-3-(2,4,5-trimethoxy-phenyl)-isothiazole |
| 294e | 4-(3,4-Dimethoxy-phenyl)-3-(2,4,5-trimethoxy-phenyl)-isothiazole |
| 295e | 4-(3-Hydroxy-4-methoxy-phenyl)-3-(2,4,5-trimethoxy-phenyl)-isothiazole |
| 296e | 4-(3,4,5-Trimethoxy-phenyl)-3-(2,4,5-trimethoxy-phenyl)-isothiazole |
| 297e | 4-(4-Methoxy-phenyl)-3-(2,3,5-trimethoxy-phenyl)-isothiazole |
| 298e | 4-(4-Methyl-phenyl)-3-(2,3,5-trimethoxy-phenyl)-isothiazole |
| 299e | 4-(4-Ethoxy-phenyl)-3-(2,3,5-trimethoxy-phenyl)-isothiazole |
| 300e | 4-(4-Ethyl-phenyl)-3-(2,3,5-trimethoxy-phenyl)-isothiazole |
| 301e | 4-(4-Propoxy-phenyl)-3-(2,3,5-trimethoxy-phenyl)-isothiazole |
| 302e | 4-(4-Propyl-phenyl)-3-(2,3,5-trimethoxy-phenyl)-isothiazole |
| 303e | 4-(4-Butoxy-phenyl)-3-(2,3,5-trimethoxy-phenyl)-isothiazole |
| 304e | 4-(4-Butyl-phenyl)-3-(2,3,5-trimethoxy-phenyl)-isothiazole |
| 305e | 4-(4-Bromo-phenyl)-3-(2,3,5-trimethoxy-phenyl)-isothiazole |
| 306e | 4-(4-Chloro-phenyl)-3-(2,3,5-trimethoxy-phenyl)-isothiazole |
| 307e | 4-(4-Fluoro-phenyl)-3-(2,3,5-trimethoxy-phenyl)-isothiazole |
| 308e | 4-(4-Nitro-phenyl)-3-(2,3,5-trimethoxy-phenyl)-isothiazole |
| 309e | 4-[4-(N,N-Dimethylamino)-phenyl]-3-(2,3,5-trimethoxy-phenyl)-isothiazole |
| 310e | 4-(3,4-Dimethoxy-phenyl)-3-(2,3,5-trimethoxy-phenyl)-isothiazole |
| 311e | 4-(3-Hydroxy-4-methoxy-phenyl)-3-(2,3,5-trimethoxy-phenyl)-isothiazole |
| 312e | 4-(3,4,5-Trimethoxy-phenyl)-3-(2,3,5-trimethoxy-phenyl)-isothiazole |
| 313e | 4-(2,3,4,5-Tetramethoxy-phenyl)-3-(4-methoxy-phenyl)-isothiazole |
| 314e | 4-(2,3,4,5-Tetramethoxy-phenyl)-3-(4-methyl-phenyl)-isothiazole |
| 315e | 4-(2,3,4,5-Tetramethoxy-phenyl)-3-(4-ethoxy-phenyl)-isothiazole |
| 316e | 4-(2,3,4,5-Tetramethoxy-phenyl)-3-(4-ethyl-phenyl)-isothiazole |
| 317e | 4-(2,3,4,5-Tetramethoxy-phenyl)-3-(4-propoxy-phenyl)-isothiazole |
| 318e | 4-(2,3,4,5-Tetramethoxy-phenyl)-3-(4-propyl-phenyl)-isothiazole |
| 319e | 4-(2,3,4,5-Tetramethoxy-phenyl)-3-(4-butoxy-phenyl)-isothiazole |
| 320e | 4-(2,3,4,5-Tetramethoxy-phenyl)-3-(4-butyl-phenyl)-isothiazole |
| 321e | 4-(2,3,4,5-Tetramethoxy-phenyl)-3-(4-bromo-phenyl)-isothiazole |
| 322e | 4-(2,3,4,5-Tetramethoxy-phenyl)-3-(4-chloro-phenyl)-isothiazole |
| 323e | 4-(2,3,4,5-Tetramethoxy-phenyl)-3-(4-fluoro-phenyl)-isothiazole |
| 324e | 4-(2,3,4,5-Tetramethoxy-phenyl)-3-(4-nitro-phenyl)-isothiazole |
| 325e | 4-(2,3,4,5-Tetramethoxy-phenyl)-3-[4-(N,N,-dimethylamino)-phenyl]- |
| | isothiazole |
| 326e | 4-(2,3,4,5-Tetramethoxy-phenyl)-3-(3,4-dimethoxy-phenyl)-isothiazole |
| 327e | 4-(2,3,4,5-Tetramethoxy-phenyl)-3-(3-hydroxy-4-methoxy-phenyl)-isothiazole |
| 328e | 4-(2,3,4,5-Tetramethoxy-phenyl)-3-(3,4,5-trimethoxy-phenyl)-isothiazole |
| 329e | 4-(2,3-Dihydro-benzo[1,4]dioxin-6-yl)-3-(3,4-dimethoxy-phenyl)-isothiazole |
| 330e | 4-(3,4-Dimethy-phenyl)-3-(2-hydroxy-4-methoxy-5-ethyl-phenyl)-isothiazole |
| 331e | 4-(4-Chloro-phenyl)-3-(2-hydroxy-4-methoxy-5-ethyl-phenyl)-isothiazole |
| 332e | 4-(4-Methyl-phenyl)-3-(2-hydroxy-4-methoxy-5-ethyl-phenyl)-isothiazole |
| 333e | 4-(4-Amino-phenyl)-3-(2-hydroxy-4-methoxy-5-ethyl-phenyl)-isothiazole |
| 334e | 4-(4-Trifluoromethyl-phenyl)-3-(2-hydroxy-4-methoxy-5-ethyl-phenyl)- |
| | isothiazole |
| 335e | 4-(4-Methoxy-phenyl)-3-(2-hydroxy-4-methoxy-5-ethyl-phenyl)-isothiazole |
| 336e | 4-(3,4,5-Trimethoxy-phenyl)-3-(4-bromo-phenyl)-isothiazole |
| 337e | 2-amino-N-(2-methoxy-5-[5-(3,4,5-trimethoxy-phenyl)-isothiazol-4-yl)-phenyl)acetamide |
| | hydrochloride |
| 338e | 2-amino-3-hydroxy-N-(2-methoxy-5-[5-(3,4,5-trimethoxy-phenyl)-isothiazol-4-yl)- |
| | phenyl)propanamide hydrochloride |
| 339e | 2-amino-N-(2-methoxy-5-[5-(3,4,5-trimethoxy-phenyl)-isothiazol-4-yl)- |
| | phenyl)propanamide |
| 340e | 2-amino-N-(2-methoxy-5-[5-(3,4,5-trimethoxy-phenyl)-isothiazol- |
| | 4-yl)-phenyl)-4-(methylthio)butanamide hydrochloride |
| 341e | 2-amino-N-(2-methoxy-5-[5-(3,4,5-trimethoxy-phenyl)-isothiazol- |
| | 4-yl)-phenyl)butanamide |
| 342e | 2-amino-N-(2-methoxy-5-[5-(3,4,5-trimethoxy-phenyl)-isothiazol- |
| | 4-yl)-phenyl)-3-phenylpropanamide hydrochloride |
| 343e | 2-amino-N-(2-methoxy-5-[5-(3,4,5-trimethoxy-phenyl)-isothiazol- |
| | 4-yl)-phenyl)-4-methylpentanamide hydrochloride |
| 344e | 2-amino-N-(2-methoxy-5-[5-(3,4,5-trimethoxy-phenyl)-isothiazol- |
| | 4-yl)-phenyl)-3-(4-methoxyphenyl)propanamide hydrochloride |
| 345e | 2-methoxy-5-[5-(3,4,5-trimethoxy-phenyl)-isothiazol-4-yl)-phenyl dihydrogen |
| | phosphate |
| 346e | Sodium 2-methoxy-5-[5-(3,4,5- |
| | trimethoxy-phenyl)-isothiazol-4-yl)-phenyl phosphate |
| 347e | 1-{2-methoxy-5-[5-(3,4,5-trimethoxy-phenyl)-isothiazol- |
| | 4-yl]-phenylcarbomoyl}-2-methyl-propyl-ammonium chloride |
| 348e | 1-{2-methoxy-5-[5-(3,4,5-trimethoxy-phenyl)-isothiazol- |
| | 4-yl]-phenylcarbomoyl}-2-methyl-propyl-ammonium chloride |
| 349e | 1-{2-methoxy-5-[5-(3,4,5-trimethoxy-phenyl)-isothiazol- |
| | 4-yl]-phenylcarbomoyl}-2-methyl-butyl-ammonium chloride |
| 350e | 1-{2-methoxy-5-[5-(3,4,5-trimethoxy-phenyl)-isothiazol- |
| | 4-yl]-phenylcarbomoyl}-2-methyl-butyl-ammonium chloride |
| 351e | 2-hydroxy-1-{2-methoxy-5-[5-(3,4,5-trimethoxy-phenyl)-isothiazol-4-yl]- |
| | phenylcarbomoyl}-propyl-ammonium chloride |
| 352e | 2-hydroxy-1-{2-methoxy-5-[5-(3,4,5-trimethoxy-phenyl)-isothiazol-4-yl]- |
| | phenylcarbomoyl}-propyl-ammonium chloride |
| 353e | 2-(4-hydroxy-phenyl)-1-{2-methoxy-5-[5-(3,4,5-trimethoxy-phenyl)-isothiazol- |
| | 4-yl]-phenylcarbomoyl}-ethyl-ammonium chloride |
| 354e | 2-(4-hydroxy-phenyl)-1-{2-methoxy-5-[5-(3,4,5-trimethoxy-phenyl)-isothiazol- |
| | 4-yl]-phenylcarbomoyl}-ethyl-ammonium chloride |
| 355e | C-{2-methoxy-5-[5-(3,4,5-trimethoxy-phenyl)-isothiazol- |
| | 4-yl]phenylcarbomoyl}-C-phenyl-methyl-ammonium chloride |
| 356e | C-{2-methoxy-5-[5-(3,4,5-trimethoxy-phenyl)-isothiazol- |
| | 4-yl]phenylcarbomoyl}-C-phenyl-methyl-ammonium chloride |
| 357e | 2-(1H-indol-2-yl)-1-{2-methoxy-5-[5-(3,4,5-trimethoxy- |
| | phenyl)-isothiazol-4-yl]-phenylcarbomoyl}-ethyl-ammonium chloride |
| 358e | 2-(1H-indol-2-yl)-1-{2-methoxy-5-[5-(3,4,5-trimethoxy-phenyl)-isothiazol- |
| | 4-yl]-phenylcarbomoyl}-ethyl-ammonium chloride |
| 359e | 2-benzofuran-2-yl-1-{2-methoxy-5-[5-(3,4,5-trimethoxy-phenyl)-isothiazol- |
| | 4-yl]-phenylcarbomoyl}-ethyl-ammonium chloride |
| 360e | 2-benzofuran-2-yl-1-{2-methoxy-5-[5-(3,4,5-trimethoxy-phenyl)-isothiazol- |
| | 4-yl]-phenylcarbomoyl}-ethyl-ammonium chloride |
| 361e | 2-carboxyl-1-{2-methoxy-5-[5-(3,4,5-trimethoxy-phenyl)-isothiazol-4-yl]- |
| | phenylcarbomoyl}-ethyl-ammonium chloride |
| 362e | 2-carboxyl-1-{2-methoxy-5-[5-(3,4,5-trimethoxy-phenyl)-isothiazol-4-yl]- |
| | phenylcarbomoyl}-ethyl-ammonium chloride |
| 363e | 3-carboxyl-1-{2-methoxy-5-[5-(3,4,5-trimethoxy-phenyl)-isothiazol-4-yl]- |
| | phenylcarbomoyl}-propyl-ammonium chloride |
| 364e | 3-carboxyl-1-{2-methoxy-5-[5-(3,4,5-trimethoxy-phenyl)-isothiazol-4-yl]- |
| | phenylcarbomoyl}-propyl-ammonium chloride |
| 365e | 3-carbamoyl-1-{2-methoxy-5-[5-(3,4,5-trimethoxy-phenyl)-isothiazol-4-yl]- |
| | phenylcarbomoyl}-propyl-ammonium chloride |
| 366e | 3-carbamoyl-1-{2-methoxy-5-[5-(3,4,5-trimethoxy-phenyl)-isothiazol-4-yl]- |
| | phenylcarbomoyl}-propyl-ammonium chloride |
| 367e | 2-carbamoyl-1-{2-methoxy-5-[5-(3,4,5-trimethoxy-phenyl)-isothiazol-4-yl]- |
| | phenylcarbomoyl}-ethyl-ammonium chloride |
| 368e | 2-carbamoyl-1-{2-methoxy-5-[5-(3,4,5-trimethoxy-phenyl)-isothiazol-4-yl]- |
| | phenylcarbomoyl}-ethyl-ammonium chloride |
| 369e | 2-(3H-imidazol-4-yl)-1-{2-methoxy-5-[5-(3,4,5-trimethoxy-phenyl)-isothiazol- |
| | 4-yl]-phenylcarbomoyl}-ethyl-ammonium chloride |
| 370e | 2-(3H-imidazol-4-yl)-1-{2-methoxy-5-[5-(3,4,5-trimethoxy-phenyl)-isothiazol- |
| | 4-yl]-phenylcarbomoyl}-ethyl-ammonium chloride |
| 371e | 5-amino-1-{2-methoxy-5-[5-(3,4,5-trimethoxy-phenyl)-isothiazol-4-yl]- |
| | phenylcarbomoyl}-pentyl-ammonium chloride |
| 372e | 5-amino-1-{2-methoxy-5-[5-(3,4,5-trimethoxy-phenyl)-isothiazol-4-yl]- |
| | phenylcarbomoyl}-pentyl-ammonium chloride |
| 373e | 4-guanidino-1-{2-methoxy-5-[5-(3,4,5-trimethoxy-phenyl)-isothiazol-4-yl]- |
| | phenylcarbomoyl}-butyl-ammonium chloride |
| 374e | 4-guanidino-1-{2-methoxy-5-[5-(3,4,5-trimethoxy-phenyl)-isothiazol-4-yl]- |
| | phenylcarbomoyl}-butyl-ammonium chloride |
| 375e | N-{2-methoxy-5-[5-(3,4,5-trimethoxy-phenyl)-isothiazol-4-yl]-phenyl}succinamic |
| | acid |
| 376e | 4-{2-methoxy-5-[5-(3,4,5-trimethoxy-phenyl)-isothiazol- |
| | 4-yl]-phenylcarbamoyl}-butyric acid |
| 377e | 2-{2-methoxy-5-[5-(3,4,5-trimethoxy-phenyl)-isothiazol- |
| | 4-yl]-phenylcarbamoyl}-ethyl-ammonium chloride |
| 378e | 3-(2-methoxy-ethoxy)-N-{2-methoxy-5-[5-(3,4,5- |
| | trimethoxy-phenyl)-isothiazol-4-yl]-phenyl}-propionamide |
| 379e | 3-(2-PEG)-N-{2-methoxy-5-[5-(3,4,5-trimethoxy-phenyl)-isothiazol- |
| | 4-yl]-phenyl}-propionamide |
| 380e | N-{2-methoxy-5-[5-(3,4,5-trimethoxy-phenyl)-isothiazol-4-yl]-phenyl}-3-(2- |
| | methylamino-ethylamino)-propionamide |
| 381e | 3-PEG-N-{2-methoxy-5-[5-(3,4,5-trimethoxy-phenyl)-isothiazol-4-yl]- |
| | phenylcarbamoyl}-methyl)-propionamide |
| 382e | N-{2-methoxy-5-[5-(3,4,5-trimethoxy-phenyl)-isothiazol- |
| | 4-yl]phenylcarbamoyl}-methyl)-succinamic acid |
| 383e | {2-methoxy-5-[5-(3,4,5-trimethoxy-phenyl)-isothiazol-4-yl]-phenyl}-carbamic |
| | acid 2-methoxy-ethyl ester |
| 384e | 2-methoxy-5-(5-(3,4,5-trimethoxyphenyl)isothiazol-4-yl)phenylcarbamate- |
| | PEG |
| 385e | 3-amino-N-[4-guanadino-1-{2-methoxy-5-[5-(3,4,5-trimethoxy-phenyl)-isothiazol- |
| | 4-yl]-phenylcarbamoyl}-butylcarbamoyl)-methyl]-succinamic acid |
| 386e | 3-amino-N-[4-guanadino-1-{2-methoxy-5-[5-(3,4,5-trimethoxy-phenyl)-isothiazol- |
| | 4-yl]-phenylcarbamoyl}-butylcarbamoyl)-methyl]-succinamic acid |
| 387e | 2-amino-N-(2-methoxy-5-[5-(3,4,5-trimethoxy-phenyl)-isothiazol- |
| | 4-yl)-phenyl)propanamide hydrochloride |
| 388e | 2-amino-N-(2-methoxy-5-[3-(3,4,5-trimethoxy-phenyl)-isothiazol- |
| | 4-yl)-phenyl)acetamide hydrochloride |
| 389e | 2-amino-3-hydroxy-N-(2-methoxy-5-[3-(3,4,5-trimethoxy- |
| | phenyl)-isothiazol-4-yl)-phenyl)propanamide hydrochloride |
| 390e | 2-amino-N-(2-methoxy-5-[3-(3,4,5-trimethoxy-phenyl)-isothiazol-4-yl)- |
| | phenyl)propanamide |
| 391e | 2-amino-N-(2-methoxy-5-[3-(3,4,5-trimethoxy-phenyl)-isothiazol- |
| | 4-yl)-phenyl)-4-(methylthio)butanamide hydrochloride |
| 392e | 2-amino-N-(2-methoxy-5-[3-(3,4,5-trimethoxy-phenyl)-isothiazol- |
| | 4-yl)-phenyl)butanamide |
| 393e | 2-amino-N-(2-methoxy-5-[3-(3,4,5-trimethoxy-phenyl)- |
| | isothiazol-4-yl)-phenyl)-3-phenylpropanamide hydrochloride |
| 394e | 2-amino-N-(2-methoxy-5-[3-(3,4,5-trimethoxy-phenyl)-isothiazol- |
| | 4-yl)-phenyl)-4-methylpentanamide hydrochloride |
| 395e | 2-amino-N-(2-methoxy-5-[3-(3,4,5-trimethoxy-phenyl)- |
| | isothiazol-4-yl)-phenyl)-3-(4-methoxyphenyl)propanamide hydrochloride |
| 396e | 2-methoxy-5-[3-(3,4,5-trimethoxy-phenyl)-isothiazol-4-yl)- |
| | phenyl dihydrogen phosphate |
| 397e | Sodium 2-methoxy-5-[3-(3,4,5- |
| | trimethoxy-phenyl)-isothiazol-4-yl)-phenyl phosphate |
| 398e | 1-{2-methoxy-3-[5-(3,4,5-trimethoxy-phenyl)-isothiazol- |
| | 4-yl]-phenylcarbomoyl}-2-methyl-propyl-ammonium chloride |
| 399e | 1-{2-methoxy-5-[3-(3,4,5-trimethoxy-phenyl)-isothiazol- |
| | 4-yl]-phenylcarbomoyl}-2-methyl-propyl-ammonium chloride |
| 400e | 1-{2-methoxy-5-[3-(3,4,5-trimethoxy-phenyl)-isothiazol- |
| | 4-yl]-phenylcarbomoyl}-2-methyl-butyl-ammonium chloride |
| 401e | 1-{2-methoxy-5-[3-(3,4,5-trimethoxy-phenyl)-isothiazol- |
| | 4-yl]-phenylcarbomoyl}-2-methyl-butyl-ammonium chloride |
| 402e | 2-hydroxy-1-{2-methoxy-5-[3-(3,4,5-trimethoxy-phenyl)-isothiazol-4-yl]- |
| | phenylcarbomoyl}-propyl-ammonium chloride |
| 403e | 2-hydroxy-1-{2-methoxy-5-[3-(3,4,5-trimethoxy-phenyl)-isothiazol-4-yl]- |
| | phenylcarbomoyl}-propyl-ammonium chloride |
| 404e | 2-(4-hydroxy-phenyl)-1-{2-methoxy-5-[3-(3,4,5-trimethoxy-phenyl)-isothiazol- |
| | 4-yl]-phenylcarbomoyl}-ethyl-ammonium chloride |
| 405e | 2-(4-hydroxy-phenyl)-1-{2-methoxy-5-[3-(3,4,5-trimethoxy-phenyl)-isothiazol- |
| | 4-yl]-phenylcarbomoyl}-ethyl-ammonium chloride |
| 406e | C-{2-methoxy-5-[3-(3,4,5-trimethoxy-phenyl)-isothiazol- |
| | 4-yl]phenylcarbomoyl}-C-phenyl-methyl-ammonium chloride |
| 407e | C-{2-methoxy-5-[3-(3,4,5-trimethoxy-phenyl)-isothiazol- |
| | 4-yl]phenylcarbomoyl}-C-phenyl-methyl-ammonium chloride |
| 408e | 2-(1H-indol-2-yl)-1-{2-methoxy-5-[3-(3,4,5-trimethoxy- |
| | phenyl)-isothiazol-4-yl]-phenylcarbomoyl}-ethyl-ammonium chloride |
| 409e | 2-(1H-indol-2-yl)-1-{2-methoxy-5-[3-(3,4,5-trimethoxy-phenyl)-isothiazol- |
| | 4-yl]-phenylcarbomoyl}-ethyl-ammonium chloride |
| 410e | 2-benzofuran-2-yl-1-{2-methoxy-5-[3-(3,4,5-trimethoxy-phenyl)-isothiazol- |
| | 4-yl]-phenylcarbomoyl}-ethyl-ammonium chloride |
| 411e | 2-benzofuran-2-yl-1-{2-methoxy-5-[3-(3,4,5-trimethoxy-phenyl)-isothiazol- |
| | 4-yl]-phenylcarbomoyl}-ethyl-ammonium chloride |
| 412e | 2-carboxyl-1-{2-methoxy-5-[3-(3,4,5-trimethoxy-phenyl)-isothiazol-4-yl]- |
| | phenylcarbomoyl}-ethyl-ammonium chloride |
| 413e | 2-carboxyl-1-{2-methoxy-5-[3-(3,4,5-trimethoxy-phenyl)-isothiazol- |
| | 4-yl]-phenylcarbomoyl}-ethyl-ammonium chloride |
| 414e | 3-carboxyl-1-{2-methoxy-5-[3-(3,4,5-trimethoxy-phenyl)-isothiazol-4-yl]- |
| | phenylcarbomoyl}-propyl-ammonium chloride |
| 415e | 3-carboxyl-1-{2-methoxy-5-[3-(3,4,5-trimethoxy-phenyl)-isothiazol-4-yl]- |
| | phenylcarbomoyl}-propyl-ammonium chloride |
| 416e | 3-carbamoyl-1-{2-methoxy-5-[3-(3,4,5-trimethoxy-phenyl)-isothiazol-4-yl]- |
| | phenylcarbomoyl}-propyl-ammonium chloride |
| 417e | 3-carbamoyl-1-{2-methoxy-5-[3-(3,4,5-trimethoxy-phenyl)-isothiazol-4-yl]- |
| | phenylcarbomoyl}-propyl-ammonium chloride |
| 418e | 2-carbamoyl-1-{2-methoxy-5-[3-(3,4,5-trimethoxy-phenyl)-isothiazol-4-yl]- |
| | phenylcarbomoyl}-ethyl-ammonium chloride |
| 419e | 2-carbamoyl-1-{2-methoxy-5-[3-(3,4,5-trimethoxy-phenyl)-isothiazol-4-yl]- |
| | phenylcarbomoyl}-ethyl-ammonium chloride |
| 420e | 2-(3H-imidazol-4-yl)-1-{2-methoxy-5-[3-(3,4,5-trimethoxy-phenyl)-isothiazol- |
| | 4-yl]-phenylcarbomoyl}-ethyl-ammonium chloride |
| 421e | 2-(3H-imidazol-4-yl)-1-{2-methoxy-5-[3-(3,4,5-trimethoxy-phenyl)-isothiazol- |
| | 4-yl]-phenylcarbomoyl}-ethyl-ammonium chloride |
| 422e | 5-amino-1-{2-methoxy-5-[3-(3,4,5-trimethoxy-phenyl)-isothiazol-4-yl]- |
| | phenylcarbomoyl}-pentyl-ammonium chloride |
| 423e | 5-amino-1-{2-methoxy-5-[3-(3,4,5-trimethoxy-phenyl)-isothiazol-4-yl]- |
| | phenylcarbomoyl}-pentyl-ammonium chloride |
| 424e | 4-guanidino-1-{2-methoxy-5-[3-(3,4,5-trimethoxy-phenyl)-isothiazol-4-yl]- |
| | phenylcarbomoyl}-butyl-ammonium chloride |
| 425e | 4-guanidino-1-{2-methoxy-5-[3-(3,4,5-trimethoxy-phenyl)-isothiazol- |
| | 4-yl]-phenylcarbomoyl}-butyl-ammonium chloride |
| 426e | N-{2-methoxy-5-[3-(3,4,5- |
| | trimethoxy-phenyl)-isothiazol-4-yl]-phenyl}succinamic acid |
| 427e | 4-{2-methoxy-5-[3-(3,4,5-trimethoxy-phenyl)-isothiazol- |
| | 4-yl]-phenylcarbamoyl}-butyric acid |
| 428e | 2-{2-methoxy-5-[3-(3,4,5-trimethoxy-phenyl)-isothiazol- |
| | 4-yl]-phenylcarbamoyl}-ethyl-ammonium chloride |
| 429e | 3-(2-methoxy-ethoxy)-N-{2-methoxy-5-[3-(3,4,5- |
| | trimethoxy-phenyl)-isothiazol-4-yl]-phenyl}-propionamide |
| 430e | 3-(2-PEG)-N-{2-methoxy-5-[3-(3,4,5-trimethoxy-phenyl)-isothiazol- |
| | 4-yl]-phenyl}-propionamide |
| 431e | N-{2-methoxy-5-[3-(3,4,5-trimethoxy-phenyl)-isothiazol-4-yl]-phenyl}-3-(2- |
| | methylamino-ethylamino)-propionamide |
| 432e | 3-PEG-N-{2-methoxy-5-[3-(3,4,5-trimethoxy-phenyl)-isothiazol-4-yl]- |
| | phenylcarbamoyl}-methyl)-propionamide |
| 433e | N-{2-methoxy-5-[3-(3,4,5-trimethoxy-phenyl)-isothiazol- |
| | 4-yl]phenylcarbamoyl}-methyl)-succinamic acid |
| 434e | {2-methoxy-5-[3-(3,4,5-trimethoxy-phenyl)-isothiazol-4-yl]-phenyl}-carbamic |
| | acid 2-methoxy-ethyl ester |
| 435e | 2-methoxy-5-(3-(3,4,5- |
| | trimethoxyphenyl)isothiazol-4-yl)phenylcarbamate-PEG |
| 436e | 3-amino-N-[4-guanadino-1-{2-methoxy-5-[3-(3,4,5-trimethoxy-phenyl)-isothiazol- |
| | 4-yl]-phenylcarbamoyl}-butylcarbamoyl)-methyl]-succinamic acid |
| 437e | 3-amino-N-[4-guanadino-1-{2-methoxy-5-[3-(3,4,5-trimethoxy-phenyl)- |
| | isothiazol-4-yl]-phenylcarbamoyl}-butylcarbamoyl)-methyl]-succinamic acid |
| 438e | 2-amino-N-(2-methoxy-5-[3-(3,4,5-trimethoxy-phenyl)-isothiazol- |
| | 4-yl)-phenyl)propanamide hydrochloride |
| 439e | methyl 2-(2-(2-methoxy-5-(5-(3,4,5-trimethoxyphenyl)isothiazol- |
| | 4-yl)phenylamino)-2-oxoethylamino)acetate |
| 440e | 4-amino-5-(2-methoxy-5-(5-(3,4,5-trimethoxyphenyl)isothiazol- |
| | 4-yl)phenylamino)-5-oxopentanoic acid hydrochloride |
| 441e | 3-amino-N-(2-methoxy-5-(5-(3,4,5-trimethoxyphenyl)isothiazol- |
| | 4-yl)phenyl)propanamide hydrochloride |
| 442e | 3-amino-N-(2-methoxy-5-(5-(3,4,5-trimethoxyphenyl)isothiazol- |
| | 4-yl)phenyl)-4-methylpentanamide hydrochloride |
| 443e | methyl 2-(2-(2-methoxy-5-(3-(3,4,5-trimethoxyphenyl)isothiazol- |
| | 4-yl)phenylamino)-2-oxoethylamino)acetate |
| 444e | 4-amino-5-(2-methoxy-5-(3-(3,4,5-trimethoxyphenyl)isothiazol- |
| | 4-yl)phenylamino)-5-oxopentanoic acid hydrochloride |
| 445e | 3-amino-N-(2-methoxy-5-(3-(3,4,5-trimethoxyphenyl)isothiazol- |
| | 4-yl)phenyl)propanamide hydrochloride |
| 446e | 3-amino-N-(2-methoxy-5-(3-(3,4,5-trimethoxyphenyl)isothiazol- |
| | 4-yl)phenyl)-4-methylpentanamide hydrochloride |

### Methods of Making the Compounds of the Invention

The compounds of the invention can be made by the methods described herein in Example 1. In addition, the compounds of the invention can be prepared using the methods described in Olivera, et al., J. Org. Chem. (2000), 65:6398-6411; Olivera, et al., Tetrahedron (2002), 58:3021-3037; Dominguez, et al., J. Org. Chem. (1996), 61:5435-5439; Olivera, et al., Tet. Let. (1999), 40:3479-3480; Khilya, et al. Ukrainskii Khimicheskii Zhurnal (Russian Edition) (1990), 56(3);280-286. The entire teachings of these references are incorporated herein by reference.

For triazole compounds, typically, an aromatic amine compound (a) is treated with a nitrite salt, such as sodium nitrite, in HCl and water followed by treatment with an azide salt, such as sodium azide, to form an aromatic azide (b). The aromatic azide (b) is then heated with an alkyne which is substituted with an aromatic group (c) to form the [1,2,3] triazole ring (I) (see scheme I).

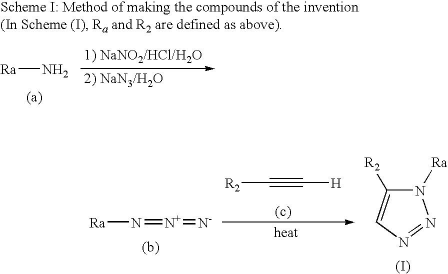

Other methods of preparing 1,2,3-triazoles are described in Pati, Hari, N., et al., (2005). “Synthesis and biological evaluation of cis-combretastatin analogs and their novel 1,2,3-triazole derivatives.” Heterocycl. Commun., 11(2), 117-120, which is incorporated by reference herein in its entirety. In general, Wittig reagents are reacted with substituted benzaldehydes and then 1,2,3-triazoles are produced according to scheme II below.

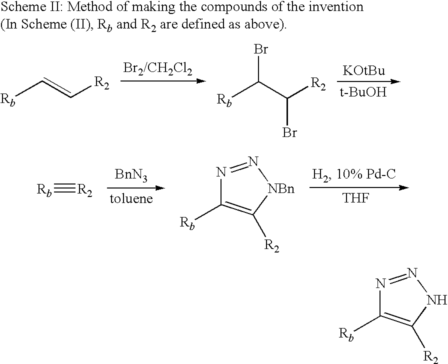

### Methods of Treatment and Prevention

In one embodiment, the invention provides a method of treating or inhibiting angiogenesis, comprising administering to a subject an effective amount of a compound of any one of formulas (I)-(XXIX), (XXXI), (XXXV)-(XL), (IA)-(XXIA), (XXVIIA)-(XXIXA), (XXXIA), (XXXVA)-(XLA), (IB)-(XXIB), (XXVIIB)-(XXIXB), (XXXIB), (XXXVB)-(XLB), or of Table 1, or a pharmaceutically acceptable salt, solvate, clathrate, and prodrug thereof, or a pharmaceutical composition comprising a compound of any one of formulas (I)-(XXIX), (XXXI), (XXXV)-(XL), (IA)-(XXIA), (XXVIIA)-(XXIXA), (XXXIA), (XXXVA)-(XLA), (IB)-(XXIB), (XXVIIB)-(XXIXB), (XXXIB), (XXXVB)-(XLB), or of Table 1, or a pharmaceutically acceptable salt, solvate, clathrate, and prodrug thereof.

### Combination Therapies

The invention also provides methods of preventing, treating, managing, or ameliorating an angiogenesis related disorder, or one or more symptoms thereof, said methods comprising administering to a subject in need thereof one or more compounds of the invention and one or more other therapies (e.g., one or more prophylactic or therapeutic agents that are currently being used, have been used, are known to be useful or in development for use in the prevention, treatment or amelioration of an angiogenesis related disorder).

The prophylactic or therapeutic agents of the combination therapies of the invention can be administered sequentially or concurrently. In a specific embodiment, the combination therapies of the invention comprise one or more compounds and at least one other therapy (e.g., another prophylactic or therapeutic agent) which has the same mechanism of action as said compounds (e.g., a therapeutic agent that inhibits tubulin polymerization). In another specific embodiment, the combination therapies of the invention comprise one or more compounds of the invention and at least one other therapy (e.g., another prophylactic or therapeutic agent) which has a different mechanism of action than said compounds. In certain embodiments, the combination therapies of the present invention improve the prophylactic or therapeutic effect of one or more compounds of the invention by functioning together with the compounds to have an additive or synergistic effect. In certain embodiments, the combination therapies of the present invention reduce the side effects associated with the therapies (e.g., prophylactic or therapeutic agents). In certain embodiments, the combination therapies of the present invention reduce the effective dosage of one or more of the therapies.

The prophylactic or therapeutic agents of the combination therapies can be administered to a subject, preferably a human subject, in the same pharmaceutical composition. In alternative embodiments, the prophylactic or therapeutic agents of the combination therapies can be administered concurrently to a subject in separate pharmaceutical compositions. The prophylactic or therapeutic agents may be administered to a subject by the same or different routes of administration.

In a specific embodiment, a pharmaceutical composition comprising one or more compounds of the invention is administered to a subject, preferably a human, to prevent, treat, manage, or ameliorate an angiogenesis related disorder, or one or more symptoms thereof. In accordance with the invention, pharmaceutical compositions of the invention may also comprise one or more other agents (e.g., prophylactic or therapeutic agents which are currently being used, have been used, or are known to be useful in the prevention, treatment or amelioration of an angiogenesis related disorder or a symptom thereof).

The invention provides methods for preventing, managing, treating or ameliorating an angiogenesis related disorder, or one or more symptoms thereof in a subject refractory (either completely or partially) to existing agent therapies for such an angiogenesis related disorder, said methods comprising administering to said subject a dose of an effective amount of one or more compounds of the invention and a dose of an effective amount of one or more therapies (e.g., one or more prophylactic or therapeutic agents useful for the prevention, treatment, management, or amelioration of an angiogenesis related disorder or a symptom thereof). The invention also provides methods for preventing, treating, managing, or ameliorating an angiogenesis related disorder or a symptom thereof by administering one or more compounds of the invention in combination with any other therapy(ies) to patients who have proven refractory to other therapies but are no longer on these therapies.

The compounds of the invention and/or other therapies can be administered to a subject by any route known to one of skill in the art. Examples of routes of administration include, but are not limited to, parenteral, e.g., intravenous, intradermal, subcutaneous, oral (e.g., inhalation), intranasal, transdermal (topical), transmucosal, and rectal administration.

Agents Useful in Combination with Compounds of the Invention

Anti-angiogenesis agents that can be co-administered with the compounds of the invention include Dalteparin, Suramin, ABT-510, Combretastatin A4 Phosphate, Lenalidomide, LY317615 (Enzastaurin), Soy Isoflavone (Genistein; Soy Protein Isolate), Thalidomide, AMG-706, Anti-VEGF Antibody (Bevacizumab; Avastin™), AZD2171, Bay 43-9006 (Sorafenib tosylate), PI-88, PTK787/ZK 222584 (Vatalanib), SU11248 (Sunitinib malate), VEGF-Trap, XL184, ZD6474, ATN-161, EMD 121974 (Cilenigtide), Celecoxib, Angiostatin, Endostatin, Regranex, Apligraf, Paclitaxel, tetracyclines, clarithromycin, lasix, captopril, aspirin, Vitamin D3 analogs, retinoids, Imiquomod, Interferon alfa2a, Minocycline, copper peptide containing dressings, Lucentis™, ATG002, Pegaptanib Sodium, Tryptophanyl-tRNA synthetase, squalamine lactate, anecortave acetate, AdPEDF, AG-013958, JSM6427, TG100801, Veglin, ascorbic acid ethers (and their analogs), and Pamidronate.

Anticancer agents that can be co-administered with the compounds of the invention include Taxol™, also referred to as “paclitaxel”, which is a well-known anti-cancer drug which acts by enhancing and stabilizing microtubule formation, and analogs of Taxol™, such as Taxotere™. Compounds that have the basic taxane skeleton as a common structure feature, have also been shown to have the ability to arrest cells in the G2-M phases due to stabilized microtubules and may be useful for treating cancer in combination with the compounds of the invention.

Other anti-cancer agents that can be employed in combination with the compounds of the invention include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer drugs that can be employed in combination with the compounds of the invention include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocannycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic kid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred anti-cancer drugs are 5-fluorouracil and leucovorin.

Other chemotherapeutic agents that can be employed in combination with the compounds of the invention include but are not limited to alkylating agents, antimetabolites, natural products, or hormones. Examples of alkylating agents useful for the treatment or prevention of T-cell malignancies in the methods and compositions of the invention include but are not limited to, nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites useful for the treatment or prevention of T-cell malignancies in the methods and compositions of the invention include but are not limited to folic acid analog (e.g., methotrexate), or pyrimidine analogs (e.g., Cytarabine), purine analogs (e.g., mercaptopurine, thioguanine, pentostatin). Examples of natural products useful for the treatment or prevention of T-cell malignancies in the methods and compositions of the invention include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents that can be employed in combination with the compounds of the invention include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites useful for the treatment or prevention of cancer in the methods and compositions of the invention include but are not limited to folic acid analog (e.g., methotrexate), or pyrimidine analogs (e.g., fluorouracil, floxouridine, Cytarabine), purine analogs (e.g., mercaptopurine, thioguanine, pentostatin). Examples of natural products useful for the treatment or prevention of cancer in the methods and compositions of the invention include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin), enzymes (e.g., L-asparaginase), or biological response modifiers (e.g., interferon alpha). Examples of hormones and antagonists useful for the treatment or prevention of cancer in the methods and compositions of the invention include but are not limited to adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions of the invention for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Examples of anti-cancer agents which act by arresting cells in the G2-M phases due to stabilized microtubules and which can be used in combination with the compounds of the invention include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-IAABE (Cytoskeleton/Mt. Sinai School of Medicine), A-204197 (Abbott), T-607 (Tularik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCD, Resverastatin phosphate sodium, BPR-0Y-007 (National Health Research Institutes), and SSR-250411 (Sanofi).

### Pharmaceutical Compositions

The present invention provides compositions for the treatment, prophylaxis, and amelioration of angiogenesis related disorders. In a specific embodiment, a composition comprises one or more compounds of the invention, or a pharmaceutically acceptable salt, solvate, clathrate, hydrate or prodrug thereof. In another embodiment, a composition of the invention comprises one or more prophylactic or therapeutic agents other than a compound of the invention, or a pharmaceutically acceptable salt, solvate, clathrate, hydrate, prodrug thereof. In another embodiment, a composition of the invention comprises one or more compounds of the invention, or a pharmaceutically acceptable salt, solvate, clathrate, hydrate or prodrug thereof, and one or more other prophylactic or therapeutic agents. In another embodiment, the composition comprises a compound of the invention, or a pharmaceutically acceptable salt, solvate, clathrate, hydrate, or prodrug thereof, and a pharmaceutically acceptable carrier, diluent or excipient.

In a preferred embodiment, a composition of the invention is a pharmaceutical composition or a single unit dosage form. Pharmaceutical compositions and dosage forms of the invention comprise one or more active ingredients in relative amounts and formulated in such a way that a given pharmaceutical composition or dosage form can be used to treat or prevent angiogenesis related disorders. Preferred pharmaceutical compositions and dosage forms comprise a compound of formulas (I)-(XXIX), (XXXI), (XXXV)-(XL), (IA)-(XXIA), (XXVIIA)-(XXIXA), (XXXIA), (XXXVA)-(XLA), (IB)-(XXIB), (XXVIIB)-(XXIXB), (XXXIB), (XXXVB)-(XLB), or of Table 1, or a pharmaceutically acceptable prodrug, salt, solvate, clathrate, hydrate, or prodrug thereof, optionally in combination with one or more additional active agents.

A pharmaceutical composition of the invention is formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings.

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form suitable for mucosal administration may contain a smaller amount of active ingredient(s) than an oral dosage form used to treat the same indication. This aspect of the invention will be readily apparent to those skilled in the art. See, e.g., Remington's Pharmaceutical Sciences (1990) 18th ed., Mack Publishing, Easton Pa.

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms.

The suitability of a particular excipient may also depend on the specific active ingredients in the dosage form. For example, the decomposition of some active ingredients can be accelerated by some excipients such as lactose, or when exposed to water. Active ingredients that comprise primary or secondary amines (e.g., N-desmethylvenlafaxine and N,N-didesmethylvenlafaxine) are particularly susceptible to such accelerated decomposition. Consequently, this invention encompasses pharmaceutical compositions and dosage forms that contain little, if any, lactose. As used herein, the term “lactose-free” means that the amount of lactose present, if any, is insufficient to substantially increase the degradation rate of an active ingredient. Lactose-free compositions of the invention can comprise excipients that are well known in the art and are listed, for example, in the U.S. Pharmocopia (USP) SP (XXI)/NF (XVI). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen (1995) Drug Stability: Principles & Practice, 2d. Ed., Marcel Dekker, NY, N.Y., 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizer” include, but are not limited to, antioxidants such as ascorbic acid, pH buffers, or salt buffers.

Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences (1990) 18th ed., Mack Publishing, Easton Pa.

Typical oral dosage forms of the invention are prepared by combining the active ingredient(s) in an admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. One specific binder is a mixture of microcrystalline cellulose and sodium carboxymethyl cellulose sold as AVICEL RC-581. Suitable anhydrous or low moisture excipients or additives include AVICEL-PH-103J and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

Controlled Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

A particular extended release formulation of this invention comprises a therapeutically or prophylactically effective amount of a compound of formulas (I)-(XXIX), (XXXI), (XXXV)-(XL), (IA)-(XXIA), (XXVIIA)-(XXIXA), (XXXIA), (XXXVA)-(XLA), (IB)-(XXIB), (XXVIIB)-(XXVIIB), (XXXIB), (XXXVB)-(XLB), or of Table 1, or a pharmaceutically acceptable salt, solvate, hydrate, clathrate, or prodrug thereof, in spheroids which further comprise microcrystalline cellulose and, optionally, hydroxypropylmethyl-cellulose coated with a mixture of ethyl cellulose and hydroxypropylmethylcellulose. Such extended release formulations can be prepared according to U.S. Pat. No. 6,274,171, the entirely of which is incorporated herein by reference.

A specific controlled-release formulation of this invention comprises from about 6% to about 40% a compound of formulas (I)-(XXIX), (XXXI), (XXXV)-(XL), (IA)-(XXIA), (XXVIIA)-(XXIXA), (XXXIA), (XXXVA)-(XLA), (IB)-(XXIB), (XXVIIB)-(XXIXB), (XXXIB), (XXXVB)-(XLB), or of Table 1, or a pharmaceutically acceptable salt, solvate, hydrate, clathrate, or prodrug thereof, by weight, about 50% to about 94% microcrystalline cellulose, NF, by weight, and optionally from about 0.25% to about 1% by weight of hydroxypropyl-methylcellulose, USP, wherein the spheroids are coated with a film coating composition comprised of ethyl cellulose and hydroxypropylmethylcellulose.

Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences (1980 & 1990) 16th and 18th eds., Mack Publishing, Easton Pa. and Introduction to Pharmaceutical Dosage Forms (1985) 4th ed., Lea & Febiger, Philadelphia. Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences (1980 & 1990) 16th and 18th eds., Mack Publishing, Easton Pa.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

Dosage & Frequency of Administration

The amount of the compound or composition of the invention which will be effective in the prevention, treatment, management, or amelioration of angiogenesis related disorders, or one or more symptoms thereof, will vary with the nature and severity of the disease or condition, and the route by which the active ingredient is administered. The frequency and dosage will also vary according to factors specific for each patient depending on the specific therapy (e.g., therapeutic or prophylactic agents) administered, the severity of the disorder, disease, or condition, the route of administration, as well as age, body, weight, response, and the past medical history of the patient. Effective doses may be extrapolated from dose-response curves derived from in vitro or animal model test systems. Suitable regiments can be selected by one skilled in the art by considering such factors and by following, for example, dosages reported in the literature and recommended in the Physician's Desk Reference (57th ed., 2003).

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

In general, the recommended daily dose range of a compound of the invention for the conditions described herein lie within the range of from about 0.01 mg to about 1000 mg per day, given as a single once-a-day dose or preferably as divided doses throughout a day. In one embodiment, the daily dose is administered twice daily in equally divided doses. Specifically, a daily dose range should be from about 5 mg to about 500 mg per day, more specifically, between about 10 mg and about 200 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Different therapeutically effective amounts may be applicable for different angiogenesis related disorders, as will be readily known by those of ordinary skill in the art. Similarly, amounts sufficient to prevent, manage, treat or ameliorate such angiogenesis related disorders, but insufficient to cause, or sufficient to reduce, adverse effects associated with the compounds of the invention are also encompassed by the above described dosage amounts and dose frequency schedules. Further, when a patient is administered multiple dosages of a compound of the invention, not all of the dosages need be the same. For example, the dosage administered to the patient may be increased to improve the prophylactic or therapeutic effect of the compound or it may be decreased to reduce one or more side effects that a particular patient is experiencing.

In a specific embodiment, the dosage of the composition of the invention or a compound of the invention administered to prevent, treat, manage, or ameliorate a proliferative disorders, such as cancer, or one or more symptoms thereof in a patient is 150 μg/kg, preferably 250 μg/kg, 500 μg/kg, 1 mg/kg, 5 mg/kg, 10 mg/kg, 25 mg/kg, 50 mg/kg, 75 mg/kg, 100 mg/kg, 125 mg/kg, 150 mg/kg, or 200 mg/kg or more of a patient's body weight. In another embodiment, the dosage of the composition of the invention or a compound of the invention administered to prevent, treat, manage, or ameliorate a proliferative disorders, such as cancer, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 to 7 mg, 0.1 mg to 5 mg, 0.1 to 2.5 mg, 0.25 mg to 20 mg, 0.25 to 15 mg, 0.25 to 12 mg, 0.25 to 8 mg, 0.25 mg to 7 m g, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

The dosages of prophylactic or therapeutic agents other than compounds of the invention, which have been or are currently being used to prevent, treat, manage, or proliferative disorders, such as cancer, or one or more symptoms thereof can be used in the combination therapies of the invention. Preferably, dosages lower than those which have been or are currently being used to prevent, treat, manage, or ameliorate a proliferative disorders, or one or more symptoms thereof, are used in the combination therapies of the invention. The recommended dosages of agents currently used for the prevention, treatment, management, or amelioration of a proliferative disorders, such as cancer, or one or more symptoms thereof, can obtained from any reference in the art including, but not limited to, Hardman et al., eds., 1996, Goodman & Gilman's The Pharmacological Basis Of Basis Of Therapeutics 9^{th} Ed, Mc-Graw-Hill, New York; Physician's Desk Reference (PDR) 57^{th} Ed., 2003, Medical Economics Co., Inc., Montvale, N.J., which are incorporated herein by reference in its entirety.

In certain embodiments, when the compounds of the invention are administered in combination with another therapy, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In one embodiment, two or more therapies (e.g., prophylactic or therapeutic agents) are administered within the same patient visit.

In certain embodiments, one or more compounds of the invention and one or more other the therapies (e.g., prophylactic or therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound of the invention may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In a specific embodiment, the invention provides a method of preventing, treating, managing, or ameliorating a proliferative disorders, such as cancer, or one or more symptoms thereof, said methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds of the invention once every day, preferably, once every 2 days, once every 3 days, once every 4 days, once every 5 days, once every 6 days, once every 7 days, once every 8 days, once every 10 days, once every two weeks, once every three weeks, or once a month.

### Other Embodiments

The compounds of the invention may be used as research tools (for example, to evaluate the mechanism of action of new drug agents, to isolate new drug discovery targets using affinity chromatography, as antigens in an ELISA or ELISA-like assay, or as standards in in vitro or in vivo assays). These and other uses and embodiments of the compounds and compositions of this invention will be apparent to those of ordinary skill in the art.

The invention is further defined by reference to the following examples describing in detail the preparation of compounds of the invention. It will be apparent to those skilled in the art that many modifications, both to materials and methods, may be practiced without departing from the purpose and interest of this invention. The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### Examples

### Materials and General Methods

Reagents and solvents used below can be obtained from commercial sources such as Aldrich Chemical Co. (Milwaukee, Wis., USA). ¹H-NMR and ¹³C-NMR spectra were recorded on a Varian 300 MHz NMR spectrometer. Significant peaks are tabulated in the order: δ (ppm): chemical shift, multiplicity (s, singlet; d, doublet; t, triplet; q, quartet; m, multiplet; br s, broad singlet), coupling constant(s) in Hertz (Hz) and number of protons.

### Example 1

### Synthesis of Representative Exemplary Compounds of this Invention

### Compound 3: 4-(4-Methoxy-phenyl)-5-(3,4,5-trimethoxy-phenyl)-isoxazole

### (1) Synthesis of 3-(4-Methoxy-phenyl)-1-(3,4,5-trimethoxy-phenyl)-propenone

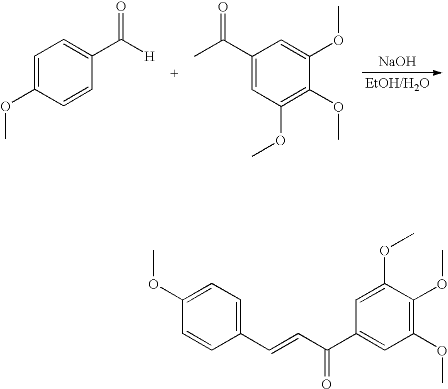

To a stirred solution of p-anislaldehyde (1.36 g, 10 mmol) and 1-(3,4,5-trimethoxy-phenyl)-ethanone (2.1 g, 10 mmol) in ethyl alcohol (EtOH) (10 mL) was added a 50% solution of NaOH in H₂O (1 mL). After the reaction had proceeded to completion, volatile components were removed under reduced pressure and the residue was taken up with ethyl acetate (EtOAc) (50 mL). The EtOAc layer was washed with H₂O (2×30 mL) and then dried with Na₂SO₄. After removal of EtOAc, the product was precipitated out from EtOH/H₂O. Solid material collected by filtration and was washed by H₂O (20 mL) and 95% ethyl alcohol (10 mL). The product,

3-(4-Methoxy-phenyl)-1-(3,4,5-trimethoxy-phenyl)-propenone (2.8 g, 85% yield), was obtained as a yellow solid. ¹H-NMR δ 3.85 (s, 3H), 3.90 (s, 3H), 3.95 (s, 6H), 6.95 (d, 2H, J=8), 7.28(s, 2H), 7.39 (d, 1H, J=15), 7.65 (d, 2H, J=8), 7.85 (d, 1H, J=15)ppm.

### (2) Synthesis of [3-(4-Methoxy-phenyl)-oxiranyl]-(3,4,5-trimethoxy-phenyl)-methanone

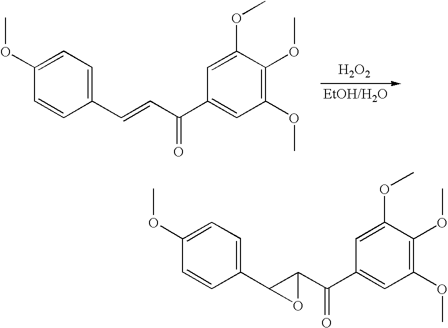

To a stirred solution of 3-(4-Methoxy-phenyl)-1-(3,4,5-trimethoxy-phenyl)-propenone (1.64 g, 5 mmol) and 1N NaOH (2.52 mL) in 95% EtOH (22 mL) was added a cold solution of 30% H₂O₂ (0.77 mL) at room temperature. After 72 h stirring, the precipitated material was collected by filtration and washed with 95% EtOH to afford [3-(4-methoxy-phenyl)-oxiranyl]-(3,4,5-trimethoxy-phenyl)-methanone as a white solid (1.38 g, yield 80%). ¹H-NMR (CDCl₃) δ 3.81 (s, 3H), 3.92 (s, 6H), 3.95 (s, 3H), 4.05 (d, 1H, J=2), 4.20 (1H, J=2), 6.95 (d, 2H, J=7), 7.25-7.35 (m, 4H)ppm.

### (3) Synthesis of 4-(4-Methoxy-phenyl)-5-(3,4,5-trimethoxy-phenyl)-isoxazole

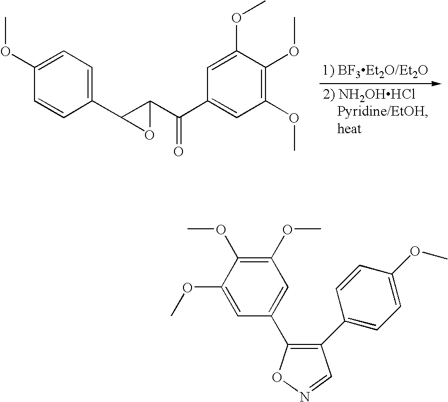

To a stirred solution of [3-(4-methoxy-phenyl)-oxiranyl]-(3,4,5-trimethoxy-phenyl)-methanone (0.5 g, 1.45 mmol) in dry ether (15 mL) was added BF₃.Et₂O (2.52 mL) slowly. After the addition, it was heated to reflux for 1 h. After the reaction mixture had cooled to room temperature, it was poured into ice-H₂O (100 mL). The etheral layer was separated and the aquous layer was extracted with ether (10 mL×3). The combined ether layers were washed with H₂O (20 mL×2) and concentrated to dryness. The residue was then transferred with EtOH (3 mL) to a flask suited for a microwave reactor, and hydroxylamine hydrochloride (0.32 g, 4.6 mmol) and pyridine (1 mL) were added. The mixture was heated and stirred in a microwave reactor at 130° C. for 30 min. The reaction mixture was then cooled to room temperature and poured into ice-H₂O (20 mL). The solid material was collected and washed with H₂O. After preparative HPLC or repeated solvating gas chromatography (SGC) (hexane to 14% Hexane/EtOAc), the product 4-(4-Methoxy-phenyl)-5-(3,4,5-trimethoxy-phenyl)-isoxazole was obtained as a light yellow solid. ¹H-NMR (CDCl₃) δ 3.70 (s, 6H), 3.82 (s, 3H), 3.85 (s, 3H), 6.85 (s, 2H), 6.94 (d, 2H, J=8), 7.33 (d, 2H, J=8), 8.30 (s, 1H) ppm; ESMS calcd for C₁₉H₁₉NO₅: 341.0; found: 342.0 (M+H⁺).

### Compound 1: 4-(4-Bromo-phenyl)-5-(3,4,5-trimethoxy-phenyl)-isoxazole

Compound 1 was synthesized in a similar way as described for Compound 3.

¹H-NMR (CDCl₃) δ 3.75 (s, 6H), 3.88 (s, 3H), 6.85 (s, 2H), 7.25 (d, 2H, J=8), 7.58 (d, 2H, J=8), 8.35 (s, 1H)ppm; ESMS calcd for C₁₈H₁₆BrNO₄: 389.0; found: 390.0 (M+H⁺).

### Compound 46: 4-(2,3-Dihydro-benzo[1,4]dioxin-6-yl)-5-(3,4,5-trimethoxy-phenyl)-isoxazol

Compound 46 was synthesized in a similar way as described for Compound 3.

¹H-NMR (CDCl₃)δ 3.75 (s, 6H), 3.85 (s, 3H), 4.28 9S, 4h), 6.80-7.30 (m, 5H), 8.28 (s, 1H)ppm; ESMS calcd for C₂₀H₁₉NO₆: 369.1; found: 370.1 (M+H⁺).

### Compound 2: 4-(Naphthalen-2-yl)-5-(2-hydroxy-4-methoxy-5-ethyl-phenyl)-isoxazole

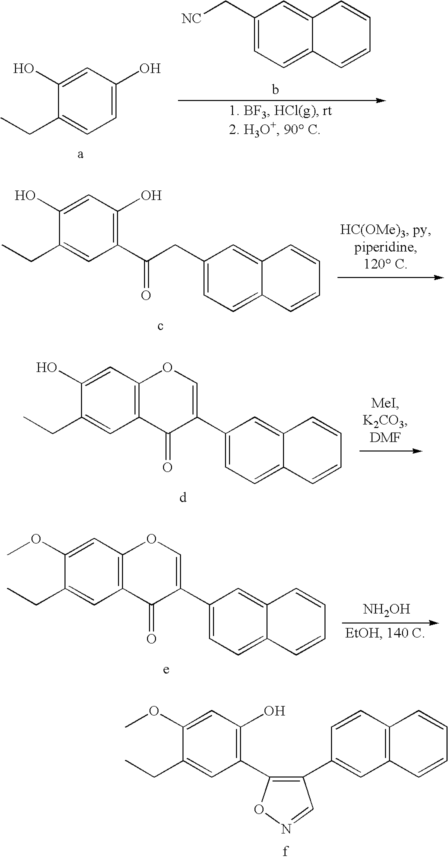

To a mixture of 4-ethyl-benzene-1,3-diol (a, 10 mmol) and naphthalen-2-yl-acetonitrile (b, 10 mmol) in boron trifluoride etherate (20 mL) was bubbled gaseous HCl for 12 h. The solids that formed were collected by filtration and heated in 6N HCl (100 mL) and EtOH (20 mL) for 6 h. The mixture was diluted with water and extracted with CH₂Cl₂. The organic extract was washed with water and dried. The oil obtained on concentration of the organic layer was crystallized in EtOAc/hexanes to give 1-(5-ethyl-2,4-dihydroxy-phenyl)-2-naphthalen-2-yl-ethanone (c, 0.74 g).

To 1-(5-ethyl-2,4-dihydroxy-phenyl)-2-naphthalen-2-yl-ethanone (c, 0.50 g) was added a mixture of pyridine (2 mL), dimethyl orthoformate (2 mL) and piperidine (2 drops), and the mixture was heated at 120° C. for 12 h. The mixture was cooled to room temperature, diluted with EtOAc (100 mL) then washed with 1 N HCl (100 mL). The organic layer was washed with water and dried. The oil obtained on concentration of the organic layer was crystallized in EtOAc/hexanes to give 6-ethyl-7-hydroxy-3-naphthalen-2-yl-chromen-4-one (d, 0.36 g).

6-Ethyl-7-hydroxy-3-naphthalen-2-yl-chromen-4-one (d, 0.36 g) was dissolved in dry dimethyl formamide (DMF) (5 mL) and treated with iodomethane (MeI) (0.5 mL) and potassium carbonate (1 g) at room temperature for 2 h. The mixture was diluted with water (50 mL) and the precipitate was collected by filtration, washed with water and dried under vacuum to give 6-ethyl-7-methoxy-3-naphthalen-2-yl-chromen-4-one (e, 0.36 g).

A mixture of 6-ethyl-7-methoxy-3-naphthalen-2-yl-chromen-4-one (e, 0.36 g), hydroxylamine hydrochloride (0.6 g), triethylamine (0.5 mL) in EtOH (10 mL) was heated at 140° C. in microwave reactor for 4 h. The mixture was diluted with water (100 mL) and extracted with CH₂Cl₂ (2×50 mL). The combined organic extracts were washed with water and dried using a drying agent. The oil obtained on concentration was flash chromatograghed on silica gel to give 4-(naphthalen-2-yl)-5-(2-hydroxy-4-methoxy-5-ethyl-phenyl)-isoxazole as white powder (f, 0.19 g). ¹H-NMR (CDCl₃) δ (ppm) 8.57 (s, 1H), 7.8 (m, 4H), 7.5 (m, 3H), 7.10 (s, 1H), 6.52 (s, 2H), 3.92 (s, 3H), 2.4 (q, 2H, J=7), 1.0 (t, 3H, J=7); ESMS clcd for C₂₂H₁₉NO₃: 345.1; Found: 346.0 (M+H)⁺.

### Compound 163: 4-(4-Chloro-phenyl)-5-(2-hydroxy-4-methoxy-5-ethyl-phenyl)-isoxazole

Compound 163 was synthesized in a similar manner as described for Compound 2.

¹H-NMR (CDCl₃) δ (ppm) 8.40 (s, 1H), 7.6 (d, 2H, J=8), 7.3 (m, 2H), 7.02 (s, 2H), 6.52 (s, 1H), 3.86 (s, 3H), 2.4 (q, 2H, J=7), 1.0 (t, 3H, J=7); ESMS clcd for C₁₈H₁₆ClNO₃: 329.1; Found: 330.0 (M+H)⁺.

### Compound 164: 4-(4-Methyl-phenyl)-5-(2-hydroxy-4-methoxy-5-ethyl-phenyl)-isoxazole

Compound 164 was synthesized in a similar manner as described for Compound 2.

¹H-NMR (CDCl₃) δ (ppm) 8.40 (s, 1H), 7.3 (d, 2H, J=8), 7.2 (d, 2H, J=8), 7.10 (s, 1H), 6.52 (s, 1H), 6.42 (s, 1H), 3.84 (s, 3H), 2.4 (q, 2H, J=7), 2.18 (s, 3H), 1.0 (t, 3H, J=7); ESMS clcd for C₁₉H₁₉NO₃: 309.1; Found: 310.0 (M+H)⁺.

### Compound 165: 4-(4-Amino-phenyl)-5-(2-hydroxy-4-methoxy-5-ethyl-phenyl)-isoxazole

Compound 165 was synthesized in a similar manner as described for Compound 2.

¹H-NMR (CDCl₃) δ (ppm) 8.28 (s, 1H), 7.2 (d, 2H, J=8), 7.0 (m, 2H), 6.88 (s, 2H), 6.32(s, 1H), 4.0 (br, 2H), 3.85 (s, 3H), 2.4 (q, 2H, J=7), 1.0 (t, 3H, J=7); ESMS clcd for C₁₈H₁₈N₂O₃: 310.1; Found: 311.0 (M+H)⁺.

### Compound 166: 4-(4-Trifluoromethyl-phenyl)-5-(2-hydroxy-4-methoxy-5-ethyl-phenyl)-isoxazole

Compound 166 was synthesized in a similar manner as described for Compound 2.

¹H-NMR (CDCl₃) δ (ppm) 8.47 (s, 1H), 7.7 (d, 2H, J=8), 7.5 (d, 2H, J=8), 6.98 (s, 1H), 6.53 (s, 1H), 6.38 (s, 1H), 3.86 (s, 3H), 2.5 (q, 2H, J=7), 1.0 (t, 3H, J=7); ESMS clcd C₁₉H₁₆F₃NO₃: 363.1; Found: 364.0 (M+H)⁺.

### Compound 167: 4-(4-Methoxy-phenyl)-5-(2-hydroxy-4-methoxy-5-ethyl-phenyl)-isoxazole

Compound 167 was synthesized in a similar manner as described for Compound 2.

¹H-NMR (CDCl₃) δ (ppm) 8.39 (s, 1H), 7.3 (d, 2H, J=8), 7.09 (s, 1H), 6.9 (d, 2H, J=8), 6.51 (s, 1H), 6.37 (s, 1H), 3.84 (s, 6H), 2.4 (q, 2H, J=7), 1.0 (t, 3H, J=7); ESMS clcd for C₁₉H₁₉NO₄: 325.1; Found: 326.0 (M+H)⁺.

### Compound 4: 4-(4-Iodo-phenyl)-5-(2-hydroxy-4-methoxy-5-ethyl-phenyl)-isoxazole

Compound 4 was synthesized in a similar manner as described for Compound 2.

¹H-NMR (CDCl₃) δ (ppm) 8.42 (s, 1H), 7.6 (d, 2H, J=8), 7.3 (m, 2H), 7.00 (s, 2H), 6.50 (s, 1H), 3.84 (s, 3H), 2.4 (q, 2H, J=7), 1.0 (t, 3H, J=7); ESMS clcd for C₁₈H₁₆INO₃: 421.0; Found: 4219 (M+H)⁺.

### Compound 162: 4-(3,4-Dimethy-phenyl)-5-(2-hydroxy-4-methoxy-5-ethyl-phenyl)-isoxazole

Compound 162 was synthesized in a similar manner as described for Compound 2.

¹H-NMR (CDCl₃) δ (ppm) 8.41 (s, 1H), 7.1 (m, 4H), 6.51 (s, 1H), 6.42 (s, 1H), 3.84 (s, 3H), 2.4 (q, 2H, J=7), 2.29 (s, 3H), 2.26 (s, 3H), 1.0 (t, 3H, J=7); ESMS clcd for C₂₀H₂₁NO₃: 323.1; Found: 324.0 (M+H)⁺.

### Compound 3b: 4-(4-Methoxy-phenyl)-3-(3,4,5-trimethoxy-phenyl)-isoxazole

### (1) Synthesis of 3-(4-Methoxy-phenyl)-1-(3,4,5-trimethoxy-phenyl)-propenone

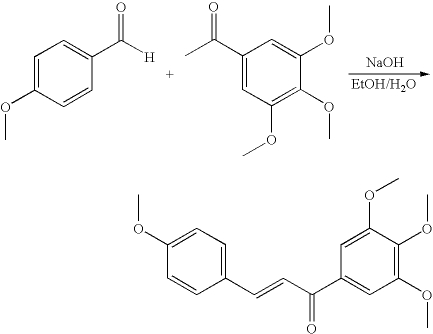

To a stirred solution of p-anislaldehyde (1.36 g, 10 mmol) and 1-(3,4,5-trimethoxy-phenyl)-ethanone (2.1 g, 10 mmol) in ethyl alcohol (EtOH) (10 mL) was added a 50% solution of NaOH in H₂O (1 mL). After the reaction had proceeded to completion, volatile components were removed under reduced pressure and the residue was taken up with ethyl acetate (EtOAc) (50 mL). The EtOAc layer was washed with H₂O (2×30 mL) and then dried with Na₂SO₄. After removal of EtOAc, the product was precipitated out from EtOH/H₂O. Solid material collected by filtration and was washed by H₂O (20 mL) and 95% ethyl alcohol (10 mL). The product,

3-(4-Methoxy-phenyl)-1-(3,4,5-trimethoxy-phenyl)-propenone (2.8 g, 85% yield), was obtained as a yellow solid. ¹H-NMR δ 3.85 (s, 3H), 3.90 (s, 3H), 3.95 (s, 6H), 6.95 (d, 2H, J=8), 7.28(s, 2H), 7.39 (d, 1H, J=15), 7.65 (d, 2H, J=8), 7.85 (d, 1H, J=15)ppm.

### (2) Synthesis of [3-(4-Methoxy-phenyl)-oxiranyl]-(3,4,5-trimethoxy-phenyl)-methanone

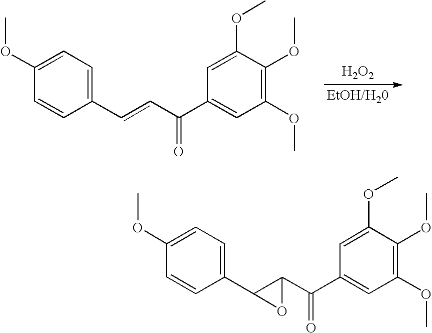

To a stirred solution of 3-(4-Methoxy-phenyl)-1-(3,4,5-trimethoxy-phenyl)-propenone (1.64 g, 5 mmol) and 1N NaOH (2.52 mL) in 95% EtOH (22 mL) was added a cold solution of 30% H₂O₂ (0.77 mL) at room temperature. After 72 h stirring, the precipitated material was collected by filtration and washed with 95% EtOH to afford [3-(4-methoxy-phenyl)-oxiranyl]-(3,4,5-trimethoxy-phenyl)-methanone as a white solid (1.38 g, yield 80%). ¹H-NMR (CDCl₃) δ 3.81 (s, 3H), 3.92 (s, 6H), 3.95 (s, 3H), 4.05 (d, 1H, J=2), 4.20 (1H, J=2), 6.95 (d, 2H, J=7), 7.25-7.35 (m, 4H)ppm.

### (3) Synthesis of 4-(4-Methoxy-phenyl)-3-(3,4,5-trimethoxy-phenyl)-isoxazole

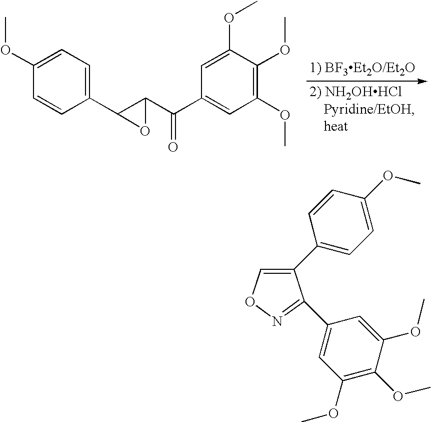

To a stirred solution of [3-(4-methoxy-phenyl)-oxiranyl]-(3,4,5-trimethoxy-phenyl)-methanone (0.5 g, 1.45 mmol) in dry ether (15 mL) was added BF₃.Et₂O (2.52 mL) slowly. After the addition, it was heated to reflux for 1 h. After the reaction mixture had cooled to room temperature, it was poured into ice-H₂O (100 mL). The etheral layer was separated and the aquous layer was extracted with ether (10 mL×3). The combined ether layers were washed with H₂O (20 mL×2) and concentrated to dryness. The residue was then transferred with EtOH (3 mL) to a flask suited for a microwave reactor, and hydroxylamine hydrochloride (0.32 g, 4.6 mmol) and pyridine (1 mL) were added. The mixture was heated and stirred in a microwave reactor at 130° C. for 30 min. The reaction mixture was then cooled to room temperature and poured into ice-H₂O (20 mL). The solid material was collected and washed with H₂O. After preparative HPLC or repeated solvating gas chromatography (SGC) (hexane to 14% Hexane/EtOAc), the product 4-(4-Methoxy-phenyl)-3-(3,4,5-trimethoxy-phenyl)-isoxazole was obtained as a light yellow solid. ¹H-NMR (CDCl₃) δ 3.70 (s, 6H), 3.82 (s, 3H), 3.85 (s, 3H), 6.85 (s, 2H), 6.94 (d, 2H, J=8), 7.33 (d, 2H, J=8), 8.30 (s, 1H) ppm; ESMS calcd for C₁₉H₁₉NO₅: 341.0; found: 342.0 (M+H⁺).

### Compound 202: 2-methoxy-5-(5-(3,4,5-trimethoxyphenyl)isoxazol-4-yl)phenylcarbamate-PEG

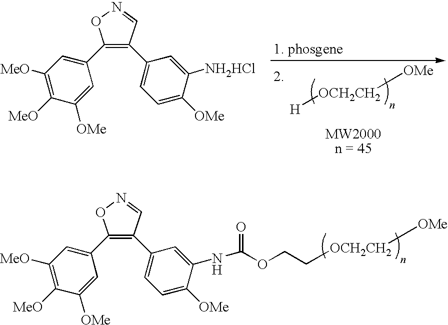

A solution of 2-methoxy-5-(5-(3,4,5-trimethoxyphenyl)isoxazol-4-yl)aniline hydrochloride (300 mg, 0.76 mmol) and triethylamine (0.22 mL, 1.60 mmol) in dichloromethane (3 mL) was added slowly to a solution of triphosgene (77 mg, 0.26 mmol) in dichloromethane (5 mL) at 0° C. under nitrogen atmosphere. The reaction mixture was stirred for 30 min at room temperature, and then cooled to 0° C. before the addition of PEG (1.53 g, 0.76 mmol) and triethylamine (0.12 mL, 0.77 mmol) in 2 ml of dichloromethane. The resulting reaction mixture was stirred for 3 h. and washed with NaHCO₃ solution. The aqueous layer was extracted with dichloromethane (2×), and the combined organic layers were washed with saturated NaCl solution, dried over Na₂SO₄ and evaporated. The crude product was purified by silica gel column chromatography (20% MeOH in EA) to give desired product 2-methoxy-5-(5-(3,4,5-trimethoxyphenyl)isoxazol-4-yl)phenylcarbamate-PEG (130 mg). ¹H NMR (CDCl₃): δ (ppm) 8.31 (s, 1H); 8.22 (s, 1H); 7.40 (s, 1H); 7.02 (d, J=9.0 Hz, 2H); 6.91 (s, 2H); 6.88 (d, J=9.0 Hz, 1H); 4.31 (m, 2H); 3.86 (m, 9H); 3.72-3.37 (m, ˜178H).

### Synthesis of Amino-Acid Derivatives

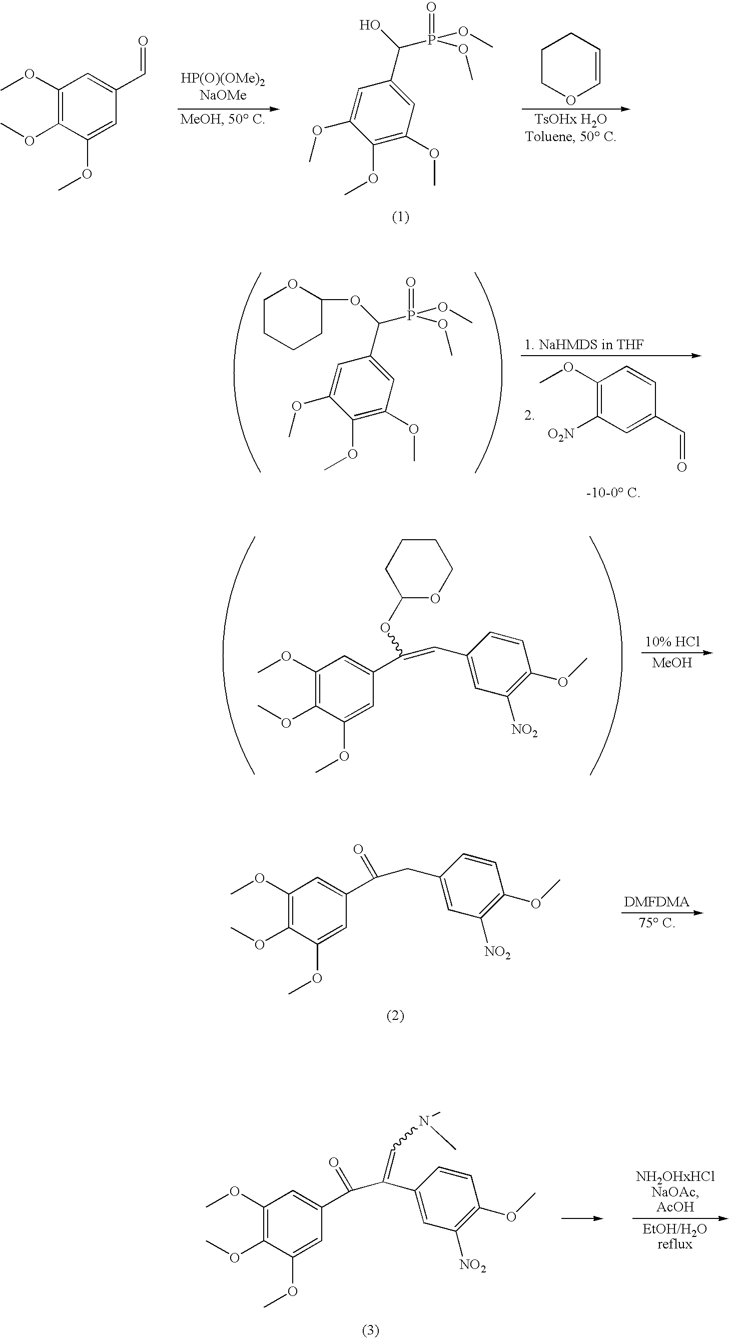

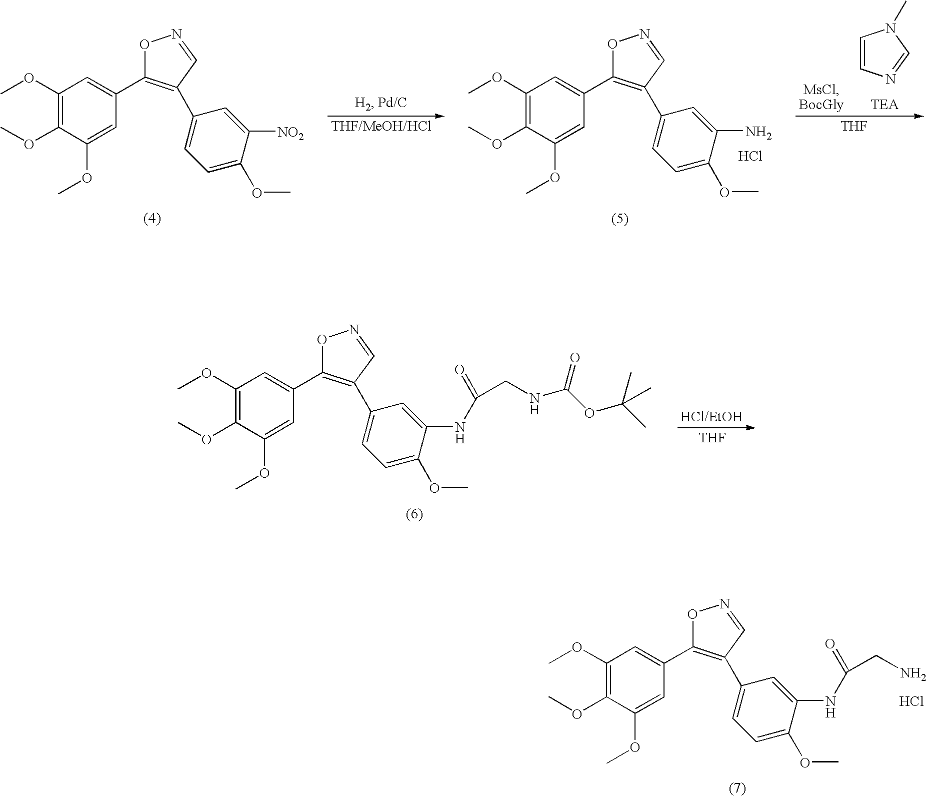

### (Hydroxy-(3,4,5-trimethoxy-phenyl)-methyl]-phosphonic acid dimethyl ester (1)

A round-bottom flask equipped with thermometer, condenser and gas inlet was charged with methanol (150 mL) and sodium methoxide (1.074 g, 20 mmol) and cooled to 0° C. under nitrogen protection. To a stirred solution were added subsequently dimethyl phosphate (19.52 g, 0.177 mol) and 3,4,5-trimethoxybenzaldehyde (30 g, 0.153 mol). A resulted solution was heated at 50° C. for 1 hour, cooled down to r.t. and treated with trifluoroacetic acid (4.6 ml). The mixture was concentrated under reduced pressure, the residue dissolved in EtOAc (300 mL), washed once with 18% brine, then once with saturated brine, dried over sodium sulfate. The filtered solution was concentrated until precipitation started. To the resulted suspension 1:1 mixture of t-butylmethyl ether and heptane was added (50 mL). Solid was filtered out, washed with two portions (20 mL) of the same solvent mixture and vacuum-dried to give 41.2 g (88%) of 1, LC purity 99.4% (AUC), m.p. 103-104° C.

### 2-(4-Methoxy-3-nitro-phenyl)-1-(3,4,5-trimethoxy-phenyl)-ethanone (2)

A round-bottom flask equipped with thermometer, condenser and gas inlet was purged and maintained under nitrogen and charged with hydroxy-(3,4,5-trimethoxy-phenyl)-methyl]phosphonic acid dimethyl ester (10 g, 32.6 mmol), 3,4-dihydro-2H-pyran (3.57 g, 42.4 mmol), toluene (100 mL) and p-toluenesolfonic acid hydrate (62.1 mg, 0.01 eq). Resulted solution was stirred at 55° C. for 1.5 hours. TLC (EtOAc) showed full conversion of starting material into a less polar compound. Reaction mixture was cooled to −10 ° C. and a solution of sodium bis(trimethylsilyl)amide in THF (1M, 33.3 mL) was added drop-wise, followed by a solution of 4-methoxy-3-nitrobenzaldehyde (5.91 g, 32.6 mmol) in THF (20 mL). Reaction mixture was stirred at 0° C. for 1 hour before allowed to warm to room temperature. TLC (Hx:EtOAc, 2:1) showed formation of Z/E isomers of 2-[2-(4-methoxy-3-nitro-phenyl)-1-(3,4,5-trimethoxy-phenyl)-vinyloxy]-tetrahydro-pyran and traces of starting materials remaining. Reaction was quenched with water (140 mL), diluted with EtOAc (60 mL) and transferred into separatory funnel. Organic layer was separated, washed with water (2×50 mL), brine, and concentrated. A residue was dissolved in methanol (100 mL) with energetic mechanical stirring, and 1M aqueous solution of HCl (10 mL) was added. Precipitation of product started soon, and a resulted suspension was stirred for 1 hour. Solid was filtered out, washed with methanol (50 mL), then with water (3×30 mL) and dried on filter followed by vacuum-drying at 60° C. until constant weight. Crude deoxybenzoin 2, 6.81 g (58%) showed LC purity of ≧97% and was used in the next step without further purification.

### 3-Dimethylamino-2-(4-methoxy-3-nitro-phenyl)-1-(3,4,5-trimethoxy-phenyl)-propenone (3)

200 mL round-bottom flask was charged with 2-(4-methoxy-3-nitro-phenyl)-1-(3,4,5-trimethoxy-phenyl)-ethanone (6.81 g, 18.8 mmol) and N,N-dimethylformamide dimethyl acetal (52 mL) and heated to 75° C. After all substrate dissolved, heating continued for 15 min, and TLC indicated completion of reaction. Excess of DMF-DMA was removed under reduced pressure to give crude enamine 3 as an oily residue still containing some of the reagent (caution! product foams and clogs adaptors). It was used in the next step without purification.

### 4-(4-Methoxy-3-nitro-phenyl)-5-(3,4,5-trimethoxy-phenyl)-isoxazole (4)

### Method A.

A flask containing crude enamine 3 (˜18.8 mmol) was charged with hydroxylamine hydrochloride 1.44 g, 20.7 mmol), sodium acetate (1.93 g, 23.6 mmol), ethanol (58 mL) and water (29 mL). A resulted clear solution was acidified with acetic acid to pH 4-5 (˜11.2 mL) and refluxed for 4 hours. Reaction was monitored by TLC (Hexane/EtOAc, 1:1) for the conversion of open-chain intermediate into less polar isoxazole derivative. Ice-water (59 mL) was added to the cooled reaction mixture, and stirring was continued for 1 hour to complete precipitation. A solid was filtered out, washed with water (2×20 mL), with ethanol (15 mL) and dried to give crude isoxazole, 5.24 g (72%). Re-crystallization from EtOAc/2-ProOH afforded 4.95 g (68%) of 4, purity >99.2% (AUC).

### Method B.

A flask containing crude enamine 3 (˜10 mmol) was charged with methanol (23 mL), hydroxylamine hydrochloride (1.39 g, 20 mmol) and triethylamine (2.02 g, 20 mmol) and refluxed for one hour. TLC showed traces of starting material remained. Reaction mixture was concentrated using vacuum pump to remove triethylamine. The residue was triturated with water, water decanted from an oil, and that operation was repeated two more times. The oil was then dissolved in ethanol with heating, ethanol was partly removed from the solution and a solid precipitated. Crude product was isolated by filtration and re-crystallized from EtOAc/2-propanol to give 4, 2.67 g (69%).

### Compound 64: 2-Methoxy-5-[5-(3,4,5-trimethoxy-phenyl)-isoxazol-4-yl]-phenylamine hydrochloride (5)

A 200-mL round-bottom flask equipped with T-bore stopcock connected to a balloon with hydrogen was charged with 4 (4.44 g, 11.5 mmol) and THF (40 mL), and a mixture was stirred to dissolve a substrate. Methanol (22 mL), solution of HCl in methanol (1M, 22 mL) and palladium, 10 wt % on activated carbon (532 mg, 12 wt %) were added to the flask, the system was evacuated and stirred under atmosphere of hydrogen for 2 hours. TLC indicated completion of reaction (Hexane/EtOAc, 2:1, developed two times). Catalyst was filtered out and washed with methanol (15 mL×3). Filtrate was diluted with 2-propanol (20 mL) and concentrated under reduced pressure to ˜20 mL volume. A resulted suspension was diluted with EtOAc (20 mL), a solid was filtered out, washed with EtOAc (20×2) and dried to give crude amine hydrochloride 5 as a yellow solid, 3.3 g (80%), LC purity 97.1%. The solid was refluxed in ethanol (50 mL) for 15 min to dissolve colored impurities. A cooled suspension was filtered out, washed with EtOAc (20×2) and vacuum-dried to obtain a creamy-colored solid, 2.9 g (70%), LC purity 99.1%.

### ({2-Methoxy-5-[5-(3,4,5-trimethoxy-phenyl)-isoxazol-4-yl]-phenylcarbamoyl}-methyl)-carbamic acid tert-butyl ester (6)

To a solution of N-t-Boc-glycine (357 mg, 2 mmol) and N-methyl-imidazole (0.162 mL, 2 mmol) in THF (16 mL) cooled with ice methanesulfonyl chloride (0.158 mL, 2 mmol) was added. Ice batch was removed, compound 5 (0.4 g, 1 mmol) was added as a solid, followed by thriethylamine (0.144 mL, 2.02 mmol), and the reaction mixture was stirred at 40-50° C. overnight. A resulted solution was decanted from a solid, a flask rinsed with EtOAc, and a combined organic solution was washed with saturated ammonium chloride solution, then twice with water, brine and dried over anhydrous sodium sulfate. The solution was filtered out through a celite pad, concentrated and the residue was dissolved in 2-propanol (3 mL) with heating, and hexane (1-2 mL) was added drop-wise to start precipitation. In 1 hour a solid was filtered out, washed with 1:1 Hexane: ether mixture (10 ml×2) and vacuum-dried to give compound 6, 0.49 g (93.7%), LC purity 99%. ¹H NMR (DMSO-d₆): δ 9.11 (s, 1H), 8.81 (s, 1H), 8.22 (s, 1H), 7.24 (m, 1H), 7.20-7.15 (m, 2H), 6.91 (s, 2H), 3.87 (s, 3H), 3.73 (m, 2H), 3.70 (s, 3H), 3.66 (s, 6H). 1.40 (s, 9H).

### Compound 169: 2-Amino-N-{2-methoxy-5-[5-(3,4,5-trimethoxy-phenyl)-isoxazol-4-yl]-phenyl}-acetamide (7)

To a solution of 6 in THF (6 mL) a 1M solution of HCl in ethanol (17 mL) was added, and a resulted solution was stirred overnight at room temperature to form a suspension with product partly precipitated out. The reaction mixture was concentrated under reduced pressure keeping temperature below 45° C. to ˜10 mL volume. A solid was filtered out, washed with ether (5 ml×2), hexane (5 mL) and vacuum-dried to give a title compound 7, 353 mg (84%), LC purity 99%.

¹H NMR (DMSO-d₆): δ 9.86 (s, 1H), 8.80 (s, 1H), 8.12 (brs, 3H), 8.07 (d, J=1.8 Hz, 1H), 7.25 (td, J=8.4 and 1.8 Hz, 1H), 7.20 (d, J=8.9 Hz, 1H), 6.90 (s, 2H), 3.89 (s, 3H), 3.83 (m, 2H), 3.70 (s, 3H), 3.67 (s, 6H).

### Compound 173: 2-amino-N-(2-methoxy-5-(5-(3,4,5-trimethoxyphenyl)isoxazol-4-yl)phenyl)butanamide hydrochloride

Compound 173 was synthesized in a similar manner as described for Compound 169. ¹H NMR (CDCl₃): δ (ppm) 8.78-8.22 (m, 5H); 7.15 (s, 1H); 6.84-6.72 (m, 3H); 3.84-3.70 (m, 12H); 3.48 (m, 2H); 1.23 (m, 3H); 0.98 (m, 2H). ESMS calcd (C₂₃H₂₈ClN₃O₆): 477.17; found: 477.2 (M+H)⁺

### Compound 174: 2-amino-N-(2-methoxy-5-(5-(3,4,5-trimethoxyphenyl)isoxazol-4-yl)phenyl)-3-phenylpropanamide hydrochloride

Compound 174 was synthesized in a similar manner as described for Compound 169. ¹H NMR (CDCl₃): δ (ppm) 8.82 (s, 1H); 8.22 (m, 2H); 7.15-6.66 (m, 8H); 3.82-3.58 (m, 14H). ESMS calcd (C₂₈H₃₀ClN₃O₆): 539.18; found: 539.2 (M+H)⁺

### Compound 172: 2-amino-N-(2-methoxy-5-(5-(3,4,5-trimethoxyphenyl)isoxazol-4-yl)phenyl)-4-(methylthio)butanamide hydrochloride

Compound 172 was synthesized in a similar manner as described for Compound 169. ¹H NMR (CDCl₃): δ (ppm) 8.79 (m, 2H); 8.36 (m, 1H); 7.08 (m, 1H); 6.84 (m, 2H); 3.86-3.72 (m, 9H); 2.75-2.15 (m, 4H); 2.07 (m, 3H), ESMS calcd (C₂₄H₃₀ClN₃O₆S): 523.15; found: 523.1 (M+H)⁺

### Compound 176: 2-amino-N-(2-methoxy-5-(5-(3,4,5-trimethoxyphenyl)isoxazol-4-yl)phenyl)-3-(4-methoxyphenyl)propanamide

Compound 176 was synthesized in a similar manner as described for Compound 169. ¹H NMR (CDCl₃): δ (ppm) 8.90-8.78 (m, 2H); 8.27 (m, 2H), 7.23-6.68 (m, 5H); 3.83-3.68 (m, 12H); 2.10 (m, 2H). ESMS calcd (C₂₉H₃₂ClN₃O₇): 569.19; found: 569.1 (M+H)⁺

### Compound 175: 2-amino-N-(2-methoxy-5-(5-(3,4,5-trimethoxyphenyl)isoxazol-4-yl)phenyl)-4-methylpentanamide hydrochloride

Compound 175 was synthesized in a similar manner as described for Compound 169. ¹H NMR (D₂O): δ (ppm) 8.15 (s, 1H); 7.37 (s, 1H); 6.78 (m. 2H); 6.31 (m, 2H); 4.02 (m, 1H); 3.62 (m, 8H); 3.31 (m, 8H); 1.56 (m, 3H); 0.71 (m, 6H). ESMS calcd (C₂₅H₃₂ClN₃O₆)_{:} 505.20; found: 505.2 (M+H)⁺

### Compound 241: methyl 2-(2-(2-methoxy-5-(5-(3,4,5-trimethoxyphenyl)isoxazol-4-yl)phenylamino)-2-oxoethylamino)acetate

Compound 274 was synthesized in a similar manner as described for Compound 169. ¹H NMR (CDCl₃): δ (ppm) 9.88 (s, 1H), 9.24 (s, 1H), 8.30 (m, 2H), 7.94 (m, 1H), 7.56 (m, 1H), 7.03-6.77 (m, 4H), 4.42-3.44 (m, 18H). ESMS calcd (C₂₄H₂₈ClN₃O₈): 521.16; found: 521.1 (M+H)⁺

### Compound 242: 4-amino-5-(2-methoxy-5-(5-(3,4,5-trimethoxyphenyl)isoxazol-4-yl)phenylamino)-5-oxopentanoic acid hydrochloride

Compound 275 was synthesized in a similar manner as described for Compound 169. ¹H NMR (CDCl₃): δ (ppm) 9.38 (s, 1H); 8.34-8.04 (m, 3H); 6.82-6.66 (m, 4H); 3.62 (m, 16H); 2.7-2.4 (m, 4H). ESMS calcd (C₂₄H₂₈ClN₃O₆): 521.16; found: 521.1 (M+H)⁺

### Compound 243: 3-amino-N-(2-methoxy-5-(5-(3,4,5-trimethoxyphenyl)isoxazol-4-yl)phenyl)propanamide hydrochloride

Compound 276 was synthesized in a similar manner as described for Compound 169. ¹H NMR (D₂O): δ (ppm) 8.23 (s, 1H); 7.47 (m, 1H); 6.82 (m, 2H); 6.45 (m, 2H); 3.68 (m, 4H); 3.56 (m, 6H); 3.39 (m, 6H); 2.65 (m, 4H). ESMS calcd (C₂₂H₂₆ClN₃O₆): 463.15; found: 463.1 (M+H)⁺

### Compound 244: 3-amino-N-(2-methoxy-5-(5-(3,4,5-trimethoxyphenyl)isoxazol-4-yl)phenyl)-4-methylpentanamide hydrochloride

Compound 277 was synthesized in a similar manner as described for Compound 169. ¹H NMR (D₂O): δ (ppm) 8.27 (s, 1H); 7.44 (m, 1H); 6.89 (m, 2H); 6.52 (m, 2H); 3.69 (m, 5H); 3.56 (m, 4H); 3.42 (m, 7H); 3.31 (m, 3H); 2.65 (m, 4H); 1.78 (m, 2H). ESMS calcd (C₂₅H₃₂ClN₃O₆): 505.20; found: 505.2 (M+H)⁺

### Compound 204: 2-amino-N-(2-methoxy-5-(5-(3,4,5-trimethoxyphenyl)isoxazol-4-yl)phenyl)propanamide hydrochloride

Compound 219 was synthesized in a similar manner as described for Compound 169. ¹H NMR (CDCl₃): δ (ppm) 8.32 (s, 1H); 8.22 (s, 1H). 7.16 (m, 2H), 6.90 (m, 3H); 3.90-3.70 (m, 15H); 1.90 (m, 4H). ESMS calcd (C₂₂H₂₆ClN₃O₆): 463.15; found: 463.1 (M+H)⁺

### Compound 249: 2-methoxy-5-(5-(3,4,5-trimethoxyphenyl)isoxazol-4-yl)aniline

¹H NMR (DMSO-d₆): δ 8.72 (s, 1H), 6.95 (s, 2H), 6.86 (d, J=7.8 Hz, 1H), 6.75 (d, J=1.8 Hz, 1H), 6.64-6.61 (m, 1H), 4.87 (s, 2H), 3.78 (s, 3H), 3.70-3.68 (m, 9H).

### Compound 3c: 1-(3,4,5-trimethoxy-phenyl)-5-(4-methoxy-phenyl)-1H-[1,2,3]triazole

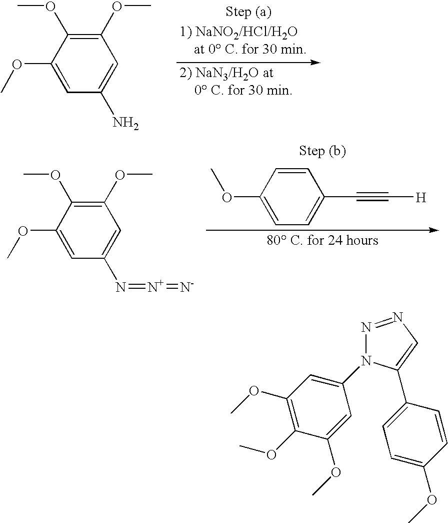

### Step (a): Synthesis of 3,4,5-Trimethoxyphenyl azide

3,4,5-Trimethoxyaniline (1.83 g; 10 mmol) is added to a 100 mL flask containing water (20 mL) and HCl (conc. aqueous solution, 5 mL). The solution is chilled to 0° C. and a solution of sodium nitrite (830 mg; 12 mmol) in water (5 mL) is added. The solution is stirred at 0° C. for 30 minutes, and then a solution of sodium azide (1.3 g; 20 mmol) in water (5 mL) is added. After another 30 minutes of stirring, dichloromethane is added (20 mL) and the organic phase was collected and filtered through a plug of silica, dried over magnesium sulfate and the solvent was evaporated to give approximately two grams of 3,4,5-trimethoxyphenyl azide.

¹H-NMR (CDCl₃) δ(ppm) 6.21 (s, 2H); 3.82 (s, 6H); 3.80 (s, 3H)

### Step (b): Synthesis of 1-(3,4,5-trimethoxy-phenyl)-5-(4-methoxy-phenyl)-1H-[1,2,3]triazole

To a scintillation vial was added 4-ethynyl anisole (660 mg.; 5 mmol) and 3,4,5-trimethoxyphenyl azide (1.05 g.; 5 mmol) and the mixture was heated at 80° C. for 24 hours. The crude mixture was purified by column chromatography to give 1-(3,4,5-trimethoxy-phenyl)-5-(4-methoxy-phenyl)-1H-[1,2,3]triazole. ¹H-NMR (CDCl₃) δ (ppm) 7.80 (s, 1H); 7.19 (d, 2H); 6.88 (d, 2H); 6.58 (s, 2H); 3.88 (s, 3H); 3.81 (s, 3H); 3.72 (s, 6H)

Expected MH+ mass ion=342, observed 342.1

### Compound 5c: 1-(3,4,5-trimethoxy-phenyl)-5-[4-(N,N-dimethylamino)-phenyl]-1H-[1,2,3]triazole

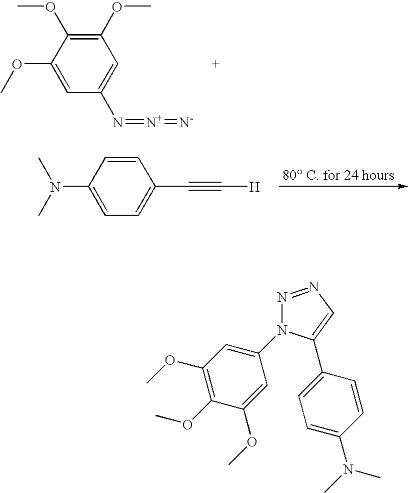

To a scintillation vial was added (4-ethynyl phenyl)-dimethyl amine (660 5 mmol) and 3,4,5-trimethoxyphenyl azide (1.05 g.; 5 mmol) and the mixture was heated at 80° C. for 24 hours. The crude mixture was purified by column chromatography to give 1-(3,4,5-trimethoxy-phenyl)-5-[4-(N,N-dimethylamino)-phenyl]-1H-[1,2,3]triazole.

¹H-NMR (CDCl₃) δ (ppm) 7.78 (s, 1H); 7.11 (d, 2H); 6.64 (d, 2H); 6.62 (s, 2H); 3.87 (s, 3H); 3.75 (s, 6H); 2.99 (s, 6H).

### Compound 216c: PEG-2-methoxy-5-(1-(3,4,5-trimethoxyphenyl)-1H-1,2,3-triazol-5-yl)phenylcarbamate

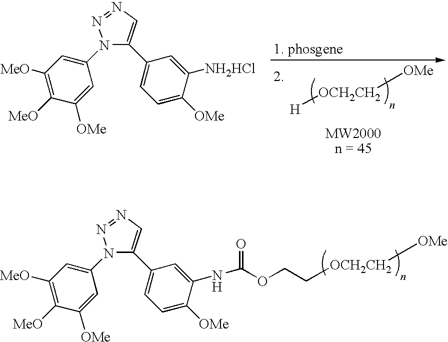

A solution of 1-(3,4,5-trimethoxy-phenyl)-5-(3-amino-4-methoxy-phenyl)-1H-[1,2,3]triazole hydrochloride (300 mg) and triethylamine (0.22 mL, 1.60 mmol) in dichloromethane (3 mL) is added slowly to a solution of triphosgene (77 mg, 0.26 mmol) in dichloromethane (5 mL) at 0° C. under nitrogen atmosphere. The reaction mixture is stirred for 30 min at room temperature, and then cooled to 0° C. before the addition of PEG (1.53 g, 0.76 mmol) and triethylamine (0.12 mL, 0.77 mmol) in 2 ml of dichloromethane. The resulting reaction mixture is stirred for 3 h. and washed with NaHCO₃ solution. The aqueous layer is extracted with dichloromethane (2×), and the combined organic layers are washed with saturated NaCl solution, dried over Na₂SO₄ and evaporated. The crude product is purified by silica gel column chromatography (20% MeOH in EA) to give desired product PEG-2-methoxy-5-(1-(3,4,5-trimethoxyphenyl)-1H-1,2,3-triazol-5-yl)phenylcarbamate.

### Synthesis of Amino-Acid Derivatives

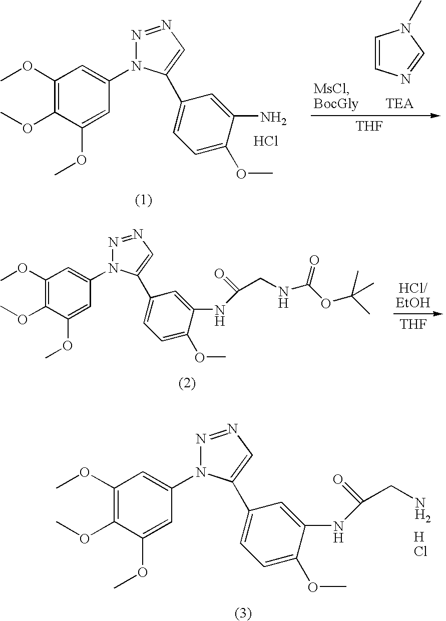

### tert-butyl 2-(2-methoxy-5-(1-(3,4,5-trimethoxyphenyl)-1H-1,2,3-triazol-5-yl)phenylamino)-2-oxoethylcarbamate (2)

To a solution of N-t-Boc-glycine (357 mg, 2 mmol) and N-methyl-imidazole (0.162 mL, 2 mmol) in THF (16 mL) cooled with ice methanesulfonyl chloride (0.158 mL, 2 mmol) is added. Ice bath is removed, compound 1 (0.4 g) is added as a solid, followed by thriethylamine (0.144 mL, 2.02 mmol), and the reaction mixture is stirred at 40-50° C. overnight. A resulted solution is decanted from a solid, a flask rinsed with EtOAc, and a combined organic solution is washed with saturated ammonium chloride solution, then twice with water, brine and dried over anhydrous sodium sulfate. The solution is filtered out through a celite pad, concentrated and the residue is dissolved in 2-propanol (3 mL) with heating, and hexane (1-2 mL) is added drop-wise to start precipitation. In 1 hour a solid is filtered out, washed with 1:1 Hexane: ether mixture (10 ml×2) and vacuum-dried to give compound 2.

### 2-amino-N-(2-methoxy-5-(1-(3,4,5-trimethoxyphenyl)-1H-1,2,3-triazol-5-yl)phenyl)acetamide hydrochloride (3)

To a solution of 2 in THF (6 mL) a 1M solution of HCl in ethanol (17 mL) is added, and a resulted solution is stirred overnight at room temperature to form a suspension with product partly precipitated out. The reaction mixture is concentrated under reduced pressure keeping temperature below 45° C. to ˜10 mL volume. A solid is filtered out, washed with ether (5 ml×2), hexane (5 mL) and vacuum-dried to give compound 3.

### Compound 3e: 4-(4-methoxy-phenyl)-5-(3,4,5-trimethoxy-phenyl)-isothiazole

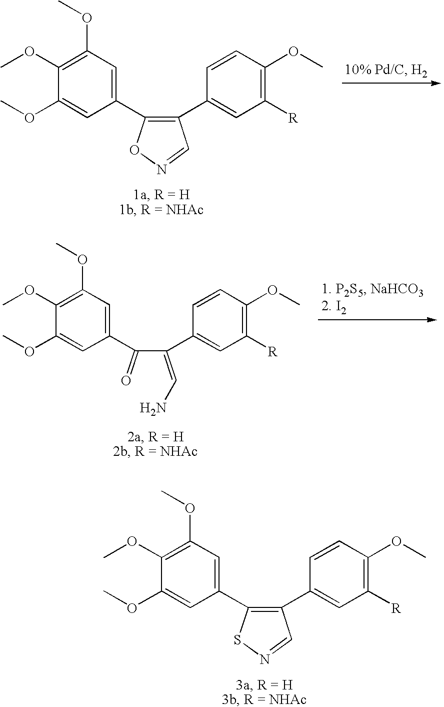

100 mg of 4-(4-methoxy-phenyl)-5-(3,4,5-trimethoxy-phenyl)-isoxazole (1a) in EyOH (10 mL) was hydrogenated under the catalysis of 10% Pd on wet carbon at rt overnight. Removal of catalyst and solvent gave 3-amino-2-(4-methoxy-phenyl)-1-(3,4,5-trimethoxy-phenyl)-propenone (2a , 75 mg) as colorless oil.

74 mg of 2a was dissolved in THF (10 mL) and sodium bicarbonate (0.2 g) and P₂S₅ (0.15 g) was added followed by iodine (0.15 g). The mixture was stirred at rt for 24 h. Removal of solvent and purified the mixture with repeated column chromatography gave 4-(4-methoxy-phenyl)-5-(3,4,5-trimethoxy-phenyl)-isothiazole (3a, 4 mg) as white solid.

### Compound 3e: 4-(4-Methoxy-phenyl)-5-(3,4,5-trimethoxy-phenyl)-isothiazole

¹H-NMR (CDCl₃) δ (ppm) 8.47 (s, 1H), 7.3 (d, 2H, J=8), 6.9 (d, 2H, J=8), 6.52 (s, 2H), 3.87 (s, 3H), 3.82(s, 3H), 3.70 (s, 6H); ESMS clcd for C₁₉H₁₉NO₄S: 357.1; Found: 358.0 (M+H)⁺.

### Compound 64e: 2-Methoxy-5-[5-(3,4,5-trimethoxy-phenyl)-isothiazol-4-yl]-phenylamine

¹H-NMR (CDCl₃) δ(ppm) 8.44 (s, 1H), 6.7 (m, 3H), 6.57 (s, 2H), 3.87 (s, 3H), 3.86(s, 3H), 3.8 (br, 2H), 3.72 (s, 6H); ESMS clcd for C₁₉H₂₀N₂O₄S: 372.1; Found: 373.1 (M+H)⁺.

### Synthesis of 4-(4-Methoxy-phenyl)-5-(3,4,5-trimethoxy-phenyl)-isoxazole

### (1) Synthesis of 3-(4-Methoxy-phenyl)-1-(3,4,5-trimethoxy-phenyl)-propenone

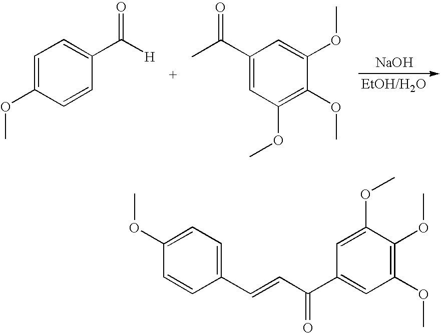

To a stirred solution of p-anislaldehyde (1.36 g, 10 mmol) and 1-(3,4,5-trimethoxy-phenyl)-ethanone (2.1 g, 10 mmol) in ethyl alcohol (EtOH) (10 mL) was added a 50% solution of NaOH in H₂O (1 mL). After the reaction had proceeded to completion, volatile components were removed under reduced pressure and the residue was taken up with ethyl acetate (EtOAc) (50 mL). The EtOAc layer was washed with H₂O (2×30 mL) and then dried with Na₂SO₄. After removal of EtOAc, the product was precipitated out from EtOH/H₂O. Solid material collected by filtration and was washed by H₂O (20 mL) and 95% ethyl alcohol (10 mL). The product, 3-(4-Methoxy-phenyl)-1-(3,4,5-trimethoxy-phenyl)-propenone (2.8 g, 85% yield), was obtained as a yellow solid. ¹H-NMR δ 3.85 (s, 3H), 3.90 (s, 3H), 3.95 (s, 6H), 6.95 (d, 2H, J=8), 7.28(s, 2H), 7.39 (d, 1H, J=15), 7.65 (d, 2H, J=8), 7.85 (d, 1H, J=15)ppm.

### (2) Synthesis of [3-(4-Methoxy-phenyl)-oxiranyl]-(3,4,5-trimethoxy-phenyl)-methanone

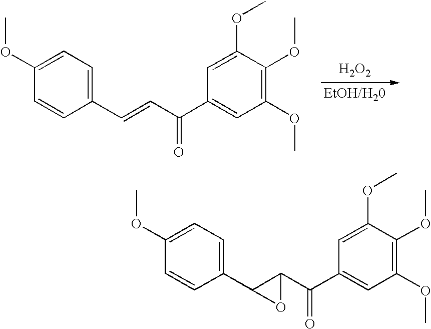

To a stirred solution of 3-(4-Methoxy-phenyl)-1-(3,4,5-trimethoxy-phenyl)-propenone (1.64 g, 5 mmol) and 1N NaOH (2.52 mL) in 95% EtOH (22 mL) was added a cold solution of 30% H₂O₂ (0.77 mL) at room temperature. After 72 h stirring, the precipitated material was collected by filtration and washed with 95% EtOH to afford [3-(4-methoxy-phenyl)-oxiranyl]-(3,4,5-trimethoxy-phenyl)-methanone as a white solid (1.38 g, yield 80%). ¹H-NMR (CDCl₃) δ 3.81 (s, 3H), 3.92 (s, 6H), 3.95 (s, 3H), 4.05 (d, 1H, J=2), 4.20 (1H, J=2), 6.95 (d, 2H, J=7), 7.25-7.35 (m, 4H)ppm.

### (3) Synthesis of 4-(4-Methoxy-phenyl)-5-(3,4,5-trimethoxy-phenyl)-isoxazole

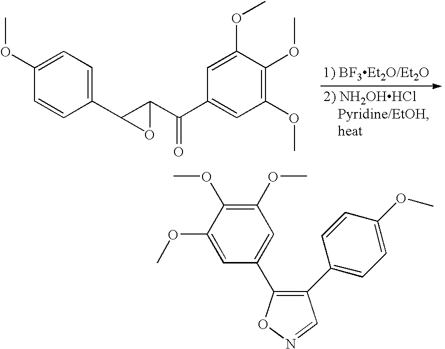

To a stirred solution of [3-(4-methoxy-phenyl)-oxiranyl]-(3,4,5-trimethoxy-phenyl)-methanone (0.5 g, 1.45 mmol) in dry ether (15 mL) was added BF₃.Et₂O (2.52 mL) slowly. After the addition, it was heated to reflux for 1 h. After the reaction mixture had cooled to room temperature, it was poured into ice-H₂O (100 mL). The etheral layer was separated and the aquous layer was extracted with ether (10 mL×3). The combined ether layers were washed with H₂O (20 mL×2) and concentrated to dryness. The residue was then transferred with EtOH (3 mL) to a flask suited for a microwave reactor, and hydroxylamine hydrochloride (0.32 g, 4.6 mmol) and pyridine (1 mL) were added. The mixture was heated and stirred in a microwave reactor at 130° C. for 30 min The reaction mixture was then cooled to room temperature and poured into ice-H₂O (20 mL). The solid material was collected and washed with H₂O. After preparative HPLC or repeated solvating gas chromatography (SGC) (hexane to 14% Hexane/EtOAc), the product 4-(4-Methoxy-phenyl)-5-(3,4,5-trimethoxy-phenyl)-isoxazole was obtained as a light yellow solid. ¹H-NMR (CDCl₃) δ 3.70 (s, 6H), 3.82 (s, 3H), 3.85 (s, 3H), 6.85 (s, 2H), 6.94 (d, 2H, J=8), 7.33 (d, 2H, J=8), 8.30 (s, 1H) ppm; ESMS calcd for C₁₉H₁₉NO₅: 341.0; found: 342.0 (M+H⁺).

### Compound 216e: 2-methoxy-5-(5-(3,4,5-trimethoxyphenyl)isothiazol-4-yl)phenylcarbamate-PEG

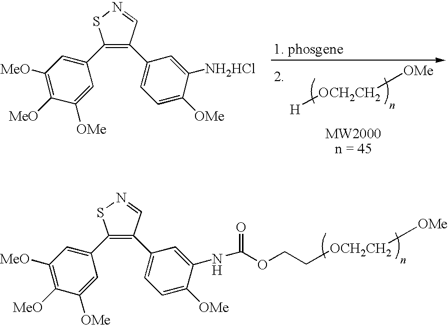

A solution of 2-methoxy-5-(5-(3,4,5-trimethoxyphenyl)isothiazol-4-yl)aniline hydrochloride (300 mg, 0.76 mmol) and triethylamine (0.22 mL, 1.60 mmol) in dichloromethane (3 mL) is added slowly to a solution of triphosgene (77 mg, 0.26 mmol) in dichloromethane (5 mL) at 0° C. under nitrogen atmosphere. The reaction mixture is stirred for 30 min at room temperature, and then cooled to 0° C. before the addition of PEG (1.53 g, 0.76 mmol) and triethylamine (0.12 mL, 0.77 mmol) in 2 ml of dichloromethane. The resulting reaction mixture is stirred for 3 h. and washed with NaHCO₃ solution. The aqueous layer is extracted with dichloromethane (2×), and the combined organic layers are washed with saturated NaCl solution, dried over Na₂SO₄ and evaporated. The crude product is purified by silica gel column chromatography (20% MeOH in EA) to give desired product 2-methoxy-5-(5-(3,4,5-trimethoxyphenyl)isothiazol-4-yl)phenylcarbamate-PEG.

### Synthesis of Amino-Acid Derivatives

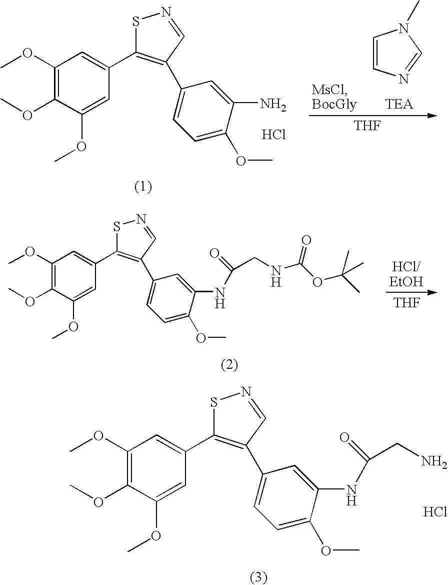

### ({2-Methoxy-5-[5-(3,4,5-trimethoxy-phenyl)-isoxazol-4-yl]-phenylcarbamoyl}-methyl)-carbamic acid tert-butyl ester (2)

To a solution of N-t-Boc-glycine (357 mg, 2 mmol) and N-methyl-imidazole (0.162 mL, 2 mmol) in THF (16 mL) cooled with ice methanesulfonyl chloride (0.158 mL, 2 mmol) is added. Ice bath is removed, 1 (0.4 g, 1 mmol) is added as a solid, followed by thriethylamine (0.144 mL, 2.02 mmol), and the reaction mixture is stirred at 40-50° C. overnight. A resulted solution is decanted from a solid, a flask rinsed with EtOAc, and a combined organic solution is washed with saturated ammonium chloride solution, then twice with water, brine and dried over anhydrous sodium sulfate. The solution is filtered out through a celite pad, concentrated and the residue is dissolved in 2-propanol (3 mL) with heating, and hexane (1-2 mL) is added drop-wise to start precipitation. In 1 hour a solid is filtered out, washed with 1:1 Hexane: ether mixture (10 ml×2) and vacuum-dried to give 2.

### 2-Amino-N-{2-methoxy-5-[5-(3,4,5-trimethoxy-phenyl)-isoxazol-4-yl]-phenyl}-acetamide (3)

To a solution of 2 in THF (6 mL) a 1M solution of HCl in ethanol (17 mL) is added, and a resulted solution is stirred overnight at room temperature to form a suspension with product partly precipitated out. The reaction mixture is concentrated under reduced pressure keeping temperature below 45° C. to ˜10 mL volume. A solid is filtered out, washed with ether (5 ml×2), hexane (5 mL) and vacuum-dried to give 3.

### Example 2

### Effect on HUVEC Cell Morphology and Migration

Compound 249 and Combretastatin A4 (CA4) (Pharmaron, LLC, Louisville, Ky.) were compared for their in vitro effect on HUVEC cell morphology and migration on a plastic surface. Compound 249 induced HUVEC cell shrinkage and migration inhibition at 1 nM. Both events happened at an early time of treatment. Compound 249 possessed a stronger effect on both shrinkage and migration than CA4.

A. Compound 249 Induces HUVEC Cell Shrinkage at 1 nM within 1 hr of Treatment

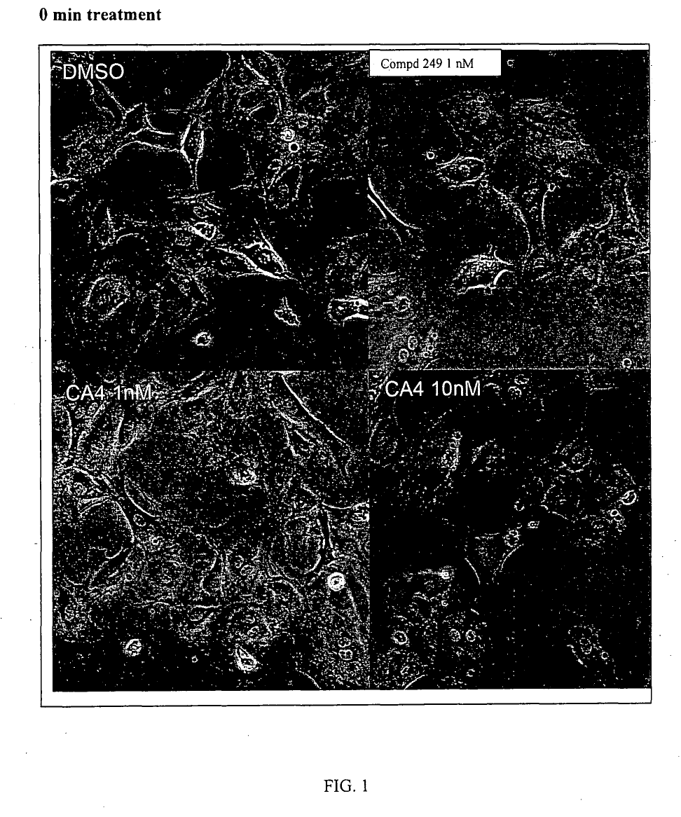

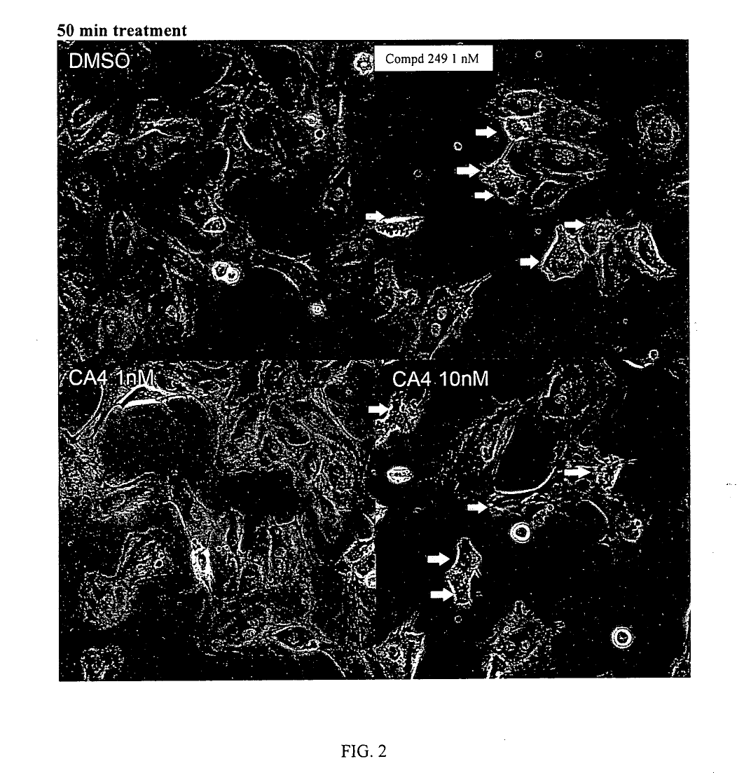

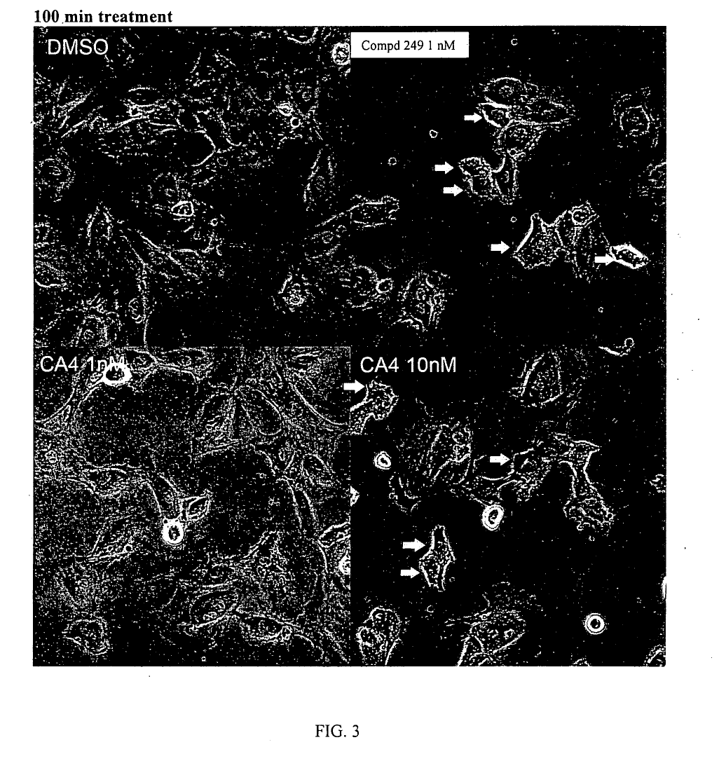

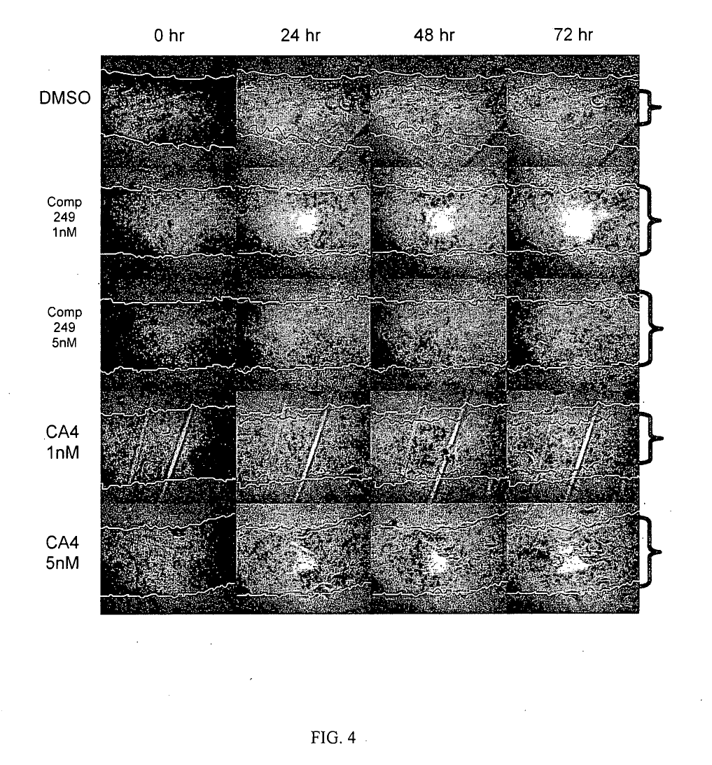

HUVEC cells (ATCC, VA) (passage number 4) were cultured in EGM2 medium (Cambrex, Mass.) on 24-well plates and time lapse imaging was performed in a cell culture system mounted on an inverted microscope supplied with 5% CO₂. The temperature was kept at 35° C. Images were taken every 60 sec using 20× objective (FIGS. 1-3) or every 30 min using 2× objective (FIG. 4) for up to 90 hr. FIGS. 1-3 show results from three time points of treatment: 0, 50, 100 min. Compared to DMSO treatment, 1 nM of Compound 249 caused HUVEC cell shrinkage (arrows point to the cells affected strongly) within 50 min of treatment. Most shrunken cells were at cell cycle interphase but not mitotic stage, and still moving on the culture surface, as observed in time-lapse movies (data not shown). The shrinking effect caused by Compound 249 (and CA4) is not identical to the apoptotic effect in mitotic cells after microtubule depolymerization. FIGS. 1-3 also show that CA4 caused a similar change on HUVEC cells at 10 nM, but did not cause obvious morphological changes at 1 nM. Compound 249 had a more potent effect on HUVEC cell morphology than CA4.

### B. Compound 249 Inhibits HUVEC Migration at 1 nM

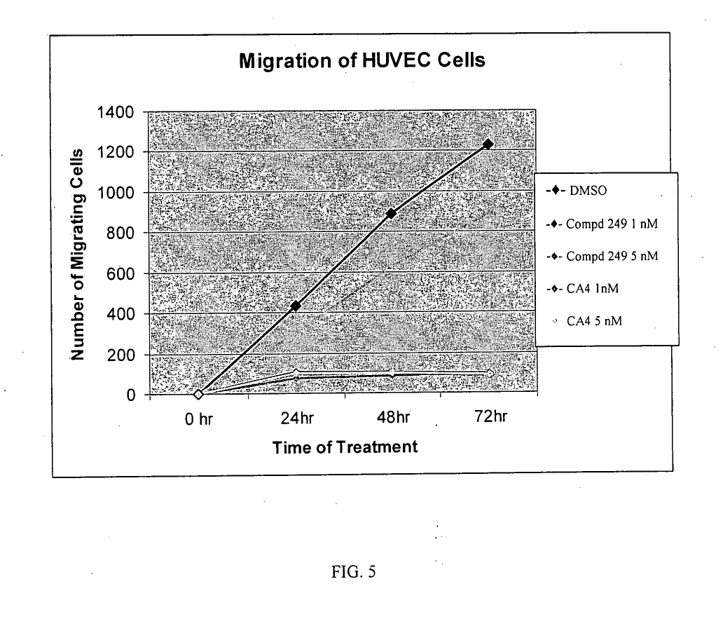

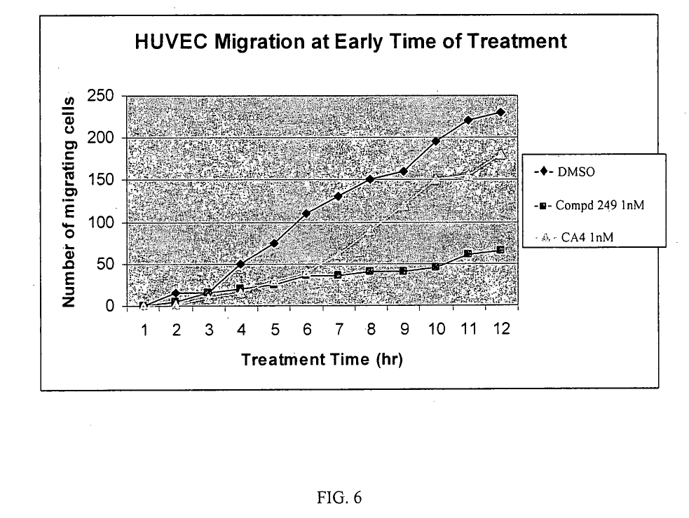

HUVEC migration inhibition can serve as an in vitro surrogate for the assessment of the inhibition of angiogenesis. Above, Compound 249 significantly inhibited HUVEC migration. Using the above described time-lapse culture system, a wound healing assay was used to analyze the effect of Compound 249 and CA4 on HUVEC cells migration. Confluent HUVEC cultures were scraped similarly to make a blank area. Migration of remaining cells under different treatment was imaged at the same time to generate time-lapse image sequences. Time-lapse movies were further analyzed to calculate the number of cells that migrated to the blank area. In FIG. 4, the gray lines show the front line of cells after scraping and the red lines show the front lines of cells after migration at 24, 48 and 72 hr after treatment. FIG. 4 shows that compared to DMSO, three treatments (1 nM Compound 249, 5 nM Compound 249 and 5 nM CA4) significantly blocked migration of HUVEC cells to the blank area, while 1 nM CA4 did not block the migration event. The quantitative analysis of FIG. 4 is shown in FIG. 5 which demonstrates that the absolute migrated cell numbers are similar during Compound 249 1 nM, 5 nM and CA4 5 nM. However, CA4 at 1 nM had much less of an effect on HUVEC migration. Additional detailed analysis is shown in FIG. 6, which shows that the difference in migration inhibition between Compound 249 and CA4 at 1 nM was noticeable as early as 4 hr after treatment. Compound 249 possessed a potent inhibitory effect on HUVEC migration in vitro. Compound 249 had a stronger effect than CA4 for the inhibition of HUVEC migration.

### Example 3

### Disengagement of VE-Cadherin Junctions in HUVEC Cells

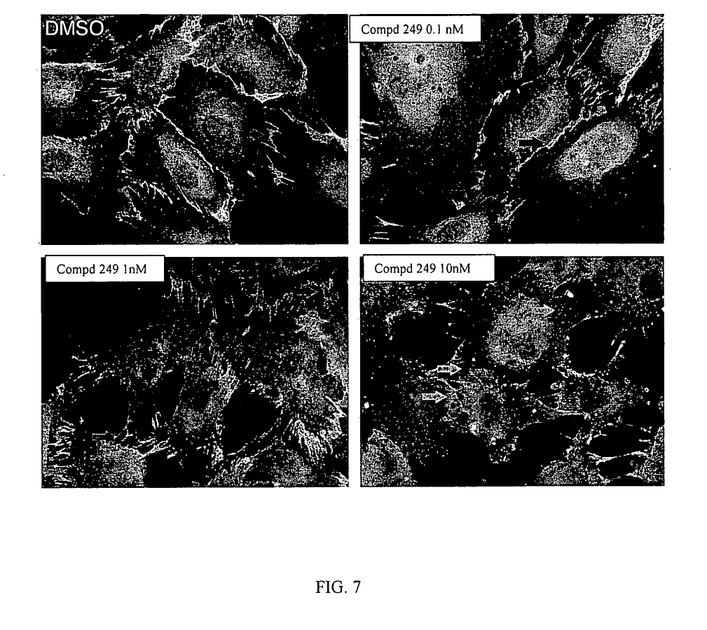

Example 2 demonstrated that 1 nM Compound 249 inhibited HUVEC cell migration, and induced shrinkage of HUVEC cells in vitro. It was thought that these phenotypic changes might be associated with alterations of endothelial cell junctions. To determine if Compound 249 induced disengagement of HUVEC cell junctions, VE-cadherin junctions of HUVEC cells were examined. HUVEC cells were treated with DMSO or Compound 249 (0.1, 1, or 10 nM) for 24 hrs and fixed for immunostaining. DMSO concentration was 1:100 for all treatment. To boost the immunofluorescence signal, cells were stained with a mixture of two polyclonal anti-human VE-cadherin Abs (1:1 mixed, obtained from Amersham Biosciences, N.J., and Santa Cruz Biotechnology, Calif.) followed by staining with a mixture of fluorescent secondary antibodies. FIG. 7 shows that VE-cadherin stained strongly at the cell-cell junctions (red arrows in DMSO), but not the non-contacted regions between cells in the DMSO control. Non-DMSO treated cells gave a similar result to those with DMSO treatment (data not shown). With 1 nM Compound 249 treatment, VE-cadherin staining was reduced (red arrow in Compound 249 1 nM) compared to that in DMSO or Compound 249 0.1 nM treatment. Most importantly, the cells appeared to be separating from each other and some of the VE-cadherin junctions were disengaged (red arrows in Compound 249 1 nM). Increasing Compound 249 concentration to 10 nM significantly abolished VE-cadherin staining (aqua arrows showing lack of VE-cadherin staining), and most of the cell junctions were disengaged. These results strongly suggest that Compound 249 affects the assembly of cell-cell junctions of activated human endothelial cells, most likely through disengagement of VE-cadherin junctions and reduction of VE-cadherin molecules at the junctions. In addition, the potent effect of Compound 249 on microtubule cytoskeletons can also contribute strongly to the cell-cell junction disruption.

All publications, patent applications, patents, and other documents cited herein are incorporated by reference in their entirety. In case of conflict, the present specification, including definitions, will control. In addition, the materials, methods, and examples are illustrative only and not intended to be limiting in any way.

1. A method of treating or inhibiting angiogenesis in a subject in need thereof, comprising administering to the subject an effective amount of a compound represented by formula (I): 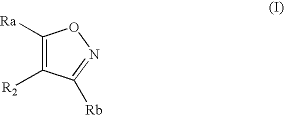
or a pharmaceutically acceptable salt thereof,
wherein:
one of Rₐ or R_{b} is —H and the other is an optionally substituted aryl or an optionally substituted heteroaryl; and
R₂ is an optionally substituted phenyl, an optionally substituted 2,3-dihydro-benzo[1,4]dioxinyl, an optionally substituted benzo[1,3]dioxolyl, an optionally substituted biphenyl, an optionally substituted 4-pyridinyl-phenyl, an optionally substituted quinolinyl, an optionally substituted isoquinolinyl, an optionally substituted 1H-indolyl, an optionally substituted pyridinyl, an optionally substituted oxazolyl, an optionally substituted isoxazolyl, an optionally substituted thiazolyl, an optionally substituted isothiazolyl, an optionally substituted imidazolyl, an optionally substituted pyrrolyl, an optionally substituted pyrazolyl, an optionally substituted furanyl, an optionally substituted thiophenyl, an optionally substituted thiadiazolyl, an optionally substituted oxadiazolyl, an optionally substituted chromanyl, an optionally substituted isochromanyl, an optionally substituted pyridazinyl, an optionally substituted pyrimidinyl, an optionally substituted pyrazinyl, an optionally substituted benzothiophenyl, an optionally substituted 2,3-dihydro-benzothiophenyl, an optionally substituted benzofuranyl, an optionally substituted 2,3-dihydro-benzofuranyl, an optionally substituted 1H-benzoimidazolyl, an optionally substituted benzothiazolyl, an optionally substituted benzooxazolyl, an optionally substituted 1H-benzotriazolyl, an optionally substituted 1H-indazolyl, an optionally substituted 9H-purinyl, an optionally substituted pyrrolopyrimidinyl, an optionally substituted pyrrolopyrazinyl, an optionally substituted pyrrolopyridazinyl, an optionally substituted imidazopyrazinyl, or an optionally substituted imidazolpyridazinyl.

2. A method of treating or inhibiting angiogenesis in a subject in need thereof, comprising administering to the subject an effective amount of a compound represented by formula (II): 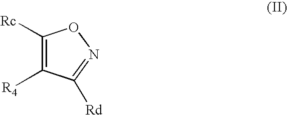
or a pharmaceutically acceptable salt thereof,
wherein:
one of R_{c} or R_{d} is —H and the other is an optionally substituted heteroaryl, an unsubstituted phenyl, or a substituted phenyl represented by one of the following formulas: 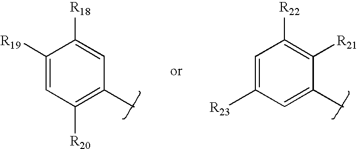
R₄ is an optionally substituted aryl or an optionally substituted heteroaryl;
R₁₈, R₁₉, R₂₂, and R₂₃, are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;
R₂₀ is an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, ‘OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;
R₂₁ is halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₁₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₇, for each occurrence, is independently, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; and
p is 1 or 2.

3. The method of claim 1, wherein the compound is represented by formula (IA): 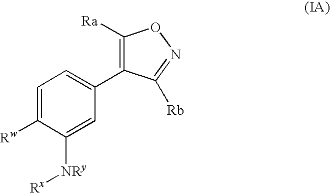
wherein:
R^{x} is (R^{aa})ₘ, —R^{aa}—C(O)(CH₂)ₙC(O)OH, —C(O)(CH₂)ₙC(O)OH, —C(O)YR^{z}, —C(O)NH—R^{aa}, or —(R^{aa})_{q}C(O)(Y₁);
R^{y} is —H or lower alkyl;
R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl;
R₇ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R^{aa} is an amino acid residue or an amino acid residue analog;
Y is CH₂, O, or NH;
R^{z} is Alk-NH₂, Alk-C(O)OH, Het, or Y₁;
Alk is an optionally substituted alkylene;
Het is an optionally substituted heteroalkyl;
Y₁ is a water soluble polymer with a molecular weight less than 60,000 daltons;
n is 1, 2, 3, or 4;
m is an integer from 1 to 10; and
q is 0 or 1.

4. The method of claim 3, wherein the compound is represented by formula (VA): 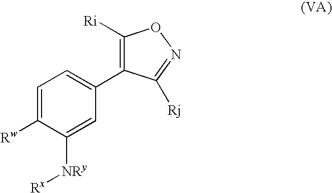
wherein:
one of Rᵢ or Rⱼ is —H and the other is represented by the following formula: 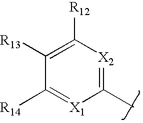
X₁ and X₂ are each, independently, CH or N;
R₁₂, R₁₃ and R₁₄ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;
R₈, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl; and
p is 1 or 2.

5. The method of claim 2, wherein the compound is represented by formula (IIA): 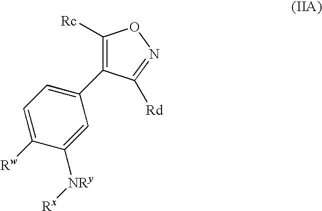
wherein:
R^{x} is (R^{aa})ₘ, —R^{aa}—C(O)(CH₂)ₙC(O)OH, —C(O)(CH₂)ₙC(O)OH, —C(O)YR^{z}, —C(O)NH—R^{aa}, or —(R^{aa})_{q}C(O)(Y₁);
R^{y} is —H or lower alkyl;
R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl;
R^{aa} is an amino acid residue or an amino acid residue analog;
Y is CH₂, O, or NH;
R^{z} is Alk-NH₂, Alk-C(O)OH, Het, or Y₁;
Alk is an optionally substituted-alkylene;
Het is an optionally substituted heteroalkyl;
Y₁ is a water soluble polymer with a molecular weight less than 60,000 daltons;
n is 1, 2, 3, or 4;
m is an integer from 1 to 10; and
q is 0 or 1.

6. The method of claim 1, wherein the compound is represented by formula (IB): 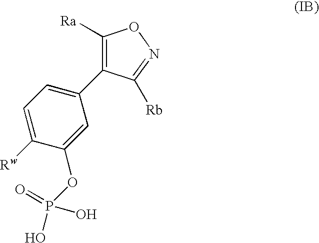
wherein:
R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl; and
R₇ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl.

7. The method of claim 6, wherein the compound is represented by formula (VB): 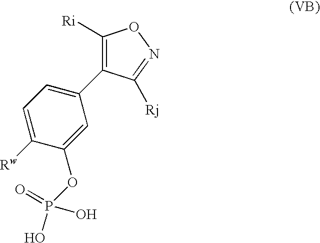
wherein:
one of Rᵢ or Rⱼ is —H and the other is represented by the following formula: 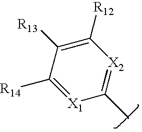
X₁ and X₂ are each, independently, CH or N;
R₁₂, R₁₃ and R₁₄ are each, independently, halo, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, cyano, nitro, guanadino, a haloalkyl, a haloalkoxy, a heteroalkyl, —OR₇, —NR₁₀R₁₁, —C(O)R₇, —C(O)OR₇, —OC(O)R₇, —C(O)NR₁₀R₁₁, —NR₈C(O)R₇, —OP(O)(OR₇)₂, —SP(O)(OR₇)₂, —SR₇, —S(O)ₚR₇, —OS(O)ₚR₇, —S(O)ₚOR₇, —NR₈S(O)ₚR₇, or —S(O)ₚNR₁₀R₁₁;
R₈, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl; and
p is 1 or 2.

8. A method of treating or inhibiting angiogenesis in a subject in need thereof, comprising administering to the subject an effective amount of a compound represented by formula (XI): 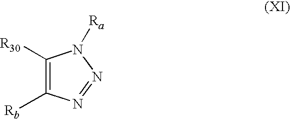
or a pharmaceutically acceptable salt, thereof, wherein:
one of Rₐ or R_{b} is —H and the other is an optionally substituted aryl or an optionally substituted heteroaryl, provided that Rₐ is not acridinyl; and
R₃₀ is an optionally substituted aryl or an optionally substituted heteroaryl.

9. The method of claim 8, wherein the compound is represented by formula (XIA): 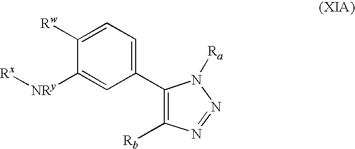
wherein:
R^{x} is (R^{aa})ₘ, —R^{aa}—C(O)(CH₂)ₙC(O)OH, —C(O)(CH₂)ₙC(O)OH, —C(O)YR^{z}, —C(O)NH—R^{aa}, or —(R^{aa})_{q}C(O)(Y₁);
R^{y} is —H or lower alkyl;
R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl;
R₇, for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R^{aa} is an amino acid residue or an amino acid residue analog;
Y is CH₂, O, or NH;
R^{z} is Alk-NH₂, Alk-C(O)OH, Het, or Y₁;
Alk is an optionally substituted alkylene;
Het is an optionally substituted heteroalkyl;
Y₁ is a water soluble polymer with a molecular weight less than 60,000 daltons;
n is 1, 2, 3, or 4;
m is an integer from 1 to 10; and
q is 0 or 1.

10. The method of claim 8, wherein the compound is represented by formula (XIB): 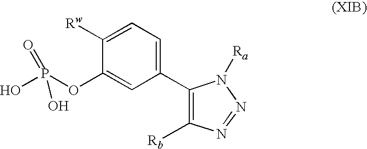
wherein:
R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl;
R₇, for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl.

11. A method of treating or inhibiting angiogenesis in a subject in need thereof, comprising administering to the subject an effective amount of a compound represented by formula (XXXI): 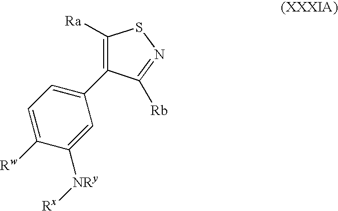
or a pharmaceutically acceptable salt, thereof, wherein:
one of Rₐ or R_{b} is —H and the other is an optionally substituted aryl or an optionally substituted heteroaryl; and
R₅₉ is an optionally substituted aryl or an optionally substituted heteroaryl, provided that R₅₉ is not an unsubstituted phenyl.

12. The method of claim 11, wherein the compound is represented by formula (XXXIA): 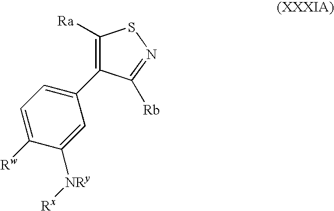
wherein:
R^{X} is (R^{aa})ₘ, —R^{aa}—C(O)(CH₂)ₙC(O)OH, —C(O)(CH₂)ₙC(O)OH, —C(O)YR^{z}, —C(O)NH—R^{aa}, or —(R^{aa})_{q}C(O)(Y₁);
R^{y} is —H or lower alkyl;
R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl;
R₇, for each occurrence, is independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R^{aa} is an amino acid residue or an amino acid residue analog;
Y is CH₂, O, or NH;
R^{z} is Alk-NH₂, Alk-C(O)OH, Het, or Y₁;
Alk is an optionally substituted alkylene;
Het is an optionally substituted heteroalkyl;
Y₁ is a water soluble polymer with a molecular weight less than 60,000 daltons;
n is 1, 2, 3, or 4;
m is an integer from 1 to 10; and
q is 0 or 1.

13. The method of claim 11, wherein the compound is represented by formula (XXXIB): 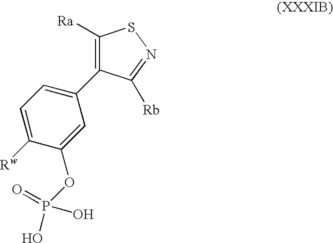
wherein:
R^{w} is —H, an alkyl, an alkenyl, an alkynyl, cyano, a haloalkyl, an alkoxy, a haloalkoxy, a halo, an amino, an alkylamino, a dialkylamino, —OP(O)(OR₇)₂, —SP(O)(OR₇)₂, nitro, an alkyl ester, or hydroxyl; and
R₇, for each occurrence, is independently -H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl.
